SEQUENCE LISTING (809 sequences)

SEQ ID NO: 1 - protein, 8 aa, synthetic construct
  REGION 1..8: note = Synthetic
GFNIKDDY

SEQ ID NO: 2 - protein, 8 aa, synthetic construct
  REGION 1..8: note = Synthetic
IDPENGDT

SEQ ID NO: 3 - protein, 10 aa, synthetic construct
  REGION 1..10: note = Synthetic
TLWLRRGLDY

SEQ ID NO: 4 - protein, 11 aa, synthetic construct
  REGION 1..11: note = Synthetic
KSLLHSNGYTY

SEQ ID NO: 5 - skipped

SEQ ID NO: 6 - protein, 9 aa, synthetic construct
  REGION 1..9: note = Synthetic
MQHLEYPFT

SEQ ID NO: 7 - protein, 5 aa, synthetic construct
  REGION 1..5: note = Synthetic
DDYMY

SEQ ID NO: 8 - protein, 17 aa, synthetic construct
  REGION 1..17: note = Synthetic
WIDPENGDTEYASKFQD

SEQ ID NO: 9 - protein, 8 aa, synthetic construct
  REGION 1..8: note = Synthetic
WLRRGLDY

SEQ ID NO: 10 - protein, 16 aa, synthetic construct
  REGION 1..16: note = Synthetic
RSSKSLLHSNGYTYLF

SEQ ID NO: 11 - protein, 7 aa, synthetic construct
  REGION 1..7: note = Synthetic
RMSNLAS

SEQ ID NO: 12 - protein, 7 aa, synthetic construct
  REGION 1..7: note = Synthetic
GFNIKDD

SEQ ID NO: 13 - skipped

SEQ ID NO: 14 - protein, 6 aa, synthetic construct
  REGION 1..6: note = Synthetic
LRRGLD

SEQ ID NO: 15 - protein, 12 aa, synthetic construct
  REGION 1..12: note = Synthetic
SKSLLHSNGYTY

SEQ ID NO: 16 - protein, 6 aa, synthetic construct
  REGION 1..6: note = Synthetic
HLEYPF

SEQ ID NO: 17 - protein, 117 aa, synthetic construct
  REGION 1..117: note = Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 18 - protein, 112 aa, synthetic construct
  REGION 1..112: note = Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 19 - protein, 8 aa, synthetic construct
  REGION 1..8: note = Synthetic
IDPETGDT

SEQ ID NO: 20 - protein, 17 aa, synthetic construct
  REGION 1..17: note = Synthetic
WIDPETGDTEYASKFQD

SEQ ID NO: 21 - skipped

SEQ ID NO: 22 - protein, 117 aa, synthetic construct
  REGION 1..117: note = Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSS

SEQ ID NO: 23 - protein, 8 aa, synthetic construct
  REGION 1..8: note = Synthetic
IDPESGDT

SEQ ID NO: 24 - protein, 17 aa, synthetic construct
  REGION 1..17: note = Synthetic
WIDPESGDTEYASKFQD

SEQ ID NO: 25 - skipped

SEQ ID NO: 26 - protein, 117 aa, synthetic construct
  REGION 1..117: note = Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSS

SEQ ID NO: 27 - protein, 9 aa, synthetic construct
  REGION 1..9: note = Synthetic
GYSITSGYY

SEQ ID NO: 28 - protein, 7 aa, synthetic construct
  REGION 1..7: note = Synthetic
ITFDGAN

SEQ ID NO: 29 - protein, 12 aa, synthetic construct
  REGION 1..12: note = Synthetic
TRSSYDYDVLDY

SEQ ID NO: 30 - protein, 6 aa, synthetic construct
  REGION 1..6: note = Synthetic
QDISNF

SEQ ID NO: 31 - skipped

SEQ ID NO: 32 - protein, 9 aa, synthetic construct
  REGION 1..9: note = Synthetic
QQGHTLPYT

SEQ ID NO: 33 - protein, 6 aa, synthetic construct
  REGION 1..6: note = Synthetic
SGYYWN

SEQ ID NO: 34 - protein, 16 aa, synthetic construct
  REGION 1..16: note = Synthetic
YITFDGANNYNPSLKN

SEQ ID NO: 35 - protein, 10 aa, synthetic construct
  REGION 1..10: note = Synthetic
SSYDYDVLDY

SEQ ID NO: 36 - protein, 11 aa, synthetic construct
  REGION 1..11: note = Synthetic
RASQDISNFLN

SEQ ID NO: 37 - protein, 7 aa, synthetic construct
  REGION 1..7: note = Synthetic
YTSRLHS

SEQ ID NO: 38 - protein, 8 aa, synthetic construct
  REGION 1..8: note = Synthetic
GYSITSGY

SEQ ID NO: 39 - skipped

SEQ ID NO: 40 - protein, 8 aa, synthetic construct
  REGION 1..8: note = Synthetic
SYDYDVLD

SEQ ID NO: 41 - protein, 7 aa, synthetic construct
  REGION 1..7: note = Synthetic
SQDISNF

SEQ ID NO: 42 - protein, 6 aa, synthetic construct
  REGION 1..6: note = Synthetic
GHTLPY

SEQ ID NO: 43 - protein, 119 aa, synthetic construct
  REGION 1..119: note = Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 44 - protein, 107 aa, synthetic construct
  REGION 1..107: note = Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 45 - protein, 8 aa, synthetic construct
  REGION 1..8: note = Synthetic
GYSFTDYC

SEQ ID NO: 46 - protein, 8 aa, synthetic construct
  REGION 1..8: note = Synthetic
IYPGSGNT

SEQ ID NO: 47 - protein, 13 aa, synthetic construct
  REGION 1..13: note = Synthetic
AREDYYPYHGMDY

SEQ ID NO: 48 - protein, 10 aa, synthetic construct
  REGION 1..10: note = Synthetic
ESVDGYDNSF

SEQ ID NO: 49 - skipped

SEQ ID NO: 50 - protein, 9 aa, synthetic construct
  REGION 1..9: note = Synthetic
QQSSEDPWT

SEQ ID NO: 51 - protein, 5 aa, synthetic construct
  REGION 1..5: note = Synthetic
DYCIN

SEQ ID NO: 52 - protein, 17 aa, synthetic construct
  REGION 1..17: note = Synthetic
WIYPGSGNTRYSERFKG

SEQ ID NO: 53 - protein, 11 aa, synthetic construct
  REGION 1..11: note = Synthetic
EDYYPYHGMDY

SEQ ID NO: 54 - protein, 15 aa, synthetic construct
  REGION 1..15: note = Synthetic
RASESVDGYDNSFMH

SEQ ID NO: 55 - protein, 7 aa, synthetic construct
  REGION 1..7: note = Synthetic
RASNLES

SEQ ID NO: 56 - protein, 7 aa, synthetic construct
  REGION 1..7: note = Synthetic
GYSFTDY

SEQ ID NO: 57 - skipped

SEQ ID NO: 58 - protein, 9 aa, synthetic construct
  REGION 1..9: note = Synthetic
DYYPYHGMD

SEQ ID NO: 59 - protein, 11 aa, synthetic construct
  REGION 1..11: note = Synthetic
SESVDGYDNSF

SEQ ID NO: 60 - protein, 6 aa, synthetic construct
  REGION 1..6: note = Synthetic
SSEDPW

SEQ ID NO: 61 - protein, 120 aa, synthetic construct
  REGION 1..120: note = Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 62 - protein, 111 aa, synthetic construct
  REGION 1..111: note = Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 63 - protein, 8 aa, synthetic construct
  REGION 1..8: note = Synthetic
GYSFTDYY

SEQ ID NO: 64 - protein, 5 aa, synthetic construct
  REGION 1..5: note = Synthetic
DYYIN

SEQ ID NO: 65 - protein, 120 aa, synthetic construct
  REGION 1..120: note = Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 66 - protein, 8 aa, synthetic construct
  REGION 1..8: note = Synthetic
GYSFTDYD

SEQ ID NO: 67 - protein, 5 aa, synthetic construct
  REGION 1..5: note = Synthetic
DYDIN

SEQ ID NO: 68 - protein, 120 aa, synthetic construct
  REGION 1..120: note = Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 69 - protein, 117 aa, synthetic construct
  REGION 1..117: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSS

SEQ ID NO: 70 - protein, 112 aa, synthetic construct
  REGION 1..112: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIK

SEQ ID NO: 71 - protein, 117 aa, synthetic construct
  REGION 1..117: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSS

SEQ ID NO: 72 - protein, 117 aa, synthetic construct
  REGION 1..117: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSS

SEQ ID NO: 73 - protein, 119 aa, synthetic construct
  REGION 1..119: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSS

SEQ ID NO: 74 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIK

SEQ ID NO: 75 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 76 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 77 - protein, 120 aa, synthetic construct
  REGION 1..120: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 78 - protein, 111 aa, synthetic construct
  REGION 1..111: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIK

SEQ ID NO: 79 - protein, 120 aa, synthetic construct
  REGION 1..120: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 80 - protein, 111 aa, synthetic construct
  REGION 1..111: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIK

SEQ ID NO: 81 - protein, 330 aa, Homo sapiens [sequence omitted]

SEQ ID NO: 82 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 107 aa, synthetic construct [sequence omitted]

SEQ ID NO: 84 - protein, 447 aa, synthetic construct
  REGION 1..447: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 219 aa, synthetic construct
  REGION 1..219: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 447 aa, synthetic construct
  REGION 1..447: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 447 aa, synthetic construct
  REGION 1..447: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 449 aa, synthetic construct
  REGION 1..449: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 449 aa, synthetic construct
  REGION 1..449: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 450 aa, synthetic construct
  REGION 1..450: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 218 aa, synthetic construct
  REGION 1..218: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 450 aa, synthetic construct
  REGION 1..450: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 108 aa, synthetic construct
  REGION 1..108: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 225 aa, synthetic construct
  REGION 1..225: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 98 - protein, 225 aa, synthetic construct
  REGION 1..225: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 99 - protein, 225 aa, synthetic construct
  REGION 1..225: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 227 aa, synthetic construct
  REGION 1..227: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 228 aa, synthetic construct
  REGION 1..228: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - protein, 228 aa, synthetic construct
  REGION 1..228: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 19 aa, synthetic construct
  REGION 1..19: note = Synthetic
MGWSCIILFLVATATGVHS

SEQ ID NO: 105 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 106 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 107 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 108 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 109 - protein, 197 aa, synthetic construct
  REGION 1..197: note = Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 110 - protein, 5 aa, synthetic construct
  REGION 1..5: note = Synthetic
SYWMH

SEQ ID NO: 111 - protein, 17 aa, synthetic construct
  REGION 1..17: note = Synthetic
EINPTNGRTNYIEKFKS

SEQ ID NO: 112 - protein, 7 aa, synthetic construct
  REGION 1..7: note = Synthetic
GTRAYHY

SEQ ID NO: 113 - protein, 11 aa, synthetic construct
  REGION 1..11: note = Synthetic
RASDNLYSNLA

SEQ ID NO: 114 - protein, 7 aa, synthetic construct
  REGION 1..7: note = Synthetic
DATNLAD

SEQ ID NO: 115 - protein, 9 aa, synthetic construct
  REGION 1..9: note = Synthetic
QHFWGTPLT

SEQ ID NO: 116 - protein, 7 aa, synthetic construct
  REGION 1..7: note = Synthetic
GYTFTSY

SEQ ID NO: 117 - protein, 6 aa, synthetic construct
  REGION 1..6: note = Synthetic
NPTNGR

SEQ ID NO: 118 - protein, 6 aa, synthetic construct
  REGION 1..6: note = Synthetic
TSYWMH

SEQ ID NO: 119 - protein, 13 aa, synthetic construct
  REGION 1..13: note = Synthetic
WIGEINPTNGRTN

SEQ ID NO: 120 - protein, 6 aa, synthetic construct
  REGION 1..6: note = Synthetic
ARGTRA

SEQ ID NO: 121 - protein, 7 aa, synthetic construct
  REGION 1..7: note = Synthetic
YSNLAWY

SEQ ID NO: 122 - protein, 10 aa, synthetic construct
  REGION 1..10: note = Synthetic
LLVYDATNLA

SEQ ID NO: 123 - protein, 8 aa, synthetic construct
  REGION 1..8: note = Synthetic
QHFWGTPL

SEQ ID NO: 124 - protein, 116 aa, Mus musculus
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 125 - protein, 107 aa, Mus musculus
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 126 - protein, 9 aa, synthetic construct
  REGION 1..9: note = Synthetic
QHFAGTPLT

SEQ ID NO: 127 - protein, 8 aa, synthetic construct
  REGION 1..8: note = Synthetic
QHFAGTPL

SEQ ID NO: 128 - protein, 116 aa, synthetic construct
  REGION 1..116: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 129 - protein, 107 aa, synthetic construct
  REGION 1..107: note = Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 130 - protein, 13993 aa, Homo sapiens
TCCTGGCATCAGTTACTGTGTTGACTCACTCAGTGTTGGGATCACTCACTTTCCCCCTACAGGACTCAGATCTGGGAGGCAATTACCTTCGGAGAAAAACGAATAGGAAAAACTGAAGTGTTACTTTTTTTAAAGCTGCTGAAGTTTGTTGGTTTCTCATTGTTTTTAAGCCTACTGGAGCAATAAAGTTTGAAGAACTTTTACCAGGTTTTTTTTATCGCTGCCTTGATATACACTTTTCAAAATGCTTTGGTGGGAAGAAGTAGAGGACTGTTATGAAAGAGAAGATGTTCAAAAGAAAACATTCACAAAATGGGTAAATGCACAATTTTCTAAGTTTGGGAAGCAGCATATTGAGAACCTCTTCAGTGACCTACAGGATGGGAGGCGCCTCCTAGACCTCCTCGAAGGCCTGACAGGGCAAAAACTGCCAAAAGAAAAAGGATCCACAAGAGTTCATGCCCTGAACAATGTCAACAAGGCACTGCGGGTTTTGCAGAACAATAATGTTGATTTAGTGAATATTGGAAGTACTGACATCGTAGATGGAAATCATAAACTGACTCTTGGTTTGATTTGGAATATAATCCTCCACTGGCAGGTCAAAAATGTAATGAAAAATATCATGGCTGGATTGCAACAAACCAACAGTGAAAAGATTCTCCTGAGCTGGGTCCGACAATCAACTCGTAATTATCCACAGGTTAATGTAATCAACTTCACCACCAGCTGGTCTGATGGCCTGGCTTTGAATGCTCTCATCCATAGTCATAGGCCAGACCTATTTGACTGGAATAGTGTGGTTTGCCAGCAGTCAGCCACACAACGACTGGAACATGCATTCAACATCGCCAGATATCAATTAGGCATAGAGAAACTACTCGATCCTGAAGATGTTGATACCACCTATCCAGATAAGAAGTCCATCTTAATGTACATCACATCACTCTTCCAAGTTTTGCCTCAACAAGTGAGCATTGAAGCCATCCAGGAAGTGGAAATGTTGCCAAGGCCACCTAAAGTGACTAAAGAAGAACATTTTCAGTTACATCATCAAATGCACTATTCTCAACAGATCACGGTCAGTCTAGCACAGGGATATGAGAGAACTTCTTCCCCTAAGCCTCGATTCAAGAGCTATGCCTACACACAGGCTGCTTATGTCACCACCTCTGACCCTACACGGAGCCCATTTCCTTCACAGCATTTGGAAGCTCCTGAAGACAAGTCATTTGGCAGTTCATTGATGGAGAGTGAAGTAAACCTGGACCGTTATCAAACAGCTTTAGAAGAAGTATTATCGTGGCTTCTTTCTGCTGAGGACACATTGCAAGCACAAGGAGAGATTTCTAATGATGTGGAAGTGGTGAAAGACCAGTTTCATACTCATGAGGGGTACATGATGGATTTGACAGCCCATCAGGGCCGGGTTGGTAATATTCTACAATTGGGAAGTAAGCTGATTGGAACAGGAAAATTATCAGAAGATGAAGAAACTGAAGTACAAGAGCAGATGAATCTCCTAAATTCAAGATGGGAATGCCTCAGGGTAGCTAGCATGGAAAAACAAAGCAATTTACATAGAGTTTTAATGGATCTCCAGAATCAGAAACTGAAAGAGTTGAATGACTGGCTAACAAAAACAGAAGAAAGAACAAGGAAAATGGAGGAAGAGCCTCTTGGACCTGATCTTGAAGACCTAAAACGCCAAGTACAACAACATAAGGTGCTTCAAGAAGATCTAGAACAAGAACAAGTCAGGGTCAATTCTCTCACTCACATGGTGGTGGTAGTTGATGAATCTAGTGGAGATCACGCAACTGCTGCTTTGGAAGAACAACTTAAGGTATTGGGAGATCGATGGGCAAACATCTGTAGATGGACAGAAGACCGCTGGGTTCTTTTACAAGACATCCTTCTCAAATGGCAACGTCTTACTGAAGAACAGTGCCTTTTTAGTGCATGGCTTTCAGAAAAAGAAGATGCAGTGAACAAGATTCACACAACTGGCTTTAAAGATCAAAATGAAATGTTATCAAGTCTTCAAAAACTGGCCGTTTTAAAAGCGGATCTAGAAAAGAAAAAGCAATCCATGGGCAAACTGTATTCACTCAAACAAGATCTTCTTTCAACACTGAAGAATAAGTCAGTGACCCAGAAGACGGAAGCATGGCTGGATAACTTTGCCCGGTGTTGGGATAATTTAGTCCAAAAACTTGAAAAGAGTACAGCACAGATTTCACAGGCTGTCACCACCACTCAGCCATCACTAACACAGACAACTGTAATGGAAACAGTAACTACGGTGACCACAAGGGAACAGATCCTGGTAAAGCATGCTCAAGAGGAACTTCCACCACCACCTCCCCAAAAGAAGAGGCAGATTACTGTGGATTCTGAAATTAGGAAAAGGTTGGATGTTGATATAACTGAACTTCACAGCTGGATTACTCGCTCAGAAGCTGTGTTGCAGAGTCCTGAATTTGCAATCTTTCGGAAGGAAGGCAACTTCTCAGACTTAAAAGAAAAAGTCAATGCCATAGAGCGAGAAAAAGCTGAGAAGTTCAGAAAACTGCAAGATGCCAGCAGATCAGCTCAGGCCCTGGTGGAACAGATGGTGAATGAGGGTGTTAATGCAGATAGCATCAAACAAGCCTCAGAACAACTGAACAGCCGGTGGATCGAATTCTGCCAGTTGCTAAGTGAGAGACTTAACTGGCTGGAGTATCAGAACAACATCATCGCTTTCTATAATCAGCTACAACAATTGGAGCAGATGACAACTACTGCTGAAAACTGGTTGAAAATCCAACCCACCACCCCATCAGAGCCAACAGCAATTAAAAGTCAGTTAAAAATTTGTAAGGATGAAGTCAACCGGCTATCAGGTCTTCAACCTCAAATTGAACGATTAAAAATTCAAAGCATAGCCCTGAAAGAGAAAGGACAAGGACCCATGTTCCTGGATGCAGACTTTGTGGCCTTTACAAATCATTTTAAGCAAGTCTTTTCTGATGTGCAGGCCAGAGAGAAAGAGCTACAGACAATTTTTGACACTTTGCCACCAATGCGCTATCAGGAGACCATGAGTGCCATCAGGACATGGGTCCAGCAGTCAGAAACCAAACTCTCCATACCTCAACTTAGTGTCACCGACTATGAAATCATGGAGCAGAGACTCGGGGAATTGCAGGCTTTACAAAGTTCTCTGCAAGAGCAACAAAGTGGCCTATACTATCTCAGCACCACTGTGAAAGAGATGTCGAAGAAAGCGCCCTCTGAAATTAGCCGGAAATATCAATCAGAATTTGAAGAAATTGAGGGACGCTGGAAGAAGCTCTCCTCCCAGCTGGTTGAGCATTGTCAAAAGCTAGAGGAGCAAATGAATAAACTCCGAAAAATTCAGAATCACATACAAACCCTGAAGAAATGGATGGCTGAAGTTGATGTTTTTCTGAAGGAGGAATGGCCTGCCCTTGGGGATTCAGAAATTCTAAAAAAGCAGCTGAAACAGTGCAGACTTTTAGTCAGTGATATTCAGACAATTCAGCCCAGTCTAAACAGTGTCAATGAAGGTGGGCAGAAGATAAAGAATGAAGCAGAGCCAGAGTTTGCTTCGAGACTTGAGACAGAACTCAAAGAACTTAACACTCAGTGGGATCACATGTGCCAACAGGTCTATGCCAGAAAGGAGGCCTTGAAGGGAGGTTTGGAGAAAACTGTAAGCCTCCAGAAAGATCTATCAGAGATGCACGAATGGATGACACAAGCTGAAGAAGAGTATCTTGAGAGAGATTTTGAATATAAAACTCCAGATGAATTACAGAAAGCAGTTGAAGAGATGAAGAGAGCTAAAGAAGAGGCCCAACAAAAAGAAGCGAAAGTGAAACTCCTTACTGAGTCTGTAAATAGTGTCATAGCTCAAGCTCCACCTGTAGCACAAGAGGCCTTAAAAAAGGAACTTGAAACTCTAACCACCAACTACCAGTGGCTCTGCACTAGGCTGAATGGGAAATGCAAGACTTTGGAAGAAGTTTGGGCATGTTGGCATGAGTTATTGTCATACTTGGAGAAAGCAAACAAGTGGCTAAATGAAGTAGAATTTAAACTTAAAACCACTGAAAACATTCCTGGCGGAGCTGAGGAAATCTCTGAGGTGCTAGATTCACTTGAAAATTTGATGCGACATTCAGAGGATAACCCAAATCAGATTCGCATATTGGCACAGACCCTAACAGATGGCGGAGTCATGGATGAGCTAATCAATGAGGAACTTGAGACATTTAATTCTCGTTGGAGGGAACTACATGAAGAGGCTGTAAGGAGGCAAAAGTTGCTTGAACAGAGCATCCAGTCTGCCCAGGAGACTGAAAAATCCTTACACTTAATCCAGGAGTCCCTCACATTCATTGACAAGCAGTTGGCAGCTTATATTGCAGACAAGGTGGACGCAGCTCAAATGCCTCAGGAAGCCCAGAAAATCCAATCTGATTTGACAAGTCATGAGATCAGTTTAGAAGAAATGAAGAAACATAATCAGGGGAAGGAGGCTGCCCAAAGAGTCCTGTCTCAGATTGATGTTGCACAGAAAAAATTACAAGATGTCTCCATGAAGTTTCGATTATTCCAGAAACCAGCCAATTTTGAGCAGCGTCTACAAGAAAGTAAGATGATTTTAGATGAAGTGAAGATGCACTTGCCTGCATTGGAAACAAAGAGTGTGGAACAGGAAGTAGTACAGTCACAGCTAAATCATTGTGTGAACTTGTATAAAAGTCTGAGTGAAGTGAAGTCTGAAGTGGAAATGGTGATAAAGACTGGACGTCAGATTGTACAGAAAAAGCAGACGGAAAATCCCAAAGAACTTGATGAAAGAGTAACAGCTTTGAAATTGCATTATAATGAGCTGGGAGCAAAGGTAACAGAAAGAAAGCAACAGTTGGAGAAATGCTTGAAATTGTCCCGTAAGATGCGAAAGGAAATGAATGTCTTGACAGAATGGCTGGCAGCTACAGATATGGAATTGACAAAGAGATCAGCAGTTGAAGGAATGCCTAGTAATTTGGATTCTGAAGTTGCCTGGGGAAAGGCTACTCAAAAAGAGATTGAGAAACAGAAGGTGCACCTGAAGAGTATCACAGAGGTAGGAGAGGCCTTGAAAACAGTTTTGGGCAAGAAGGAGACGTTGGTGGAAGATAAACTCAGTCTTCTGAATAGTAACTGGATAGCTGTCACCTCCCGAGCAGAAGAGTGGTTAAATCTTTTGTTGGAATACCAGAAACACATGGAAACTTTTGACCAGAATGTGGACCACATCACAAAGTGGATCATTCAGGCTGACACACTTTTGGATGAATCAGAGAAAAAGAAACCCCAGCAAAAAGAAGACGTGCTTAAGCGTTTAAAGGCAGAACTGAATGACATACGCCCAAAGGTGGACTCTACACGTGACCAAGCAGCAAACTTGATGGCAAACCGCGGTGACCACTGCAGGAAATTAGTAGAGCCCCAAATCTCAGAGCTCAACCATCGATTTGCAGCCATTTCACACAGAATTAAGACTGGAAAGGCCTCCATTCCTTTGAAGGAATTGGAGCAGTTTAACTCAGATATACAAAAATTGCTTGAACCACTGGAGGCTGAAATTCAGCAGGGGGTGAATCTGAAAGAGGAAGACTTCAATAAAGATATGAATGAAGACAATGAGGGTACTGTAAAAGAATTGTTGCAAAGAGGAGACAACTTACAACAAAGAATCACAGATGAGAGAAAGCGAGAGGAAATAAAGATAAAACAGCAGCTGTTACAGACAAAACATAATGCTCTCAAGGATTTGAGGTCTCAAAGAAGAAAAAAGGCTCTAGAAATTTCTCATCAGTGGTATCAGTACAAGAGGCAGGCTGATGATCTCCTGAAATGCTTGGATGACATTGAAAAAAAATTAGCCAGCCTACCTGAGCCCAGAGATGAAAGGAAAATAAAGGAAATTGATCGGGAATTGCAGAAGAAGAAAGAGGAGCTGAATGCAGTGCGTAGGCAAGCTGAGGGCTTGTCTGAGGATGGGGCCGCAATGGCAGTGGAGCCAACTCAGATCCAGCTCAGCAAGCGCTGGCGGGAAATTGAGAGCAAATTTGCTCAGTTTCGAAGACTCAACTTTGCACAAATTCACACTGTCCGTGAAGAAACGATGATGGTGATGACTGAAGACATGCCTTTGGAAATTTCTTATGTGCCTTCTACTTATTTGACTGAAATCACTCATGTCTCACAAGCCCTATTAGAAGTGGAACAACTTCTCAATGCTCCTGACCTCTGTGCTAAGGACTTTGAAGATCTCTTTAAGCAAGAGGAGTCTCTGAAGAATATAAAAGATAGTCTACAACAAAGCTCAGGTCGGATTGACATTATTCATAGCAAGAAGACAGCAGCATTGCAAAGTGCAACGCCTGTGGAAAGGGTGAAGCTACAGGAAGCTCTCTCCCAGCTTGATTTCCAATGGGAAAAAGTTAACAAAATGTACAAGGACCGACAAGGGCGATTTGACAGATCTGTTGAGAAATGGCGGCGTTTTCATTATGATATAAAGATATTTAATCAGTGGCTAACAGAAGCTGAACAGTTTCTCAGAAAGACACAAATTCCTGAGAATTGGGAACATGCTAAATACAAATGGTATCTTAAGGAACTCCAGGATGGCATTGGGCAGCGGCAAACTGTTGTCAGAACATTGAATGCAACTGGGGAAGAAATAATTCAGCAATCCTCAAAAACAGATGCCAGTATTCTACAGGAAAAATTGGGAAGCCTGAATCTGCGGTGGCAGGAGGTCTGCAAACAGCTGTCAGACAGAAAAAAGAGGCTAGAAGAACAAAAGAATATCTTGTCAGAATTTCAAAGAGATTTAAATGAATTTGTTTTATGGTTGGAGGAAGCAGATAACATTGCTAGTATCCCACTTGAACCTGGAAAAGAGCAGCAACTAAAAGAAAAGCTTGAGCAAGTCAAGTTACTGGTGGAAGAGTTGCCCCTGCGCCAGGGAATTCTCAAACAATTAAATGAAACTGGAGGACCCGTGCTTGTAAGTGCTCCCATAAGCCCAGAAGAGCAAGATAAACTTGAAAATAAGCTCAAGCAGACAAATCTCCAGTGGATAAAGGTTTCCAGAGCTTTACCTGAGAAACAAGGAGAAATTGAAGCTCAAATAAAAGACCTTGGGCAGCTTGAAAAAAAGCTTGAAGACCTTGAAGAGCAGTTAAATCATCTGCTGCTGTGGTTATCTCCTATTAGGAATCAGTTGGAAATTTATAACCAACCAAACCAAGAAGGACCATTTGACGTTCAGGAAACTGAAATAGCAGTTCAAGCTAAACAACCGGATGTGGAAGAGATTTTGTCTAAAGGGCAGCATTTGTACAAGGAAAAACCAGCCACTCAGCCAGTGAAGAGGAAGTTAGAAGATCTGAGCTCTGAGTGGAAGGCGGTAAACCGTTTACTTCAAGAGCTGAGGGCAAAGCAGCCTGACCTAGCTCCTGGACTGACCACTATTGGAGCCTCTCCTACTCAGACTGTTACTCTGGTGACACAACCTGTGGTTACTAAGGAAACTGCCATCTCCAAACTAGAAATGCCATCTTCCTTGATGTTGGAGGTACCTGCTCTGGCAGATTTCAACCGGGCTTGGACAGAACTTACCGACTGGCTTTCTCTGCTTGATCAAGTTATAAAATCACAGAGGGTGATGGTGGGTGACCTTGAGGATATCAACGAGATGATCATCAAGCAGAAGGCAACAATGCAGGATTTGGAACAGAGGCGTCCCCAGTTGGAAGAACTCATTACCGCTGCCCAAAATTTGAAAAACAAGACCAGCAATCAAGAGGCTAGAACAATCATTACGGATCGAATTGAAAGAATTCAGAATCAGTGGGATGAAGTACAAGAACACCTTCAGAACCGGAGGCAACAGTTGAATGAAATGTTAAAGGATTCAACACAATGGCTGGAAGCTAAGGAAGAAGCTGAGCAGGTCTTAGGACAGGCCAGAGCCAAGCTTGAGTCATGGAAGGAGGGTCCCTATACAGTAGATGCAATCCAAAAGAAAATCACAGAAACCAAGCAGTTGGCCAAAGACCTCCGCCAGTGGCAGACAAATGTAGATGTGGCAAATGACTTGGCCCTGAAACTTCTCCGGGATTATTCTGCAGATGATACCAGAAAAGTCCACATGATAACAGAGAATATCAATGCCTCTTGGAGAAGCATTCATAAAAGGGTGAGTGAGCGAGAGGCTGCTTTGGAAGAAACTCATAGATTACTGCAACAGTTCCCCCTGGACCTGGAAAAGTTTCTTGCCTGGCTTACAGAAGCTGAAACAACTGCCAATGTCCTACAGGATGCTACCCGTAAGGAAAGGCTCCTAGAAGACTCCAAGGGAGTAAAAGAGCTGATGAAACAATGGCAAGACCTCCAAGGTGAAATTGAAGCTCACACAGATGTTTATCACAACCTGGATGAAAACAGCCAAAAAATCCTGAGATCCCTGGAAGGTTCCGATGATGCAGTCCTGTTACAAAGACGTTTGGATAACATGAACTTCAAGTGGAGTGAACTTCGGAAAAAGTCTCTCAACATTAGGTCCCATTTGGAAGCCAGTTCTGACCAGTGGAAGCGTCTGCACCTTTCTCTGCAGGAACTTCTGGTGTGGCTACAGCTGAAAGATGATGAATTAAGCCGGCAGGCACCTATTGGAGGCGACTTTCCAGCAGTTCAGAAGCAGAACGATGTACATAGGGCCTTCAAGAGGGAATTGAAAACTAAAGAACCTGTAATCATGAGTACTCTTGAGACTGTACGAATATTTCTGACAGAGCAGCCTTTGGAAGGACTAGAGAAACTCTACCAGGAGCCCAGAGAGCTGCCTCCTGAGGAGAGAGCCCAGAATGTCACTCGGCTTCTACGAAAGCAGGCTGAGGAGGTCAATACTGAGTGGGAAAAATTGAACCTGCACTCCGCTGACTGGCAGAGAAAAATAGATGAGACCCTTGAAAGACTCCAGGAACTTCAAGAGGCCACGGATGAGCTGGACCTCAAGCTGCGCCAAGCTGAGGTGATCAAGGGATCCTGGCAGCCCGTGGGCGATCTCCTCATTGACTCTCTCCAAGATCACCTCGAGAAAGTCAAGGCACTTCGAGGAGAAATTGCGCCTCTGAAAGAGAACGTGAGCCACGTCAATGACCTTGCTCGCCAGCTTACCACTTTGGGCATTCAGCTCTCACCGTATAACCTCAGCACTCTGGAAGACCTGAACACCAGATGGAAGCTTCTGCAGGTGGCCGTCGAGGACCGAGTCAGGCAGCTGCATGAAGCCCACAGGGACTTTGGTCCAGCATCTCAGCACTTTCTTTCCACGTCTGTCCAGGGTCCCTGGGAGAGAGCCATCTCGCCAAACAAAGTGCCCTACTATATCAACCACGAGACTCAAACAACTTGCTGGGACCATCCCAAAATGACAGAGCTCTACCAGTCTTTAGCTGACCTGAATAATGTCAGATTCTCAGCTTATAGGACTGCCATGAAACTCCGAAGACTGCAGAAGGCCCTTTGCTTGGATCTCTTGAGCCTGTCAGCTGCATGTGATGCCTTGGACCAGCACAACCTCAAGCAAAATGACCAGCCCATGGATATCCTGCAGATTATTAATTGTTTGACCACTATTTATGACCGCCTGGAGCAAGAGCACAACAATTTGGTCAACGTCCCTCTCTGCGTGGATATGTGTCTGAACTGGCTGCTGAATGTTTATGATACGGGACGAACAGGGAGGATCCGTGTCCTGTCTTTTAAAACTGGCATCATTTCCCTGTGTAAAGCACATTTGGAAGACAAGTACAGATACCTTTTCAAGCAAGTGGCAAGTTCAACAGGATTTTGTGACCAGCGCAGGCTGGGCCTCCTTCTGCATGATTCTATCCAAATTCCAAGACAGTTGGGTGAAGTTGCATCCTTTGGGGGCAGTAACATTGAGCCAAGTGTCCGGAGCTGCTTCCAATTTGCTAATAATAAGCCAGAGATCGAAGCGGCCCTCTTCCTAGACTGGATGAGACTGGAACCCCAGTCCATGGTGTGGCTGCCCGTCCTGCACAGAGTGGCTGCTGCAGAAACTGCCAAGCATCAGGCCAAATGTAACATCTGCAAAGAGTGTCCAATCATTGGATTCAGGTACAGGAGTCTAAAGCACTTTAATTATGACATCTGCCAAAGCTGCTTTTTTTCTGGTCGAGTTGCAAAAGGCCATAAAATGCACTATCCCATGGTGGAATATTGCACTCCGACTACATCAGGAGAAGATGTTCGAGACTTTGCCAAGGTACTAAAAAACAAATTTCGAACCAAAAGGTATTTTGCGAAGCATCCCCGAATGGGCTACCTGCCAGTGCAGACTGTCTTAGAGGGGGACAACATGGAAACTCCCGTTACTCTGATCAACTTCTGGCCAGTAGATTCTGCGCCTGCCTCGTCCCCTCAGCTTTCACACGATGATACTCATTCACGCATTGAACATTATGCTAGCAGGCTAGCAGAAATGGAAAACAGCAATGGATCTTATCTAAATGATAGCATCTCTCCTAATGAGAGCATAGATGATGAACATTTGTTAATCCAGCATTACTGCCAAAGTTTGAACCAGGACTCCCCCCTGAGCCAGCCTCGTAGTCCTGCCCAGATCTTGATTTCCTTAGAGAGTGAGGAAAGAGGGGAGCTAGAGAGAATCCTAGCAGATCTTGAGGAAGAAAACAGGAATCTGCAAGCAGAATATGACCGTCTAAAGCAGCAGCACGAACATAAAGGCCTGTCCCCACTGCCGTCCCCTCCTGAAATGATGCCCACCTCTCCCCAGAGTCCCCGGGATGCTGAGCTCATTGCTGAGGCCAAGCTACTGCGTCAACACAAAGGCCGCCTGGAAGCCAGGATGCAAATCCTGGAAGACCACAATAAACAGCTGGAGTCACAGTTACACAGGCTAAGGCAGCTGCTGGAGCAACCCCAGGCAGAGGCCAAAGTGAATGGCACAACGGTGTCCTCTCCTTCTACCTCTCTACAGAGGTCCGACAGCAGTCAGCCTATGCTGCTCCGAGTGGTTGGCAGTCAAACTTCGGACTCCATGGGTGAGGAAGATCTTCTCAGTCCTCCCCAGGACACAAGCACAGGGTTAGAGGAGGTGATGGAGCAACTCAACAACTCCTTCCCTAGTTCAAGAGGAAGAAATACCCCTGGAAAGCCAATGAGAGAGGACACAATGTAGGAAGTCTTTTCCACATGGCAGATGATTTGGGCAGAGCGATGGAGTCCTTAGTATCAGTCATGACAGATGAAGAAGGAGCAGAATAAATGTTTTACAACTCCTGATTCCCGCATGGTTTTTATAATATTCATACAACAAAGAGGATTAGACAGTAAGAGTTTACAAGAAATAAATCTATATTTTTGTGAAGGGTAGTGGTATTATACTGTAGATTTCAGTAGTTTCTAAGTCTGTTATTGTTTTGTTAACAATGGCAGGTTTTACACGTCTATGCAATTGTACAAAAAAGTTATAAGAAAACTACATGTAAAATCTTGATAGCTAAATAACTTGCCATTTCTTTATATGGAACGCATTTTGGGTTGTTTAAAAATTTATAACAGTTATAAAGAAAGATTGTAAACTAAAGTGTGCTTTATAAAAAAAAGTTGTTTATAAAAACCCCTAAAAACAAAACAAACACACACACACACACATACACACACACACACAAAACTTTGAGGCAGCGCATTGTTTTGCATCCTTTTGGCGTGATATCCATATGAAATTCATGGCTTTTTCTTTTTTTGCATATTAAAGATAAGACTTCCTCTACCACCACACCAAATGACTACTACACACTGCTCATTTGAGAACTGTCAGCTGAGTGGGGCAGGCTTGAGTTTTCATTTCATATATCTATATGTCTATAAGTATATAAATACTATAGTTATATAGATAAAGAGATACGAATTTCTATAGACTGACTTTTTCCATTTTTTAAATGTTCATGTCACATCCTAATAGAAAGAAATTACTTCTAGTCAGTCATCCAGGCTTACCTGCTTGGTCTAGAATGGATTTTTCCCGGAGCCGGAAGCCAGGAGGAAACTACACCACACTAAAACATTGTCTACAGCTCCAGATGTTTCTCATTTTAAACAACTTTCCACTGACAACGAAAGTAAAGTAAAGTATTGGATTTTTTTAAAGGGAACATGTGAATGAATACACAGGACTTATTATATCAGAGTGAGTAATCGGTTGGTTGGTTGATTGATTGATTGATTGATACATTCAGCTTCCTGCTGCTAGCAATGCCACGATTTAGATTTAATGATGCTTCAGTGGAAATCAATCAGAAGGTATTCTGACCTTGTGAACATCAGAAGGTATTTTTTAACTCCCAAGCAGTAGCAGGACGATGATAGGGCTGGAGGGCTATGGATTCCCAGCCCATCCCTGTGAAGGAGTAGGCCACTCTTTAAGTGAAGGATTGGATGATTGTTCATAATACATAAAGTTCTCTGTAATTACAACTAAATTATTATGCCCTCTTCTCACAGTCAAAAGGAACTGGGTGGTTTGGTTTTTGTTGCTTTTTTAGATTTATTGTCCCATGTGGGATGAGTTTTTAAATGCCACAAGACATAATTTAAAATAAATAAACTTTGGGAAAAGGTGTAAAACAGTAGCCCCATCACATTTGTGATACTGACAGGTATCAACCCAGAAGCCCATGAACTGTGTTTCCATCCTTTGCATTTCTCTGCGAGTAGTTCCACACAGGTTTGTAAGTAAGTAAGAAAGAAGGCAAATTGATTCAAATGTTACAAAAAAACCCTTCTTGGTGGATTAGACAGGTTAAATATATAAACAAACAAACAAAAATTGCTCAAAAAAGAGGAGAAAAGCTCAAGAGGAAAAGCTAAGGACTGGTAGGAAAAAGCTTTACTCTTTCATGCCATTTTATTTCTTTTTGATTTTTAAATCATTCATTCAATAGATACCACCGTGTGACCTATAATTTTGCAAATCTGTTACCTCTGACATCAAGTGTAATTAGCTTTTGGAGAGTGGGCTGACATCAAGTGTAATTAGCTTTTGGAGAGTGGGTTTTGTCCATTATTAATAATTAATTAATTAACATCAAACACGGCTTCTCATGCTATTTCTACCTCACTTTGGTTTTGGGGTGTTCCTGATAATTGTGCACACCTGAGTTCACAGCTTCACCACTTGTCCATTGCGTTATTTTCTTTTTCCTTTATAATTCTTTCTTTTTCCTTCATAATTTTCAAAAGAAAACCCAAAGCTCTAAGGTAACAAATTACCAAATTACATGAAGATTTGGTTTTTGTCTTGCATTTTTTTCCTTTATGTGACGCTGGACCTTTTCTTTACCCAAGGATTTTTAAAACTCAGATTTAAAACAAGGGGTTACTTTACATCCTACTAAGAAGTTTAAGTAAGTAAGTTTCATTCTAAAATCAGAGGTAAATAGAGTGCATAAATAATTTTGTTTTAATCTTTTTGTTTTTCTTTTAGACACATTAGCTCTGGAGTGAGTCTGTCATAATATTTGAACAAAAATTGAGAGCTTTATTGCTGCATTTTAAGCATAATTAATTTGGACATTATTTCGTGTTGTGTTCTTTATAACCACCAAGTATTAAACTGTAAATCATAATGTAACTGAAGCATAAACATCACATGGCATGTTTTGTCATTGTTTTCAGGTACTGAGTTCTTACTTGAGTATCATAATATATTGTGTTTTAACACCAACACTGTAACATTTACGAATTATTTTTTTAAACTTCAGTTTTACTGCATTTTCACAACATATCAGACTTCACCAAATATATGCCTTACTATTGTATTATAGTACTGCTTTACTGTGTATCTCAATAAAGCACGCAGTTATGTTAC

SEQ ID NO: 131 - protein, 118 aa, Homo sapiens
GCAACAATGCAGGATTTGGAACAGAGGCGTCCCCAGTTGGAAGAACTCATTACCGCTGCCCAAAATTTGAAAAACAAGACCAGCAATCAAGAGGCTAGAACAATCATTACGGATCGAA

SEQ ID NO: 132 - protein, 446 aa, synthetic construct
  REGION 1..446: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
  REGION 1..214: note = Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 446 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, Homo sapiens
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 226 aa, synthetic construct
  REGION 1..226: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 137 - protein, 226 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 138 - protein, 8 aa, synthetic construct
  REGION 1..8: note = Synthetic
GYSFTSYW

SEQ ID NO: 139 - protein, 8 aa, synthetic construct
  REGION 1..8: note = Synthetic
IYPGDSDT

SEQ ID NO: 140 - protein, 15 aa, synthetic construct
  REGION 1..15: note = Synthetic
ARFPYDSSGYYSFDY

SEQ ID NO: 141 - protein, 6 aa, synthetic construct
  REGION 1..6: note = Synthetic
QSISSY

SEQ ID NO: 142 - skipped

SEQ ID NO: 143 - protein, 9 aa, synthetic construct
  REGION 1..9: note = Synthetic
QQSYSTPLT

SEQ ID NO: 144 - protein, 5 aa, synthetic construct
  REGION 1..5: note = Synthetic
SYWIG

SEQ ID NO: 145 - protein, 18 aa, synthetic construct
  REGION 1..18: note = Synthetic
IIYPGDSDTRYSPSFQGQ

SEQ ID NO: 146 - protein, 13 aa, synthetic construct
  REGION 1..13: note = Synthetic
FPYDSSGYYSFDY

SEQ ID NO: 147 - protein, 11 aa, synthetic construct
  REGION 1..11: note = Synthetic
RASQSISSYLN

SEQ ID NO: 148 - protein, 7 aa, synthetic construct
  REGION 1..7: note = Synthetic
AASSLQS

SEQ ID NO: 149 - protein, 7 aa, synthetic construct
  REGION 1..7: note = Synthetic
GYSFTSY

SEQ ID NO: 150 - skipped

SEQ ID NO: 151 - protein, 11 aa, synthetic construct
  REGION 1..11: note = Synthetic
PYDSSGYYSFD

SEQ ID NO: 152 - protein, 7 aa, synthetic construct
  REGION 1..7: note = Synthetic
SQSISSY

SEQ ID NO: 153 - protein, 6 aa, synthetic construct
  REGION 1..6: note = Synthetic
SYSTPL

SEQ ID NO: 154 - protein, 122 aa, synthetic construct
  REGION 1..122: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 155 - protein, 107 aa, synthetic construct [sequence omitted]

SEQ ID NO: 156 - protein, 452 aa, synthetic construct
  REGION 1..452: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 232 aa, synthetic construct
  REGION 1..232: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 159 - protein, 230 aa, synthetic construct
  REGION 1..230: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 160 - RNA, 24 nt, synthetic construct
  misc_feature 1..24: note = Synthetic
  source 1..24: mol_type = other RNA
gagtcatggaaggagggtccctat

SEQ ID NO: 161 - RNA, 23 nt, synthetic construct
  misc_feature 1..23: note = Synthetic
  source 1..23: mol_type = other RNA
ggagggtccctatacagtagatg

SEQ ID NO: 162 - RNA, 24 nt, synthetic construct
  misc_feature 1..24: note = Synthetic
  source 1..24: mol_type = other RNA
aggagggtccctatacagtagatg

SEQ ID NO: 163 - RNA, 21 nt, synthetic construct
  misc_feature 1..21: note = Synthetic
  source 1..21: mol_type = other RNA
gcttgagtcatggaaggaggg

SEQ ID NO: 164 - RNA, 22 nt, synthetic construct
  misc_feature 1..22: note = Synthetic
  source 1..22: mol_type = other RNA
agcttgagtcatggaaggaggg

SEQ ID NO: 165 - RNA, 20 nt, synthetic construct
  misc_feature 1..20: note = Synthetic
  source 1..20: mol_type = other RNA
gcttgagtcatggaaggagg

SEQ ID NO: 166 - RNA, 21 nt, synthetic construct
  misc_feature 1..21: note = Synthetic
  source 1..21: mol_type = other RNA
agcttgagtcatggaaggagg

SEQ ID NO: 167 - RNA, 24 nt, synthetic construct
  misc_feature 1..24: note = Synthetic
  source 1..24: mol_type = other RNA
caacacaatggctggaagctaagg

SEQ ID NO: 168 - RNA, 25 nt, synthetic construct
  misc_feature 1..25: note = Synthetic
  source 1..25: mol_type = other RNA
tcaacacaatggctggaagctaagg

SEQ ID NO: 169 - RNA, 20 nt, synthetic construct
  misc_feature 1..20: note = Synthetic
  source 1..20: mol_type = other RNA
agcttgagtcatggaaggag

SEQ ID NO: 170 - RNA, 21 nt, synthetic construct
  misc_feature 1..21: note = Synthetic
  source 1..21: mol_type = other RNA
atcagtgggatgaagtacaag

SEQ ID NO: 171 - RNA, 23 nt, synthetic construct
  misc_feature 1..23: note = Synthetic
  source 1..23: mol_type = other RNA
gaatcagtgggatgaagtacaag

SEQ ID NO: 172 - RNA, 20 nt, synthetic construct
  misc_feature 1..20: note = Synthetic
  source 1..20: mol_type = other RNA
tgagtcatggaaggagggtc

SEQ ID NO: 173 - RNA, 21 nt, synthetic construct
  misc_feature 1..21: note = Synthetic
  source 1..21: mol_type = other RNA
ttgagtcatggaaggagggtc

SEQ ID NO: 174 - RNA, 22 nt, synthetic construct
  misc_feature 1..22: note = Synthetic
  source 1..22: mol_type = other RNA
cttgagtcatggaaggagggtc

SEQ ID NO: 175 - RNA, 23 nt, synthetic construct
  misc_feature 1..23: note = Synthetic
  source 1..23: mol_type = other RNA
gcttgagtcatggaaggagggtc

SEQ ID NO: 176 - RNA, 25 nt, synthetic construct
  misc_feature 1..25: note = Synthetic
  source 1..25: mol_type = other RNA
gggtccctatacagtagatgcaatc

SEQ ID NO: 177 - RNA, 24 nt, synthetic construct
  misc_feature 1..24: note = Synthetic
  source 1..24: mol_type = other RNA
ggagggtccctatacagtagatgc

SEQ ID NO: 178 - RNA, 20 nt, synthetic construct
  misc_feature 1..20: note = Synthetic
  source 1..20: mol_type = other RNA
aagcatgggacacacaaagc

SEQ ID NO: 179 - RNA, 21 nt, synthetic construct
  misc_feature 1..21: note = Synthetic
  source 1..21: mol_type = other RNA
caagcatgggacacacaaagc

SEQ ID NO: 180 - RNA, 22 nt, synthetic construct
  misc_feature 1..22: note = Synthetic
  source 1..22: mol_type = other RNA
acaagcatgggacacacaaagc

SEQ ID NO: 181 - RNA, 23 nt, synthetic construct
  misc_feature 1..23: note = Synthetic
  source 1..23: mol_type = other RNA
aacaagcatgggacacacaaagc

SEQ ID NO: 182 - RNA, 24 nt, synthetic construct
  misc_feature 1..24: note = Synthetic
  source 1..24: mol_type = other RNA
taacaagcatgggacacacaaagc

SEQ ID NO: 183 - RNA, 20 nt, synthetic construct
  misc_feature 1..20: note = Synthetic
  source 1..20: mol_type = other RNA
gagtcatggaaggagggtcc

SEQ ID NO: 184 - RNA, 25 nt, synthetic construct
  misc_feature 1..25: note = Synthetic
  source 1..25: mol_type = other RNA
ggtccctatacagtagatgcaatcc

SEQ ID NO: 185 - RNA, 22 nt, synthetic construct
  misc_feature 1..22: note = Synthetic
  source 1..22: mol_type = other RNA
tgagtcatggaaggagggtccc

SEQ ID NO: 186 - RNA, 25 nt, synthetic construct
  misc_feature 1..25: note = Synthetic
  source 1..25: mol_type = other RNA
aagtttgtcctgaaaggtgggttac

SEQ ID NO: 187 - RNA, 20 nt, synthetic construct
  misc_feature 1..20: note = Synthetic
  source 1..20: mol_type = other RNA
gaatcagtgggatgaagtac

SEQ ID NO: 188 - RNA, 23 nt, synthetic construct
  misc_feature 1..23: note = Synthetic
  source 1..23: mol_type = other RNA
gttcatcatcctagccataacac

SEQ ID NO: 189 - RNA, 20 nt, synthetic construct
  misc_feature 1..20: note = Synthetic
  source 1..20: mol_type = other RNA
cagtgggatgaagtacaaga

SEQ ID NO: 190 - RNA, 21 nt, synthetic construct
  misc_feature 1..21: note = Synthetic
  source 1..21: mol_type = other RNA
tcagtgggatgaagtacaaga

SEQ ID NO: 191 - RNA, 22 nt, synthetic construct
  misc_feature 1..22: note = Synthetic
  source 1..22: mol_type = other RNA
atcagtgggatgaagtacaaga

SEQ ID NO: 192 - RNA, 23 nt, synthetic construct
  misc_feature 1..23: note = Synthetic
  source 1..23: mol_type = other RNA
aatcagtgggatgaagtacaaga

SEQ ID NO: 193 - RNA, 24 nt, synthetic construct
  misc_feature 1..24: note = Synthetic
  source 1..24: mol_type = other RNA
gaatcagtgggatgaagtacaaga

SEQ ID NO: 194 - RNA, 24 nt, synthetic construct
  misc_feature 1..24: note = Synthetic
  source 1..24: mol_type = other RNA
agaacaccttcagaaccggaggca

SEQ ID NO: 195 - RNA, 25 nt, synthetic construct
  misc_feature 1..25: note = Synthetic
  source 1..25: mol_type = other RNA
aagaacaccttcagaaccggaggca

SEQ ID NO: 196 - RNA, 20 nt, synthetic construct
  misc_feature 1..20: note = Synthetic
  source 1..20: mol_type = other RNA
agcatgggacacacaaagca

SEQ ID NO: 197 - RNA, 21 nt, synthetic construct
  misc_feature 1..21: note = Synthetic
  source 1..21: mol_type = other RNA
aagcatgggacacacaaagca

SEQ ID NO: 198 - RNA, 22 nt, synthetic construct
  misc_feature 1..22: note = Synthetic
  source 1..22: mol_type = other RNA
caagcatgggacacacaaagca

SEQ ID NO: 199 - RNA, 23 nt, synthetic construct
  misc_feature 1..23: note = Synthetic
  source 1..23: mol_type = other RNA
acaagcatgggacacacaaagca

SEQ ID NO: 200 - RNA, 24 nt, synthetic construct
  misc_feature 1..24: note = Synthetic
  source 1..24: mol_type = other RNA
aacaagcatgggacacacaaagca

SEQ ID NO: 201 - RNA, 25 nt, synthetic construct
  misc_feature 1..25: note = Synthetic
  source 1..25: mol_type = other RNA
taacaagcatgggacacacaaagca

SEQ ID NO: 202 - RNA, 21 nt, synthetic construct
  misc_feature 1..21: note = Synthetic
  source 1..21: mol_type = other RNA
gaatcagtgggatgaagtaca

SEQ ID NO: 203 - RNA, 20 nt, synthetic construct
  misc_feature 1..20: note = Synthetic
  source 1..20: mol_type = other RNA
taacaagcatgggacacaca

SEQ ID NO: 204 - RNA, 24 nt, synthetic construct
  misc_feature 1..24: note = Synthetic
  source 1..24: mol_type = other RNA
gttcatcatcctagccataacaca

SEQ ID NO: 205 - RNA, 22 nt, synthetic construct
  misc_feature 1..22: note = Synthetic
  source 1..22: mol_type = other RNA
agtgggatgaagtacaagaaca

SEQ ID NO: 206 - RNA, 23 nt, synthetic construct
  misc_feature 1..23: note = Synthetic
  source 1..23: mol_type = other RNA
cagtgggatgaagtacaagaaca

SEQ ID NO: 207 - RNA, 24 nt, synthetic construct
  misc_feature 1..24: note = Synthetic
  source 1..24: mol_type = other RNA
tcagtgggatgaagtacaagaaca

SEQ ID NO: 208 - RNA, 25 nt, synthetic construct
  misc_feature 1..25: note = Synthetic
  source 1..25: mol_type = other RNA
atcagtgggatgaagtacaagaaca

SEQ ID NO: 209 - RNA, 21 nt, synthetic construct
  misc_feature 1..21: note = Synthetic
  source 1..21: mol_type = other RNA
cagtgggatgaagtacaagaa

SEQ ID NO: 210 - RNA, 22 nt, synthetic construct
  misc_feature 1..22: note = Synthetic
  source 1..22: mol_type = other RNA
tcagtgggatgaagtacaagaa

SEQ ID NO: 211 - RNA, 23 nt, synthetic construct
  misc_feature 1..23: note = Synthetic
  source 1..23: mol_type = other RNA
atcagtgggatgaagtacaagaa

SEQ ID NO: 212 - RNA, 24 nt, synthetic construct
  misc_feature 1..24: note = Synthetic
  source 1..24: mol_type = other RNA
aatcagtgggatgaagtacaagaa

SEQ ID NO: 213 - RNA, 25 nt, synthetic construct
  misc_feature 1..25: note = Synthetic
  source 1..25: mol_type = other RNA
gaatcagtgggatgaagtacaagaa

SEQ ID NO: 214 - RNA, 22 nt, synthetic construct
  misc_feature 1..22: note = Synthetic
  source 1..22: mol_type = other RNA
gaatcagtgggatgaagtacaa

SEQ ID NO: 215 - RNA, 21 nt, synthetic construct
  misc_feature 1..21: note = Synthetic
  source 1..21: mol_type = other RNA
taacaagcatgggacacacaa

SEQ ID NO: 216 - RNA, 25 nt, synthetic construct
  misc_feature 1..25: note = Synthetic
  source 1..25: mol_type = other RNA
gttcatcatcctagccataacacaa

SEQ ID NO: 217 - RNA, 20 nt, synthetic construct
  misc_feature 1..20: note = Synthetic
  source 1..20: mol_type = other RNA
acaagcatgggacacacaaa

SEQ ID NO: 218 - RNA, 21 nt, synthetic construct
  misc_feature 1..21: note = Synthetic
  source 1..21: mol_type = other RNA
aacaagcatgggacacacaaa

SEQ ID NO: 219 - RNA, 22 nt, synthetic construct
  misc_feature 1..22: note = Synthetic
  source 1..22: mol_type = other RNA
taacaagcatgggacacacaaa

SEQ ID NO: 220 - RNA, 23 nt, synthetic construct
  misc_feature 1..23: note = Synthetic
  source 1..23: mol_type = other RNA
atgtctcctccagactagcattt

SEQ ID NO: 221 - RNA, 24 nt, synthetic construct
  misc_feature 1..24: note = Synthetic
  source 1..24: mol_type = other RNA
aatgtctcctccagactagcattt

SEQ ID NO: 222 - RNA, 25 nt, synthetic construct
  misc_feature 1..25: note = Synthetic
  source 1..25: mol_type = other RNA
aaatgtctcctccagactagcattt

SEQ ID NO: 223 - RNA, 24 nt, synthetic construct
  misc_feature 1..24: note = Synthetic
  source 1..24: mol_type = other RNA
aaagtttgtcctgaaaggtgggtt

SEQ ID NO: 224 - RNA, 24 nt, synthetic construct
  misc_feature 1..24: note = Synthetic
  source 1..24: mol_type = other RNA
ccttcagaaccggaggcaacagtt

SEQ ID NO: 225 - RNA, 22 nt, synthetic construct
  misc_feature 1..22: note = Synthetic
  source 1..22: mol_type = other RNA
gagtcatggaaggagggtccct

SEQ ID NO: 226 - RNA, 23 nt, synthetic construct
  misc_feature 1..23: note = Synthetic
  source 1..23: mol_type = other RNA
agtcatggaaggagggtccctat

SEQ ID NO: 227 - RNA, 22 nt, synthetic construct
  misc_feature 1..22: note = Synthetic
  source 1..22: mol_type = other RNA
ggagggtccctatacagtagat

SEQ ID NO: 228 - RNA, 23 nt, synthetic construct
  misc_feature 1..23: note = Synthetic
  source 1..23: mol_type = other RNA
aggagggtccctatacagtagat

SEQ ID NO: 229 - RNA, 21 nt, synthetic construct
  misc_feature 1..21: note = Synthetic
  source 1..21: mol_type = other RNA
accaaggttagtatcaaagat

SEQ ID NO: 230 - RNA, 22 nt, synthetic construct
  misc_feature 1..22: note = Synthetic
  source 1..22: mol_type = other RNA
aaccaaggttagtatcaaagat

SEQ ID NO: 231 - RNA, 24 nt, synthetic construct
  misc_feature 1..24: note = Synthetic
  source 1..24: mol_type = other RNA
tcatcatcctagccataacacaat

SEQ ID NO: 232 - RNA, 25 nt, synthetic construct
  misc_feature 1..25: note = Synthetic
  source 1..25: mol_type = other RNA
ccttcagaaccggaggcaacagttg

SEQ ID NO: 233 - RNA, 22 nt, synthetic construct
  misc_feature 1..22: note = Synthetic
  source 1..22: mol_type = other RNA
agaacaccttcagaaccggagg

SEQ ID NO: 234 - RNA, 23 nt, synthetic construct
  misc_feature 1..23: note = Synthetic
  source 1..23: mol_type = other RNA
aagaacaccttcagaaccggagg

SEQ ID NO: 235 - RNA, 24 nt, synthetic construct
  misc_feature 1..24: note = Synthetic
  source 1..24: mol_type = other RNA
caagaacaccttcagaaccggagg

SEQ ID NO: 236 - RNA, 22 nt, synthetic construct
  misc_feature 1..22: note = Synthetic
  source 1..22: mol_type = other RNA
acacaatggctggaagctaagg

SEQ ID NO: 237 - RNA, 23 nt, synthetic construct
  misc_feature 1..23: note = Synthetic
  source 1..23: mol_type = other RNA
aacacaatggctggaagctaagg

SEQ ID NO: 238 - RNA, 23 nt, synthetic construct
  misc_feature 1..23: note = Synthetic
  source 1..23: mol_type = other RNA
caagaacaccttcagaaccggag

SEQ ID NO: 239 - RNA, 25 nt, synthetic construct
  misc_feature 1..25: note = Synthetic
  source 1..25: mol_type = other RNA
tacaagaacaccttcagaaccggag

SEQ ID NO: 240 - RNA, 23 nt, synthetic construct
  misc_feature 1..23: note = Synthetic
  source 1..23: mol_type = other RNA
caacacaatggctggaagctaag

SEQ ID NO: 241 - RNA, 24 nt, synthetic construct
  misc_feature 1..24: note = Synthetic
  source 1..24: mol_type = other RNA
tcaacacaatggctggaagctaag

SEQ ID NO: 242 - RNA, 20 nt, synthetic construct
  misc_feature 1..20: note = Synthetic
  source 1..20: mol_type = other RNA
tcagtgggatgaagtacaag

SEQ ID NO: 243 - RNA, 22 nt, synthetic construct
  misc_feature 1..22: note = Synthetic
  source 1..22: mol_type = other RNA
aatcagtgggatgaagtacaag

SEQ ID NO: 244 - RNA, 23 nt, synthetic construct
  misc_feature 1..23: note = Synthetic
  source 1..23: mol_type = other RNA
atacagtagatgcaatccaaaag

SEQ ID NO: 245 - RNA, 24 nt, synthetic construct
  misc_feature 1..24: note = Synthetic
  source 1..24: mol_type = other RNA
ggtccctatacagtagatgcaatc

SEQ ID NO: 246 - RNA, 23 nt, synthetic construct
  misc_feature 1..23: note = Synthetic
  source 1..23: mol_type = other RNA
gagggtccctatacagtagatgc

SEQ ID NO: 247 - RNA, 20 nt, synthetic construct
  misc_feature 1..20: note = Synthetic
  source 1..20: mol_type = other RNA
aatgtctcctccagactagc

SEQ ID NO: 248 - RNA, 21 nt, synthetic construct
  misc_feature 1..21: note = Synthetic
  source 1..21: mol_type = other RNA
aaatgtctcctccagactagc

SEQ ID NO: 249 - RNA, 22 nt, synthetic construct
  misc_feature 1..22: note = Synthetic
  source 1..22: mol_type = other RNA
aaaatgtctcctccagactagc

SEQ ID NO: 250 - RNA, 23 nt, synthetic construct
  misc_feature 1..23: note = Synthetic
  source 1..23: mol_type = other RNA
taaaatgtctcctccagactagc

SEQ ID NO: 251 - RNA, 21 nt, synthetic construct
  misc_feature 1..21: note = Synthetic
  source 1..21: mol_type = other RNA
tgagtcatggaaggagggtcc

SEQ ID NO: 252 - RNA, 22 nt, synthetic construct
  misc_feature 1..22: note = Synthetic
  source 1..22: mol_type = other RNA
ttgagtcatggaaggagggtcc

SEQ ID NO: 253 - RNA, 24 nt, synthetic construct
  misc_feature 1..24: note = Synthetic
  source 1..24: mol_type = other RNA
gtccctatacagtagatgcaatcc

SEQ ID NO: 254 - RNA, 20 nt, synthetic construct
  misc_feature 1..20: note = Synthetic
  source 1..20: mol_type = other RNA
agtcatggaaggagggtccc

SEQ ID NO: 255 - RNA, 23 nt, synthetic construct
  misc_feature 1..23: note = Synthetic
  source 1..23: mol_type = other RNA
ttgagtcatggaaggagggtccc

SEQ ID NO: 256 - RNA, 20 nt, synthetic construct
  misc_feature 1..20: note = Synthetic
  source 1..20: mol_type = other RNA
aacttaagttcatatatccc

SEQ ID NO: 257 - RNA, 25 nt, synthetic construct
  misc_feature 1..25: note = Synthetic
  source 1..25: mol_type = other RNA
agtttgtcctgaaaggtgggttacc

SEQ ID NO: 258 - RNA, 24 nt, synthetic construct
  misc_feature 1..24: note = Synthetic
  source 1..24: mol_type = other RNA
agtttgtcctgaaaggtgggttac

SEQ ID NO: 259 - RNA, 21 nt, synthetic construct
  misc_feature 1..21: note = Synthetic
  source 1..21: mol_type = other RNA
tcatcatcctagccataacac

SEQ ID NO: 260 - RNA, 22 nt, synthetic construct
  misc_feature 1..22: note = Synthetic
  source 1..22: mol_type = other RNA
ttcatcatcctagccataacac

SEQ ID NO: 261 - RNA, 21 nt, synthetic construct
  misc_feature 1..21: note = Synthetic
  source 1..21: mol_type = other RNA
gttcatcatcctagccataac

SEQ ID NO: 262 - RNA, 24 nt, synthetic construct
  misc_feature 1..24: note = Synthetic
  source 1..24: mol_type = other RNA
atgtctcctccagactagcattta

SEQ ID NO: 263 - RNA, 25 nt, synthetic construct
  misc_feature 1..25: note = Synthetic
  source 1..25: mol_type = other RNA
aatgtctcctccagactagcattta

SEQ ID NO: 264 - RNA, 24 nt, synthetic construct
  misc_feature 1..24: note = Synthetic
  source 1..24: mol_type = other RNA
aagtttgtcctgaaaggtgggtta

SEQ ID NO: 265 - RNA, 25 nt, synthetic construct
  misc_feature 1..25: note = Synthetic
  source 1..25: mol_type = other RNA
aaagtttgtcctgaaaggtgggtta

SEQ ID NO: 266 - RNA, 23 nt, synthetic construct
  misc_feature 1..23: note = Synthetic
  source 1..23: mol_type = other RNA
gagtcatggaaggagggtcccta

SEQ ID NO: 267 - RNA, 22 nt, synthetic construct
  misc_feature 1..22: note = Synthetic
  source 1..22: mol_type = other RNA
accaaggttagtatcaaagata

SEQ ID NO: 268 - RNA, 23 nt, synthetic construct
  misc_feature 1..23: note = Synthetic
  source 1..23: mol_type = other RNA
tacagtagatgcaatccaaaaga

SEQ ID NO: 269 - RNA, 25 nt, synthetic construct
  misc_feature 1..25: note = Synthetic
  source 1..25: mol_type = other RNA
gtccctatacagtagatgcaatcca

SEQ ID NO: 270 - RNA, 20 nt, synthetic construct
  misc_feature 1..20: note = Synthetic
  source 1..20: mol_type = other RNA
aatcagtgggatgaagtaca

SEQ ID NO: 271 - RNA, 21 nt, synthetic construct
  misc_feature 1..21: note = Synthetic
  source 1..21: mol_type = other RNA
catcatcctagccataacaca

SEQ ID NO: 272 - RNA, 22 nt, synthetic construct
  misc_feature 1..22: note = Synthetic
  source 1..22: mol_type = other RNA
tcatcatcctagccataacaca

SEQ ID NO: 273 - RNA, 23 nt, synthetic construct
  misc_feature 1..23: note = Synthetic
  source 1..23: mol_type = other RNA
ttcatcatcctagccataacaca

SEQ ID NO: 274 - RNA, 21 nt, synthetic construct
  misc_feature 1..21: note = Synthetic
  source 1..21: mol_type = other RNA
gtgggatgaagtacaagaaca

SEQ ID NO: 275 - RNA, 20 nt, synthetic construct
  misc_feature 1..20: note = Synthetic
  source 1..20: mol_type = other RNA
agtgggatgaagtacaagaa

SEQ ID NO: 276 - RNA, 20 nt, synthetic construct
  misc_feature 1..20: note = Synthetic
  source 1..20: mol_type = other RNA
atcagtgggatgaagtacaa

SEQ ID NO: 277 - RNA, 21 nt, synthetic construct
  misc_feature 1..21: note = Synthetic
  source 1..21: mol_type = other RNA
aatcagtgggatgaagtacaa

SEQ ID NO: 278 - RNA, 20 nt, synthetic construct
  misc_feature 1..20: note = Synthetic
  source 1..20: mol_type = other RNA
aacaagcatgggacacacaa

SEQ ID NO: 279 - RNA, 23 nt, synthetic construct
  misc_feature 1..23: note = Synthetic
  source 1..23: mol_type = other RNA
tcatcatcctagccataacacaa

SEQ ID NO: 280 - RNA, 23 nt, synthetic construct
  misc_feature 1..23: note = Synthetic
  source 1..23: mol_type = other RNA
aagtttgtcctgaaaggtgggtt

SEQ ID NO: 281 - RNA, 25 nt, synthetic construct
  misc_feature 1..25: note = Synthetic
  source 1..25: mol_type = other RNA
aaaagtttgtcctgaaaggtgggtt

SEQ ID NO: 282 - RNA, 25 nt, synthetic construct
  misc_feature 1..25: note = Synthetic
  source 1..25: mol_type = other RNA
cctagccataacacaatgaataatt

SEQ ID NO: 283 - RNA, 20 nt, synthetic construct
  misc_feature 1..20: note = Synthetic
  source 1..20: mol_type = other RNA
aaggattcaacacaatggct

SEQ ID NO: 284 - RNA, 22 nt, synthetic construct
  misc_feature 1..22: note = Synthetic
  source 1..22: mol_type = other RNA
taaaggattcaacacaatggct

SEQ ID NO: 285 - RNA, 25 nt, synthetic construct
  misc_feature 1..25: note = Synthetic
  source 1..25: mol_type = other RNA
accaaggttagtatcaaagatacct

SEQ ID NO: 286 - RNA, 22 nt, synthetic construct
  misc_feature 1..22: note = Synthetic
  source 1..22: mol_type = other RNA
gtcatggaaggagggtccctat

SEQ ID NO: 287 - RNA, 24 nt, synthetic construct
  misc_feature 1..24: note = Synthetic
  source 1..24: mol_type = other RNA
atatatgtattctgacctgaggat

SEQ ID NO: 288 - RNA, 23 nt, synthetic construct
  misc_feature 1..23: note = Synthetic
  source 1..23: mol_type = other RNA
aaaccaaggttagtatcaaagat

SEQ ID NO: 289 - RNA, 23 nt, synthetic construct
  misc_feature 1..23: note = Synthetic
  source 1..23: mol_type = other RNA
ctagccataacacaatgaataat

SEQ ID NO: 290 - RNA, 24 nt, synthetic construct
  misc_feature 1..24: note = Synthetic
  source 1..24: mol_type = other RNA
cctagccataacacaatgaataat

SEQ ID NO: 291 - RNA, 25 nt, synthetic construct
  misc_feature 1..25: note = Synthetic
  source 1..25: mol_type = other RNA
tcctagccataacacaatgaataat

SEQ ID NO: 292 - RNA, 25 nt, synthetic construct
  misc_feature 1..25: note = Synthetic
  source 1..25: mol_type = other RNA
ttcatcatcctagccataacacaat

SEQ ID NO: 293 - RNA, 24 nt, synthetic construct
  misc_feature 1..24: note = Synthetic
  source 1..24: mol_type = other RNA
agctgaaatgaacagtagactttg

SEQ ID NO: 294 - RNA, 23 nt, synthetic construct
  misc_feature 1..23: note = Synthetic
  source 1..23: mol_type = other RNA
taaaggattcaacacaatggctg

SEQ ID NO: 295 - RNA, 22 nt, synthetic construct
  misc_feature 1..22: note = Synthetic
  source 1..22: mol_type = other RNA
gagggtccctatacagtagatg

SEQ ID NO: 296 - RNA, 24 nt, synthetic construct
  misc_feature 1..24: note = Synthetic
  source 1..24: mol_type = other RNA
tgaaaagtttgtcctgaaaggtgg

SEQ ID NO: 297 - RNA, 21 nt, synthetic construct
  misc_feature 1..21: note = Synthetic
  source 1..21: mol_type = other RNA
aggattcaacacaatggctgg

SEQ ID NO: 298 - RNA, 22 nt, synthetic construct
  misc_feature 1..22: note = Synthetic
  source 1..22: mol_type = other RNA
aaggattcaacacaatggctgg

SEQ ID NO: 299 - RNA, 23 nt, synthetic construct
  misc_feature 1..23: note = Synthetic
  source 1..23: mol_type = other RNA
aaaggattcaacacaatggctgg

SEQ ID NO: 300 - RNA, 24 nt, synthetic construct
  misc_feature 1..24: note = Synthetic
  source 1..24: mol_type = other RNA
taaaggattcaacacaatggctgg

SEQ ID NO: 301 - RNA, 24 nt, synthetic construct
  misc_feature 1..24: note = Synthetic
  source 1..24: mol_type = other RNA
gaaaagtttgtcctgaaaggtggg

SEQ ID NO: 302 - RNA, 25 nt, synthetic construct
  misc_feature 1..25: note = Synthetic
  source 1..25: mol_type = other RNA
tgaaaagtttgtcctgaaaggtggg

SEQ ID NO: 303 - RNA, 20 nt, synthetic construct
  misc_feature 1..20: note = Synthetic
  source 1..20: mol_type = other RNA
cttgagtcatggaaggaggg

SEQ ID NO: 304 - RNA, 21 nt, synthetic construct
  misc_feature 1..21: note = Synthetic
  source 1..21: mol_type = other RNA
agaacaccttcagaaccggag

SEQ ID NO: 305 - RNA, 22 nt, synthetic construct
  misc_feature 1..22: note = Synthetic
  source 1..22: mol_type = other RNA
aagaacaccttcagaaccggag

SEQ ID NO: 306 - RNA, 23 nt, synthetic construct
  misc_feature 1..23: note = Synthetic
  source 1..23: mol_type = other RNA
ggattcaacacaatggctggaag

SEQ ID NO: 307 - RNA, 24 nt, synthetic construct
  misc_feature 1..24: note = Synthetic
  source 1..24: mol_type = other RNA
aggattcaacacaatggctggaag

SEQ ID NO: 308 - RNA, 25 nt, synthetic construct
  misc_feature 1..25: note = Synthetic
  source 1..25: mol_type = other RNA
aaggattcaacacaatggctggaag

SEQ ID NO: 309 - RNA, 24 nt, synthetic construct
  misc_feature 1..24: note = Synthetic
  source 1..24: mol_type = other RNA
agcttgagtcatggaaggagggtc

SEQ ID NO: 310 - RNA, 21 nt, synthetic construct
  misc_feature 1..21: note = Synthetic
  source 1..21: mol_type = other RNA
gggtccctatacagtagatgc

SEQ ID NO: 311 - RNA, 21 nt, synthetic construct
  misc_feature 1..21: note = Synthetic
  source 1..21: mol_type = other RNA
taaaggattcaacacaatggc

SEQ ID NO: 312 - RNA, 24 nt, synthetic construct
  misc_feature 1..24: note = Synthetic
  source 1..24: mol_type = other RNA
ttaaaatgtctcctccagactagc

SEQ ID NO: 313 - RNA, 23 nt, synthetic construct
  misc_feature 1..23: note = Synthetic
  source 1..23: mol_type = other RNA
gattcaacacaatggctggaagc

SEQ ID NO: 314 - RNA, 24 nt, synthetic construct
  misc_feature 1..24: note = Synthetic
  source 1..24: mol_type = other RNA
ggattcaacacaatggctggaagc

SEQ ID NO: 315 - RNA, 25 nt, synthetic construct
  misc_feature 1..25: note = Synthetic
  source 1..25: mol_type = other RNA
aggattcaacacaatggctggaagc

SEQ ID NO: 316 - RNA, 23 nt, synthetic construct
  misc_feature 1..23: note = Synthetic
  source 1..23: mol_type = other RNA
cttgagtcatggaaggagggtcc

SEQ ID NO: 317 - RNA, 24 nt, synthetic construct
  misc_feature 1..24: note = Synthetic
  source 1..24: mol_type = other RNA
gcttgagtcatggaaggagggtcc

SEQ ID NO: 318 - RNA, 25 nt, synthetic construct
  misc_feature 1..25: note = Synthetic
  source 1..25: mol_type = other RNA
agcttgagtcatggaaggagggtcc

SEQ ID NO: 319 - RNA, 24 nt, synthetic construct
  misc_feature 1..24: note = Synthetic
  source 1..24: mol_type = other RNA
cttgagtcatggaaggagggtccc

SEQ ID NO: 320 - RNA, 25 nt, synthetic construct
  misc_feature 1..25: note = Synthetic
  source 1..25: mol_type = other RNA
gcttgagtcatggaaggagggtccc

SEQ ID NO: 321 - RNA, 23 nt, synthetic construct
  misc_feature 1..23: note = Synthetic
  source 1..23: mol_type = other RNA
gtctcctccagactagcatttac

SEQ ID NO: 322 - RNA, 25 nt, synthetic construct
  misc_feature 1..25: note = Synthetic
  source 1..25: mol_type = other RNA
gtaagttttttaacaagcatgggac

SEQ ID NO: 323 - RNA, 20 nt, synthetic construct
  misc_feature 1..20: note = Synthetic
  source 1..20: mol_type = other RNA
agctgaaatgaacagtagac

SEQ ID NO: 324 - RNA, 20 nt, synthetic construct
  misc_feature 1..20: note = Synthetic
  source 1..20: mol_type = other RNA
catcatcctagccataacac

SEQ ID NO: 325 - RNA, 25 nt, synthetic construct
  misc_feature 1..25: note = Synthetic
  source 1..25: mol_type = other RNA
gattcaacacaatggctggaagcta

SEQ ID NO: 326 - RNA, 23 nt, synthetic construct
  misc_feature 1..23: note = Synthetic
  source 1..23: mol_type = other RNA
aaccaaggttagtatcaaagata

SEQ ID NO: 327 - RNA, 22 nt, synthetic construct
  misc_feature 1..22: note = Synthetic
  source 1..22: mol_type = other RNA
aggattcaacacaatggctgga

SEQ ID NO: 328 - RNA, 23 nt, synthetic construct
  misc_feature 1..23: note = Synthetic
  source 1..23: mol_type = other RNA
aaggattcaacacaatggctgga

SEQ ID NO: 329 - RNA, 24 nt, synthetic construct
  misc_feature 1..24: note = Synthetic
  source 1..24: mol_type = other RNA
aaaggattcaacacaatggctgga

SEQ ID NO: 330 - RNA, 25 nt, synthetic construct
  misc_feature 1..25: note = Synthetic
  source 1..25: mol_type = other RNA
taaaggattcaacacaatggctgga

SEQ ID NO: 331 - RNA, 23 nt, synthetic construct
  misc_feature 1..23: note = Synthetic
  source 1..23: mol_type = other RNA
atatatgtattctgacctgagga

SEQ ID NO: 332 - RNA, 23 nt, synthetic construct
  misc_feature 1..23: note = Synthetic
  source 1..23: mol_type = other RNA
gaacaccttcagaaccggaggca

SEQ ID NO: 333 - RNA, 20 nt, synthetic construct
  misc_feature 1..20: note = Synthetic
  source 1..20: mol_type = other RNA
gttcatcatcctagccataa

SEQ ID NO: 334 - RNA, 24 nt, synthetic construct
  misc_feature 1..24: note = Synthetic
  source 1..24: mol_type = other RNA
ttcatcatcctagccataacacaa

SEQ ID NO: 335 - RNA, 24 nt, synthetic construct
  misc_feature 1..24: note = Synthetic
  source 1..24: mol_type = other RNA
atagggaccctccttccatgactc

SEQ ID NO: 336 - RNA, 23 nt, synthetic construct
  misc_feature 1..23: note = Synthetic
  source 1..23: mol_type = other RNA
catctactgtatagggaccctcc

SEQ ID NO: 337 - RNA, 24 nt, synthetic construct
  misc_feature 1..24: note = Synthetic
  source 1..24: mol_type = other RNA
catctactgtatagggaccctcct

SEQ ID NO: 338 - RNA, 21 nt, synthetic construct
  misc_feature 1..21: note = Synthetic
  source 1..21: mol_type = other RNA
ccctccttccatgactcaagc

SEQ ID NO: 339 - RNA, 22 nt, synthetic construct
  misc_feature 1..22: note = Synthetic
  source 1..22: mol_type = other RNA
ccctccttccatgactcaagct

SEQ ID NO: 340 - RNA, 20 nt, synthetic construct
  misc_feature 1..20: note = Synthetic
  source 1..20: mol_type = other RNA
cctccttccatgactcaagc

SEQ ID NO: 341 - RNA, 21 nt, synthetic construct
  misc_feature 1..21: note = Synthetic
  source 1..21: mol_type = other RNA
cctccttccatgactcaagct

SEQ ID NO: 342 - RNA, 24 nt, synthetic construct
  misc_feature 1..24: note = Synthetic
  source 1..24: mol_type = other RNA
ccttagcttccagccattgtgttg

SEQ ID NO: 343 - RNA, 25 nt, synthetic construct
  misc_feature 1..25: note = Synthetic
  source 1..25: mol_type = other RNA
ccttagcttccagccattgtgttga

SEQ ID NO: 344 - RNA, 20 nt, synthetic construct
  misc_feature 1..20: note = Synthetic
  source 1..20: mol_type = other RNA
ctccttccatgactcaagct

SEQ ID NO: 345 - RNA, 21 nt, synthetic construct
  misc_feature 1..21: note = Synthetic
  source 1..21: mol_type = other RNA
cttgtacttcatcccactgat

SEQ ID NO: 346 - RNA, 23 nt, synthetic construct
  misc_feature 1..23: note = Synthetic
  source 1..23: mol_type = other RNA
cttgtacttcatcccactgattc

SEQ ID NO: 347 - RNA, 20 nt, synthetic construct
  misc_feature 1..20: note = Synthetic
  source 1..20: mol_type = other RNA
gaccctccttccatgactca

SEQ ID NO: 348 - RNA, 21 nt, synthetic construct
  misc_feature 1..21: note = Synthetic
  source 1..21: mol_type = other RNA
gaccctccttccatgactcaa

SEQ ID NO: 349 - RNA, 22 nt, synthetic construct
  misc_feature 1..22: note = Synthetic
  source 1..22: mol_type = other RNA
gaccctccttccatgactcaag

SEQ ID NO: 350 - RNA, 23 nt, synthetic construct
  misc_feature 1..23: note = Synthetic
  source 1..23: mol_type = other RNA
gaccctccttccatgactcaagc

SEQ ID NO: 351 - RNA, 25 nt, synthetic construct
  misc_feature 1..25: note = Synthetic
  source 1..25: mol_type = other RNA
gattgcatctactgtatagggaccc

SEQ ID NO: 352 - RNA, 24 nt, synthetic construct
  misc_feature 1..24: note = Synthetic
  source 1..24: mol_type = other RNA
gcatctactgtatagggaccctcc

SEQ ID NO: 353 - RNA, 20 nt, synthetic construct
  misc_feature 1..20: note = Synthetic
  source 1..20: mol_type = other RNA
gctttgtgtgtcccatgctt

SEQ ID NO: 354 - RNA, 21 nt, synthetic construct
  misc_feature 1..21: note = Synthetic
  source 1..21: mol_type = other RNA
gctttgtgtgtcccatgcttg

SEQ ID NO: 355 - RNA, 22 nt, synthetic construct
  misc_feature 1..22: note = Synthetic
  source 1..22: mol_type = other RNA
gctttgtgtgtcccatgcttgt

SEQ ID NO: 356 - RNA, 23 nt, synthetic construct
  misc_feature 1..23: note = Synthetic
  source 1..23: mol_type = other RNA
gctttgtgtgtcccatgcttgtt

SEQ ID NO: 357 - RNA, 24 nt, synthetic construct
  misc_feature 1..24: note = Synthetic
  source 1..24: mol_type = other RNA
gctttgtgtgtcccatgcttgtta

SEQ ID NO: 358 - RNA, 20 nt, synthetic construct
  misc_feature 1..20: note = Synthetic
  source 1..20: mol_type = other RNA
ggaccctccttccatgactc

SEQ ID NO: 359 - RNA, 25 nt, synthetic construct
  misc_feature 1..25: note = Synthetic
  source 1..25: mol_type = other RNA
ggattgcatctactgtatagggacc

SEQ ID NO: 360 - RNA, 22 nt, synthetic construct
  misc_feature 1..22: note = Synthetic
  source 1..22: mol_type = other RNA
gggaccctccttccatgactca

SEQ ID NO: 361 - RNA, 25 nt, synthetic construct
  misc_feature 1..25: note = Synthetic
  source 1..25: mol_type = other RNA
gtaacccacctttcaggacaaactt

SEQ ID NO: 362 - RNA, 20 nt, synthetic construct
  misc_feature 1..20: note = Synthetic
  source 1..20: mol_type = other RNA
gtacttcatcccactgattc

SEQ ID NO: 363 - RNA, 23 nt, synthetic construct
  misc_feature 1..23: note = Synthetic
  source 1..23: mol_type = other RNA
gtgttatggctaggatgatgaac

SEQ ID NO: 364 - RNA, 20 nt, synthetic construct
  misc_feature 1..20: note = Synthetic
  source 1..20: mol_type = other RNA
tcttgtacttcatcccactg

SEQ ID NO: 365 - RNA, 21 nt, synthetic construct
  misc_feature 1..21: note = Synthetic
  source 1..21: mol_type = other RNA
tcttgtacttcatcccactga

SEQ ID NO: 366 - RNA, 22 nt, synthetic construct
  misc_feature 1..22: note = Synthetic
  source 1..22: mol_type = other RNA
tcttgtacttcatcccactgat

SEQ ID NO: 367 - RNA, 23 nt, synthetic construct
  misc_feature 1..23: note = Synthetic
  source 1..23: mol_type = other RNA
tcttgtacttcatcccactgatt

SEQ ID NO: 368 - RNA, 24 nt, synthetic construct
  misc_feature 1..24: note = Synthetic
  source 1..24: mol_type = other RNA
tcttgtacttcatcccactgattc

SEQ ID NO: 369 - RNA, 24 nt, synthetic construct
  misc_feature 1..24: note = Synthetic
  source 1..24: mol_type = other RNA
tgcctccggttctgaaggtgttct

SEQ ID NO: 370 - RNA, 25 nt, synthetic construct
  misc_feature 1..25: note = Synthetic
  source 1..25: mol_type = other RNA
tgcctccggttctgaaggtgttctt

SEQ ID NO: 371 - RNA, 20 nt, synthetic construct
  misc_feature 1..20: note = Synthetic
  source 1..20: mol_type = other RNA
tgctttgtgtgtcccatgct

SEQ ID NO: 372 - RNA, 21 nt, synthetic construct
  misc_feature 1..21: note = Synthetic
  source 1..21: mol_type = other RNA
tgctttgtgtgtcccatgctt

SEQ ID NO: 373 - RNA, 22 nt, synthetic construct
  misc_feature 1..22: note = Synthetic
  source 1..22: mol_type = other RNA
tgctttgtgtgtcccatgcttg

SEQ ID NO: 374 - RNA, 23 nt, synthetic construct
  misc_feature 1..23: note = Synthetic
  source 1..23: mol_type = other RNA
tgctttgtgtgtcccatgcttgt

SEQ ID NO: 375 - RNA, 24 nt, synthetic construct
  misc_feature 1..24: note = Synthetic
  source 1..24: mol_type = other RNA
tgctttgtgtgtcccatgcttgtt

SEQ ID NO: 376 - RNA, 25 nt, synthetic construct
  misc_feature 1..25: note = Synthetic
  source 1..25: mol_type = other RNA
tgctttgtgtgtcccatgcttgtta

SEQ ID NO: 377 - RNA, 21 nt, synthetic construct
  misc_feature 1..21: note = Synthetic
  source 1..21: mol_type = other RNA
tgtacttcatcccactgattc

SEQ ID NO: 378 - RNA, 20 nt, synthetic construct
  misc_feature 1..20: note = Synthetic
  source 1..20: mol_type = other RNA
tgtgtgtcccatgcttgtta

SEQ ID NO: 379 - RNA, 24 nt, synthetic construct
  misc_feature 1..24: note = Synthetic
  source 1..24: mol_type = other RNA
tgtgttatggctaggatgatgaac

SEQ ID NO: 380 - RNA, 22 nt, synthetic construct
  misc_feature 1..22: note = Synthetic
  source 1..22: mol_type = other RNA
tgttcttgtacttcatcccact

SEQ ID NO: 381 - RNA, 23 nt, synthetic construct
  misc_feature 1..23: note = Synthetic
  source 1..23: mol_type = other RNA
tgttcttgtacttcatcccactg

SEQ ID NO: 382 - RNA, 24 nt, synthetic construct
  misc_feature 1..24: note = Synthetic
  source 1..24: mol_type = other RNA
tgttcttgtacttcatcccactga

SEQ ID NO: 383 - RNA, 25 nt, synthetic construct
  misc_feature 1..25: note = Synthetic
  source 1..25: mol_type = other RNA
tgttcttgtacttcatcccactgat

SEQ ID NO: 384 - RNA, 21 nt, synthetic construct
  misc_feature 1..21: note = Synthetic
  source 1..21: mol_type = other RNA
ttcttgtacttcatcccactg

SEQ ID NO: 385 - RNA, 22 nt, synthetic construct
  misc_feature 1..22: note = Synthetic
  source 1..22: mol_type = other RNA
ttcttgtacttcatcccactga

SEQ ID NO: 386 - RNA, 23 nt, synthetic construct
  misc_feature 1..23: note = Synthetic
  source 1..23: mol_type = other RNA
ttcttgtacttcatcccactgat

SEQ ID NO: 387 - RNA, 24 nt, synthetic construct
  misc_feature 1..24: note = Synthetic
  source 1..24: mol_type = other RNA
ttcttgtacttcatcccactgatt

SEQ ID NO: 388 - RNA, 25 nt, synthetic construct
  misc_feature 1..25: note = Synthetic
  source 1..25: mol_type = other RNA
ttcttgtacttcatcccactgattc

SEQ ID NO: 389 - RNA, 22 nt, synthetic construct
  misc_feature 1..22: note = Synthetic
  source 1..22: mol_type = other RNA
ttgtacttcatcccactgattc

SEQ ID NO: 390 - RNA, 21 nt, synthetic construct
  misc_feature 1..21: note = Synthetic
  source 1..21: mol_type = other RNA
ttgtgtgtcccatgcttgtta

SEQ ID NO: 391 - RNA, 25 nt, synthetic construct
  misc_feature 1..25: note = Synthetic
  source 1..25: mol_type = other RNA
ttgtgttatggctaggatgatgaac

SEQ ID NO: 392 - RNA, 20 nt, synthetic construct
  misc_feature 1..20: note = Synthetic
  source 1..20: mol_type = other RNA
tttgtgtgtcccatgcttgt

SEQ ID NO: 393 - RNA, 21 nt, synthetic construct
  misc_feature 1..21: note = Synthetic
  source 1..21: mol_type = other RNA
tttgtgtgtcccatgcttgtt

SEQ ID NO: 394 - RNA, 22 nt, synthetic construct
  misc_feature 1..22: note = Synthetic
  source 1..22: mol_type = other RNA
tttgtgtgtcccatgcttgtta

SEQ ID NO: 395 - RNA, 23 nt, synthetic construct
  misc_feature 1..23: note = Synthetic
  source 1..23: mol_type = other RNA
aaatgctagtctggaggagacat

SEQ ID NO: 396 - RNA, 24 nt, synthetic construct
  misc_feature 1..24: note = Synthetic
  source 1..24: mol_type = other RNA
aaatgctagtctggaggagacatt

SEQ ID NO: 397 - RNA, 25 nt, synthetic construct
  misc_feature 1..25: note = Synthetic
  source 1..25: mol_type = other RNA
aaatgctagtctggaggagacattt

SEQ ID NO: 398 - RNA, 24 nt, synthetic construct
  misc_feature 1..24: note = Synthetic
  source 1..24: mol_type = other RNA
aacccacctttcaggacaaacttt

SEQ ID NO: 399 - RNA, 24 nt, synthetic construct
  misc_feature 1..24: note = Synthetic
  source 1..24: mol_type = other RNA
aactgttgcctccggttctgaagg

SEQ ID NO: 400 - RNA, 22 nt, synthetic construct
  misc_feature 1..22: note = Synthetic
  source 1..22: mol_type = other RNA
agggaccctccttccatgactc

SEQ ID NO: 401 - RNA, 23 nt, synthetic construct
  misc_feature 1..23: note = Synthetic
  source 1..23: mol_type = other RNA
atagggaccctccttccatgact

SEQ ID NO: 402 - RNA, 22 nt, synthetic construct
  misc_feature 1..22: note = Synthetic
  source 1..22: mol_type = other RNA
atctactgtatagggaccctcc

SEQ ID NO: 403 - RNA, 23 nt, synthetic construct
  misc_feature 1..23: note = Synthetic
  source 1..23: mol_type = other RNA
atctactgtatagggaccctcct

SEQ ID NO: 404 - RNA, 21 nt, synthetic construct
  misc_feature 1..21: note = Synthetic
  source 1..21: mol_type = other RNA
atctttgatactaaccttggt

SEQ ID NO: 405 - RNA, 22 nt, synthetic construct
  misc_feature 1..22: note = Synthetic
  source 1..22: mol_type = other RNA
atctttgatactaaccttggtt

SEQ ID NO: 406 - RNA, 24 nt, synthetic construct
  misc_feature 1..24: note = Synthetic
  source 1..24: mol_type = other RNA
attgtgttatggctaggatgatga

SEQ ID NO: 407 - RNA, 25 nt, synthetic construct
  misc_feature 1..25: note = Synthetic
  source 1..25: mol_type = other RNA
caactgttgcctccggttctgaagg

SEQ ID NO: 408 - RNA, 22 nt, synthetic construct
  misc_feature 1..22: note = Synthetic
  source 1..22: mol_type = other RNA
cctccggttctgaaggtgttct

SEQ ID NO: 409 - RNA, 23 nt, synthetic construct
  misc_feature 1..23: note = Synthetic
  source 1..23: mol_type = other RNA
cctccggttctgaaggtgttctt

SEQ ID NO: 410 - RNA, 24 nt, synthetic construct
  misc_feature 1..24: note = Synthetic
  source 1..24: mol_type = other RNA
cctccggttctgaaggtgttcttg

SEQ ID NO: 411 - RNA, 22 nt, synthetic construct
  misc_feature 1..22: note = Synthetic
  source 1..22: mol_type = other RNA
ccttagcttccagccattgtgt

SEQ ID NO: 412 - RNA, 23 nt, synthetic construct
  misc_feature 1..23: note = Synthetic
  source 1..23: mol_type = other RNA
ccttagcttccagccattgtgtt

SEQ ID NO: 413 - RNA, 23 nt, synthetic construct
  misc_feature 1..23: note = Synthetic
  source 1..23: mol_type = other RNA
ctccggttctgaaggtgttcttg

SEQ ID NO: 414 - RNA, 25 nt, synthetic construct
  misc_feature 1..25: note = Synthetic
  source 1..25: mol_type = other RNA
ctccggttctgaaggtgttcttgta

SEQ ID NO: 415 - RNA, 23 nt, synthetic construct
  misc_feature 1..23: note = Synthetic
  source 1..23: mol_type = other RNA
cttagcttccagccattgtgttg

SEQ ID NO: 416 - RNA, 24 nt, synthetic construct
  misc_feature 1..24: note = Synthetic
  source 1..24: mol_type = other RNA
cttagcttccagccattgtgttga

SEQ ID NO: 417 - RNA, 20 nt, synthetic construct
  misc_feature 1..20: note = Synthetic
  source 1..20: mol_type = other RNA
cttgtacttcatcccactga

SEQ ID NO: 418 - RNA, 22 nt, synthetic construct
  misc_feature 1..22: note = Synthetic
  source 1..22: mol_type = other RNA
cttgtacttcatcccactgatt

SEQ ID NO: 419 - RNA, 23 nt, synthetic construct
  misc_feature 1..23: note = Synthetic
  source 1..23: mol_type = other RNA
cttttggattgcatctactgtat

SEQ ID NO: 420 - RNA, 24 nt, synthetic construct
  misc_feature 1..24: note = Synthetic
  source 1..24: mol_type = other RNA
gattgcatctactgtatagggacc

SEQ ID NO: 421 - RNA, 23 nt, synthetic construct
  misc_feature 1..23: note = Synthetic
  source 1..23: mol_type = other RNA
gcatctactgtatagggaccctc

SEQ ID NO: 422 - RNA, 20 nt, synthetic construct
  misc_feature 1..20: note = Synthetic
  source 1..20: mol_type = other RNA
gctagtctggaggagacatt

SEQ ID NO: 423 - RNA, 21 nt, synthetic construct
  misc_feature 1..21: note = Synthetic
  source 1..21: mol_type = other RNA
gctagtctggaggagacattt

SEQ ID NO: 424 - RNA, 22 nt, synthetic construct
  misc_feature 1..22: note = Synthetic
  source 1..22: mol_type = other RNA
gctagtctggaggagacatttt

SEQ ID NO: 425 - RNA, 23 nt, synthetic construct
  misc_feature 1..23: note = Synthetic
  source 1..23: mol_type = other RNA
gctagtctggaggagacatttta

SEQ ID NO: 426 - RNA, 21 nt, synthetic construct
  misc_feature 1..21: note = Synthetic
  source 1..21: mol_type = other RNA
ggaccctccttccatgactca

SEQ ID NO: 427 - RNA, 22 nt, synthetic construct
  misc_feature 1..22: note = Synthetic
  source 1..22: mol_type = other RNA
ggaccctccttccatgactcaa

SEQ ID NO: 428 - RNA, 24 nt, synthetic construct
  misc_feature 1..24: note = Synthetic
  source 1..24: mol_type = other RNA
ggattgcatctactgtatagggac

SEQ ID NO: 429 - RNA, 20 nt, synthetic construct
  misc_feature 1..20: note = Synthetic
  source 1..20: mol_type = other RNA
gggaccctccttccatgact

SEQ ID NO: 430 - RNA, 23 nt, synthetic construct
  misc_feature 1..23: note = Synthetic
  source 1..23: mol_type = other RNA
gggaccctccttccatgactcaa

SEQ ID NO: 431 - RNA, 20 nt, synthetic construct
  misc_feature 1..20: note = Synthetic
  source 1..20: mol_type = other RNA
gggatatatgaacttaagtt

SEQ ID NO: 432 - RNA, 25 nt, synthetic construct
  misc_feature 1..25: note = Synthetic
  source 1..25: mol_type = other RNA
ggtaacccacctttcaggacaaact

SEQ ID NO: 433 - RNA, 24 nt, synthetic construct
  misc_feature 1..24: note = Synthetic
  source 1..24: mol_type = other RNA
gtaacccacctttcaggacaaact

SEQ ID NO: 434 - RNA, 21 nt, synthetic construct
  misc_feature 1..21: note = Synthetic
  source 1..21: mol_type = other RNA
gtgttatggctaggatgatga

SEQ ID NO: 435 - RNA, 22 nt, synthetic construct
  misc_feature 1..22: note = Synthetic
  source 1..22: mol_type = other RNA
gtgttatggctaggatgatgaa

SEQ ID NO: 436 - RNA, 21 nt, synthetic construct
  misc_feature 1..21: note = Synthetic
  source 1..21: mol_type = other RNA
gttatggctaggatgatgaac

SEQ ID NO: 437 - RNA, 24 nt, synthetic construct
  misc_feature 1..24: note = Synthetic
  source 1..24: mol_type = other RNA
taaatgctagtctggaggagacat

SEQ ID NO: 438 - RNA, 25 nt, synthetic construct
  misc_feature 1..25: note = Synthetic
  source 1..25: mol_type = other RNA
taaatgctagtctggaggagacatt

SEQ ID NO: 439 - RNA, 24 nt, synthetic construct
  misc_feature 1..24: note = Synthetic
  source 1..24: mol_type = other RNA
taacccacctttcaggacaaactt

SEQ ID NO: 440 - RNA, 25 nt, synthetic construct
  misc_feature 1..25: note = Synthetic
  source 1..25: mol_type = other RNA
taacccacctttcaggacaaacttt

SEQ ID NO: 441 - RNA, 23 nt, synthetic construct
  misc_feature 1..23: note = Synthetic
  source 1..23: mol_type = other RNA
tagggaccctccttccatgactc

SEQ ID NO: 442 - RNA, 22 nt, synthetic construct
  misc_feature 1..22: note = Synthetic
  source 1..22: mol_type = other RNA
tatctttgatactaaccttggt

SEQ ID NO: 443 - RNA, 23 nt, synthetic construct
  misc_feature 1..23: note = Synthetic
  source 1..23: mol_type = other RNA
tcttttggattgcatctactgta

SEQ ID NO: 444 - RNA, 25 nt, synthetic construct
  misc_feature 1..25: note = Synthetic
  source 1..25: mol_type = other RNA
tggattgcatctactgtatagggac

SEQ ID NO: 445 - RNA, 20 nt, synthetic construct
  misc_feature 1..20: note = Synthetic
  source 1..20: mol_type = other RNA
tgtacttcatcccactgatt

SEQ ID NO: 446 - RNA, 21 nt, synthetic construct
  misc_feature 1..21: note = Synthetic
  source 1..21: mol_type = other RNA
tgtgttatggctaggatgatg

SEQ ID NO: 447 - RNA, 22 nt, synthetic construct
  misc_feature 1..22: note = Synthetic
  source 1..22: mol_type = other RNA
tgtgttatggctaggatgatga

SEQ ID NO: 448 - RNA, 23 nt, synthetic construct
  misc_feature 1..23: note = Synthetic
  source 1..23: mol_type = other RNA
tgtgttatggctaggatgatgaa

SEQ ID NO: 449 - RNA, 21 nt, synthetic construct
  misc_feature 1..21: note = Synthetic
  source 1..21: mol_type = other RNA
tgttcttgtacttcatcccac

SEQ ID NO: 450 - RNA, 20 nt, synthetic construct
  misc_feature 1..20: note = Synthetic
  source 1..20: mol_type = other RNA
ttcttgtacttcatcccact

SEQ ID NO: 451 - RNA, 20 nt, synthetic construct
  misc_feature 1..20: note = Synthetic
  source 1..20: mol_type = other RNA
ttgtacttcatcccactgat

SEQ ID NO: 452 - RNA, 21 nt, synthetic construct
  misc_feature 1..21: note = Synthetic
  source 1..21: mol_type = other RNA
ttgtacttcatcccactgatt

SEQ ID NO: 453 - RNA, 20 nt, synthetic construct
  misc_feature 1..20: note = Synthetic
  source 1..20: mol_type = other RNA
ttgtgtgtcccatgcttgtt

SEQ ID NO: 454 - RNA, 23 nt, synthetic construct
  misc_feature 1..23: note = Synthetic
  source 1..23: mol_type = other RNA
ttgtgttatggctaggatgatga

SEQ ID NO: 455 - RNA, 23 nt, synthetic construct
  misc_feature 1..23: note = Synthetic
  source 1..23: mol_type = other RNA
aacccacctttcaggacaaactt

SEQ ID NO: 456 - RNA, 25 nt, synthetic construct
  misc_feature 1..25: note = Synthetic
  source 1..25: mol_type = other RNA
aacccacctttcaggacaaactttt

SEQ ID NO: 457 - RNA, 25 nt, synthetic construct
  misc_feature 1..25: note = Synthetic
  source 1..25: mol_type = other RNA
aattattcattgtgttatggctagg

SEQ ID NO: 458 - RNA, 20 nt, synthetic construct
  misc_feature 1..20: note = Synthetic
  source 1..20: mol_type = other RNA
agccattgtgttgaatcctt

SEQ ID NO: 459 - RNA, 22 nt, synthetic construct
  misc_feature 1..22: note = Synthetic
  source 1..22: mol_type = other RNA
agccattgtgttgaatccttta

SEQ ID NO: 460 - RNA, 25 nt, synthetic construct
  misc_feature 1..25: note = Synthetic
  source 1..25: mol_type = other RNA
aggtatctttgatactaaccttggt

SEQ ID NO: 461 - RNA, 22 nt, synthetic construct
  misc_feature 1..22: note = Synthetic
  source 1..22: mol_type = other RNA
atagggaccctccttccatgac

SEQ ID NO: 462 - RNA, 24 nt, synthetic construct
  misc_feature 1..24: note = Synthetic
  source 1..24: mol_type = other RNA
atcctcaggtcagaatacatatat

SEQ ID NO: 463 - RNA, 23 nt, synthetic construct
  misc_feature 1..23: note = Synthetic
  source 1..23: mol_type = other RNA
atctttgatactaaccttggttt

SEQ ID NO: 464 - RNA, 23 nt, synthetic construct
  misc_feature 1..23: note = Synthetic
  source 1..23: mol_type = other RNA
attattcattgtgttatggctag

SEQ ID NO: 465 - RNA, 24 nt, synthetic construct
  misc_feature 1..24: note = Synthetic
  source 1..24: mol_type = other RNA
attattcattgtgttatggctagg

SEQ ID NO: 466 - RNA, 25 nt, synthetic construct
  misc_feature 1..25: note = Synthetic
  source 1..25: mol_type = other RNA
attattcattgtgttatggctagga

SEQ ID NO: 467 - RNA, 25 nt, synthetic construct
  misc_feature 1..25: note = Synthetic
  source 1..25: mol_type = other RNA
attgtgttatggctaggatgatgaa

SEQ ID NO: 468 - RNA, 24 nt, synthetic construct
  misc_feature 1..24: note = Synthetic
  source 1..24: mol_type = other RNA
caaagtctactgttcatttcagct

SEQ ID NO: 469 - RNA, 23 nt, synthetic construct
  misc_feature 1..23: note = Synthetic
  source 1..23: mol_type = other RNA
cagccattgtgttgaatccttta

SEQ ID NO: 470 - RNA, 22 nt, synthetic construct
  misc_feature 1..22: note = Synthetic
  source 1..22: mol_type = other RNA
catctactgtatagggaccctc

SEQ ID NO: 471 - RNA, 24 nt, synthetic construct
  misc_feature 1..24: note = Synthetic
  source 1..24: mol_type = other RNA
ccacctttcaggacaaacttttca

SEQ ID NO: 472 - RNA, 21 nt, synthetic construct
  misc_feature 1..21: note = Synthetic
  source 1..21: mol_type = other RNA
ccagccattgtgttgaatcct

SEQ ID NO: 473 - RNA, 22 nt, synthetic construct
  misc_feature 1..22: note = Synthetic
  source 1..22: mol_type = other RNA
ccagccattgtgttgaatcctt

SEQ ID NO: 474 - RNA, 23 nt, synthetic construct
  misc_feature 1..23: note = Synthetic
  source 1..23: mol_type = other RNA
ccagccattgtgttgaatccttt

SEQ ID NO: 475 - RNA, 24 nt, synthetic construct
  misc_feature 1..24: note = Synthetic
  source 1..24: mol_type = other RNA
ccagccattgtgttgaatccttta

SEQ ID NO: 476 - RNA, 24 nt, synthetic construct
  misc_feature 1..24: note = Synthetic
  source 1..24: mol_type = other RNA
cccacctttcaggacaaacttttc

SEQ ID NO: 477 - RNA, 25 nt, synthetic construct
  misc_feature 1..25: note = Synthetic
  source 1..25: mol_type = other RNA
cccacctttcaggacaaacttttca

SEQ ID NO: 478 - RNA, 20 nt, synthetic construct
  misc_feature 1..20: note = Synthetic
  source 1..20: mol_type = other RNA
ccctccttccatgactcaag

SEQ ID NO: 479 - RNA, 21 nt, synthetic construct
  misc_feature 1..21: note = Synthetic
  source 1..21: mol_type = other RNA
ctccggttctgaaggtgttct

SEQ ID NO: 480 - RNA, 22 nt, synthetic construct
  misc_feature 1..22: note = Synthetic
  source 1..22: mol_type = other RNA
ctccggttctgaaggtgttctt

SEQ ID NO: 481 - RNA, 23 nt, synthetic construct
  misc_feature 1..23: note = Synthetic
  source 1..23: mol_type = other RNA
cttccagccattgtgttgaatcc

SEQ ID NO: 482 - RNA, 24 nt, synthetic construct
  misc_feature 1..24: note = Synthetic
  source 1..24: mol_type = other RNA
cttccagccattgtgttgaatcct

SEQ ID NO: 483 - RNA, 25 nt, synthetic construct
  misc_feature 1..25: note = Synthetic
  source 1..25: mol_type = other RNA
cttccagccattgtgttgaatcctt

SEQ ID NO: 484 - RNA, 24 nt, synthetic construct
  misc_feature 1..24: note = Synthetic
  source 1..24: mol_type = other RNA
gaccctccttccatgactcaagct

SEQ ID NO: 485 - RNA, 21 nt, synthetic construct
  misc_feature 1..21: note = Synthetic
  source 1..21: mol_type = other RNA
gcatctactgtatagggaccc

SEQ ID NO: 486 - RNA, 21 nt, synthetic construct
  misc_feature 1..21: note = Synthetic
  source 1..21: mol_type = other RNA
gccattgtgttgaatccttta

SEQ ID NO: 487 - RNA, 24 nt, synthetic construct
  misc_feature 1..24: note = Synthetic
  source 1..24: mol_type = other RNA
gctagtctggaggagacattttaa

SEQ ID NO: 488 - RNA, 23 nt, synthetic construct
  misc_feature 1..23: note = Synthetic
  source 1..23: mol_type = other RNA
gcttccagccattgtgttgaatc

SEQ ID NO: 489 - RNA, 24 nt, synthetic construct
  misc_feature 1..24: note = Synthetic
  source 1..24: mol_type = other RNA
gcttccagccattgtgttgaatcc

SEQ ID NO: 490 - RNA, 25 nt, synthetic construct
  misc_feature 1..25: note = Synthetic
  source 1..25: mol_type = other RNA
gcttccagccattgtgttgaatcct

SEQ ID NO: 491 - RNA, 23 nt, synthetic construct
  misc_feature 1..23: note = Synthetic
  source 1..23: mol_type = other RNA
ggaccctccttccatgactcaag

SEQ ID NO: 492 - RNA, 24 nt, synthetic construct
  misc_feature 1..24: note = Synthetic
  source 1..24: mol_type = other RNA
ggaccctccttccatgactcaagc

SEQ ID NO: 493 - RNA, 25 nt, synthetic construct
  misc_feature 1..25: note = Synthetic
  source 1..25: mol_type = other RNA
ggaccctccttccatgactcaagct

SEQ ID NO: 494 - RNA, 24 nt, synthetic construct
  misc_feature 1..24: note = Synthetic
  source 1..24: mol_type = other RNA
gggaccctccttccatgactcaag

SEQ ID NO: 495 - RNA, 25 nt, synthetic construct
  misc_feature 1..25: note = Synthetic
  source 1..25: mol_type = other RNA
gggaccctccttccatgactcaagc

SEQ ID NO: 496 - RNA, 23 nt, synthetic construct
  misc_feature 1..23: note = Synthetic
  source 1..23: mol_type = other RNA
gtaaatgctagtctggaggagac

SEQ ID NO: 497 - RNA, 25 nt, synthetic construct
  misc_feature 1..25: note = Synthetic
  source 1..25: mol_type = other RNA
gtcccatgcttgttaaaaaacttac

SEQ ID NO: 498 - RNA, 20 nt, synthetic construct
  misc_feature 1..20: note = Synthetic
  source 1..20: mol_type = other RNA
gtctactgttcatttcagct

SEQ ID NO: 499 - RNA, 20 nt, synthetic construct
  misc_feature 1..20: note = Synthetic
  source 1..20: mol_type = other RNA
gtgttatggctaggatgatg

SEQ ID NO: 500 - RNA, 25 nt, synthetic construct
  misc_feature 1..25: note = Synthetic
  source 1..25: mol_type = other RNA
tagcttccagccattgtgttgaatc

SEQ ID NO: 501 - RNA, 23 nt, synthetic construct
  misc_feature 1..23: note = Synthetic
  source 1..23: mol_type = other RNA
tatctttgatactaaccttggtt

SEQ ID NO: 502 - RNA, 22 nt, synthetic construct
  misc_feature 1..22: note = Synthetic
  source 1..22: mol_type = other RNA
tccagccattgtgttgaatcct

SEQ ID NO: 503 - RNA, 23 nt, synthetic construct
  misc_feature 1..23: note = Synthetic
  source 1..23: mol_type = other RNA
tccagccattgtgttgaatcctt

SEQ ID NO: 504 - RNA, 24 nt, synthetic construct
  misc_feature 1..24: note = Synthetic
  source 1..24: mol_type = other RNA
tccagccattgtgttgaatccttt

SEQ ID NO: 505 - RNA, 25 nt, synthetic construct
  misc_feature 1..25: note = Synthetic
  source 1..25: mol_type = other RNA
tccagccattgtgttgaatccttta

SEQ ID NO: 506 - RNA, 23 nt, synthetic construct
  misc_feature 1..23: note = Synthetic
  source 1..23: mol_type = other RNA
tcctcaggtcagaatacatatat

SEQ ID NO: 507 - RNA, 23 nt, synthetic construct
  misc_feature 1..23: note = Synthetic
  source 1..23: mol_type = other RNA
tgcctccggttctgaaggtgttc

SEQ ID NO: 508 - RNA, 20 nt, synthetic construct
  misc_feature 1..20: note = Synthetic
  source 1..20: mol_type = other RNA
ttatggctaggatgatgaac

SEQ ID NO: 509 - RNA, 24 nt, synthetic construct
  misc_feature 1..24: note = Synthetic
  source 1..24: mol_type = other RNA
ttgtgttatggctaggatgatgaa

SEQ ID NO: 510 - DNA, 24 bp, synthetic construct
  misc_feature 1..24: note = Synthetic
  source 1..24: mol_type = other DNA
atagggaccctccttccatgactc

SEQ ID NO: 511 - DNA, 23 bp, synthetic construct
  misc_feature 1..23: note = Synthetic
  source 1..23: mol_type = other DNA
catctactgtatagggaccctcc

SEQ ID NO: 512 - DNA, 24 bp, synthetic construct
  misc_feature 1..24: note = Synthetic
  source 1..24: mol_type = other DNA
catctactgtatagggaccctcct

SEQ ID NO: 513 - DNA, 21 bp, synthetic construct
  misc_feature 1..21: note = Synthetic
  source 1..21: mol_type = other DNA
ccctccttccatgactcaagc

SEQ ID NO: 514 - DNA, 22 bp, synthetic construct
  misc_feature 1..22: note = Synthetic
  source 1..22: mol_type = other DNA
ccctccttccatgactcaagct

SEQ ID NO: 515 - DNA, 20 bp, synthetic construct
  misc_feature 1..20: note = Synthetic
  source 1..20: mol_type = other DNA
cctccttccatgactcaagc

SEQ ID NO: 516 - DNA, 21 bp, synthetic construct
  misc_feature 1..21: note = Synthetic
  source 1..21: mol_type = other DNA
cctccttccatgactcaagct

SEQ ID NO: 517 - DNA, 24 bp, synthetic construct
  misc_feature 1..24: note = Synthetic
  source 1..24: mol_type = other DNA
ccttagcttccagccattgtgttg

SEQ ID NO: 518 - DNA, 25 bp, synthetic construct
  misc_feature 1..25: note = Synthetic
  source 1..25: mol_type = other DNA
ccttagcttccagccattgtgttga

SEQ ID NO: 519 - DNA, 20 bp, synthetic construct
  misc_feature 1..20: note = Synthetic
  source 1..20: mol_type = other DNA
ctccttccatgactcaagct

SEQ ID NO: 520 - DNA, 21 bp, synthetic construct
  misc_feature 1..21: note = Synthetic
  source 1..21: mol_type = other DNA
cttgtacttcatcccactgat

SEQ ID NO: 521 - DNA, 23 bp, synthetic construct
  misc_feature 1..23: note = Synthetic
  source 1..23: mol_type = other DNA
cttgtacttcatcccactgattc

SEQ ID NO: 522 - DNA, 20 bp, synthetic construct
  misc_feature 1..20: note = Synthetic
  source 1..20: mol_type = other DNA
gaccctccttccatgactca

SEQ ID NO: 523 - DNA, 21 bp, synthetic construct
  misc_feature 1..21: note = Synthetic
  source 1..21: mol_type = other DNA
gaccctccttccatgactcaa

SEQ ID NO: 524 - DNA, 22 bp, synthetic construct
  misc_feature 1..22: note = Synthetic
  source 1..22: mol_type = other DNA
gaccctccttccatgactcaag

SEQ ID NO: 525 - DNA, 23 bp, synthetic construct
  misc_feature 1..23: note = Synthetic
  source 1..23: mol_type = other DNA
gaccctccttccatgactcaagc

SEQ ID NO: 526 - DNA, 25 bp, synthetic construct
  misc_feature 1..25: note = Synthetic
  source 1..25: mol_type = other DNA
gattgcatctactgtatagggaccc

SEQ ID NO: 527 - DNA, 24 bp, synthetic construct
  misc_feature 1..24: note = Synthetic
  source 1..24: mol_type = other DNA
gcatctactgtatagggaccctcc

SEQ ID NO: 528 - DNA, 20 bp, synthetic construct
  misc_feature 1..20: note = Synthetic
  source 1..20: mol_type = other DNA
gctttgtgtgtcccatgctt

SEQ ID NO: 529 - DNA, 21 bp, synthetic construct
  misc_feature 1..21: note = Synthetic
  source 1..21: mol_type = other DNA
gctttgtgtgtcccatgcttg

SEQ ID NO: 530 - DNA, 22 bp, synthetic construct
  misc_feature 1..22: note = Synthetic
  source 1..22: mol_type = other DNA
gctttgtgtgtcccatgcttgt

SEQ ID NO: 531 - DNA, 23 bp, synthetic construct
  misc_feature 1..23: note = Synthetic
  source 1..23: mol_type = other DNA
gctttgtgtgtcccatgcttgtt

SEQ ID NO: 532 - DNA, 24 bp, synthetic construct
  misc_feature 1..24: note = Synthetic
  source 1..24: mol_type = other DNA
gctttgtgtgtcccatgcttgtta

SEQ ID NO: 533 - DNA, 20 bp, synthetic construct
  misc_feature 1..20: note = Synthetic
  source 1..20: mol_type = other DNA
ggaccctccttccatgactc

SEQ ID NO: 534 - DNA, 25 bp, synthetic construct
  misc_feature 1..25: note = Synthetic
  source 1..25: mol_type = other DNA
ggattgcatctactgtatagggacc

SEQ ID NO: 535 - DNA, 22 bp, synthetic construct
  misc_feature 1..22: note = Synthetic
  source 1..22: mol_type = other DNA
gggaccctccttccatgactca

SEQ ID NO: 536 - DNA, 25 bp, synthetic construct
  misc_feature 1..25: note = Synthetic
  source 1..25: mol_type = other DNA
gtaacccacctttcaggacaaactt

SEQ ID NO: 537 - DNA, 20 bp, synthetic construct
  misc_feature 1..20: note = Synthetic
  source 1..20: mol_type = other DNA
gtacttcatcccactgattc

SEQ ID NO: 538 - DNA, 23 bp, synthetic construct
  misc_feature 1..23: note = Synthetic
  source 1..23: mol_type = other DNA
gtgttatggctaggatgatgaac

SEQ ID NO: 539 - DNA, 20 bp, synthetic construct
  misc_feature 1..20: note = Synthetic
  source 1..20: mol_type = other DNA
tcttgtacttcatcccactg

SEQ ID NO: 540 - DNA, 21 bp, synthetic construct
  misc_feature 1..21: note = Synthetic
  source 1..21: mol_type = other DNA
tcttgtacttcatcccactga

SEQ ID NO: 541 - DNA, 22 bp, synthetic construct
  misc_feature 1..22: note = Synthetic
  source 1..22: mol_type = other DNA
tcttgtacttcatcccactgat

SEQ ID NO: 542 - DNA, 23 bp, synthetic construct
  misc_feature 1..23: note = Synthetic
  source 1..23: mol_type = other DNA
tcttgtacttcatcccactgatt

SEQ ID NO: 543 - DNA, 24 bp, synthetic construct
  misc_feature 1..24: note = Synthetic
  source 1..24: mol_type = other DNA
tcttgtacttcatcccactgattc

SEQ ID NO: 544 - DNA, 24 bp, synthetic construct
  misc_feature 1..24: note = Synthetic
  source 1..24: mol_type = other DNA
tgcctccggttctgaaggtgttct

SEQ ID NO: 545 - DNA, 25 bp, synthetic construct
  misc_feature 1..25: note = Synthetic
  source 1..25: mol_type = other DNA
tgcctccggttctgaaggtgttctt

SEQ ID NO: 546 - DNA, 20 bp, synthetic construct
  misc_feature 1..20: note = Synthetic
  source 1..20: mol_type = other DNA
tgctttgtgtgtcccatgct

SEQ ID NO: 547 - DNA, 21 bp, synthetic construct
  misc_feature 1..21: note = Synthetic
  source 1..21: mol_type = other DNA
tgctttgtgtgtcccatgctt

SEQ ID NO: 548 - DNA, 22 bp, synthetic construct
  misc_feature 1..22: note = Synthetic
  source 1..22: mol_type = other DNA
tgctttgtgtgtcccatgcttg

SEQ ID NO: 549 - DNA, 23 bp, synthetic construct
  misc_feature 1..23: note = Synthetic
  source 1..23: mol_type = other DNA
tgctttgtgtgtcccatgcttgt

SEQ ID NO: 550 - DNA, 24 bp, synthetic construct
  misc_feature 1..24: note = Synthetic
  source 1..24: mol_type = other DNA
tgctttgtgtgtcccatgcttgtt

SEQ ID NO: 551 - DNA, 25 bp, synthetic construct
  misc_feature 1..25: note = Synthetic
  source 1..25: mol_type = other DNA
tgctttgtgtgtcccatgcttgtta

SEQ ID NO: 552 - DNA, 21 bp, synthetic construct
  misc_feature 1..21: note = Synthetic
  source 1..21: mol_type = other DNA
tgtacttcatcccactgattc

SEQ ID NO: 553 - DNA, 20 bp, synthetic construct
  misc_feature 1..20: note = Synthetic
  source 1..20: mol_type = other DNA
tgtgtgtcccatgcttgtta

SEQ ID NO: 554 - DNA, 24 bp, synthetic construct
  misc_feature 1..24: note = Synthetic
  source 1..24: mol_type = other DNA
tgtgttatggctaggatgatgaac

SEQ ID NO: 555 - DNA, 22 bp, synthetic construct
  misc_feature 1..22: note = Synthetic
  source 1..22: mol_type = other DNA
tgttcttgtacttcatcccact

SEQ ID NO: 556 - DNA, 23 bp, synthetic construct
  misc_feature 1..23: note = Synthetic
  source 1..23: mol_type = other DNA
tgttcttgtacttcatcccactg

SEQ ID NO: 557 - DNA, 24 bp, synthetic construct
  misc_feature 1..24: note = Synthetic
  source 1..24: mol_type = other DNA
tgttcttgtacttcatcccactga

SEQ ID NO: 558 - DNA, 25 bp, synthetic construct
  misc_feature 1..25: note = Synthetic
  source 1..25: mol_type = other DNA
tgttcttgtacttcatcccactgat

SEQ ID NO: 559 - DNA, 21 bp, synthetic construct
  misc_feature 1..21: note = Synthetic
  source 1..21: mol_type = other DNA
ttcttgtacttcatcccactg

SEQ ID NO: 560 - DNA, 22 bp, synthetic construct
  misc_feature 1..22: note = Synthetic
  source 1..22: mol_type = other DNA
ttcttgtacttcatcccactga

SEQ ID NO: 561 - DNA, 23 bp, synthetic construct
  misc_feature 1..23: note = Synthetic
  source 1..23: mol_type = other DNA
ttcttgtacttcatcccactgat

SEQ ID NO: 562 - DNA, 24 bp, synthetic construct
  misc_feature 1..24: note = Synthetic
  source 1..24: mol_type = other DNA
ttcttgtacttcatcccactgatt

SEQ ID NO: 563 - DNA, 25 bp, synthetic construct
  misc_feature 1..25: note = Synthetic
  source 1..25: mol_type = other DNA
ttcttgtacttcatcccactgattc

SEQ ID NO: 564 - DNA, 22 bp, synthetic construct
  misc_feature 1..22: note = Synthetic
  source 1..22: mol_type = other DNA
ttgtacttcatcccactgattc

SEQ ID NO: 565 - DNA, 21 bp, synthetic construct
  misc_feature 1..21: note = Synthetic
  source 1..21: mol_type = other DNA
ttgtgtgtcccatgcttgtta

SEQ ID NO: 566 - DNA, 25 bp, synthetic construct
  misc_feature 1..25: note = Synthetic
  source 1..25: mol_type = other DNA
ttgtgttatggctaggatgatgaac

SEQ ID NO: 567 - DNA, 20 bp, synthetic construct
  misc_feature 1..20: note = Synthetic
  source 1..20: mol_type = other DNA
tttgtgtgtcccatgcttgt

SEQ ID NO: 568 - DNA, 21 bp, synthetic construct
  misc_feature 1..21: note = Synthetic
  source 1..21: mol_type = other DNA
tttgtgtgtcccatgcttgtt

SEQ ID NO: 569 - DNA, 22 bp, synthetic construct
  misc_feature 1..22: note = Synthetic
  source 1..22: mol_type = other DNA
tttgtgtgtcccatgcttgtta

SEQ ID NO: 570 - DNA, 23 bp, synthetic construct
  misc_feature 1..23: note = Synthetic
  source 1..23: mol_type = other DNA
aaatgctagtctggaggagacat

SEQ ID NO: 571 - DNA, 24 bp, synthetic construct
  misc_feature 1..24: note = Synthetic
  source 1..24: mol_type = other DNA
aaatgctagtctggaggagacatt

SEQ ID NO: 572 - DNA, 25 bp, synthetic construct
  misc_feature 1..25: note = Synthetic
  source 1..25: mol_type = other DNA
aaatgctagtctggaggagacattt

SEQ ID NO: 573 - DNA, 24 bp, synthetic construct
  misc_feature 1..24: note = Synthetic
  source 1..24: mol_type = other DNA
aacccacctttcaggacaaacttt

SEQ ID NO: 574 - DNA, 24 bp, synthetic construct
  misc_feature 1..24: note = Synthetic
  source 1..24: mol_type = other DNA
aactgttgcctccggttctgaagg

SEQ ID NO: 575 - DNA, 22 bp, synthetic construct
  misc_feature 1..22: note = Synthetic
  source 1..22: mol_type = other DNA
agggaccctccttccatgactc

SEQ ID NO: 576 - DNA, 23 bp, synthetic construct
  misc_feature 1..23: note = Synthetic
  source 1..23: mol_type = other DNA
atagggaccctccttccatgact

SEQ ID NO: 577 - DNA, 22 bp, synthetic construct
  misc_feature 1..22: note = Synthetic
  source 1..22: mol_type = other DNA
atctactgtatagggaccctcc

SEQ ID NO: 578 - DNA, 23 bp, synthetic construct
  misc_feature 1..23: note = Synthetic
  source 1..23: mol_type = other DNA
atctactgtatagggaccctcct

SEQ ID NO: 579 - DNA, 21 bp, synthetic construct
  misc_feature 1..21: note = Synthetic
  source 1..21: mol_type = other DNA
atctttgatactaaccttggt

SEQ ID NO: 580 - DNA, 22 bp, synthetic construct
  misc_feature 1..22: note = Synthetic
  source 1..22: mol_type = other DNA
atctttgatactaaccttggtt

SEQ ID NO: 581 - DNA, 24 bp, synthetic construct
  misc_feature 1..24: note = Synthetic
  source 1..24: mol_type = other DNA
attgtgttatggctaggatgatga

SEQ ID NO: 582 - DNA, 25 bp, synthetic construct
  misc_feature 1..25: note = Synthetic
  source 1..25: mol_type = other DNA
caactgttgcctccggttctgaagg

SEQ ID NO: 583 - DNA, 22 bp, synthetic construct
  misc_feature 1..22: note = Synthetic
  source 1..22: mol_type = other DNA
cctccggttctgaaggtgttct

SEQ ID NO: 584 - DNA, 23 bp, synthetic construct
  misc_feature 1..23: note = Synthetic
  source 1..23: mol_type = other DNA
cctccggttctgaaggtgttctt

SEQ ID NO: 585 - DNA, 24 bp, synthetic construct
  misc_feature 1..24: note = Synthetic
  source 1..24: mol_type = other DNA
cctccggttctgaaggtgttcttg

SEQ ID NO: 586 - DNA, 22 bp, synthetic construct
  misc_feature 1..22: note = Synthetic
  source 1..22: mol_type = other DNA
ccttagcttccagccattgtgt

SEQ ID NO: 587 - DNA, 23 bp, synthetic construct
  misc_feature 1..23: note = Synthetic
  source 1..23: mol_type = other DNA
ccttagcttccagccattgtgtt

SEQ ID NO: 588 - DNA, 23 bp, synthetic construct
  misc_feature 1..23: note = Synthetic
  source 1..23: mol_type = other DNA
ctccggttctgaaggtgttcttg

SEQ ID NO: 589 - DNA, 25 bp, synthetic construct
  misc_feature 1..25: note = Synthetic
  source 1..25: mol_type = other DNA
ctccggttctgaaggtgttcttgta

SEQ ID NO: 590 - DNA, 23 bp, synthetic construct
  misc_feature 1..23: note = Synthetic
  source 1..23: mol_type = other DNA
cttagcttccagccattgtgttg

SEQ ID NO: 591 - DNA, 24 bp, synthetic construct
  misc_feature 1..24: note = Synthetic
  source 1..24: mol_type = other DNA
cttagcttccagccattgtgttga

SEQ ID NO: 592 - DNA, 20 bp, synthetic construct
  misc_feature 1..20: note = Synthetic
  source 1..20: mol_type = other DNA
cttgtacttcatcccactga

SEQ ID NO: 593 - DNA, 22 bp, synthetic construct
  misc_feature 1..22: note = Synthetic
  source 1..22: mol_type = other DNA
cttgtacttcatcccactgatt

SEQ ID NO: 594 - DNA, 23 bp, synthetic construct
  misc_feature 1..23: note = Synthetic
  source 1..23: mol_type = other DNA
cttttggattgcatctactgtat

SEQ ID NO: 595 - DNA, 24 bp, synthetic construct
  misc_feature 1..24: note = Synthetic
  source 1..24: mol_type = other DNA
gattgcatctactgtatagggacc

SEQ ID NO: 596 - DNA, 23 bp, synthetic construct
  misc_feature 1..23: note = Synthetic
  source 1..23: mol_type = other DNA
gcatctactgtatagggaccctc

SEQ ID NO: 597 - DNA, 20 bp, synthetic construct
  misc_feature 1..20: note = Synthetic
  source 1..20: mol_type = other DNA
gctagtctggaggagacatt

SEQ ID NO: 598 - DNA, 21 bp, synthetic construct
  misc_feature 1..21: note = Synthetic
  source 1..21: mol_type = other DNA
gctagtctggaggagacattt

SEQ ID NO: 599 - DNA, 22 bp, synthetic construct
  misc_feature 1..22: note = Synthetic
  source 1..22: mol_type = other DNA
gctagtctggaggagacatttt

SEQ ID NO: 600 - DNA, 23 bp, synthetic construct
  misc_feature 1..23: note = Synthetic
  source 1..23: mol_type = other DNA
gctagtctggaggagacatttta

SEQ ID NO: 601 - DNA, 21 bp, synthetic construct
  misc_feature 1..21: note = Synthetic
  source 1..21: mol_type = other DNA
ggaccctccttccatgactca

SEQ ID NO: 602 - DNA, 22 bp, synthetic construct
  misc_feature 1..22: note = Synthetic
  source 1..22: mol_type = other DNA
ggaccctccttccatgactcaa

SEQ ID NO: 603 - DNA, 24 bp, synthetic construct
  misc_feature 1..24: note = Synthetic
  source 1..24: mol_type = other DNA
ggattgcatctactgtatagggac

SEQ ID NO: 604 - DNA, 20 bp, synthetic construct
  misc_feature 1..20: note = Synthetic
  source 1..20: mol_type = other DNA
gggaccctccttccatgact

SEQ ID NO: 605 - DNA, 23 bp, synthetic construct
  misc_feature 1..23: note = Synthetic
  source 1..23: mol_type = other DNA
gggaccctccttccatgactcaa

SEQ ID NO: 606 - DNA, 20 bp, synthetic construct
  misc_feature 1..20: note = Synthetic
  source 1..20: mol_type = other DNA
gggatatatgaacttaagtt

SEQ ID NO: 607 - DNA, 25 bp, synthetic construct
  misc_feature 1..25: note = Synthetic
  source 1..25: mol_type = other DNA
ggtaacccacctttcaggacaaact

SEQ ID NO: 608 - DNA, 24 bp, synthetic construct
  misc_feature 1..24: note = Synthetic
  source 1..24: mol_type = other DNA
gtaacccacctttcaggacaaact

SEQ ID NO: 609 - DNA, 21 bp, synthetic construct
  misc_feature 1..21: note = Synthetic
  source 1..21: mol_type = other DNA
gtgttatggctaggatgatga

SEQ ID NO: 610 - DNA, 22 bp, synthetic construct
  misc_feature 1..22: note = Synthetic
  source 1..22: mol_type = other DNA
gtgttatggctaggatgatgaa

SEQ ID NO: 611 - DNA, 21 bp, synthetic construct
  misc_feature 1..21: note = Synthetic
  source 1..21: mol_type = other DNA
gttatggctaggatgatgaac

SEQ ID NO: 612 - DNA, 24 bp, synthetic construct
  misc_feature 1..24: note = Synthetic
  source 1..24: mol_type = other DNA
taaatgctagtctggaggagacat

SEQ ID NO: 613 - DNA, 25 bp, synthetic construct
  misc_feature 1..25: note = Synthetic
  source 1..25: mol_type = other DNA
taaatgctagtctggaggagacatt

SEQ ID NO: 614 - DNA, 24 bp, synthetic construct
  misc_feature 1..24: note = Synthetic
  source 1..24: mol_type = other DNA
taacccacctttcaggacaaactt

SEQ ID NO: 615 - DNA, 25 bp, synthetic construct
  misc_feature 1..25: note = Synthetic
  source 1..25: mol_type = other DNA
taacccacctttcaggacaaacttt

SEQ ID NO: 616 - DNA, 23 bp, synthetic construct
  misc_feature 1..23: note = Synthetic
  source 1..23: mol_type = other DNA
tagggaccctccttccatgactc

SEQ ID NO: 617 - DNA, 22 bp, synthetic construct
  misc_feature 1..22: note = Synthetic
  source 1..22: mol_type = other DNA
tatctttgatactaaccttggt

SEQ ID NO: 618 - DNA, 23 bp, synthetic construct
  misc_feature 1..23: note = Synthetic
  source 1..23: mol_type = other DNA
tcttttggattgcatctactgta

SEQ ID NO: 619 - DNA, 25 bp, synthetic construct
  misc_feature 1..25: note = Synthetic
  source 1..25: mol_type = other DNA
tggattgcatctactgtatagggac

SEQ ID NO: 620 - DNA, 20 bp, synthetic construct
  misc_feature 1..20: note = Synthetic
  source 1..20: mol_type = other DNA
tgtacttcatcccactgatt

SEQ ID NO: 621 - DNA, 21 bp, synthetic construct
  misc_feature 1..21: note = Synthetic
  source 1..21: mol_type = other DNA
tgtgttatggctaggatgatg

SEQ ID NO: 622 - DNA, 22 bp, synthetic construct
  misc_feature 1..22: note = Synthetic
  source 1..22: mol_type = other DNA
tgtgttatggctaggatgatga

SEQ ID NO: 623 - DNA, 23 bp, synthetic construct
  misc_feature 1..23: note = Synthetic
  source 1..23: mol_type = other DNA
tgtgttatggctaggatgatgaa

SEQ ID NO: 624 - DNA, 21 bp, synthetic construct
  misc_feature 1..21: note = Synthetic
  source 1..21: mol_type = other DNA
tgttcttgtacttcatcccac

SEQ ID NO: 625 - DNA, 20 bp, synthetic construct
  misc_feature 1..20: note = Synthetic
  source 1..20: mol_type = other DNA
ttcttgtacttcatcccact

SEQ ID NO: 626 - DNA, 20 bp, synthetic construct
  misc_feature 1..20: note = Synthetic
  source 1..20: mol_type = other DNA
ttgtacttcatcccactgat

SEQ ID NO: 627 - DNA, 21 bp, synthetic construct
  misc_feature 1..21: note = Synthetic
  source 1..21: mol_type = other DNA
ttgtacttcatcccactgatt

SEQ ID NO: 628 - DNA, 20 bp, synthetic construct
  misc_feature 1..20: note = Synthetic
  source 1..20: mol_type = other DNA
ttgtgtgtcccatgcttgtt

SEQ ID NO: 629 - DNA, 23 bp, synthetic construct
  misc_feature 1..23: note = Synthetic
  source 1..23: mol_type = other DNA
ttgtgttatggctaggatgatga

SEQ ID NO: 630 - DNA, 23 bp, synthetic construct
  misc_feature 1..23: note = Synthetic
  source 1..23: mol_type = other DNA
aacccacctttcaggacaaactt

SEQ ID NO: 631 - DNA, 25 bp, synthetic construct
  misc_feature 1..25: note = Synthetic
  source 1..25: mol_type = other DNA
aacccacctttcaggacaaactttt

SEQ ID NO: 632 - DNA, 25 bp, synthetic construct
  misc_feature 1..25: note = Synthetic
  source 1..25: mol_type = other DNA
aattattcattgtgttatggctagg

SEQ ID NO: 633 - DNA, 20 bp, synthetic construct
  misc_feature 1..20: note = Synthetic
  source 1..20: mol_type = other DNA
agccattgtgttgaatcctt

SEQ ID NO: 634 - DNA, 22 bp, synthetic construct
  misc_feature 1..22: note = Synthetic
  source 1..22: mol_type = other DNA
agccattgtgttgaatccttta

SEQ ID NO: 635 - DNA, 25 bp, synthetic construct
  misc_feature 1..25: note = Synthetic
  source 1..25: mol_type = other DNA
aggtatctttgatactaaccttggt

SEQ ID NO: 636 - DNA, 22 bp, synthetic construct
  misc_feature 1..22: note = Synthetic
  source 1..22: mol_type = other DNA
atagggaccctccttccatgac

SEQ ID NO: 637 - DNA, 24 bp, synthetic construct
  misc_feature 1..24: note = Synthetic
  source 1..24: mol_type = other DNA
atcctcaggtcagaatacatatat

SEQ ID NO: 638 - DNA, 23 bp, synthetic construct
  misc_feature 1..23: note = Synthetic
  source 1..23: mol_type = other DNA
atctttgatactaaccttggttt

SEQ ID NO: 639 - DNA, 23 bp, synthetic construct
  misc_feature 1..23: note = Synthetic
  source 1..23: mol_type = other DNA
attattcattgtgttatggctag

SEQ ID NO: 640 - DNA, 24 bp, synthetic construct
  misc_feature 1..24: note = Synthetic
  source 1..24: mol_type = other DNA
attattcattgtgttatggctagg

SEQ ID NO: 641 - DNA, 25 bp, synthetic construct
  misc_feature 1..25: note = Synthetic
  source 1..25: mol_type = other DNA
attattcattgtgttatggctagga

SEQ ID NO: 642 - DNA, 25 bp, synthetic construct
  misc_feature 1..25: note = Synthetic
  source 1..25: mol_type = other DNA
attgtgttatggctaggatgatgaa

SEQ ID NO: 643 - DNA, 24 bp, synthetic construct
  misc_feature 1..24: note = Synthetic
  source 1..24: mol_type = other DNA
caaagtctactgttcatttcagct

SEQ ID NO: 644 - DNA, 23 bp, synthetic construct
  misc_feature 1..23: note = Synthetic
  source 1..23: mol_type = other DNA
cagccattgtgttgaatccttta

SEQ ID NO: 645 - DNA, 22 bp, synthetic construct
  misc_feature 1..22: note = Synthetic
  source 1..22: mol_type = other DNA
catctactgtatagggaccctc

SEQ ID NO: 646 - DNA, 24 bp, synthetic construct
  misc_feature 1..24: note = Synthetic
  source 1..24: mol_type = other DNA
ccacctttcaggacaaacttttca

SEQ ID NO: 647 - DNA, 21 bp, synthetic construct
  misc_feature 1..21: note = Synthetic
  source 1..21: mol_type = other DNA
ccagccattgtgttgaatcct

SEQ ID NO: 648 - DNA, 22 bp, synthetic construct
  misc_feature 1..22: note = Synthetic
  source 1..22: mol_type = other DNA
ccagccattgtgttgaatcctt

SEQ ID NO: 649 - DNA, 23 bp, synthetic construct
  misc_feature 1..23: note = Synthetic
  source 1..23: mol_type = other DNA
ccagccattgtgttgaatccttt

SEQ ID NO: 650 - DNA, 24 bp, synthetic construct
  misc_feature 1..24: note = Synthetic
  source 1..24: mol_type = other DNA
ccagccattgtgttgaatccttta

SEQ ID NO: 651 - DNA, 24 bp, synthetic construct
  misc_feature 1..24: note = Synthetic
  source 1..24: mol_type = other DNA
cccacctttcaggacaaacttttc

SEQ ID NO: 652 - DNA, 25 bp, synthetic construct
  misc_feature 1..25: note = Synthetic
  source 1..25: mol_type = other DNA
cccacctttcaggacaaacttttca

SEQ ID NO: 653 - DNA, 20 bp, synthetic construct
  misc_feature 1..20: note = Synthetic
  source 1..20: mol_type = other DNA
ccctccttccatgactcaag

SEQ ID NO: 654 - DNA, 21 bp, synthetic construct
  misc_feature 1..21: note = Synthetic
  source 1..21: mol_type = other DNA
ctccggttctgaaggtgttct

SEQ ID NO: 655 - DNA, 22 bp, synthetic construct
  misc_feature 1..22: note = Synthetic
  source 1..22: mol_type = other DNA
ctccggttctgaaggtgttctt

SEQ ID NO: 656 - DNA, 23 bp, synthetic construct
  misc_feature 1..23: note = Synthetic
  source 1..23: mol_type = other DNA
cttccagccattgtgttgaatcc

SEQ ID NO: 657 - DNA, 24 bp, synthetic construct
  misc_feature 1..24: note = Synthetic
  source 1..24: mol_type = other DNA
cttccagccattgtgttgaatcct

SEQ ID NO: 658 - DNA, 25 bp, synthetic construct
  misc_feature 1..25: note = Synthetic
  source 1..25: mol_type = other DNA
cttccagccattgtgttgaatcctt

SEQ ID NO: 659 - DNA, 24 bp, synthetic construct
  misc_feature 1..24: note = Synthetic
  source 1..24: mol_type = other DNA
gaccctccttccatgactcaagct

SEQ ID NO: 660 - DNA, 21 bp, synthetic construct
  misc_feature 1..21: note = Synthetic
  source 1..21: mol_type = other DNA
gcatctactgtatagggaccc

SEQ ID NO: 661 - DNA, 21 bp, synthetic construct
  misc_feature 1..21: note = Synthetic
  source 1..21: mol_type = other DNA
gccattgtgttgaatccttta

SEQ ID NO: 662 - DNA, 24 bp, synthetic construct
  misc_feature 1..24: note = Synthetic
  source 1..24: mol_type = other DNA
gctagtctggaggagacattttaa

SEQ ID NO: 663 - DNA, 23 bp, synthetic construct
  misc_feature 1..23: note = Synthetic
  source 1..23: mol_type = other DNA
gcttccagccattgtgttgaatc

SEQ ID NO: 664 - DNA, 24 bp, synthetic construct
  misc_feature 1..24: note = Synthetic
  source 1..24: mol_type = other DNA
gcttccagccattgtgttgaatcc

SEQ ID NO: 665 - DNA, 25 bp, synthetic construct
  misc_feature 1..25: note = Synthetic
  source 1..25: mol_type = other DNA
gcttccagccattgtgttgaatcct

SEQ ID NO: 666 - DNA, 23 bp, synthetic construct
  misc_feature 1..23: note = Synthetic
  source 1..23: mol_type = other DNA
ggaccctccttccatgactcaag

SEQ ID NO: 667 - DNA, 24 bp, synthetic construct
  misc_feature 1..24: note = Synthetic
  source 1..24: mol_type = other DNA
ggaccctccttccatgactcaagc

SEQ ID NO: 668 - DNA, 25 bp, synthetic construct
  misc_feature 1..25: note = Synthetic
  source 1..25: mol_type = other DNA
ggaccctccttccatgactcaagct

SEQ ID NO: 669 - DNA, 24 bp, synthetic construct
  misc_feature 1..24: note = Synthetic
  source 1..24: mol_type = other DNA
gggaccctccttccatgactcaag

SEQ ID NO: 670 - DNA, 25 bp, synthetic construct
  misc_feature 1..25: note = Synthetic
  source 1..25: mol_type = other DNA
gggaccctccttccatgactcaagc

SEQ ID NO: 671 - DNA, 23 bp, synthetic construct
  misc_feature 1..23: note = Synthetic
  source 1..23: mol_type = other DNA
gtaaatgctagtctggaggagac

SEQ ID NO: 672 - DNA, 25 bp, synthetic construct
  misc_feature 1..25: note = Synthetic
  source 1..25: mol_type = other DNA
gtcccatgcttgttaaaaaacttac

SEQ ID NO: 673 - DNA, 20 bp, synthetic construct
  misc_feature 1..20: note = Synthetic
  source 1..20: mol_type = other DNA
gtctactgttcatttcagct

SEQ ID NO: 674 - DNA, 20 bp, synthetic construct
  misc_feature 1..20: note = Synthetic
  source 1..20: mol_type = other DNA
gtgttatggctaggatgatg

SEQ ID NO: 675 - DNA, 25 bp, synthetic construct
  misc_feature 1..25: note = Synthetic
  source 1..25: mol_type = other DNA
tagcttccagccattgtgttgaatc

SEQ ID NO: 676 - DNA, 23 bp, synthetic construct
  misc_feature 1..23: note = Synthetic
  source 1..23: mol_type = other DNA
tatctttgatactaaccttggtt

SEQ ID NO: 677 - DNA, 22 bp, synthetic construct
  misc_feature 1..22: note = Synthetic
  source 1..22: mol_type = other DNA
tccagccattgtgttgaatcct

SEQ ID NO: 678 - DNA, 23 bp, synthetic construct
  misc_feature 1..23: note = Synthetic
  source 1..23: mol_type = other DNA
tccagccattgtgttgaatcctt

SEQ ID NO: 679 - DNA, 24 bp, synthetic construct
  misc_feature 1..24: note = Synthetic
  source 1..24: mol_type = other DNA
tccagccattgtgttgaatccttt

SEQ ID NO: 680 - DNA, 25 bp, synthetic construct
  misc_feature 1..25: note = Synthetic
  source 1..25: mol_type = other DNA
tccagccattgtgttgaatccttta

SEQ ID NO: 681 - DNA, 23 bp, synthetic construct
  misc_feature 1..23: note = Synthetic
  source 1..23: mol_type = other DNA
tcctcaggtcagaatacatatat

SEQ ID NO: 682 - DNA, 23 bp, synthetic construct
  misc_feature 1..23: note = Synthetic
  source 1..23: mol_type = other DNA
tgcctccggttctgaaggtgttc

SEQ ID NO: 683 - DNA, 20 bp, synthetic construct
  misc_feature 1..20: note = Synthetic
  source 1..20: mol_type = other DNA
ttatggctaggatgatgaac

SEQ ID NO: 684 - DNA, 24 bp, synthetic construct
  misc_feature 1..24: note = Synthetic
  source 1..24: mol_type = other DNA
ttgtgttatggctaggatgatgaa

SEQ ID NO: 685-717 - skipped (33 entries)

SEQ ID NO: 718 - DNA, 11 bp, synthetic construct
  misc_feature 1..11: note = Synthetic
  source 1..11: mol_type = other DNA
tcctttgcagc

SEQ ID NO: 719-751 - skipped (33 entries)

SEQ ID NO: 752 - DNA, 11 bp, synthetic construct
  misc_feature 1..11: note = Synthetic
  source 1..11: mol_type = other DNA
gctgcaaagga

SEQ ID NO: 753 - DNA, 60 bp, Homo sapiens
  source 1..60: mol_type = genomic DNA
aagaggctagaacaatcattacggatcgaagtaagttttttaacaagcatgggacacaca

SEQ ID NO: 754 - DNA, 50044 bp, Homo sapiens
  source 1..50044: mol_type = genomic DNA
gtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatacatttttaaatcaattcagggcttatatagttgcaaagcatgcattgatgggtgtggtgaccacagtgtggcagaacatttgtggcagaacatttgttctttagttgtcatctgggctggcatccatggagatgccagtctctccctcatatccttggctgttggtccaagcaggcagtggcttcttcctgggccatctttcattcccatgtgcagtgactttcagatctggatatctctccgctactttgatgcccccattttgtaatatcaaaaatcatcgtactgtaccttatgccgtagtagggtgggcaggaactttggtaagacccatctgactagacgctgtgcatattcttttcttctgacatacactcctatccatttaatggggagagtgattcgcagtgattgtgtgttgtgtcagtgagtttccatggggtcaggaagagtgacagacgaaggagtaggggaaactcgccacccggtttccctcagagattctcctcagaaatgaggtccaagtcagctctgctttcaggttctctcagatctctctcggtttcttcacttctctctaccttccttccctccagggacacacactggtattgaattttcttgcttcctctgcaatatcccctcattttccttcccacaacccgaagaatcctttgtagtgcaggaaggaggaaaacctttcagccatctttttttttctctttgaaatcttttgtcttttaccaggcttagacttttcaaactcggaaaccatgagagtctatatcttcataatttatattctgctatgttaacccttccctaaggaaatgactagttgtcaatatgttggggaaagtgaaagagtaaccagagtaaaaggttaatattttaaaatattattagtcatacttccacatattgggtaagtacttattgataatagctagtatttattcagtacctcataagcattagatggtgtgtgcacatgtgtgtgtttctgtgtgtttctgtgtgtgtgtacaaaatctttacagaatcttgtgagctatattttataatccccattttatagacgagaaaacaggttccaagaacagttactcggccgggcgcggtggctcacgcctgtaatcccagcacttggggaggccgaggcgggcggatcacgaggtcaggagatcgagaccatcctggctaacacggtgaaaccccgtctctactaaaaatacaaaaaaaaattagccgggcgtggtggcgggcgcctgtagtcccagctacttgggaggctgaggcaggagaatggcgtgaacccgggaggcggagcttgcagtgagccgagattgcgccactgcactccagcctgggcgacagagcgagactgcgtcgtttacaaacaagcaaagaacagagcttggaatttgtaaatgtctctttctgacttgaaaatctgcgctctttctagtatgtttaccatttcccatcttgttttgttgcttttgttaatgaccttaatcattgtactaagactaaatacttcttttgtctgaaattatgtatgttttgattcacttcctaaagacatgtcttctttcagttgtagtgattgctaattaaaataggctgttcttggttttgaaagtttaactctttattgttgcttaaacaatgaatgtggatgtatgctaatgtattatttcagtaacactagccactataacaggtaatctcccaaatcttggaggcttactacagcagaaatttacttcttattttagtgcagtccaaaataagcagccctctatgaagtcattcaggaacccaagctccttccatcttttgtgtccagagcataagactcatatgcatttaatgggcagatgaagaatgagtattactcatgaaaatttgtcatgagccacgcctggaaatggtgtgtgttaattttgttcatattctgttggatggatcttgtcacatggtcacacctaactgcgagggaattagggaaatagtgtacactgttgagcctaggaagaggaacagatttggtaagatagccatacaattgatttagcagagcattcctccaccatactggaaccttgagggttctccaacaagttgcaacacactgacccaaaagagtgaatcttagtgagtgatttactcaatatgagactgaattcccattacacaggtaagtgatcagtttcttgcctgagaaatatggaaatttggcagtgaggtttatgcaaatctgaacatactatacagagagtcatttgttattttactaatgaaaaatcacttcaattttttccctaagaggaagacaatatgaatgtattatacagtatttgttcaacattgtctgaacattttccattatcccctgcatttttttttcatattgcattgactttttcatgatagagattaaaattgaatgaccggagggcaagtttgtatctctctgccaatattcagtgatttgaatagttcttcttttctaagcttttgtcttttaggaatgaatacctttatgaatatttgcagcctagtggaaaggctgtaatccaaatggtccaagaaagcattttcttaaaagcaagtgtctgtcgagatgtgtcatagcgcttattaagagtctaagctggaatcttagttccaaatatgcctggagcctctaaatggtaccaggataattctgacaagatatgtttatctaaacaatgtcatttgcagccctgctacacttcagtttatctctccctttgaaatctataaaatgggatggaaattcaatactcataaacttttcgatcgtgttcaaaatagaatttttcttagtaaagatttggttttcaggaaaaggacagaaataaaatacttgctcataaaattgtattttctcatttgatgattttggtcttcctttttattgccatgaaacttctagaaatgctcaaaaagaaatcagctaaataaagaaaaaatagttaatatatgtatgtaatactatattgaaacatttttctttctctggtaaatcccatttcataactttgaacagttgggaaaatctatacatagttattgcagtctatcaagagaaaagttcagtacaaagctatttatgtctactagaaatattcatgttaaacttcaagtaattgggtgtgcaagccaccaccatgttttactatatgaaactattaccgtggtatctgttgtattcaggtaattatattgatggaaatcatgcagtaataatctaggtaagagagtaaattttgtctaaatcagatcaaatgaaaaattctccctctttctaatattcgaattgctcatttttctttaactctttggtgtctgaatttgtcaatcattcctggccattttcttctgcaaaagggctgggtcaggggaccaaaagcagataagattagaagaatttaaattttcttccttggaggcgtctgaattacatgaaactcttgttcgtgtctgttaatactgcaaggcataataccataataccttgcatagcagtgaagaggatttggaaagataaaactgcttccttttatcattctgtttatttcacaaacaatattggtgaatgtcgttcctgtaacatttggatttaagagccttgtttctgtagcttctccctccgtaacccccaccactaccatttcggggctatacagcaatagcatgcattactttaaaaggcaggctgcctagactggccacttgttagctttgtggccttgagcaaatgactaatctcagtaaactatctgctcttagtttccttctctgtaaaataggctcacttataactatctcatgggttgggaggattagatgaaataattaatgtagagcccttagatcagggccatagtaaaagctgaatgaatgttagcatttgttattttaattataatctattggggtgctttgaaggcttaatgcaaaatacttaatgagctttttggtagctgtttagttatttcgccccccaccaccaccccaaaaggagagatttaaaagaccgacaggagaaggttgcttggaaaagatggaataagatctataaatagaattaaacaaatattcaggaaagccttttgtgggaaatactgcaaaatttttattatctataaatttaataggtagataaaattactactcccattttagagacagaaaaccgagactcggagagctaacgtaacttgtctagggtcttaggaagatgacaagtgaggaagtagaattcaagcccacgtctatatgattttaaagcccgaggcacatcaaatggaaaaggctggttagtcagaaaaataggaaggtatatttatctgacaacttaaaatattaggactaacctcaggtaattatagtctggatatacatttttgctgctcctgtttatacttttgacttctgtgtatttgagtgtctaatcaaaggattgtcttttacatgtgttggagatgtacagactagtggaccccaatgatctattagctgtgtaaccttagccaagttaattctctttcctaaactgtggttctctcatctgtcatgtggggctaataatagtacttatgctggtagggtgattaagaaagtaaaataattggtgtttctaaagtaaatatgtggcacatattagatgctcattaaatggtacatattgttatggtgagatggatttggtacagagagaactggagatgggagaatatgaagggtgtataatgtggccttttattagctaaaccaagggaaggacttctgaaacagaattccaagttttaagagggagtcgtttattttggaattattttttcagctaaggatttttcaacccagtccagaattcttagagaaatttagtgatagcttataaattttaagaaaaggaattcacattatattgcataaagaactggtatacagggccatagaaggggagaatgttcttctgtatgagaataaaaaaaaacatctctcacacgatttttgaattaactgacagttttatagcagctttgtcaacccatcattcattgctgcaaccaaatctatgaaatccttcatggcgaaataaaaaggctctgttgttctccacatttgtatgaaatctctgttgctaatgaaatgccagccagtatcttcctctcaggtattgtctattagatggttgcttattttagaagaagtggagtcaaccatataaatttccttcttttgacatctagcacctgctgtcaacctgttatagctacaagcagctctcaaaattcacatccactaggatgccgctggcaaccaaagagttcagttcagttcagcgaacgtttgaatgcctactctgtgctatctaatatcagagatggtagaggggatacaggaaaaaagtaagattcagcctttgtctttaaagagctcacaatcaaatgtgggtatttggacaagtatatttaggcaaggcagtttaggataggtgcttcagtagagcagtattacaaaatgttgagagaaaactagaggaggagttttaaatgagggcttagtggaatgcttccaggaggaggctgaatttgacctggtcatgaataagactttgaaaagcagaaggaagctggagagggaagggtatttcaggaattgatgacaggagaatacataattagacctgttaacagtggggtggaagacgaatatcagcatgggagtgtgtgtgtgtgtgtgtgtgtgtgtgtgtgtgtgtgtgtgtgtgttgtggaggataagggagtaaatgaagtcagtaagaccagttagaaggcaagagagcccaagctggagcagtgaccatggagtaaaaagaaagaagtgaaattgagacataaggtggagctagaattaacaggattttgacattgattgcatatataaaactgtcaatctaagagaaggcctgcctctaagagttggagattgaattctttggaagattaatgtcataaccagagacaaactcaggataaggagttgattatgggagacaaagtaatgtgcttaacatgaagctagatgaccatactgagattttaaagaaatggttgaaaacatagagattgcaggagtgatgttacttttggacaagaattcaaaagttacctggaatgtctgagcttgcacagagagagggtacctggtgcaaagaaaagtgaacctaggaaaaagcctagaagttggtttacatttaaggaatttgagaagaggtagcaccacccaccccccacacaacccccaaccctgccaacttacaatatagaagcatttagacacacactgaataataattttttttttgagacagagtcctgctccattgcccaggctggagtgcagtggtgccatctttgctcactgcaactccgcctcccagattcaagctattctcttgcctcagcctaccgagtagctggcattacaggctcccaccatcatgcccagctaatttttttgtatttttagtagggacggggtttcgtcatgttggccaggctggtctcaaactcttgacttcaggtgatccacccgcctcggcctcccaaagtgctgggattacagacctgagccaccgcgcccagcctaaataatgaatttatagatgctacactgtatggtttcctttttctgctgctgtacaaccattcaagtaacataagtttcatcctggttcttaatgataccatgaataaagtatagaaactctttagctgaggattaaagattgttgtgtttaggtagaccaggtttctgatgcagccctcagtataccagggaaattagccattactgtttgccttgtaggtctaatgaaccctttagctttttattttgtatatgtctaagttactctacaaattcttatggaagtataaagagatagtgaaagacaatcttatgagaaattttaataagaattgaaatacaggctgggcacagtggctcactcctgtaattccagcactttgggaggctgaagcgatggatcacctgaggtcaggagttcgaaaccagcctggctaacatggtgaagccccatctctactaaaaatacaaaaaaattagccaggcatggtggcgggcacctgtaatcccagctactcgggaggctgaggcaggagaattgcttgaacccgggaggcggaggttgcactgagccgagatcacgccattgcactctaacctgggcaacaagagtgaaactccatcgtaaataaataaataaataaataataaaaaagaaatacaacttactttttgtatcaaataaattttggtgcacaaacatcataaatgacataatatctgtcacacatcctttagcaaaacaggattgttaaaattcttacagctaatattatgtggtgaagttcttcctgttgaataattaagtggattaaaatatcatatttcctttctgtcattagaaatatattgtgcattaatcagtccttgggcctgagaacatttatctaggttttcatcatctgaaaactcacagtccaatatttcacctgtattatcaattcaccattatcattagagtttagaatgctgctgtctctttccatctatcttctgttgtatctaataattaggagatatatatatatatatatatatatatagagagagagagagagagagagagagagagagagtgagagagagagagagattggtaataatactaatatatataataatattatatatatacacacatacagagagagagagagagagagagagagagattgattttaagccattaactcatatgattgtgggagctgaaaagtcaaaaatctgtaaaatccaaaagcctggcaggatggagaccaagggaagagttgatgttgcaatcttgagtccaaaggtaatccgatagcagaattccttccttttctggggacttcagtcttttctcttaaggacttcaaaattgattggagaaggcccacccacattgtggagggtaatctgctttactcaaagtctgctgattgaaatgttaatcacatctaaaaaataccttcacagcaacatctagattggtgtttgaccaaacaactgggaaccatagcccagccaagttaacacataaaatttaccatcatcagccatcatcatgaggttcataatgtcatgtgtttcttcacatataaatctcgtggttcttttatttttttagtagtacctagttaataaggtacctggaaatatacgttaaataatagaaatggtctttaatatcttaatgaatttcagagttctatcagagtaattgtaacttgtatataaaaactgatatataaaaaacaagtatgttttttcttcctatacgttgtgttaaattaatactaattacctatcatgaaatgaatgttttccatttttcataaatgtatttcatttttaaattaagaggattaatatcaaaacatatttgaataggatttaaaatccaaattacctgttgcgattatatttaattataaatctttcaagtgagtttataataggaaaaatattttttattaaagtgatgtaccataattatgaacataaatagtgttttctactaattgtttttgtcttcattatgtttctttttaaggggaaagatctgggttaacaaatgttttattcatccaccttgaaaattaaacataataaacaaaactattaaataaataaaccaattaaagaaaatagtttttcttccttttttgtgataaagatgattatgagtaaatatattaaagaattttatactcagacaaagtgagctaataagacaaatacaaacagagatgagcccagaaagagatactctacatgtttataggctagcatttttatcttatgacctacagggtcattgtaactaaagtcttgaatttctgttatattttggtacctgtgggcataaataggaatattactccctgatggcatcatgcattttagataaaactaagccaagggttaaacacgtgccatggactgttcctaaaaaatgctctcagctctcaagttatagaaaattgtagggattattattttgtacctactacttctttctttaccaaaatttacttatacatgatttgaaaattgcttgccacctgtctttattctgttgtacttgtcttaagtaaaacatttataaagtaagtaggaaggattaatagattgtgacctttctacatgaaaaggggaaagccgagcttgttctacttttgtcaggaaagagtttgaatagttacctgttactgtaaaacaatcaccccaaacttagtgtcctaaagtagcaataatcatttattatcactcataggttctgtaggtcaggattcatgcattgcttggttgaggggttctgtctcacgatcactcatgatgttgcactcagatgtcaattaggctgcagtcatatgatggcttgactggggctgaaggattcactttcacagtgagttacttgtatagcttgtgagttggtgctgtctagttggtttccctctaaacgagttcctccatgggactacttgagtgttcttatgatgtggtggctggcttaccccaggacaagcaacctatgagttttgccctgagttttctgaaacatgatcccatgttgctttgaatatccccattagacactcccacaaatgtcaatcatactctttgccctaaaagttgcagactacttgtacatgtcctttatcctacatgtgtacaggaaacacgtgcagaataacatgtgtttttgtttcttgaaagaaacaagaacccaccaaaactttatagctccgggacatgatgataattatctcaggtccactcagcctggctccacatggaaaatatgggtattgtaaagaaagcttttgaattttggaggcctaacaaaaagacctaggaagctaatagttggttatgagataattataacaatggtgatgaaagcaattgcgattattacttttatgttagtgagataaatacataagaatgtccttctttaatttcattccttaatttccttaatccccattcttgatttctggattagaggaaagctatactatctacctgattacacaaaaagactggactgaggggtaaccattaggactcttgttagttgaaggtaataaactatattacttcagtggctaaaacaaacaggagcttttttctcatataaaactagaagtcaggaaacaaatggttgctgatgttggtttatccgtttgacaggttctacctttatcctggagattctttggcctttatttctttagtaatatagtagtaacagcactgaatctaaaataagagcacttggtccaagcactggctctgcccccttagtgacagtgggacccatggcaaattatttaatctctccaacagttaattttctcatctgtaaaactgggatcctgataacttccatactagtttggtgttaggattcaatgaaatgacgcatggaaaagtccttttcaaatggctactcactatacaaatattaatgtcttagtaagctcaggctgctataaaataatgccatagactgggtggcttaaacaacaggcagttgtttttcacggttctggaggttgagaagtacaagatcatggtgctgacagatgtggttcctggtgagaaccttcttcttgctgtatcctcgtatgttggggagagaggtggtgcagagaggtactctagtctctcttcttgctcttcttaaaaggacactaatccaattatggaggcctcaacctatgacctcatctaacccttattatttctcaaaggccccacctccaaatatccattgggggttagcacttcaacatacaaattttagccggcgggggggatgccagcattcagtccataacaatcaactatcctcattattgagagttccactaggccttctcaagggttgacaatgatagtgccctgctagctataagagtaaaaaaattttcacatctagaaaatgggtgcacgaatattggtaactgttctaatgtctatgcacagaatttttagaacttgctcgagaaatttgtctttcctgacagtgttgcttctcttaaattccagtcgagggccttgaaaaaaataggttttccatcaagattctctgaatgaataaatgaaatgctctgatattttctttcaatattaagataaagcaaaatgtatacagaattttctattttcagtgtttccaattactgcatggtttggcttattaccatctctaagtctaaccctgggtcataggagtaaagccattgggggtcccctacaaaggatacaaggcagtggtagatacaataagcctgacaattggagtcagatttttctctttcatacaggaaccatggaactgttgccctgggccacagctggctctcagaaaaacgtagaactggtcgaggaaaggaaaggcagggagtccttgattcacttgttatcagaaaattgtctgttcaaacaacagaatgtttgaaaagataaaatcaagtctcaaatgcacagaataagaatgaggggaaaacacctttctctgtaattaaacagaaaagtgtgccatgaagtgataaaacccagtcaacctgaaaatgtgaacatcaagtgtaattgtgcagaacgataaggtaatatgaacagcacaggaatatataggctcacgttggcattttaaagcaatggcactttacctgttcctcatgaaggatttggtttagtctaatcccttgtaggagccactaagaggagaaggcacagtccttttgggatgtaaaatggggaattctttctatgattacaagaatttgtaaattgctcagagttttaggacagcaggttatgtgtctctcaatgttgggtaccatgccaaacgtactttaagagacatagcaataaatagaatgggattcatttttttcttaatgtctggcagggcaaccaaaatgcccacgtttcccttcagtagcttggtattttggtaactaaaaacatgttccagggaactccagaatatgaaacatttcagacaatttgaaactgtcaaaattttcacttctttatgggacaaataaaatctaactttattcagattttaaagtatctcataaaagagtaatactttagatttgtgctgtgctttatacgaattggatgaggaactcttatacatatataagtggattttattttcacaacagtccctatggtagatattgtccttattaagtaaatgagagaaccaactatcaaagatattaatattttgcataagatcacacagatagcggaaccaggatttaatccaactcctctgattctaaaaataggtgttagatgggtattctttccccaaacctatgctgaaaggaggctacatttggagtcagttattgaaagtttaaatatatgtgtatatatatatatatatgtgtgtgtgtgtgtgtgtgtgtgtgtgtgtattcttatatactcatgcatgtagatagttataaagtacatgcttatatgtcaattttatatatacgactgatatataaactatcaatttgtgatagtttatttcaattaagttcaaacatattgggtatttattgactcatgggacttaaaatttctcagaggcattaatttctatgcctttaagcaaaataatgtttagcccgttttagaaagataaaaggctaacatatgttttgtttttgtttgttttgttttgttttgttttgttttgttgagatggagttttgctcttgttgcccaggctggagtgcaatggtgcgatcttggctcacagcaacctccgcctcctgggttcaagcgattctcctgcctcagccatctgagtagctgggattacagacacgtgccaccatgcccggctaattttgtatttttagtagagacagagtttctccatgttggtcaggctggtctcgagctcccaacctcaggtgatccacccgcctcggcctcccaaagtgctgggattacaggcgtgagccacggctcctggccgctaacacatgtttttaaaaaataaacaccattcgagtaggaaaagtgggagaactggtggttcagctagagatacggagatgggagggcaagttgctttctcatgttgccacatctgtaagttacaaatgccatagattaggaatttcaacgttgttgtgtctctctgaggacattctccatgtagtatatgtttcgcctccctgatggacagtaagcatcaaacggttactcctttatatttgaattattttaattctaacatcttgttctgaaaataacgggaatgccataaatacttactgattgattggactgtctcgcaggggagctggcagctagtgtaggctacaagctgccatcatcatctccatgaccatgagtcatcactggaattaccctaaagaggttaataaaacgccttgtttgaaaagcaaacaaaaagccagaagaaacccaaagcaaacagacccagactttgcaatgggtagtgaattttcagtcttaagggattgagagtccttttagtacaatctctcataaacatataacagattttaaaagcaccttggtctgattccagttcacatacctcatccttgcagaatacaacatactaggccatttgaaatgttcctcagtttacttttggaggatctgctgatttgagcaaaatggaaatgaaatgtatatagaaagctcttaatttttttttttttttttttttgagacggagtttcgctctgtcgcccaggctggagtgcagtggcgggatctcggctcactgcaagctccgcctcccgggttcacgccattctcctgcctcagcctcccgtgtagctgggactacaggcgcgcgccaccatgcccggctaatttttgtatttttagtagagacggggtttcaccgtgttagccaggatggtctcgatctcctgacctcgtgatccgcccgtctcggcctcccaaagtgctgggattacaggcgtgagccaccgcgcccggccagctcttaatcttaagaagttcagtttcgcagctccccccccaccaacccccaatcttgaaagcaggaatttggattagggttttttaataaaactttctttttcttattaccatgatgttttttgagagggtacagagaactcattaaaatgttatgttataacttaagacaaaatgggaatgagaatttgcattatataaacaaagtgtatgactaagtaacctgttacatataaagtgtttgactaagtaacctgttgtccctaggagaagtagaataaatatcaacatgtggcaactaacagaatgtgttgtgatcactaaagcagcacattcattgactttacacttcattaagtaggtaacgaattttacaaattttaggacttaaccaggctgtccataatactttgcatcagtaagtaccaaaatctacgatagggcacttcggagttccttaatttaataatattgataatattttgtttacattttgatttaattgtatcatttcattatttttgtgtcctgattctaaatatatacgtcaacaagctatatggccattcaaaacaggtaaactttaaatatgtttttgacatagaaattatgtaacagtcttcatcatgagtataggagtcagatttccaattgaaaatttcatcctctaaaatacttggcatatgtatacagttgtctagttcagacaaaaaagtattgaatataaagttgattttgtaaaaccagaattggcatccttaaggaatacaaataaagggaatcttaagataatataataatgctgatttgaaatgaaaattaataaaattgttattctattttagtatgctaaattggacagatccttatttcattgaaaaattagattcttcagtatatcagtgcactgtgctaataacacaagaagattaaattatgtccatatgtgatttggtagaatttaattataaatataataaatccttcaatatgtacagcagtgaatattccaaatttatctctcatataagttgtgagcaacttaaggataggcaccatcccttaacatacctagattaagaacagtacctagaaaacagtcatcaaatgcttcctaaataaattttcaccttcaagcttatggcagcaaattaaacatagattgacccttttctaatagagacagaatagatgagactgatgacttttttttataaatgtgtaagtctgcaaatatttagtaaggatctaatttacacaaagaaatcaacctagagtgagattattttattcatttcccggaggctgtgactgaattatatttgcaacttgccctaatttttatacttaataatttgcattagtatttaaattatgtgaaggaaaaatataagtttaaaatactgaaaagtatattcacacattcatcaaatgttgattatgtgcttacagtgtgcaaggtattggggctaggaattatgtggtgttccaaggaaaccatgatacagcctctttcattacagtgtttattattaggtagaaaaagtaaaattagtataatattagataggatatatgtgtcataggagtaatataaacaatattttatgaacaatgtatgaaggtataattttgttcatttatccatttatcgctctgtacatccatccgtttattcattgttcattacttcatttaataatactttagtgaccattggaaaagggatttataaaagtgagtaaaatgttaatcattttctaaagaagttgataatctagggaaagacaaatgtgtaccaacaataacttggatataaagcataaattagaggtacaagcaaagaaaaatgaaagtacaaaggagaaaaatatttggctgcatgagagcagggactgtgtcttgtttcatcatagattttccagaacctagatcagtgtttggcacaaagtaggttcagatgagtttgtgttgactgactgtctactgaagtaattgtggaacatatcatagaagaagtagatcagaggctggatcttaaaactgggagaaatacatttcagctccgtgaaaccccagaagtgatatttgacattagaaagcacaaagtcacactctaacagtgacagtactgagtgaagtaagaattttctgcaccccttcttcttcttccactcccattaacccttgttagggtgagaaatagcagctggtgaatggtagagaagactagcaagacagaaagaggaagcccaccatgcccactaccccaggtagtaaaggacctgtccacttccatcctcttctaattgaggaagaaatcatatatgtgagttcagattgaagtattgattaatatattgagctggatattctaattttaatgtctcgatatggagcaggactttgtttccttaatgatgaccagaaaagacatgaaacttacccatattttcacccaagaacagaacagaacaacctacttgaacagatttaaagggagaaccaaaggcagataaagttgtctttattcttacacagcataagtcctgattattcatcaatttatacattttatgaatttaagaaaaaaggaaaggaagactataaagggattcatatcaggatacacatgaaggtaatgtagtcagttttttagtggaaccaaaatattacagtcatcactgaacgaaaatattacaatcattacatcattcactagattaagtgaataaattaaaaatatattaaataaaattaacaaagacatgagcttttcaaagtgtgtggtaacctggagcaataagcagtgtagttggaatgttacctcttaaggattatcaaagaggctggctgttcagtaggacagtggtatggtaaagcaagcttgctacattgcagaactccagagggcaacattctaattatctttagctatggggtctgttcccataggataactataacttagtaggttgacagagcctcccaagaaaccacagcagtgctcaattgtggcgaattacttctctatcccatcaaaatgtcgtgattggacccacaactgtgcatacatttttttggcattttcctgccagaagcatatttacatttctttaatatccagtcatatccttcccttatttaaaaataattattcagagaattccagttttttgtaatcctctcttctatgaattttagcaacacttatatacagatacttgatattctggagaaatatgcttcaagaccatgtggattaacagattttcaaatgccgttatagtatatgtatatactatatatgtataagtatgtgtagtccccaaaatgcgtactttaaaatcctcataatagatctcttacagatgaggaaaagaagatgtagaaaggttacataaagtagctaacaagtattccaaataatattgaacttcagtctgactattccaaaattgcattcttaactctgatttctatatttgttttccattctaaactgtgtattgtgatataagttcctcactaaggctctttttcagggtcttcctaatactaaagtcactcttacaatgagtattttctttacgtgtgaaatccaataggcaaaaaaaaaaacttggcaataaattttaggcattaacctcatgccaagtaattatcagaaggctgtaatgctttgaaacttcacaagtctgattttaagataatggaatgaggcttgcattgtgaactttcttgatgctttacattgcaatatgctgtatagattgacttcctaaaaataaaaaataaaaaaaaagatttgtagagcatactgggaaggtcttgccaattaaaaaccggagattggctgaaggcttgcagcaattgaattttgaatacagatggtgtcaaatcgagtgtttccatagcaacagactcttccttaataacttttagatgggagggggtacataaaagagaaaaccatcttttagcagatgtatgttttcagcatgttttcagattgattttggaagctaatttgtacttaactagtgattgttttaagtggatctaaatattactaaattctcctgaggaaacattttgagaataacagaaataaactctaggaactttataaagacatgaaaagggcacaattttacaaaacctttttttttttggtctggagccaatcaaacagtattttatattgagtatgacctatcaatagtcaaagagttcttgattcttaatgcctgttaatattgaatgtttagaatatgggtaaaatcaaggaaaaagtgctatgtatctcatggatttggaatatcttaatatactctttcttcaggttaatgattatttttaaaaaatgatgatataaatcatttcaggaaggactgcataaagaggtcattgatagagttcatatcatatagcagagttgtcgtagatttaatgtccaatcctcagttttgcatagccaacaactaaattttagtagtttacctagagatgatcaggctctgggagactgattcatgctacacaaactcacaaggcactggtttataaaccatttgaaagaagaaataataattcaaactattattttacaacaagactggcaggaaaatcaataatggtaatgtgttcggggtgtcctccccagtgaggactatccagaagaccaaatgatagattattgttcaaagtgatagaattgggagaagggtaataaagcattatgaaaggatgcttccttaagtgagaaaagctacataaactacccatctttactattctagatgaccttaaaaatattaatagaaacagagaaacagacaattcatctcagatggtacaaaaaacataatagcacatgtttgctccctggcttccttttagattccctgcaagaggttttcccccaaccaccaccatatacagatatacagctttaccttcactctcctattatctgtcacagaacaatataaaactcagatatgttagtataagttaatagttggtagctttatgttaatgacattgctacctgttacccccagattcatctgggttacttcatccccaaaaccctttcaaaacgacatttcccacaaagatttctagtattactggccattcccggacatcttgcttccaacataagaaattctgacataatagggatataagaaggccttgggaatctgtatttataacaagctccccaggctatctgaggctcagccagtttatttttgcagatagctattactatccacaatctcttttatcctattgtaattgcagaagaaaatctttctttctagtttctttgaaatttctggaatgttaggacttagagcctctaggaacttgtacataaaaagagagacttaaagagatatcaaagtagagatagagatagatacagataagtgcatagacatagaagtagacatatataatagaaacatatatgtatacatagaaatagaaatatgccatttagaactcttaagaaatatgtgcactcttaaaatatagtttaaaaatgtaaagaacttgctattaattgaaaatagcataataaacttaaatgaaataaggaccccaaacgcaaatatttctctctgacacaccacacacacacacacacacacacacacacacagacagagagagagagagagagagagagagctgtacaataacaaccaattcctgacccagataaagaaaaaatttttatttatcttctttctcattctattattattttttctagattaaattaaaatggtaattatgtgtaagaacaagcattattgccatataaacaatgcaacaaaggaatcaattgaaaattaagtgaaaataaaaggtacagcttcgttaaagacccagttcttagagcttagtctcaaactctttgtactctgcactcttttcttgatttgactcgattgattgattgattgattgattgagtcagagtctcactctgtcacccaggctagagtgtagtggctcaatctcggctcactgcaacctccacctcctgtttcgagtgattctcctgcctcagcctcctgagtagctgagactacagacatgtaccaccatgcccagctaatttttgtatttttagtagagatgggacttcaccatgttggtcaggctggtcctgaactcctgaattcaagtgatctgtctgccttgttcccccaaagagctgggattacaggtgtgagccaccacgcccagccgattttactctctttagaactgcaaaagtaggaatctagctcatatgcagacattctagaaagtttgatttcaaaagtcttctcaaaagaaagagagcaagagcaagaaagaaagcagagagagaaagcagaagataaaatggcattgtttgaacagggatggaaactgagtaagaaatttggtcactaaacactttagtgtctatcatttaagattgtaatttggttatttatcactggaaagtgattaataatctaaaatgcattttataatactaatactattaaaacattaatttttggagaaagtttattatagattgatttatacttaccactgaatattaaaatgtttaatggaagtagtttcaaatagtatttaatgatatagggaattattacaacattaatgctcaggaaaaaaagtaggatatggaattatttatgccataggatcctaattttgtaaaaaacagagaaaaccagcaaaagaaattataactggaaggaaatacatcaaagtggtttgcagttatcactcatgaaattgaggtgaacttgatttttttccttttatatccatccgtgttttaataccacagacatgctttagatctgcccgtatattgtggctttcagagggtaataaaccattgtgactcagaaatagctaagaatttttatctctcaagaacaaattttcactcccttggggtgtcattatctgttgagaatgcatgcaatagttcaagagccaaaagactctgattcagtaagtttagggtagaaaaaaattaagtacatttttaaaatagagctctggtgtttcaaaggcaatgtgcaaatatacctacttaatattattctaattttttcaggatagctgaaatataaacatctatttttagtaataacacaaatgatggaatgcttttacatatttataaatcattaaggatttgctttttgtttctactgtctggaacatataaatttgaacacaatttagaacaacattcaggaaatatgatttatttattaccttcttgacctttattttatttttaccatctacttttatgtaagtcttttttttcaatcattgtttatttctttacttttccttccacatagaattaaagggagattcgggcttagtgtccttaattcatagttccattgtggctattaaaaggtgaactgaaagcttgcaaacacgtggtaccttgtagataattttctccagtcagacagttagataaaggcctctgaggtttctggggtcattgttgaagctatgttttaaaatcctatatccttctctcattgttggttccttttcacaacacagaagttttcttttttttaatttcaacttttagatacagaaggtgcatgtgcagatttgtcacgtgggagtattgcatgatgctgaggtttggagtacggatcccatcgccatgttagtgagcatagtaactgataggtcgtttttttaacccactcccctccctcctccctctagtagtccccagggtctattgttcccgtatttatgtccatgtgtgctcagtgcttagctcccacttataagtaagtgagaacatgtgatatttggtagaacttttcatttttaagttaaaaaacaaaacaaaatgaggatgagtggaagaactattcagcacagcaggttataaaccaattaggatgatgacgccctgaatggagattttcatgaacatctcatattagctatttcagctttggtttttttaatgttcaaagtaaatagaataatgaagaggctatttaggaaggtttagactgagggaaaaaaatcctttcattaggttccaaataacggttagcttattaaacagcaagaggcagagatttagcagagaaaaaataaaaagatttaaaaaaaaacaacgagattaaaaggtcagtaataccattggaactggcagcatggcaagtttatatcagctatcttttgttttggaacacaactatgctaattctgttctgaacctcttgctaatgcctgtctcaagaaaatttaacatactttatctgtgtgtacaaaaatacctaaggacaaagctattacccaaactgtattcagattgaaagaatccatatagaaatttgcagctaacgtattagtcagtgtatgtaatttctactgcttcacagcacaactctttctaattttcaggagcaatatagcaactgcttgccagccaagagaaaaccataggagcattcttatcattggagccaacattagttctgcctacagtgactaacatagatgcgtttattgctagctggaattttccattggcactagttacatgtaataagttagtgctttcaaatggaccgtggaatataggaaaactagagtctgacgtaaccaaaaaaaaatgttgataaactcagagattatgaaagagaggagaggagtggtttttgtggaaatgatagaaaagcaaaaaaagatggcaggatttggaaaaaagaaaatcaggttagagatttaattagtaaaggagctccttttaataattatataagagtatgagtttagagtaactgcctggctaatatgtacaatcttcaagttcagtcgttttccaaaattctaacttttagcattttttttttgtaaattttaagtggaaatcttccacttttgttgactaacttggctacctgatattttactcaacctcctactttcttgttctctacttcttctgagtctttgcttccacaatgaggtagctcatattccttaagtttcctgtctggttttgcttttttttttttttttaacagctaaaataaactctacaagcatttctatcattttctttacatccaatctcttgtcagccatgtcctaatcactttggtaaagtatcagtgaccacccaagaggttcactttgcttcgtcaaaggaccacctgtcatgctttatctccagagcttctgccttgaaagtaagaataatataacctttcctgaatgtcttatttcaatttcatgcatttgatctgcctcttcagaccattcccaaggctttgcccttgttctttaccctcatggcttcagttattcatcactatatgaatgacagctctcaaactgacagctctggcctctcacctatttcatctattccatatggcaaactgtctatattacatttcatcatgagggataattataattccacattcaacaagaaattgtgggtccgtgttgatttccaacagcaacctttattttttagactatgatagagaaagggtcaactttctctcttacttatttatctcaaataactcaaagtcaagctagaaaagccaagcaagtaatatttcccaacaaagcagctttaagcaaaatgattttcagacacgacatgcagcctgtattgtggaagaagcacagtgacctaggtgtactttgctgaaaagcagtggtcacagtccattagtttcatctttttcctgcaagagagaaaagtatggcagggccaagactcctaagaccccttaaaaaattgtgaggtttttataaccctgttcatttcccatcaaaagcattcatgaagcacctgccatgtaccagatgatgcattctaggcagagggaagaacttgggcaaagtcctgtacattttatcttctaaatcttactaatcttttcttatctttccatctaccacccaccatcaggctgtgcctggactcctatgctaacatcttactctgtttttctgcatcttttttgccctcccctccaatctactctccaattagcaacatgactaatgttttcaagcccagattagacggtgtcacttccctgctttaaacctttcagtggattcccattgcactgaggttgaagaccaaaatctttaccataactcacaaggccactgggagctatgaaaattttcttttttagtttttccttttaattagtgatgcattcaataagtggagtctatcgtgaaagcatagattccaaaggcatgtgaatactcaattatgtaatttacttacagaatgtttattacttcttttttagctttgattttgtatacatcctaactcaatctcagttatataagagattataaaaaagtttgtgatgaatgcatacataaaggaacaaataattaacaaatatataaagacaaatcagtattgaatgaccaaatggctctttatttattaaagaacttagaaaaatatagtcttgcttgagaccaagttctggcaacgtatgtgtgtctttttgtttgggttaaaaagcattagttttatatgttttaaaaggataggatcttcccttggctgcaaatagtataggcaaaatgctattctgtatcttattttaaagaaaataacaatcataccttattatttgctgacagttttgcagtttacaaagcactgttcacatggattaccttattttaaattcctcaattttttttccttttactatgggaattctgtttcaggagaatttccctttaaaattaaggtgattatactattctgttcctgtatttcgtagccatatgtttcaccaaggtattgtctctctccaggtctttcttctctggatgtatagttatgattttccaggaggtaaatgaaaacagtgggagcaattgacagagtgttttttgtttcccataaacccaagcattgcatttaaggagctgctgtactattgtaagagttcctatactaattttaaaagttcatttacatattttgtagatttttaggataagtcatcagaaaaaaattaaaaataaatttacatattttgtattaagctgttttgttacttgaatggggattattggaacgaggaaagaattacttttatctccccatttttcaaatcatttactataacattcatgaattgctgaagtttaaacaatcaaaaatatctgaaaatggagcctaggaagggtaacttatgtctttcatactccttcctttttggtttttctaccagcattttagtctaaaatatattttattttcactgatcctgtgtttgtcttaataatatagtatgtatgttaaatgaaccagaattcattgatctttttttttattattcctctttttgcttttctggagaagcatttgaagaaatcagttcaggctacacttcatcaattcatttcctcatcttccccttaatacaaaagccccattttcactcttcaaaacatttgatcacatacttaattttcatagcattgactaaatgtatgtgtttattttgtttatatattgcatatttgtgatgattttcacttttacagtttaggcatgggtaggaaggacacttacatgagtacttaagttcttggttatgacttgagaaactcacaaggtagcatggaatggcacgtaaacaaggctttgatagggtgaaagggaattatcaggacaaaactgactagtggcagtgacttttgagcagagtgctgcaaactgaaaaggcagagagcattgtaggcagagagaaagcatagacacatgtgagatacatgtgaaatacttagacaaatgtgaggtgtgtggtaacattgtacatacctagggctccttcctgtgtagctgtaaagctggagtataggcatcacaacaggcagcagaagccaagaaactagaaaagtagacaaaggccacgttataaaagatcttgtaggataggtaaagagttttaatactatcctgaaagcagtgtggggtgggaggcatgacattaaggaaatttaataagtgatgaaagaccaagattttcatcttcagaagacttctctggaggtgggtggaccacttgatgtcaggagttcaagaccagcctggtcaacttgatgaaaccccatctctactaaaaatacaaaaaaatagctgggcatggtggtgcatgcctgtaatcccagctactcaggaggctgaggcaggagaatagcttgaacccaagaggcagaggtggcagtgagccaagatggtgccactgcacttcagcctggatgacagagggagactccctattaaaaaaaaaaaaaaaaaagacttctctgactagagttttaaagaaggatggggagtgcaaaaggctaacattaaatcagtggttctcatccctggctgcacgttagaatcactagggggagtttaaaaaaaatgccaatacttggactccaccccaaaccagttaaatcagagccttaatagggcccagacattcgtagtttttaaagcagccctcttgattcaaatgcacagacaaggttgtgttccgctgaactagagagaccatcaggaggcttttaactaaagcaatctagggaagagagagatgaatgatatagaggccagaagagctattaaggagaagtgactcggcatcttcttggttaaagtataaggaagaaagaagaatgaaggataatttgcatgtttctccttcagtgactaagtagaggaacagatgtgtatcagtgattaggggagaggaagaatgaagatgatgtaatccagtttggatatgttggctatttcatagagatttagtaggcgtttggctctatggatacagagttcaagagacaattcttagcttaaaacacagattttactctgattattttagagatagtaaatgaagcaatatacatacatgaactcaccagagagaatatgtaaagtgaaaacaaaaagatcaaagggagacccctagggaatagcaacatttaaggaatgggcctaagagagtgaggagtggccaaataggcagaataaaatccaagaaggaatcttccatattaatcaaaagagaaggtttcaaagagggttcgtcaattggcaggtactgaagggatatctgaaaacataaagactttagactctctaatactgtagcaactttaaggtcactgctatcacctacaaaagtaatttcagtggcaaaagccaaaacgtaatggtttagaagggtatgtgaggtgtggatgtggaaatagtgggtatagactattgcttctcaaaatgtaatcacctgaaaaatcttattttaaaatgcagattttgattcattagctctagagtggagcctgagattctgcatttctaacaagttatcaggtgatgctgatgctgctcatacacaaaccatcctttaagtagcactggtgtaagccactctcccacaaaggaggaagacatagggtttccatcaggggacgcaggtggggtgtaggaccaaaggaaataatactcttttgtcttttgtttggttggttttggttgttttcttcttaaaataaacatggttgtagacggagattgaagacataggaggggtaaaagatggaatcacttctcatagaagatggaagagactggaacattgagtacagcgagggaattaggcatgggtaggagggacacctaaatctgaagagaaggaggtaaggaagaattgaaatacagaaaagttctgtcagtaagcaatgtatggaattgtgctttttagccatagtttctgttcaagaaattgttttccatttatttttatttcttaacttgaatagttggatggtggagtatgcttgtaaaaataagagtgaggccactttgtttttcggggttaatttccgttttctgcactcatcaagtaaaagttgaagtggcaaggtgtagatatgaaattcagtgtgtgctaagggagaaaaaaatgctttttattctacatgattttaaaaatatttatattccaacaaatgcatcaaatttgatgtgcaaatttacagtgatgaatgagttttattgtgtgcattgcatgctggtgacatggtaataaatctgtggtgctagaattataatggtcccctttagcttcgctttaatgaactcttgctgaacacttttgagttgttagtactttatttgctacatttggcacttaattagttaatgactgagatgctggaccgatggatcatctaatgatatgttaggcctattatcacatctagatagtttcttttctgtgacttgtaagtgacctaagatgataaactgaaatatttttgcatagatatacatcaagcttttctcctaacttcaggctttcatcttaagcaatagtttccaacacccccaaaagagaagtcattatgtttttaaaaaaattattcattttaatgtgatcaaataatatcacatttcagcattcacctatttaattaataaaacaacttacatgtttcattatgactggatgttgatattttttcataatctattatcctccaaccagtggtaaaaacccaatcctcctctcacccagctcatctttccgtatgggaagcaatacatacttccctatgttttattaccaaaacaggagaatgagctttctttagaaggttaactcattttctctattagaatattcagcatacttttaaggaggtaatctggtctttgacagtctgttgattagaaaattaagagacctgcctaaattccatttccaactcctctccatacacattgtgactttgagcaaaacgttttgccatttccactcgtataagttctgtttagtatctttaaacttccatatcccacagatggtatttttttcttcattggaaagtggtgttagtgattcagaaaactgcttaaataatacactgctttgtgttttctgtgagagaatttttttttttttttgagacagagtctcactatgttgcctaggctggagtgcagtggcgcaatctcggctcactgcaacctctgccgcccgggttcaagcgattctcctgcctcagcctcccgagtagctgggattacaggtgcctgccactgcgcctggctaatttttgtatttttagtacagacagggtttcaccatcttggccaggttgatcttaagctcctgatctcatgatccgttcacctcggcctcccaaagtgctgggattacaggtgtgagccaccgtgcccagccgagagaacattttatctaacattctattttaaaatttttcaaatacacagaaagctgaaagaattgtacagtgagtgctcatgaacccactctctagattttgttgtatcactttacttctttctctacccgccaacccctattattttctgatgcccttcaaagtaagttggtgacatcggttccctttacccctaagttcttcaacatgcatgtcattaaccagagctcaatatttgttcacatttcttttgtttgtttgtttgtggcaaaatttgtatagactagaatgtgtaaatctcatgtgtattatgaaatgagttttgatgaatgggtatacctgtgtgacacatgcctctcttaagatacaaaaaaatagccttggcccagacaggagggaacattttttaggttggcttgagtttcctttaactgacatgtagcatgactgaatatatgaccataagattgccaagttgaaatttaccaaaggtccatccaggggaacagtatggctattgatacgtcatttgttcatctaggcactgggctgggtgagttcttcacaaaaaccttgtaaggtgagctttcagctctgacagaggcatcaagaaacgtatttcacaaactgttagagctgaaaacatctccagggatcatccaatctatcctcctttgctttatgactcaggatttaagaagccccagaaaagtccaacagtgttaccaaagtacacgattgcctatttacgcccctataattgagagctgtctaggggtcaggtacttttctcagtgccttgtgtacattatttccttttatctttctagattattaattttcttgttattatccccatataatagaggagtaaacagtgtgaaaatagcactaaactgttgatcttttccatgttccaaactaaccttaaatgttaggctaatattataaaattttaaaatttagtcatttgttctgggtctggcttccatgaacttaaccaaaatgcttggctgtttctgcattctagctttttgggatttagagaaaaagcaacttggtcatcatagtggccaggatttatttatgtgccaataactcccctctgtttttagctttttgatctgttacatctcagatactttattctctatttgtaaaacaaccacattcctccttttctatatctaagtgaaaaaaaatgcccgttcatactagctacatggttagcatatatgtttcttcctctttgtgggtttgagttgaaacttcttttgcaaagcttaactatagataggtcttccacagaaccctgtttaaatgccttgtggaagtagaagggacatatcttaatattccttataatctgtgttattatttggactttcctcagttccttatatgtctttgtatgtcaaacattgagttatgcttacattactctaggtatagttgtgtcctagctttgtacaaggttacaataatggtggtttccttgttttcagcttcattcttgatcatgcctaacattttttgatggttttagcctcggagaacagtctactgctatttctccctttttcctgatcccaacaaatagtttagaaatctttgtcttaactgcaattgggatcatttttcctttagagttgcccacagttagtcatgtccgtcactcttcactcactcaaatgacctcaaaagatttacctgcaatttaagcctttcaccttggcattttctacctggaactctggcgtactctgtgggatcagtgagatccatagtgtgcactccctagcagatcgtgaatgtttgttgataactataattcattgtttagtcttgtctatacagagaagcagtagcaccttgctttagtaggatcctttatgcttttattcattattgaattttatcttcacaataattctgtggttctgcagttgtcattatttcagtttgggagttgaggaaactgagatcgtggcaaatcaacttttcccatttaaatccactatttcatcgctaatccagggttataacccaagactaactgctagtcaaagttgctttctaaaaatcagttacacagacttctaattctcgtgctgtacagtcagtgcagaaattgcaacagaactgttctttgttctgtagcaatgtgtattcaaaaggtaatcatttcttgtgtataaataacattcctccctaagagagatgttgtgggaatttatatcagtcaagaatgtatatcgataattaaacatagctagtctaaaaaacttgaagtgaaagattggttcgtattcaatcaactgaactgtttctttctctgcaatattgtggttcttgacatgattgctgagtttccaatttgacacttctggaggttcgtaaatcaggacacatgctgggtctcagtctgtcatcctgagtcttagcattggttatttatttccctttattacccaacatctatttactgcacaaattagtcgagaagctttccatgacggacctttgtgagaaaaaaaaaatgtgtacattgggtagctcttcaattacaaacatgcacagattgtctgggtttatgtgcatatactttattattatttcacttttgtatgtcagatgaatttggtgtaaatgtaaatctaacaatatgggctctagtggccaaatacattgccataatatgtttttgatctgtcagtctcctgggtgaagtgtcgcaagtgtggtaaaattctgtagtataaattgatgctcagttatggacaattaccaggtctatatgatgtcagactaccacactgatccacttttaggataacagcttcgctctgatgatcttcagattttagatttgctttcattccttgacaaaatgaaatacatttaaaacttctaaaattgtcttttgtatatatttctgcagggtctactcttgggactactataacaatcaattgtactgctttgtttcttttattaaatagcagaatagctgcttgattcgtctgtatgtgctgatgtgtgtctgtgaatctagtccagttcactgtccaataagaatttctgaaatgttctttgtcacatcaatgtacagtcaaaatcgtattgtgtttgtagcagaatgattgcatcttttattattcacagcagccaaaatgacacatatttcacacgtgacaccttttttaaaaaagatgaattgtccaaagttgtatgtagaatatatttcacaaatcaaattccctatttaataaatggtgctgggagaactggctagccatatgcagaagagtgaatttctataaatagaatgcaactgtgtaaatagcatccaaatcaagaaacataatataatatagccagaagctcaggatcccccttcatgtcttctccagtcatgaacatttcacaagggtgactactatccttacttcaaaagttttctattactttttcctgtttttgtgtattatatgaaggaatcgaacagtcatatctacttgtctgtggctgtatttttccccccaacaatatgtttgtgagattcatccatattattgtatgtagttgtggattgttcacttttcttactgaataatattctattgataccacagtgtatttacacatgttgatatagatgagaaattgagtagtttccaacatgaggaaattgctgtcaacaattctgcaatccatagccatgctatcaacagtctagtacatggtttttggtgaatatattaataactatgcatttctgttggatatacacctgggagtggaattacaactcaaataatattcattttaaaaattttatttcgtttttagtcgacaactatattatggggtacattgtgatgtttcaatccatatatatacattgtggaataatcaaatcaggctaatagcatatctatcacctcaaatacttctcatttttgtggtgagaatatttaaaatcctcctttttagctatttggaaatatacaatatgacaatattagctatagttcctgtgctgtgcaaaagaacaccagaacttattcctcctgtctaactggaactttgtacccattgataaacgtctctcatttttccatccacccaccactgcagcttttgatcaccaccataatactctccatttctatgagttcaactttttttaaactgcacatacaaatgagattatatgatatgtgtctctctgtgccaagtttatttcacttaacctaatgtcctccaggctcatccttattattccaaatgacagaatttcctaggtttttaaaattttttttttatttttaattttttggggtacatagtaggtatatatatttacggggtacatgaggtgttttgatataggcatgtaatgtgaaacaagcacatcatggagaatgaggcatccatcctgtcaagcatttatcctttgtgctacaaacaatgtaattgtacttttagttattttttaatgtacaattaaattattattgactatagtccccctgttgcactatcaaatactaggtcttactcattctttctaactattttttgtagccgctaacaatccccacctatcccctacctccacactaccctttgtagcctctggtaaccatccttttattctgtctccatgagttcaatagttttaatttttagatcccacaaataagtgagaacatgcagtgattttcgttctgtgactggcttatttcatttaatgtaatgacctccagttccatccaagatgttgcaaatgacaggatataattcttttttattgctaaatagtaccgcatcatttatatgagccacattttctgtatccattcgcatgttgatggacagttagcttgcttccaaatcttgcctgttgtgaacagtgctacaacaatgtgagagtgcagatagctcttcaatacactccctccttttcttttgagtatgtacccagaagtgggatttctggaacatatggtcattctctttttattattttgagaaacattcatactgtctttatggaggccgttactaattcacaatactaccaatagtggataaggtttccttattctctgtatcctcatgaacacttgttatctttcaactttttgataatagccaatccaaaagatatgaggtgatatctcattgtgattttaatttgcattttttgatgattagagatgttgagtattatacatatatgtgtgtgtatatatatatatatatatatatatatatatatgctgtttgtcatcttttgagaatgtctattcatatatttgcccatttttttattagggttatttgttttcttgttattgagtagcttgagttccttgtatattttggatattagcaccttatctaatgtatgatttgcaaatatcttctcccaatctgtgggttgtctctttattccattaattgtttcctttgctgtgcaaagctttttagtttgatgcaatcttacttacctatttttgtgttgattgtgtttgggggtcatatgcaagaaaccactgcccagaccaatgtcatggagctcttctcttatgtttttgtagtttttagtttcaggtattacatttaaggctttaatccattttgagttgattcttgtataaggggtgagataagggtccagttttattctgtatgtgaacattcagttttcccaataccatttattgaagagactgtcctctccctattgtgtgttcttgctacctttgtcaaaaatcaattgatcaaaggtgtgtaggtttattttagtcctctttgtcttattccattggtctgtttttatgtacttgccatgctgttttgattattatagctttgtaatacattttgaaatccagtagtgacatacttccaattttattctttttagtaaagacagctttggctatccagggtcttttgtggttccatgcaaattttaggattttttaaaaaaaattctataaagaacaatatgcagattttgttagtattgtgtcgaatctttagattgctttatgtttaacaatattaatttttccaatttatgaacacagaaatctttccatttatttgtgtcatcttcaatttctttcgtcagtgtcttatagtttcaacacgcagatctttcactttcttggttaaattcactccaaatattttttcatgctattataagtgagattgtttccttaatttctattttagacagtttgttgttattgtacaaacaataacaattgttatcgctactgatttttgtaagttgattttgtaccctgcaactttactaaacttgtgtatgaattctaacagttttcagtggagtccttaggatttgctgtataagattatgtcatcagcaagaaggggcaattttacttcatccttttcagtttggttgccttttatttctttctcctgcctaattgctctggctaggactcccagtactaagttaaacaagggtggggagagtgggcatctttgtcttgctcctgatcttagagaaaagccttctacgttttactgttgtgcatgatgttagctgtgggcttgtaatttatggcttttattcttttggagaacatttcttctatacctaatttgctaagagtttttctcataaaaggatgttgaattttgtcaaatgctctttctgagtttattaaaatgatcatacggtttttgtacttcattctgttatatgttgaatcacatttattaatttgcatatattgaaacaaccttctatcccagggataaatccctcttggtcatggtgaataatccttctaataaactattaaatatggttcactagtatttcattgagaatttttgcatctaatttcattcgtgatattggcctatagttttctttccttgtagtgtctttgcctggctttgggatcagggtattgctggccttgtaaaataaatttggacgaatcccttcctctttagttttccaaaagagtttgagaaagatttgtgttaggtcttctttaaatgtttgtagaattctcccatgaagccatctggtcttgagctttcctttgatgtgagaacttttaaatactgatgcaatctccttaactctttccttagctgttactggtcttttcagatttccaattttcattattcagttttggtagattatgtatttctaagaattcatccatttctgttaggatgtccaatttcttggtatataattgttcatcgtagtctcttaggatcctttgtatttctgtgttatcagtcataatgtcttctctttgatttctgatttgatttatttgagcctgctctcttcattcttagtctagctaaggatttgtcaattgtgtttagcttttcaaaaaaccaacttttagttttattgactttttttctattgtttctctagtctctatttcatttatttctgctctgatctttgttattttcttctttctgctaactttgggcttaattcattcttctttttgtagtttcctgaggtataatgttaggtatttcatttgagatatttcttcttttttgatgtaggaatttattgatataaacttccctcttagcactgcttttgctacccccagaagtttttctatgttgtgttttcatttctgtttgtctcaagacttttaaaaaatttcctcttgaatttcttcttttgacccaataattgtttaggagcatattgtttagtttccacatatttcttaattttatatgatttctcatgtaattgatttctaattttatattgtggtcagaaaagatacacgatgggttttcttaaatttgttgagacttgtctgtggcctaacatatgatctatcctggagaatgttacatgtgtacttgagaagaatctgtattttcctactgttcagggcacaatgttctgtatatgtctgttaggtccatttggtctaaaatgtcattcaagtccaatgttttcttatgaatttttctgtctattgcttaaagtggaatattgaaattgcctgctattattatgttataggctatgtttcccttcagatcccttaatgtttgctttatatatttaggtgctctgatttgggatgcttatatacttgttatgtcctcttgatgaaataacctttatcaatatataatgatgttctttgtcactttgaacagatttgacctaaagattattttgtctgaagtaagtgtaactaccctgctctctttttgttctcatgtacatggagtatcttttttcatccctttactttcagtctatgcatgtcctttaaggtgaaatgagccacttgtaggcagcacatatttgggtcttgttttttgttgttgtcgttaatccactcaaccactctatgccttttgattggagagtttaatctatttacattcaaaataatgatggatgggtaaggacttactagtgtcattttgttcattgtttcctggttgtcttacagattctttgttcctttcttcctctattgctgtcttcctttgtgttttgatggttttgtgtagtagtatactttgggtcttttgtttttatcattcatgtatgtattataagtttgtgctttgtggatactccgaggcttacataaaacattttataagctgataataacttaaatttgattgtgtgcatatactcaacactttgactctccctcctcccacattttatgtttctaacatcacaacttacttttttttttaattatactttaagttctagggtacatgtgcacaacttgcaggtttgttacatatctatacatgtgccgtgttggtatgctccacccattaacttgtcatttacattaggtatatctcccaatgctatccctcccccgtcccctcaccccacgacaggccccggtgtgtgatgttccccttcctgcgtccaggtgttctcattgttcaattcccacctatgagtgagaacatgcggtgtttggttttctgtccttgtgatagtttgctgagaaagatgatttccagcttcatccatgtccctacaaaggatgtgaactcatccttttttatggttgcatagtattccatggtgtatatgtgccacattttcttaatccagtctatcattgatggacatttggattggttccaggtctttgctattgtgaatagtgcagcaataaacatacctgtgcatgtatctttacagcagcaggatttataatcctttgggtatatacccagtaatgggatagctgggtcaaatggtatttctagttctagatccctgaggaatcgccacactgacttccacaatggttgaactagtttacagtctcactaacagtgtaaagtgttcctatttctccacatcctctccagcacctgttgtttcctgactttttaatgattgccattctaactggtgtgagatggtatctcattgtggttttgatttgcgtttctctgatggccagtgatgatgagcattttttcatgtgtcttttggctgcataaatgtcttcttttgagaagtgtctgttcatatccttcgcccactttttgatggggttgtttgttttttcttgtaagtttgtttgagttctttgtagattctggatattagccctttgtcagatgagtagattgcaaaaattttctcccattctgtaggctgcctgttcactctgatggtagtttcttttgctgtgcagaagctctttagtttcattagatcccatttgtccgttttggcttttgttgccattgcttttggtgttttagacatgaagtccttgcccatgcctacgtcctgaatggtattgcctaggttttcttctagggtttttatggttttaggtctaacattaagtcttcaatccatcttgaattaatttttgtatatggtataaggaagggatccagtttcagctctctacatatggctagccagttttcccagcaccatttattaaatagggaatcctttccccatttcttgtttttgtcaggtttgtcaaagatcagatggttgtagatgtgtggtattatttctgagagctctgttctgttccattggtgtatatctctgttttggtaccagtaccatgctgttttggttaccgtagccttgtagtatagtttgaagtcaggtagcgtgatgcctccagctttgttcttttggctcaggattgtcttggcaatgtgggctcttttttggttccatatgaactttaaagtagttttttccaattctgtgaagaaagtcattggtagcttgatggggatggcattgaatctataaatttccttgggcagtatggccattttcacgatattgattcttcctatccatgagcatggaatgttcttccatttgtttgtgtcctcttttatttccttgagcagtggtttgtagttctccttgaagaggtccttcacatcccttgtaagttgtatttgtaggtattttattctctttgaagcaattgtgaatgagagttcactcatgatttggctctctgtttgtctgttattggtgtataagaatgcttgtgatttttgcacattgattttatatcctgagactttgctgaagttgcttatcagcttaaggagattttgggctgagacgatggggttttctaaatatacaatcatgtcgtctgcaaacagggacaatttgacttcctcttttcctaattgaataccctttatttctttttcctgcctgatcgccctggccagaacttccaacactatgttgaataggagtggtgagagagggcatccctgtcttgtgccagttttcaaagggaatgcttccagttttgcccattcagtatgatattggctgtgggtttgtcataaatagctcttactattttgagatacgtcccatcaatacctaatttatcgagagtttttagcatgaagggctgttgaatttggtcaagggccttttctgcatctattgagataaccatgtggtttttgtcgttggttctgtttatatgctggattacatttattgatttgcgtatgttgaaccagccttgcatcccagggatgaagcccacttgatcatggtggataagctttttgatgtgctgctggattcggtttgccagtattttattgaggatttttgcatcgatgttcatcagggatatttgtctaaaattctctttttttgttgtgtctctgccaggctttggtatcaggatgatgctggcctcataaaatgagttagggaggattccctctttttctattgattggaatagttttagaaggaatgataccagttcctctttgcacctctggtagaattcggctgtgaatccgtctggtcctggactttttttggtttgtaggctattaattattgcctcaatttcagagcctgttattggtctattcagggattcaacttcttcctggtttagtcttgggaaggtgtatgtgtgcaggaatttatccatttcttctagattttctagtttatttgcgtagaggtgtttatagtattctctgatggtagtttgtatttctgtggtgtcggtggtgatatcccctttatcattttttattgcatctatttgattcttctctcttttcttctttattagtcttactagcggtctatcaattctgttgatcttttcaaaaaactggctcctggattcattgatttttttgaagggttttttgtgtctggatcttgtaggtatgcttcattgtttcttattatttttttcttttgtcttctctggctgtgtattttcaaataggctgtctgcctacaagatccagaaaatagcctcaaaagggtcaatctaagagttattggccttaaacaggaggtagagaaagagatagggatagaaagttgatacaaagggacaatatcagagaacttcagaaacagagaaagataccaacattcaagtacgacaaagttatagaacaccaagcagaattatctcagagactacctcaaggcatgcaataatcaaactcccacaggtcaaggataaagaaagaatcctaaaagcagcaagagaaaggaaacaaataacatgcagtggagcttcaatacatctggcagcagatttttcggtggaaatcttaggccccgggcatatccagagatgctgtctgagggccagtcattggagtcaaaaaccttagcagtttacctcatgttctattctattgtggctaagctagcactcacaccacaatataaagtgctccctgctcttccttgcccttttaaaaggcagaggatcctctccctgtggccctcaccaccatgagggttctgcttggcctccactggtgttcacttaaagcccaagggctcttccatcagcttgtggtgaatgctgagagaactgggacccatattttagggccttgggctcccctctggcccaaggcaggaccaaaaatgctgtccaagaacctaggccaggactcagaaatcccagaagcctgcctgcttctctgcctttctgtggctgagctggtacctaaggggcaagacaaagtcccctttacttttctgtctacttttctcaaacagaaggggtctttcaccataaccaccacagctgggaatttgctgggtgacctatgaagccagcacatctcagaggccaagtcccacagtgtactccctgggtattgcaactggttattcaacgttcaaggcctctttagttagtagctgatgaatcctgataggactgagtccttccctttaaggtagcagattcccttttggcccagggtgtgtctagaaatgctatccaggaactagggcctggaatgggggcctcatgactctgcccatgccccatcctactgtggctgagctggtatccaagatgcaagacaaagtcttctttacttttcgctctcttctccttaacgagaagtaaggagtcacttctgttgctgcaagcttcactgctgggagtaggggaggtatggtgcaaccactcccttagccatgccagctggtgtctccctaggtcatgtgggagaccctaatccactggcttcaatatatagagggacgtttctaaattatttatctgtaatttgattaagaagatagacacaggcaataggatatgccaacagattctctctaacctatagttattttaagaaagttagggaaagagaggtcattgattagttttggcttactgaattatttgaccctcccatatcttttaatttatggattttatataaggcacagatattctactagtaaacatgacattaaagatgttttatacaaatgaatgtggttgatacaaaggcattaaataagaaacaaaggaaattcagaggacatttgttcgcctggaatagagatcattagcataagcataagaggaaaataaggaaggaaatgggaaagtcttgagtccattttcaaattatgaaaacttgaaatgcaacaaacaaatagtggttacttaagaagaaagactgaagatgctgggcaaaggttaactttagaaatggcatttttatatcattataaagagaaagtggggataatggaaataatctacatgtacaactcatttgacagacatttattgaaggtattccaaatgccaaacactagtttgtgcactaaggatgtttttaatttttactttttactttatttttattttctagcttcttccgctttgccagagaaggatttttttaaatggataaaatactaacttaggctagcttctttcagaaacagacctttaaacaatgattaatggtgaaagtggtttggttgtaatgtgatcccaggaagtaccagtaggagagtgggacaatgagataataaatgaaagaagccaataattaagccagttatggaactagttattgctttgggccactggggtttgatcctgatgaaggagctctgggagatagtaaacaaacaaacaaacaaaaaacatgcctcaaagttgtcaaccacaagggggtaaaggcaaggaaccagggctaactattccaactcttatccatcattggctaaatgcttcctggtacataaactttccagcacttctggccagcgcacctagcaagctgaggaaaatctctcaggtttgcagtagggcaaatgcttgtactaggacactgctggcatatacttgaaggatgagtgccaacggcagatagatgggccctgacagcatcttctacagattctgtccttgcctttgaggcaaggacactgtctagagtcagagacagaaatgtaaaatgtaaaaatgtaaatcatagtagcacacagtacaagttttaaaatcttaacaaagtgttatgggagaaccacggtgagagaaaatgactgcacatatgagagcaggggaagtctttatatgtgatgatgacatttgagtgggtgttccgttatcaaagatctacttaaaagggaaaaagagtagaattattttaaaccatggatgtaaccaggcgtggccaccctatatgtgaatgtcctgggataattttgtccccagatgtctctctttttgagtgtcattttcatttatgtattcataatataaagaaattgaatagaaagaggtaaacattggtggcaaaaatcccttcctacccataaggctcacagaataatgttgtctaaatgccataacatttttggtaagttttggttctatgggctttggaaattttttatgtaggaactcctccaaggtgatataagcattcagtgtatttatctagataccctagttaaagctactttaagcaggatcaaagtcctcttttaaacttttgtatttgaaagctattcccatgtacaattcccatgattcttgtacaattcctaggagacctttgtttacatcaaaagagtttgtctctctctataagttagaacttgtacattgtagtaatggaaaatccatttcaaagtagcttagacttactcagacaaaatgtattgatcagtgtcattaaacagttgaaagatagacgaggcttgattcaggaattaaatagcatgaccagattctagttttccttctccattgcatggctctgctccccagtatttgttccgtttaattcctcttaattgttctaagatggatatcaggatactgcagaactacgtgctttctcatccatcttaaacaagaagcaatgggttttctcttttagaatcacgaacaacaacaacaacaacaaaaatcctagagttgtttcattggttctgattgaccttacttggaaccataagtccatctttgaattaaccactaaagccagggagatgagttaaaactaagtacacaccatccacagagcaaagagaggtctattgtgggaaaactgcacaaatggaaaaataacaggggaaacacaggagaaggaggtagttagttaatgttcaatacacgcaccttctcagggacttttcttccatctggcatcatctttgtcttttgaccattttccccaatcaatacctatattcatactcaagttttccatatttgggaaaaaacttctcttgacaccaaactcccctctgacttccttcttataatgatctatggttggatagtctacaatttactgtccttactttttcataggtattttttaatctcctacagtaagatttctaccactgcactgaaatcattttgtgtatggtcttcagggaattccatgtggccaaagccaatggatacttttggcatgggctttggaggtctaacttcttgaatccaagtgtcggctctaacaatcactatctatgtgaccttagaccagttatttaacgctgtctttatttcctcatttgttaaatgaggataattgcagcaatagaaaaagaacgtaaacaattatacttataggattgtatgatgattaagtgattaatgcaaggaaagaacaacacagagcttcagacagtgtagatattcaaataaatgttagttatgatttttattatgtcttgtgacatctatgagatttgaaagtgttaatcactcttgtgtttttgaaaatctttcttttccatgttctataaaagtacactttcttgatattcctcctccttctctagtctttcattcatcttatttgctctttgtgttttatttattctttttacgttgtattttccaggagtccattcccagcccggtgttgggctcagtctatgtgccatataaatgagaatgcacttgcatttgaggctttgctttttatttctctgtagtgacggccaatcccagatttctgtctctcccaaaatacacagctatgctatagatccttcagtcctcctgactaaaatgttagttttatgtgtgtgttcctcacaacctcccagtgaataggtccaaaactgaactcattatcttctccatgctcactctgcattctttggataggcagtgaatagcaccactatctaacaggcacctaaaccagaagccaggtaatctgtcttaaacccttttgttttccaagttcttatcataatggctggtgtctaacagatggtaaataaaatagagactagctggctacatggatggatgtgtggggatgcacagagggatagatggattattgagtgagtctctagcctataaatatttttaattaactaataaattatgataatgtacagttgaaggtcaagggtgaaaaagcataccctcagtgggatgcacaccccaaggagccattttaacttgctacataacacacatatgaccacttttttgctgaaagccttctattattgagcaagcattcaaatcccatgtttgcctgaaataatggttcaggttataaaagtttccttctttttccaggattaaaattatcttcctacataataggaagaactgctttatcttttcctaatatctagagatggccttttaaaaatatagactgttttccctattgaaatgaactgtaggatgtacaaaatatttactggcatgaatcaaaagagcttgctatgtttatgtgaaaaccactaggcattctaaaaaatattgctagcatagtaaaatgttagtaattaagactaacgaaagcgaaggcaaattggaatcagagactatttttaaggaatgtcaactgtattattttcaaatacacatggtacataacagtaggatatgagaaaaagtcccaagtatgtgtactaaagtagcctgctatgataagttgaaaaagggtttgtaattggaatatccacagaatatttcagaacacttaaagacattttcatttacactttatacagctttcttataagagcatttacaccatttattttataaaccaaagattaattagaagactaaacaattacaaggcctcaactacgaaagctgttccactacctagtggaacaacaacaatgagacacacaaaacaatggcgttcaaagattagagagagacttacggttaaacagaggttgacatgttaactgaagttgcaatataatatgtcgactagttttgcaatacatagcaaacacccaaacagaaataaacctgataaaaaaacagtagtctataatgtgtgccacttactgagttttaattattctggggactatatttttgatttcatgttacaatcactagttttgtggggtctttctagtcctgatgcttatttacaaaatatctgaagtatttctttctatgtatttatttttgagatggagttttgctctgtcacccaggctggagtgcagcggcatgatctcggctcactgcaacctctgcctcccgggttcaagtgattctcctgcctcagcctcctgagtagctgggattacaggcgtgtgctaatttttgtatttttagtagagactgggtttcagcatgtcggtcaggctggtctcgaactcctgacctcatgatccacccgctttggcctcccagagacctgggattacaggcgtgagccaccgcacctggccatctcaagtatttctttaacttataacttcacataactttgtggaggcaacagggttaattaaaaaggactttacttacataacaaaataagaagcatagttttatattcctgtgccatatacattttgtttgtccatctgtagccattctttgaccttctctcccttgctctttattctgggaggctgacctctgtcatcattgggctcccatgccctttggcttccagttggtttaggcacccaagagccctagaaggaaattgaagacaggaggtaaagtgaggtcaagatatttattctcctaattccctccctttgaggttgccacaggctgactatgtcctttgacaaaaggttattgctcttctcagggtggtttctcattccaattctctgcttttggccacttttccctcccctcatcccttgggcctagatgtagtaacagctctactgttgcaaggttcttgtattatctgtgatggtttcttataccctgcttatcttgtgatttgttgctttgtagataaacctctcagattatctaggcaagatcatagaagaacacgtatgtccagctaagatattccagaagacagtagggaaacattgaagggttttataagagggagtgcagtgatcagatttatgttagttttaatctttaattggggtagaatttacattctaaaacagagatttgggtctgggaggatgatatagagcctcttatggatgtgagggcaaaaaatgatagaggtttgcagtgccaatagaaagaggaaagaagttatatatgagagaaatttgtaactaattatatgaggtgttgggtgacgcttaggaaagaatctagaatgtctatcaggtttcatgcttaagggataaagtagatggcagttttattacttgttttctgtattatttttatttcataaaaccagcttagagaagttgcatagaaaaaataatgtagtcctgtttattttaatatttgaaaagaacatatttcagagtagaatctatatagtacctccctcttggacttccaatgataccagtgatagcctcaatataagccagtcttacaaaatgcacccagcgtgaattcttaggtattgttaaaagaagttggccaggcgcggtggctcacgcctgtaatcccagcactttgggaggccgaggcaggtggatcacctgaggtcaggagtttgagaccagcccggccaacatggtgaaaccctgtctctattaaaaatacaaaaaaaaaaaattagctgggcatgatggcacgtgcctgtagttcctgctactcgggaggctgagacaggaggatagcttgaacccgggaggcggaggttgcagtgagcggagattgcaccattgcactccagcctgggttacaagagcaaaactccatcaaaaaaaaaaaagaaagaaaaagaagaagtttctaatacacttatcttcccttgggttcactcagaagacccttggaaaaggttttaagagcaagtgatttatttggggggtaaattaatcagtagaagagtggaaaaatgagacaggtgaggcaaggcagccagtaaagagtggtgcattatcaagccagctgctgttgtgggtcactggagctttatcccttgggaaactctggaacccttgtaaaatacatgcctcagagttatttcccctagcatcaagggagctagtgtaacaatatcccaattcctacaattagtcattatatacaggctgcctctgggagctggaggggaggcatcagttgcctggtatgtctagcctgtcacatggatggcaaagcaaactcctgtggcaacagaaagccttcaggcaatgaaatgctggcactgggaaatcaggctgatgggtgctgaagtggcaaggatgaggggatatggatattctgctgtagtgcttttctaacagatgattcatatttggttctagggatcaagaattgagttaaaattttatatatatgttgatgttctatgtcaccttcaggaaaataatttaacagaaactaatatttgccatcaaaaaagcaaagaatcctgttgttcatcatcctagccataacacaatgaataattttttaaataagcaacataaatgtgagataacgtttggaagttacatttaaaatgtctcctccagactagcatttactactatatatttatttttccttttattctag

SEQ ID NO: 755 - DNA, 50 bp, Homo sapiens
  source 1..50: mol_type = genomic DNA
gtaagttttttaacaagcatgggacacacaaagcaagatgcatgacaagt

SEQ ID NO: 756 - DNA, 45 bp, Homo sapiens
  source 1..45: mol_type = genomic DNA
gtaagttttttaacaagcatgggacacacaaagcaagatgcatga

SEQ ID NO: 757 - DNA, 40 bp, Homo sapiens
  source 1..40: mol_type = genomic DNA
tttcaataaaaacttaagttcatatatccccctcacattt

SEQ ID NO: 758 - DNA, 54 bp, Homo sapiens
  source 1..54: mol_type = genomic DNA
gaatcctgttgttcatcatcctagccataacacaatgaataattttttaaataa

SEQ ID NO: 759 - DNA, 50 bp, Homo sapiens
  source 1..50: mol_type = genomic DNA
gaagttacatttaaaatgtctcctccagactagcatttactactatatat

SEQ ID NO: 760 - DNA, 250 bp, Homo sapiens
  source 1..250: mol_type = genomic DNA
tcaagaattgagttaaaattttatatatatgttgatgttctatgtcaccttcaggaaaataatttaacagaaactaatatttgccatcaaaaaagcaaagaatcctgttgttcatcatcctagccataacacaatgaataattttttaaataagcaacataaatgtgagataacgtttggaagttacatttaaaatgtctcctccagactagcatttactactatatatttatttttccttttattctag

SEQ ID NO: 761 - DNA, 60 bp, Homo sapiens
  source 1..60: mol_type = genomic DNA
actatatatttatttttccttttattctagttgaaagaattcagaatcagtgggatgaag

SEQ ID NO: 762 - DNA, 212 bp, Homo sapiens
  source 1..212: mol_type = genomic DNA
ttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaag

SEQ ID NO: 763 - DNA, 47 bp, Homo sapiens
  source 1..47: mol_type = genomic DNA
aaagaattcagaatcagtgggatgaagtacaagaacaccttcagaac

SEQ ID NO: 764 - DNA, 55 bp, Homo sapiens
  source 1..55: mol_type = genomic DNA
tgggatgaagtacaagaacaccttcagaaccggaggcaacagttgaatgaaatgt

SEQ ID NO: 765 - DNA, 53 bp, Homo sapiens
  source 1..53: mol_type = genomic DNA
aatgaaatgttaaaggattcaacacaatggctggaagctaaggaagaagctga

SEQ ID NO: 766 - DNA, 49 bp, Homo sapiens
  source 1..49: mol_type = genomic DNA
gccagagccaagcttgagtcatggaaggagggtccctatacagtagatg

SEQ ID NO: 767 - DNA, 56 bp, Homo sapiens
  source 1..56: mol_type = genomic DNA
gagtcatggaaggagggtccctatacagtagatgcaatccaaaagaaaatcacaga

SEQ ID NO: 768 - DNA, 60 bp, Homo sapiens
  source 1..60: mol_type = genomic DNA
gcaatccaaaagaaaatcacagaaaccaaggttagtatcaaagatacctttttaaaataa

SEQ ID NO: 769 - DNA, 47 bp, Homo sapiens
  source 1..47: mol_type = genomic DNA
aaaatcacagaaaccaaggttagtatcaaagatacctttttaaaata

SEQ ID NO: 770 - DNA, 51512 bp, Homo sapiens
  source 1..51512: mol_type = genomic DNA
gttagtatcaaagatacctttttaaaataaaatactggttacatttgataaaattataccatagattgtaatttaatgatgtttaatgtaaagttattaacagaaaatcacgttaaagctgaaatgaacagtagactttgtatatttattttcttagagacagagtctcactgtcacccaggctaaagtgcagtggcacaatcatagctcactgagccttgaactctggggctcaagcagtcctcctgcctcagcctccctagtagctgggactactagccaggcgtgtaccaccacgcctggctaattttttaaaaatttttgttttctgtagagatgggttcttgaactcttggcctcaagcaattctccttccttggcctcccaaagcactaggattacaggcatgagttagcatgcctagccagtagacttttgagtcaaggtagagaatagaggaaaattgacagctaatgtcaatgttaaagttaattttgtttagtaatctggatatagttggctggttttctgttgcaccatctttaagatcacttcaaaattggtatacgtatttatgtagttgcatgaagtcaaccattcgggcttaattccttcctagaaaaagtaacaggtctactctttcatatcttatttggaggccctggatcaagaaatgggggtgagtgggtatcaggattggggaggaataaggagtactctcactttctccccccattctcttacctccagcccagctcattcttctcttctttctctttcctctgaaatttcctcaaatatctgtctgcttagtagtctataattcctcaggataaaagaaaaaggaaaaggaagaaaattgcttaataactatgttttatcaagcagcatgcatagtgctttgcatgcgtttaatcccttcaaacttagtatttactagtcagtttgaaaagcccttaaatgttaaacaatggttaagctttagggtattttgagcagggaagtaacatggtaggaggttgatcagattgggcacttgatcaaaaccctctatagatattttatctgacagaaaaggccttttgctaaaccagtatctgattctcaaattggttatgtcatctaagaaggaaaccgtatcattttcttggatgaaatggcttccatttccaaattagtcaggttaggctatgctaagctatagtaagaaatgaactctgaaatctcaatagcttaacacaataaagactgctttcttggttatatcatagtccagtagaggttgacaagccattcttaggagctgtcccccaacatggtcacttggaaacccagacttttccaattttgtgacacttgtcattttcaacacactgccaaaagccactgcagaaaaggaagacagaatgtggtcagtcaatccatggtgttttatggccaggcctggaaattatgtatatcacttccaccacatcccatcacacagaacttgttctcgattgaatttcaggaagagttaatggaattagtgagcttttagccaatctctgtcatagccattttatcgaagggacctgagctgctatgaggacaaggttttagtttatatgatgaggatgttagaaagccacatagaattttacagtcttatcattagctctgttggagtcaaaatctaggtgaataaaaattctttcaagagacacatgttaccactaatggttttttcaatggatgagcatagatgaattaggaatctccaatggtcattttcctaattcctggtctgactatgtaatatatagaatcatgtggttattcttgaaagctctagtcatattcgtgaggtagtcagcatgttatgctacttctttttcttctctataagtttcggatactatgaatctattggatcacaaaccaaagtagttaactggtaagaaggtataagcagaaaatacgaagtttggactggatccttgagttagatgagaagaaggagagatgagaggcatacagtgatagatttgactagggcaagaggactctggtagttagcaaatgagaaataaatttaacctaagaaaatcctaaagtgatggtaagaatggttagggtttaattgctcttagaagagcaagtagctttaaataagagacaaaaatatctgcagaaggaaatatgaaagtagaaaacaaaacaattgcattgtcaaaacatgtaagatgttttaatcacctaaaataaaaccagaattgtattataattgctatcctttgtcaaacttttgaaattaaaaaaaaaaaacgctttggaatggtctaaaactacatcataaaaatggctcagccactatctatagagattgacattttttgtttgtgctctgtgtttaggaatttaatgatgtgtattgctgcagattcaatgtaagttcccgatacagataaagatggccaaagctgtaattttttcatccatttcttgaatgatgtatgctaaaattaaagtaatcctaatgttaataataacttttagtgaatgcttgctgtgtgtcaaaaagtatgctagtcactttatatatattggtttatttcatcctttcaaaaacactctttgaaaagtactcttattattcttagtttgcagagaagaaaactgaaggctagagaagttcagaatcttcttttgaaagtagtagagttgtgatgcgaatgcatagcaattactcttgtagatgacctcacaggcttcctcttccaattgtctacgtggctaaaacaaagcaagaaaaccccataagaatctgaaaaagactcatgtaattattaattgacaaataagattatattacattactgataaaccaacaggtagaaaatgaaagcaatgaaatagtatactgcgaaaaaaaatttcatattatattctttgatgaaaaaataaaacaataattgcaaactgcatttcatttttaatgaaacgacaatgtttaattcttctaaaaggtagggagcaaataaaatggttatctggtctttacatcgaatgtgtcatcactactaagtgatattagctggcttattacttaaaattttcttttaggaaaaaagtgtgaaaatagacaggaggagacattaaatggctaaagcaacacccccttgctccgtaagttctttctgagtctgtgtgcctaagattaacaagagagcctgtgagcctcacaaggcaatcacaaagctatgtctacagccttggagagctgtgattacagcagccagcctcctttgcagcacctcagtgatgactacactaactctcactactgagcaacaatgaaatgtttcaaacccaaccaccaacactgttataactcttaaaatttgagccagtcaccatacctgaacgtaaaaagaaaattaccaaaaatgcttgataccaaaaagcaattaatgattcataattgctaatctttattgacagtctcttctagttcttatctacatatattttaattgtctacataatgtaaattaaattttaaattctgatttgtaaacttaatattgcataaatatttatcaatgttgttaaacatcttttaataattattttaacttctttatattatttcattaatttccatctttatgtccttgctggtctgacatttagaaaaaactcctccttttatttagaaaaaaagaatggtgaggccgggcgcggtggctcatacctgtaatcccagcactttgggaggccgaggcaggcagatcacgagatcaggagtttgagaccggcctgaccaacatggtgaaaccctgtctctactaaaaatacaaaaactagcctggcgtggtgctgcatgcctgtagtcccagctacttgggaggctgaggcaggggaatcgcttgaacccgggaggcggaggttgcagtgagccaagatcacgccactgcactccagcctgggtgacagagcaagactctgtctcaaaaatacataaataaggagaataaatggggacaataacaacctattcacagaacagaaataaaaccggagatgtcatgtggaaaaagcttatcttgaaagcgtggtcagagtggaaagaaacagcaacaggagagtcttgatttggtgaaaaaacagaatgcagatggcatagagtatcaacaattaattagcaggccatcaagagacggccataacatattggtaattgttttctttcacccttaattttttctaaaacaaaaacatgaagttagaaagtagttaattttgttagccacatggcttcatggtttaattttatcttctctatgggcctggtacaacaataaagttcttttgagatagaaagacagatgttaggcaaaaacatacaagattttcattctaatcaaaatctttgccaataggctatgacagaatcctatgagagatgatgccatttagaactttcacaagaggcaaaaatagtatgccattggcacctaaaactgccaacagtgatcatcataggatttatttcatttggcggattcttggttgagttcataacctgttccctaggcttttaagattggtccaaacttggcaatggattctttggaaaaattatttcttgatcgttttaaactttcttttccagtgtcaagagagattttcccaggctccgagttttcatttattttttcccctgtcagtttttgaaagagaaatcccctttttgtcggctttgtgattgcttttgtgtcaaattcagcgtaaaagcttttcaaacgtgacagcattagctgttttgtgcttggctgagctaagagtacatgaattcaatagagaagtgaaatcttgtttgagcctggctgatttggctatataatattgaaaactatctgaagcaagaaattcaatacttttcaaattattgaataaacatttctaaggctgcaaaatctctgtgtttttgaaggcattctttaggaaacttgaaacacagacattctataatcacataattaattttaaaagttgaacttattgtatgtagtaactaaggcagctatttgagtgtctcaaattattttactaaaaagggagacatttttcaaatatggaagtagtgccttacatttttagttatctgtggaaggatcgatggcatcatatatctcatccaccataaaaatatgtacggtagtggaaactgaatttttcttttatatattttgtgtattttttggagttgtacaaaattaaatcagcaaattgacccacactagtagtttgaggtttgggtttctggtacaataatgaaaagcattattagaaatcttcttgccaggacctaccatttcttcatgtattttctttattcccttttgttctcccctaaagcttctatgtgattaaagagaaaagatcaataacccaatagtacttttccttctaaagtatgcatgaaactcttaaagttctcaaagcatacaaacagagtgctctctacattgctgatagaacacaggtagtaatgggctagtttccccaggtaactattatattatataaatatttaatgcagagtcaccaagccttgaataactttatgctttgattgtaccatattttctctagtttgcttttcacaatttaacttatcccgtgcttgaaaatataatgtggtaactgtgtaaatctgctcctcaaaaaattttcgaaatcatcttttcttttttcttttctttctttttttcctttttttgagatggagtctcactctgtcacccaggctggagtgcagtggcatgatcttggcttagtgcaacctccacctcctgtgatcaagtgagtctctcagcatcccaagtagatggaactacaggcacattccaccacgcccaactatttctcgtgtttttagtagagacagggtttcactatgttggccaggctggtctcaaactcctgacctcgagtgttccgcccacttcagcctcccaaagtgctgggatacaggcgtgagccaacatgcctggccccacatcgccttttctgaaccctgggaattaaccaaaggctggcaacaatttttgaaatgctgactctcaataaaaatggcaaagtttgaggttttttaagttggcctttcccgtcactttctcccagctccacagtagccttgaaaatcagtagccttgcaaccataatagctgtgaaaactagcagccttttagccaccagagagtgtagaccaggattggaattcttcaaaaaggccaattttattccaagaaccttgccactgtttgacctatcttgcagctccctggaataaacctatatacatggccttgcctttatttggcccgacttagagataactttctaggaaaagccctagctccagggtgttggtccaaaccaattagcagcaattctttaacataacaggttcctaaggctgtgatgctagttgagataaacataggtctggagaaaaacttccagtgaagctctggagaatgaggtgtccataggggattttggcaacctctgacatgttcttgcgggatctagaagcccatgtgcttgtcttaacaaatagagagtatcaataaagagatagaaattgtaaataagaactcagtggaatatctggagttggaaagcacaataactaaaattaaattttcacttgacgtcagattcaagctgcaaaagaaagaatcagcaaacttgaaaataggtaaatgggataatccaatccaaagaaaagaaagaaaaaatattaaggaaatacagaaagcaaaaactgactgaactgaaagaagaattaggtaattcaacaaaaatagttggagacttcaacatctcacttttaataacaggtaaaagaactaggcaaattaacaaggaaatagacacttgaaaaagactataaagactataaacttacaagacctaacagatatctatacagcgtttcatccaataatagcagaatactcattcttttcaactgtacacgaaatattctctagggtaggccatatgctaggtcataaaataagtctcactacattcaaagggattaaaatcatacaatgcagattttatgaccataatggagtacaattaaaaatcagtaacagatgtaaatttgggagttcataaatatgagaaaattatacaacacactcttaaatagccaatgggacaaagaagaaataagaaaatacaaaaatatcttgagataaatggaaataaaaatacaatataccaaaacttatgggaggaggctgaagcagtgcttagagtaaaatgtataactataaatacctatatttaaaaaggaaaaagatctgaaatcagtagcctaagcttctaccttaagaaactagaaaaagaagagaaaagtaaatctacagtaagcagcaggaagtaataacaattagtgtgaaaaaaatcagatagaggaagaaaatataatagaaaaaatcaacaaaaccaaaatctagaaatttagttatttgaaaaggtcaacaaaattgacaaagttttagctgattggttaggaaagaaatggtcaaattactaatattagagaaaaagagggtgcattactaccaacattacagaaataaaaagaagtaaaagaggatattatgaacaactaaacaaatagataacctggatagacctataacaagaaattgaattggtattaaaaacttctcccaatgaaaggctcaggcccagaagtcttcccccgatgaattctaccaaacatataaacaactcttccaaaaaatagaagagaaaggaacacttcctaaccagacaaagatatcacaagaaaacaagctacagagcaatatgccttatgaatatagacttgaaacgcctcaaaaaattatcaaacctaatacaacaacatattaacatgattgctaccataaccaggtaagatttatcacaggaatgtaaggttggtttaacatccaaaatttaatcaatgtattataccatatcaatagaataaaggacaaaaatcgtatggtcatcacaataaatatagaaaattcatgtgacaaagtacaaaaccctttcaagcttgaaaaataaagaacacttaacaaatgaagaatagaaggtattggccaggtgtggtggctcacgcctgtaatcccagcactttgggaggccgaggcagttggatcacgaggtcaggagattgagaccatcctggctaacacggtaaaaccccgtctctgctaaaaatacaaaaaattagctgggcacggtggcgggcgcctgtagtcccagctactcaggaggctgaggcaggagaatggcgcgaaccctggaggcagagtttgcagtgagccgagacagcagcactgcagtccggcctgggcgaaagagcgagactccgtctaaaaaaaaaaaaaaaaagaaggttttgtggattgaatagtgtcccccgctcccaaaagacatactaaaagcctgagcccaggtatctgcaaatgtgaccttatgtagaaatagagtctttgcagatgcagtcaagttaagatggtatttttataagaagaggaaaagaggcatacacagagagaagaatgccacataaaggcactgaccacgggaaaaacaccatgtgatgttacagttagagattaaagtgctacagttgcaagacaagtaaaactaatgattaacggccatcattagaagcaaggaagaggcaaggaaaaattctcccctacaggtttcagcaggagtatgggcctgctggaatcttgattttggatctagcctccagaagtctgagacaatgagttttgtctacccagctacgcaaattgtgacacttaattacagaagcccgaggaaacgaatacagaagaggatttcctcaacctgataaaaggcagctacaaaaattccacagctaatgttattgtgaaacactgaatgctttcccctaagatcaggaaatagtgaaggatgcctggtctcaccacttctattcagtattgtactggaagttcaggccagggaaattaggcaagaaaaataaataaaagacatccagattagaaaggaataagtaaaattatctctacttgcagacaacatgaccttgtaattagaaaattttgaagaatctatgaaaaaatattagaaataataaaataattcagcaaatttgtaggatacaagatcaatatacaaaatgaattattttatacattaaaaatgaataatctaaaagtgaaattaagaaaatatttacaatggcatcaaaaaatgttaaaaacttaggaataaaagagatgcaaggcttgtatactgaagaatacaaagcagtgttgaaagaaattaaagataaaaaaatagaaaaatatcacatgttctcagatcaaaagacttaatattattaagatatcaatatccccccaattgatctacagactctatggaatcattatcaaaatcacaactggcttttttttttttttcagaaattgacaagtggatcctaccatttctattgaaatgcaagggatgtagaatagtcaaaacaaccttgaaaaaagaacaacattggaggagttaactttccaatttcaaaaactactacaaagctacaattattaagtcaatgtggtactagaatgtgaatagacatatagatcaatgaaatagaattgaaagttcaaaagtaaacctttgcatttatggtcaactgatttttgacaagggtaccaagagaattcaatggaaaaagaatagtctttcacaacctggtgctagaataactggatatacacatgaaaaataatgaaatttggccccacctcacaccatacacaaaaattagcttaaatgtatcataaaagaaaacaaataatctgattaaaatgggcaaaacacttaaacatttctcaaaagaagatgtacaaatagctaacgggtttgtgaaaaatgctcaacatcactaatcattggggaaatgcatattacaatcgcagtgagatgtcaccttacacctgttagaatggctgttataaaaagagaagaatgataacaagtgttgatcaaggatatggaaaaaaggtaacacttgtatatcattggtgggaatgtatattagtacaaccattatggagaacagtatggtagctcctcaaaaaactgaaaacagaattaccataggatccagcaatcccacttccgggtatatatccaaaagaatttaaaccagtatgtcaaagagatatcttcactcctgtgtttattgcagcattagtcacaaaagccatgatatggaatcaacctaagtttccatcagtggatgaatggataaagaaaatgtggtacatatacacaatggagtattattcagccttaaataaagaatatccttttgtttatgacagcgtggatgaacctggaggacattatactaaatgaaataagccaggcacagaaagtcaaatactgcgtgatctcacttatgtgtggaatctaaaaaagtcaaactcattgaagtagagagtagaatggtggttaccagaggctgtggatttaggggtggaaaatggggagatgttgatcaaaaggggttaaaaactttagtcaggaggaataagtttttgagactttatgacacaacctggtgaccatagttaataatattatattgtatatttcaaaattgctaaaataacagattttaaatgttcttgctataaaatatgataaatatatgcagtgatagatacgttaaattgctcgatataatcattccacagtgtatacatatatcagaacatacatgtaccccataaatgtatataattatttgtcaaagcaaaataaaattaaaaataaaatgttttaaaacttttgtaaaataaactttataataataatatatttttaatgaatcatagagctaaatatattaataagaactaaaactttaaaactcttctaagaaaatgtaggaatacaatggattatgcagtgttttcttagatatgacaccaaaagcaaaagtaaccgaaaaatacacaatttggactttaacaaaattaaaacttttgtactgcaagcaatattgtaaagaaagggaaaagacaatcctcagaatgggtaaaaatatttgtaaatcatatatttgataagatacttgtatcaagaatatataaaaactcataattcaacaacaaaaaggataaatgacttaattacaagtgaagaaacagtttgaataggcatttctccaaagtagatgtatagatctccataagcacatgaaaagatggttaacatcattgattgtttaaaaaatgcaaatgaaaaccacaaggagatagtactttttacccactaggatggctaatacaaaaaatacagacaataacaagtgttgagaagatgaagaatgcttggaattttcacattactggtggaaatgtaaaatggtgaaacaactttgaaaacagtttggaagttcctggaaatatttttttaatttttaatttttgtgggtacacagtaggtatatatatttatgggatacaggaaatattttgatataggcatacaatgtgtaacagtcacattggggtacctggcctatccctcacctcaagcaattatcttttctgttacaaacgacaatctaataataatcagttatttttaaattcacaataaattttttgactgtagtcatcctcttgtgctatcaaatactagatcttattcattctatcaaactatatttttgtattataaatttcactacccattagccatccccccctttccccctaccactacccttcccagtctcctggaaatattaaacatgaagctaccatatgacaaaaccattctactcccaggtgtatgctgaacagaaataaaaatacatatgtacacagaaaacctttatacagatgttcatagcagcattatttataatagccaaaaagtagaaccaacccaaatgtccatcagctaatgaataaattttaaaatgtcctctacatccaaaaatggaatattatttagcaataaaaagaaatgaaatactgatatgcactgtaacatgagtgaacctcaaaaatatgctaagtgatggaatccagtcacaaaagaccccaaattgtatcaatacattcatataaaacatccagtaaagactgaaggaggaggagaatagacaatagaaagggactgttaattggtatgggtttcattgggggcaaataaaatgttctcaaattagatcataataatggttgcacaaccctgaatacattaagaaccagaaaattatgcactttaagtgggagaattttatggtatgttaaaaagtatatctgcatgtcattgactacagatcttttcttattttttcaagttttatgaacacagttttagaaacatagtctccatttttctctttcatgaagctgcttgccagctatagaaatgaaaaaatagccatgtctgtggaacataactgctacctagatgggagcctccatcacacaatggatagggagctattttcttttatggtcaagataggcaggttctgtcctatagaatatattctgagatgttacctgtagttcagaaaaacagggatattgttgttctaaacatttcctcaacatttatgtggcttcctatttattaataagagaacctgttctttatgtgtcattatactagtttattctagccctggtcctggtccaggactgtctggaggtattagaattgccctactctatgtgaaataaggtcatatctgaataggtatgtcagtcagcataggctaagctacagtaataagcaacctcaaatctagaggcttaatagtctatattttactcatgttacatgttcgttgtagactgatgagagcattctacttagcttagtcactcggggaccatggctgatggtatctccatcttggtacattctttcatgatcatagaggcaaggaacatggttcttgagccttctgcctggaagtgaccagtagacaaaaccagtcacatggccattcctgagttcaatgggaatgccgatgtataatcttctggcgtgaaaaggcactggatatttgagaacaataatgcagtctaccacaatatgctcatggggtctgaaaaggagctgaaaaaacgccattttctggggttctgtcctctggcattgtttctggaatcgtttccacccatccatttctacaccctctcaactgacttttctcagatgcctggggtagctgggtgatttgttaattacagtattgcttaagccttttaataaagtcagcaaattaaagagagagcccagataaagagaatgctttcaaaaatgtcataatgaaattaaggtaacgaagcacttggagacctctcctcccttctttttgtatgaaacttattccaaagcagctggaagagaaatggggcctgaaagtaaagaggggaaaaatatattgtatgccattagtttaattataaaaattcattacatgaagccatgtaagacagaaaataatggaactctaatgggttggccgataacacatttactagtctgctctcagcttgtagtcattgttgaaaggacagaaaagtaacgacgttggtatgtagtcttcaaagctattgtaaagattaatcattttcagtaagccggaaaatgagggccattttcttaaatagtatgccctgtatagagtgttaccttctgaaacagagttgtttggtaatgtcagaggtttaatagatgagtcttgaagttaatggtgagaggctttacttttgaaatggctttcttacagctattttaacatttctattataaatccataaagaggttttataggtcgttttaattcacacaaggagggaagaaattgtgcttctcttttcccaataaggatttatagaacattaattcattcttgggaccaaattgtttagtgattttcaaaaataaactatgtatttttctgctgtctttttttcagaaatgccttactttattatacataataattttccagtcatgctataatcatcaagattgttttatattcttcaaagctcagttaaaataaacccagtccttttccttaaatttctttatatggatgcataacttctttaactgaattgaaatgaaacctgaattgagagtccagaaacctgcatttttatgatgactctttcactgactagctatgtcaccttgaacaagtcacttaacctttcagagcatttatattctcatgtggacaattatggaattgctcaaaaaggaagaagatatcaaaagatgaaagtgaatatgtaggtaggggtcatatattcgagggccttgtaatccaaagagagaggttaggactttgtcttgcaggtaatagcaaaccattgaaaggttttacacagaggagaaatgaggttctatttgcattacaaaaagatcatttacgcagttgtatgtaggtggaagggtggtttgagagtggagggagggagccaataaaaaagattattttagtacctgagcaagagatgatagggacgtaaattaaggtagtgtcaatggaagtagaagatttggataaaagttaaaaaaaaaaaaaaaaaaacttatgatacaactacccagggagtatgttccctgcctcctgctccagttgaggaccaacatatcagattgatccagagacagaattttggagaacattaatatttaaaggacaggcaaaaaaaaaaaaaaaaaagcttgaacactcagagaatacagaggcgaccaaaagagtggaagccaagaaagcagagaacttcgagaagagaatagtcagcagcaccagttgctgcaaataaatcaagtaaataagaacttaaatctttccataggcattggcttttggtaactgaatcagttaactattttcacaaatgtgagtggcatatagcaataagcccatattgcctttcatgaattttcagcttgggtggggtggcacttcttcatgttgcaggttgattggggtcgctctgctccatatgtcactcattctcctcctgagatgagtaggctagtccagagcatgtttgtcttaaggtaatggtgaagtgcaaaagggatagccacactgcataaatacatttcaagcctctgcttgtgttacttctagtaatatcccactgggattaggcaaagcaaggcgcatgaccaagctcaatgtcaagggcatgctgtctttcgtaggaagaatttcagagttacttagcaaagggcatggatacagggagaggtaaagaattggggccagtaattcaatttgccatagtgacttctgtcagaatagttttagtggaacgatgatggcagaagcctgattttcatgaattggagagtggcacgaatgtagtcaagttgagataatgagtgaatatctgccttctaaagagtgtgactgtgaaaggaaagaaaatgatagagtgcagattattgttgttgttgttgtttaaagatgagagagacactcatgtttgaagtctctagagaaggtccccgcagagaggcaagaagttggaacacacaaagagagagggagtatgtggaggaggatcccagaatagatgggtgcccagatggtgaaatcaacttccaatagcagaaaagtcgcctcttcctctgagactagagagaaggatgcaagatgtaaagtctgatatttcatgaaatgtatgtttgatatattaaatttccccctgtggaattgtaggtaggatcatttactgtgagatttctttcagttcgtgttgtcaggaattagtctgtaaaccacattcgatatttcaaacagaggaaattcaatacagaggactggctcctccataggaatgaaggagctgacaaacccaacagaggacagtgaggcaacccagagtttagcaacttcaggaaactattaccacctcaagggctggggaaccgacaagttttaccaaagcccagaaaccagaaccatcgggtgctaattcacccaatgtaaggtctgcacagcaggtaatggaattacagagggagcagccgtccagtgggaaacggagacaaaggagatactgccaagacagagagagaagggggataattatcttgtttcaccctttctcatgctcttctgtctcccactagtgtgtctcatgacttaacctagttagaagccagctagcaagaaatcccaggaaattctgacctcctatgggagttagtacatctcaatactgagtggagtaggaaatgggtagagaatggatctaagagcaaacgacctcttggtggttagctaacagagttaaatttttccatgaatcccttggttcatgaatgtaaatacaggatccaaggaaaaataacaaatccaacatgtagaactttttgttcataagcatattaggatcctgttttacaattttccgagcaattgcttgattttgacacttcaggacaagccggaggcatggtcagagcaagtaaccccatcctagtttacagatgacaaaactgggacttacaaggttaatgacttccttaagatcaaactgcagaacaatggacaagcaaaacttcctgggaagatgtcctttcgttactctgcactgcctttcttgaagttcctttgagacacagtcattaaaaatttaagtaatagttcacccggaacaaacatttattttacttgtaggtgaatatcacaaaacacattttaaaaggaaatttaaccaatattcaaccttaaaattttaatgatagagctatgcacttgcttttatcttccaagttaaagggagaattttagagttgcagcatttctagacaatgtccataaaacagcgagctacttgctttgcattcagaaggggctcacactgtggggaatatagtttgactcattaaaattagtagtagatgtgctagcagtagtaatgatgatgatgatgctgatgctgatgatgattaatatttatttaatccttcgggcacgccaggcacctttgtaaatgttctccctgaattctctaagttaacccacctaatcatatttccacataaatactgttaatattcccatttagcaaaggaggaaactgaggcacagaaaagataattaactctcccatttgccactaagcgatcacgccaggattcaaacccagcaggctcattgtattgctcaggcttttagctactctttctcttcctcctctcttttctaccatgtagggtcaatatcactaaaataagttcttacacataaaagaaatgagagaagacaaacaattaaagtagaaaaaaatactcaaaattcttgaaattaattcaggttgtctcatagtccacaatcactttttctacctaagatactgtgaatatatatcaagcacatagtgcagtagcagcataatgggtttgaatgtgaaacatttctttcctggttggggaaacaatcttttggggatttagcattttatattcttcatagaaaacacacacacatacacacacacacacatacacacacacacatatacaaaggcaatctttttttataagctagtgctcacagtattttaaatttcttcacacaccatgatcaccacacaagcaattccctgtggtgtacaccattgtcaatggaattcagaaagagaaaagggtgtgtgtgaagtctgttgtatttttgaatttacactccttctgaggtctggagttatcccagtagtaaatttcacagcagtaggcaaagctggtcaaggtcatggttcctagtgtgaatgacacttgggtaaaacccaaggtgggcgggcagaatattaccgtaggagaaagggaaaacattctgtgtggggaatgtctttaagaagggagaaaaaaaaaaaaggctacctttgagaacctttgtgtttatataagggcctgagccccctcaatgtcagtcaaggtcagtgggagttttactttttaatcctacatcctcacaaaacctgggcagtattcagtgagggttagcactcagtcaagatttccttaggtataacagtcctaactttcgctgtgaatacaattttgcagaaaaatatatggcacgcgtatttctacctttcaagcattaaagtacatcaaaaagttctggtttgacgtcaagcctcactctttactaccttgtttagtaggaaatctgtcatttaactctagcttactatttctttcagagattttggaaaattctaggatatgttataatttataattttaggcatttttatgatctattgattatgtttttagattcattggcaccttctgcattttatgttttttaattttaacagctttattgaagtgtaattgatatacaaaaactgcataaaattatatgggctagaacatgtgcatatacccatgaaaccatccaaatggtataacctcttatatctagacttttcaaacaggaacctatgagtgatgggtggcttccaaggttatttaacttgcttgagtaaaataaatgattttaattatatatttactaaaaattttgtttttattttgtattcctgagcttttacatatatgctctgatactggtccatcataagtattttattaacaattcagtgttttataaaaggaattcatttgaaaaaaaggtaaccaagttattggtctcagttttgtggatgttcatgctattaattagcaatttaatagatgagagataataagctcttcttttaaaatgcaaatagcaccctctggatactaagggccaattacatgttctttcgtatcttggaatacatttcttttatcactttaggtttggcttaagtgaggaggagaaagaaagaagtggtaattttgtaccctaggatatttctagaaaaggcaagcctttggcagaagttccctcagaagggctttcaaatagtgcgaggatctgaaaagtggagaatagaaactgtaatgcttgataaaaggagtatggaagaaaaggcaggcattccacgcggataaatgttaattgttttgtaatagcagcaactatgctctgtgcctttagacactgtcctctgctttcatagcaaaagtcagtgatattggcattagcttcttgataatagtttggtaatctgggtctatttgctatatgcagtaaatactattacttggtttgattctaggatgttttagttaactgagaaattacattatatatagattgtatatgttcaataggagttcaccaaacacgctgaaattatgctaagaacactgtttgatttttattctgtaaatatcatactaacgcaaaagaacaaatattaaaacaaaaatgatttttctcctcagaagcattattttcaatttcttccatttcgtgttcagctcatgattatgtattcttagagataaacacagtgtagttaaaaattgtgttctgttttttacacaagaaaattgtgctctttttacagtgattactgtgcacatttgctacatattggaccattggaatgatgtattatgttcttttaatgtccctgcattaaacatcgacattgtttctctttttttttttttttttttttttttttttccgagatggagtctcactctgtcgcccaggctggagtgcagtggcgtgatcttggctcactgcaacttccatctcctgggttcaagcaattctctgcctcagcctcccgagtagctgggattacaggagcctaccagcacacccggctaatttttatatttttagtagagacggtgtttcaccatcttggccagactggtcttgaactcctgacctcgtgatccacccgccttggcctccctaagtgctgggattacaggcatgagccaccgcacccggcctgtttctcatttttttactgctttggttaatgaagattttaagtcatatagtatattgatcttcttttgaagattttatcaggataaagtaccaaaagtgagattattgagtcaatgggaataagcatttgtatgtcacttggtatctattgccaaattgtcctaatagataacggtccccagcagtaatacgtgaatgtgctagttttactggtcttttcagctttggattttattttttgttaatttaattggtgtccaattgtaccttttcaatttaatactctattttttattacttgaaagttaacttttttctttttaaataaaatcatcccactctttctcttgtttgaactacctgttcctattcctgtgtccatttatcaatgacttaacccatcctattgaatttgatataactatcttaacaattagtattatcttaatgcttagaagcctcctctctgcacttgcagaaatgtttaatagaaatttcaggttgatatgcttcgagataaaacctaccattaattgacttctttctttgaaatatgtttcaagtgatgagatagcaagtttgaaaacactgaattgccaaagacaaacaacgaagaaaatgtaggtgatttggtttagtaagattcttatattttgactttttttctaattttaaagtaaggtattctaaaagtattttgatccttcaaaaaattcgtgactagaataacagtactattcttttgttgttcaatattttctgtatgcaggtttagttgaacgccagtagatggcactaattacataaacaattcaacaagttaatgaagagggaaagaaatgtatgaggtttttttcgttcaaatgttgttatatgtcacatattcaacaattatatatgagcttatttttgtagtttttttctcttgtgataaaaacaattaagcccactttattgccaattaattgctactaagttgaaatacttgatactggttattgctcaagatgctgcatttgaaaagtttgtcctgaaaggtgggttaccttatactgtcatgattgactaaatcatatggtaggttaaaagcaatctaatatatgtattctgacctgaggattcagaagctgtttacgaagtattttaagacactccaactagagatttcataaaaaaaactgacattcattctctttctcataaaaatctatagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaaggtatgaattacattatttctaaaactactgttggctgtaataatggggtggtgaaactggatggaccatgaggatttgtttttccaatccagctaaactggagcttgggagggttcaagacgataaataccaactaaactcacggacttggctcagacttctattttaaaaacgaggaacataagatctcatttgcccgctgtcacaaaagtagtgacataaccaagagattaaacaaaaagcaaaatactgatttatagctagaagagccatttatcagtctactttgataactctatccaaaggaatatctttctatctcatcatggcgcacactgccttacctgttatctgataaataagtcactttgggattcatgatagagttatagctgtacatggtctcatcctagtatctcactccacacacccaatgggaaaatttgtggagggcaatatgactcgtcacttcatttcccattatatatgaatggaaattaacagcgcttatagacagtatctcctcaaactaagccttgtatccttattatacctctcttgatctctagtgcttttttcactagcatttattccaatcataaataaaaatataaattatgtaactaattgttaaatatttgtcctttaaattaatctaaatgccatgagggcagagattttgtctttctcatttgatacatccccaggtcctgaaccacgtgatataatagggagctagtaaatgttttttgaatgatgactccctttgcagaatgtacaattaccttgtgcaagctgaaaaaatagcacctgtacaatatgaggaagaccacggtgaaaaataattgagttccaaaatatgacatcaattactgaaaaaataagctcggtgatttttaacaagaagtaaaagtcaccactggggccaaaacagattttgaactaagagtaggaagtcttaggagaaatgagataatgatatatggaaattaagcggccaactaaattttgaaactgagctagacattagagagtaaaaactcctgtgaagctgaatttaagctggtcaccctggggaatagagcaactctaatcctgaattccagacagtaggtgtatagatggaaaagaccatggaaaagaagattcaacctaaagttgggaagttttaattggagccctatgaaaaagaccctggtggagaaagggcaaacttgaatatggagctgatatttggaaaaattctcatagtaactactttttctcaatggcaaggcttggactttcttctcaaaatacagatcttatatgtgttcaattaaacagggacagattaggttcaggaagaattattcacatggaatcaattggtatcagagagtcaaccattagatcttagtgggaaatatctgcttctcaaagagaagtcttttggggaaagcaaattaaagtcagagattaatttgatgagtttaggtaatataaactaaggggccaagaaaaaagcttgctcatggtatgaaactagagcttgaggacactgatctagtctatctatactactctttctgacagacccctctcttcattctcatgctccttgatggcccaagccactctctcagttttttaaaaaattgttttatcaaggtctctggattcttcatgggaatgacttccagtttatattttttggcttggttccaaaaagctatcagctaaggaatgcatatacttacttcccctatgggtaaagtaaatgagaattttagaagccaactcacatttttagcctgtacagaatctgcaattcaccaagctacttctgactcatgtctataaagttcttccctgttcttttctcacttcacatgtactctttgcaagaattcatccacttgtgtagtttcagtctgttgatgactacccatctataattccagctgagaatgatcttttgagttttagacatgtagatcctgctgctttctttcgatgttaatgtcccacaggaacttcacattgaagaggtccaaagctaaactcatctttgccttcttccaatctctttctccaaatgcaacctacttctgttgtccttgtcttagtccttttcgtgcttccgtaacaaaataccacagactgggtaatttataatgaacagggatttgttggctcatagttctggaggctgcgaagtccaagatcaaggggctggaatctggtaagggccttcttgttgtgtcatgattccatgatggaaggtggaagaccaaaagagagaaaaaatggggccaaacttgtccttatatgaaactcactcccacaataatgatgctaatccgttcatgaaggcagagccttcatgtcctaatcacctcttcaaggtcacatttactactgttgcaatggcaattaaattttaccataagtttgggaagggaaaaacattaaaccatagcattctgcccccttttccccaaaattcttgttcttctcaaagacaaaatacattcatttcatccccaaagccccaaaaatcttatttcagcataaactcaaaagtgcaatctaatataaattagatatgggtgagactcaaggcacaattcatcgtgaggcaaattcccttccatctctgagcctgcaaaatcgaatcaagttcatcccctcaccccctacccttcccagcatcaggtaaccaccaatcacagaaagttttactgatagtcctgctctagatcatctttgtctatgttcactttagctatttatcctagtgttccattattggaatactaagcatgtgggaattatttatattctactgttcaaggtcctcaccaaggtctgattgcaaaaattcaaaaaattgcaaccttaggcataaatgggttaagcagtttagggtacatttataataattatttactgtgctacttcaaaaatcttattgcctctatttataaataaaaagtgttgtctctacacagtggcttgttgtaatgcatttacttgtttctgcctgattttttctatttatacattttcttttttatttttatttttattttttcacttttaagttcaggggtacatgtgcaggtttgttacataggtaaacttgtgtcatgggggtctgttgtacagattatttcatcacctaggtattaatcctggtacccgttagttgactttcctgatcctctcgctcctcccaccctccacactctaatagtccctagcatgtgttgttcccctctacgtgtccatgtgttctcatcatttagctcccacttataaatgagaacatggggtatttggttttttgttcctgtattagtttgataaggacaatggcctccagatccatctatgtccctgcaaaggacatgatctcattctttttttatggctacgtagtattccatggtatttgtgttggtctcaaaaactacaactatgacaggatggcattttcacttttgttgttatattaaactcatcttaaaaaggaaagattaataatgtcaatatttgggttatggagaaaaagtatctcatatctttgaaaaagttctgtaactatagctttttaggtaggagggattctgtggaaagttttctgattacatcatttctcacagttcaggttagacaccattttactatgaaacactaatgcattgcctgcactgagactttcagtcacatggagaaacctaggcaaaatttttgtacacttggaagaatatttaaattagtaataaaatctttagttttaaactgttgaatgttaaataagatataaaatgtacttgaaagaaatttgctttgatatcagacactgccatgttgcagtttcaagacataataaaaaagtaaactaatgtttatattttgctgtttaagtttattaatacatcagatgagtcttcaaattctacagtggcttttgatatgatcatttttacttgccattttatatagaataaatataaataggcatttatgcttaaaaggaactaatctatctatggaaaaaagagaaggctgcttctcaactaaattgtacagtttagaaacccagatctgaacatagattattgttgtgacctatgtaggaaaatatgttgttttccttatcgtagtccttacagagtccatgataacatataaagccagaaatgtgagcctctgcaagttcatttctttgtcttcaatctctgtgaatagatatgagtttgtgaataagataatattagatgtgatattacaaattattgtgagaagcctctaaggattagatttcaaggactgccatctggctgatgactttatgatgacactgtcatgagatttcatttccttatttctgttccaggatcactctttaaacaagaaataagcattaactctgaattgtctgcttgtagctgtatgagggcttccacaactgccaactagccaggtacaaactcatcaagcagaggagatggtccttgcatcagagggttaaacatgcctagaagttccttagctaagctcccagatactaaaaaatccctctaggttctaagaaagattcagcatgtacatgtgtgtacatgtatgtgtgtacatatatacatatacgtgtatatgcatatgcatgcatatacatacaaacacattttcttccataacatctcagtattctctgttctttataatactgttttgtattttaatgatcaaaattaatagttgatcatctgaaaacattttgacctgttttctccgtctttgacaaccttgaaggcacttgtaagtcactctttgcttctctattcctaggtcctttctcatcttcattgcaacaagaaaagagaaaacaattgagccctattttgtgtgtagcaaggagctactctagttaaacactagatctcttttacattctccaacatgttgttttagtaattattctactttcctttttttgggatattcaatttcttctttctttttgctcctcccctttagcaggccaacatactcaagtctccctcatcctaagagaacttttttagtatatcattttttttctatccagctgtacttgcttctgcttactatatcatttttaagcagtagttggcattactgtttcctgttctttagctactagttgtactttgacccactccagtctcacttccccagcaccaccactttatgaaaacaaggacttactaagatcatcagtgactttgtaatagctaattagtgtattttaattcgtccatcttcttgactatattttaacattgatcctgttggtcaactctgctaatcaaaactttatcctccttggttcccagaacaatattatcttgaatatctcatttctctaatcatataataattgtgaggtgcttggcacaatgcctagtgcgtagtaagaactcagtaaaatatcatctgccatcgacaccataaaaattaatttacttactcaacaaatacttttgtatgaagtttgtgctaggtaggcccagtaattggtacttggtatagagcaatgaaaagccctaccctcataaagcttatattcttggaagcagaagttggaagacagacattgacaaataaaaattaaatacatgatgtgtcagatggtcatacacacagtgtggaagaacaaagaggaaaacaagtggagagagagagggaggtggaagaggagtgctgccatgaaaatgtggtaatcaaaaaaggtcttactgaaaaggtggcatttaagcaaattctaaaagacctgaggatgtgggccatatgtataattgggggggaaaaagtagtccaggagagtcctaataagttaaaatgccccaaagcaggaatattcttggcatgttgaaggaaccttaaaagggagatcagttaggcagaaaaggatcaagcgagcaggaaggtagttgacaataaatttagaggggtaactggcatctgattatattggccttttaggcctgtggactttagcttttaatctgaatgagatgggagttattggagggttttgaatggaggagtgacatgttttgtcttatctggctcctctgttacaatagactaaacagaagtagtgagaccattaggaaactgttgtcataattcagtcaagagatgactgtggctgggatcagaatgggagaggtgaatgtggtgaggagtggttggattctactatattttgggtacagagcacaacagattttataatggaataaatttaggtgtgagagaaagagtcaagaagactcaagaatttttagcctgagcaacggaaagatggggtcatcatttactgagatggggaaggctccaggagtaacatattttgggaggaagatgtggatatgttacatttgaaatgcctattatacatctaggagatgtgtggagtagatagctggatatatgaatcttaagttatggggagtagctcaagatacaaagttgggagttgtaacaatgatcagtgcaagttctctgtcttcaatgcaattttaaatgttgatgttccattcttaattgtctctcttctttctctctgcacattttgagtagctttgtctgttggcttcagttaacattaagactcctcagtgtcaacttccatcttacactcttctcctgatctccagaactgtactttctgccacctaacctacattaccacctggatatgctacaggctgcaaaatgtgtcaagtagaatgcattatcttgcccctaaaagaaagttaaattttctgtgttttcagtgtagtgtaattgtctaacttaattgtctctaaaactggaaacctaagaattaccttctacctttctcttgatctctctttcccaatctactgacacatgtattaaactggcttccaaattctgtgaattctacttcaaaaattgctctagaaacaattccctctctttatccctattgtcacctcatcctaaagcctcttcatcctttgtagatttctgggagattgtaaccaacttttctctattctgccagttatcaagtctttacgctcatttgacattcacaacagccttggatctgtcttccttgaaatgaatcttcttgcttccctttgattccagtgctttttttttaccctcctgagacttgatgcatgatatttacatgtatgacatgtttccaaaagcattctcaaatttttctgaaagtaaaaacaaatgaaaaagtaaaacattttcctgggaagaaaagcaaatagtgttatacatttttgcttgttcatttgtttgtttatttaggagagggacaagcattagaacttcataagagtcttatatgctgtatctacaaataccgtcccttggcaatataattttagagttccttttctggaactacttaaggactgttttatgatcctcagcagactgttatattattttatagccataccttttatttgctgagtaattgtactcaataattgtttgtaattgaatgaaacaattcatcagatgttgggcactgaatggctttggattatttccaaaaatttaaaggataaagatttgctgccttcaaagctatgtacaaaaatatgatagaatgctagcgggatatttgtttaaaatacaacctttattacattggggcctgctcataatatatatgtggcacattttatttaaaatattaaagttcctggtgggacatgtccccataatcccagcactttgggaggccgaggtgggggtgggaggatcactagaggccaagagtttgagaccagcctgggcaacatagtgagataccatttctacaaaacataaaaaaaaaaaaaaaaaagccaagtttgtagtcccagctacttgggaagctgaggcaagaggatttcttgaacctaggagttcagttcaaggctgcagtgagctatgatcatgccagtgtactccagcctgggtgacgtagtgagactccatctcttaaaattaaattaaatttaaagctacaaatgaccccaaagccaccagttcaaccctctcaattttgaataccctattttaaattcctcttatgcgaaatgtaccttgtagtccattttaaggactgagaggatttggtatgttaaaaaattcaatccattatcaactcctttaggtacacttagcagtatgaaaatgtgtctttcggctcttcaggagagagtcatatgtatagttacaagacaatcccatttttatattgctgagacccaaatcttcccaactgattatgaagcataagaactcttcggaggtttaagtgagctgagattgtgccactgcactacagcctgggcgacagagcaagactttgtctcaaaaaaaaaaaaaaattctctgcattctacagtagggtaatataacatctatgatgtgaaatcttggggctccgggccagagagtgtcatgatccatatggatctaaaaggttcatagtggtaacagcctgcttcattttatgtcatctcctttcaagtaattagaatgtttctagcttgcagggattgcacacaaagggagacatttggaaccatgtcattggtgatttactggtgtggaaaattacctggtgatgtagccaagtagccattttcattctaacccagtcctacagtcctgaactgggctgaaccaacgcaccaaaatatatgcttagaaatgctcctatgtatcagttttcccaggaaaaacaatagtattatcgaaaacttaccattgtttcctaataaaaaattataggataccaacagactgttttttgttcataaatttaatattacagtatcaaatattaaagcaaatgggagaaagtttttcttatttggtttaattgaaccattaatgttagctacaatacccatcatgttacttttcaattatatttatattttcattttatttctatctgtatcattctcagaaagacttctttaaaacattcaataaaaatagaatttaggtagatttatttttagaaagttgagtttttttaataaatgaatataatcatcacttgacttaatttttttctgcacaattctagaaatcttatagttttgggatcctttggctttattcagtatgtaacagggatctgtttcctttctctaaatcattaattcaaatgatttcttatattaaaaatgtttggacatataggtattaatgagttttatgaaatctaatctttccaatttccccctaaaaagggatgtcatttaatcagttctaggttgtgatcaatagcagatttttttttttttttttttttttttttttttttttttttttttttttgcagctccctttcaccccgtagggaaacctgatatcatccttgactaattgcagcaaagagcctggctcaggtcctttgtcttataccgagtgtttatagattcttgagcccagcagaatctgaactcctggctactgctacctacttcccagcccaggccccaaaagccctatgtctgcagccccgtgcaccactgtgtgtttttgtggcatttctgaaacacagagctacttaacttgtttctaagcccagattgtgcctttttgattttctattttggtattttatctaccatttttctgtgtttggatgtttcttctatattttgaaataacttctttcctttagtacaagtgattcttattgtagaaactatcaaaaatttacaaataaagaatcattctcaacattcttagcaattccttctatcatatttttgcaaatatatttttgcctatttttattttacttactccctgtttattaacagttaaaagcattttcagatagttttattttttcatttaaaaaaatcttaccacatttttattaggaaggaaatggacaggtgtttatcttttcaataaaaaacatgggggaaataatttcttgaagtacatagtgacattcttccagccaatgttttatgctgtggtcattccgtctgtcatcagtattcatagaaagagatgaaaattatttaaattaactaggaaatcaattccccattcaaagcagtagttgtgtgtttcaaatatcttctaatagtcagtttcacacttagctttatcaaattcctaattatgatactcattacatcactctgtgtccagtcagtgtgtttatgccacagagcaattaaagcaaatcaggtgaaccaaattcaatcacctttgtagataataacctacgttgcttaaacttatggccgctcatacaattactgatggattgcctttttcttttatattgccagtattttaaatgtcctagtgaagttggggtagctgttgaacttcaactttatcacaacctcttttttaaaatgtgtaaacgaaaaaaccctccatgaaatgaccaaatacagttttcatgctgggacaaattagatgaataataatcataaattcataatgattatttatgattttatgtttttatagtgagatatgttttgttgaaatgtgttatataagtgatacttaagtttcctattaaaatagaaatgctaaaatggcattgttctctttagctgtgagtctagcttttgacctctgcttaaacggaactgttgttccatcccaaatctgcaactctgaggcctatgctcccttcactgctgtctaatggatacctatcaatttggaaggaggtttcaggcagctattcccggtaatctaatctcagctctgtccttttcaatattttcatcagtggcttggatgaagacatagataacattcttatcaaatcaatgccacaaagcagggagaaatagcaaatatagcagacaagagtatcaggagccaaaaagttttcaacaagttggactggtaggctgaatactgaaagatgtaatgtaaatgcaaggtgctacatgtgggttcaaaagaaacatgaaacaaaaaacccatctaacttagactgggctccctggaaatagactaagatagagagttgtgtgcataaggtttgttgaggagtgttcccatgagatacatgtgtaaggttgtaagataggcaagattgcacagacgaagaagtgcagtgaagcctgcagtgcgttgcggcctcatcagattttcaggggagttctggaaattgcatggccctttagagacacgctgaattgaagcaagggatctggacctttgaacccaatactagagagttaatggtcctgggtcaccccatgggaaagagcagactggagtaagattgttacctacagctgaaggcaatttccagggagggaggcagctgtgagctgttagtagtcaatattccaaccagctagggcatgaggtcttggcagagcaacagtgtacccaagaccgcagtgttacccaaagtatggtcctctgactggcagcattggtatcacctatgagctcactagaaatttaaatttgtaggtcctaccccatccaactaaatcagaatctctggggatgggacttggggaacttttaacaagctttcaggcctccaagttatttctatgcatattaaaatttgagaaccactgcctacaccaaccaaaaacattccaaatatggagataacatagagtttttagcaacaataatctccttctgtttcacttctctctttacacacacacacacacacacacacacacaacacacaacacacaatgtgatagaacagtgggaaaggaaagccaaaggggatcttaggccgaataaatttaagcatataacctagtcctaagaacgtatatttcagcttaatagagagaggaatattgttataaagctgtccaaagatggaacaggctgccttgtaaagttgtagaagtattcaggaacaggttggtgataccttggtggttgtatggtataacatcctgatcttcacatactcatcatctagagtgggagttttctttttccaaatggggttttggcagaactagttccactgtatcttaataagtaataactcaagaaagggttctatggatgaaaaaatgattaggtaatatcaagttaaatcaaagcgaacagacttctttcccataggagtaatcagacccttattacagtgcatgcttggtgaatcaacaaagtatgtgtatttatgaaagtatggggggaagggataatctatacagtatgcatcccttctaaaagtttgaccatgaaaacaatttctcaagaatcttatacaacactacagtatctggtccaatactatgcatagaacatgcactcagtaagtgtttgtaagatagatagcatagcatataggccaggccactgaagggaaatcatctcaccgtgagttacctgaatagtattctctagtgccattagctcaattcttcacgtaggcataagcctatacatttgccatgctaaccaagggaatttgtgttacgtgaattttgactctattcagacatttttttctatgactcctccaaggctgttattcttacctcatattctggtagaagtttaaggacttttttctgggaatattgattaattagctagctagctagagacagagagaggatagagattgattctctggcagagcctatttgaatcatattgaatctttttttttcctgagacttcccacaaggaggatggaggagaaattttttagaaatccaccgaagtaatcagggatatcttcagtaaaagaagctatacttaataaagtctctattttagcagatggcaatcaacaatagaggcaatagacaatagagtctattaaaattgctgggatctgctaataacgtttttcttttccctgaaacaaatgccattaaccctccttgacactctgtcttcatcaacattctaatagaatggaagtaactcataattttgaggatttttttcccacacaaaacctataaaccacaccacgctagtgattacttttagcctagttgctaggttgctgctggtaacagtaaaacttatcctgacaggtaggcaattccagaagcccagccaagcacttggtgtgtgtgagtaaacccccatacacttctcatgtagagtaaccctggccaacccataactcttagcaactattcctggtggacggacctggtctactctaagaagaggccaaggttctttaatagtgcagttgcaagaaccagaattgaaagtcaaagttctagcaagattttgcagactccttggcaaaccagtggcttgggactcattcttgacttcaagcccttaattgataatggtaggacagcttgcttgcgctgggttctgctccctgggatatgcactgtttgccaaatgagtagcaggtggacagacatctttacaatttgctgtcccatattctaaatgaacgtgacattctataggtctgagttaacctatgaagtcaccaatttcaatatcaaaatatttatgacagagaaaaggatactgaggcacagagagtctgtgactttcctaagctcaaaacaccagtttgtgttaattctgacacagaaattcttgtatttgctatcagtctcctttttctgtgtgtgtgtgtgtttttacattgcagcatcacctatatgatgttaggttctgtaactttttgagaattttctcacatacagtgatgtgttactttttgatatttcaaatagttctagtaagtcttttctacttttattagcgtattaacatactggctctaagagggcatctcaccacatctttgccattcttcctggaaaggcaagtttctctccatcttcttttttgtattccaaagttttgccaaagtttgcttttgaaaatgggttacctggcagagctttattattctaactttgaaagtacaagtcagaatcagacagtggcagttatatatgcactactgtgattactatataatgaaagtatctatggtgaaaatactgatactgacatatatttgccattttctaattaagtgcttcagtaaaaattaagcactcactctttgccagatactgcaatagatattgagcacattgaacaaaattctccatatacatatatatgagtccacattctatgaaagtataatgtttttctgagaaaaggcataatattctattaatatcagcttttgcttcttccaccatatattgaaagaattctgaatactgttataatttaatgggagaatctagagaattctgtatttgctttcactgcattgatgaactaagatttttaaaaaatgtattcttcatagaactacttttccatatttacctaatattattcttatatcatttgagcacatatttcactaacaaaacaaatgtgcaatgttattagttctaacatcaaaattacactgatactttaatttttatcctattatttttcatgcagattaaaataattatagctacatcacatgttgcaagttttaagagctactttaaaaatatatgcttcaggaaagacatgattagatggggaaatggatgatgttcatattttcaaatgaaaagttttaaaaaagtgcctatcacaaacactaaatttttacataaattatcaactactaatatatctacaagaaataccatttttccctacaaaaactcttaacaataattgttaaacttagtcctggaacctgctaatataatcggacaaatgttgtcaataagaaggtgaaaaagaaagcatatatagtttatcaaactataaaatatagtttatcaaaaccaatttttcctattgacatttattcaggaaggaaaatggatgagtgaaatgaacaatggtctctaagagaggtgggagatagcaataaattcagaccacgtttcctgtcattacagcagggaagtaaaagagctacagtcaactctcgaaagtacttgggggaactaatgattccctgtagacctgtgatgtttttgaaatttaattcaacaatttgatatacaccgcaaagcgaacagatagtcagatcaaaatcggaagaacgattgtctgaatggcatccatttttcctagatgtgctgtcccatcctgtgtcaattaaactttcaggtgatcttcaaacatatttccaagtaaaaggtattgcagttatcctataaactggcctcttccccagcactgcttttgctgtggtcaactttatttctttgggctcacaaaactgatagagcaaaataaggaaaacggaacattggattaaaataaattaattcccattctgtgactcactaaaaaaaaaatgataactatgcttctgtgagcattaataaggaaatgaataaggaaatgaccaaattgttcagtggacaacttgtatgggatttttaagtattgtgtcatcatcaatgttgtcaattagcatatactttgaaatcaactaaagcaaatcagttgactaatcattaagggtctttttaaatgacaacatctaaacagcaaatgttttattttggaaaatcatgacagcacaagaatgagccagatgttttacaacatgatatccataatttaaagtatgtagtagtcactcaaaggatttctatttcagtttccttatgatttggctaagctagaatttggaaaaacactttaaggtaatgtgagaaacagcaaaattcaacatgtggattttttcactaaagcttatttctgattattttttacaaactttactaggtatatgttaacttcatgacacttatagcagtggaccgtagttttaataaaatgtgaatgtatactcttttctcaataatattaaagaatgttgactttcgtgaggatatttttatttttctcaacattaagaactgtcaaagatttaattctacaacagaagacgtgaattttgttttctaaaggagaacagaatctatagaagaagtgttgctcatagtactcagattgttgaccaatcttaaaggagaaaccgtcaattaatttaccgagaagtaataacattatctttttcttcaattatgcacatccacaaagatttggggcaaaatccacttaaatgatattatacataatagatgagtattcatatgttgtaagagtcctggcttctttcctgcaaaatgattaaaacttggatcagaaaccaattaaaaatccattctaattcccaaatgtatgtaactgtactataagaaaaataaatatttcttcttgagggatatccattagttaaggatattcataacatggtgtcttgtaggaaatgttaatctttgggtgaatagggatgtttgggaataacaagactcaaagagatgttgcacttactcacttttctctgagttgttatttctgtcatttccccagtgcgcctgtcctcaactttgcctctctccttattcctttttttttttttttttttttgagacggagtctcgctctcttgcccaggctgtagtgcagtggtgcgatcttggctcactgcaacctctccctcctgggttcaagcaattctctgtctcagcctcccgagtggctgggattacaggcacccaccaccacgccctgctaattttttttgtatttttagtagagacagggtttcaccatcttggccaggctggtcttgaactcctgacctcgtgatccacccaccttggcctcccaaagtgctgggattacaggcgtgagccaccatggccgatccctccttatttctttttatctctacctctgcctcaatggtatttctctattactgttagcatttgctttctgtgagctcttgcacactgtcagcttatatacatgttcctgttcacatgttttcctgtccccagtggttacaacatgtcttctatctcagcccactctagaattgtcttacttttccaggtctcctgctcctcagtatttttcccacttttctagattcatgttttcccatctgcatatttctcttccatgtctgcactgtcatccgcttagaagacagcgcataaggacactgttatctgagcaaatcttcagcacagccaccatgaagcatggttaccttgtcactttccatttttcccatagtgtgtgcaaactgccctgatctgcatagaaaggtatcataattgaggaaacaaaatgcacaaaaatgtccttggttattccacccctcagaaatataggagagaagtaatttacagaattacacagaataacgctatgtcacatggacatggagttatcgggttagcatataattggaaaatatttcctaggaccttgacatttactcactttttgttttcaaattacatgtccctatctattagttgcaaattattttaatgcaccgtttaccaaagaaaggctgtttcttctgaaagctttcatttgacaagtaacttgtaaaaatattcacattgtgtatctgttttccccttctagtccaaactctagttatcttaaactttgcgcagttataaaaaatcataacaaaaaaagcttcctcgttgtcattcttgtcaaaacaggtttaccagacttaggtaaacttaaaatagttagtgtaaaagttaaaaagctgatttgctccttccagcgtgtttgttgcctttttgccacagcaaaattgtaaatgtaaacgtattccctaggagatgagctgggctgcaattttcagctaattgggagaagcagccctgagttgagcactgtcaggctgatttgagtcttaagatatgatgatgattattgtgtcaaatgtaatcaagaacgtgggctctgaactgactcaagggctggctgtttttaattcaggttcgtatatgaagtagacctccggttcaccgatagtcacagctggttgtagaagagagcaatttttaaaatgctatttcattctctatggagctctagggatcagagattggatgcacagggaggggacacatcctcattctctcctgaaaaattctattaattttcagtataataaactttctcttgagattccccagtggctctgtatcggtggttttcaaacttctcagacccaatgccacccctcttttcttttttaaataacaaatactttgtaataccttctttacgattataagccaaaatatgtagacaacataccctacttatacaggcaatagtttaaatgatgccgtaactctattttaaagagaaataagagtcatttataataaaataatatgtgttgtagtatgcagttattcaggcaggatcacactggaacacaagtgaagtttttagatcacgagactatcaatgcagtataaacaaatgcagaatgacaccattgtgttgtatggagactcaaataccatgaggggcattggtcatccatagcgtaattttccaaaatgctgaacaactcttggcaaaattcctaacaccatgaaataaattttttcttggatcgttatggcagttagttgcatggctgaaaaattcaatgtcttaaaatcatgaggaaaatatcttatgtttacgtgtaaaattgagttacgttccaggtttaggtgtttataaacagggtttccacatacatgcatgtccagtgggatattccaaagtgctgtcagacttgggagagttctttgttgtataagaagtctaccatcttcattccctctccacagaatgctattatagtaacactcttcaatcactgtgatagtcaaatgtcctccctcaatttctaggatgcctcttttttttgtggtctgtataatttggttaaatatctttccagacaaatactgatttgtgaattaatgaaatagcagtattttcggagcacctaacctatttctgagtgatacagttgccatttttacaagactaaatgaaattaccatttcagacctgccagattgtctagcccagtcttttacaattctgtgattatcactgcaattataatctattttcaccacttgaatggcatgatctctataaaagggtggtgataacactcatctattctccttcccctcacatagctatatcaatcgccccctaaccagttgttgataaatgcagttgaattttatgtaaaaattataagagatattattgtagctgtccaagacatttaaaatgctaaatgcaacttacgtggaggctataagagaaatatgaacccatttattgaagagattagctaatttagtaaaacaacacagatatacctgcatacagggataaatccctattgtctaaattattgagataaaataatgttttacaatgaaaaacttttagacaagtaggtaagtaaaatgcagcagtctatttgcatttcatctgggcatttgacaaagtctttcgttatactcttgtgaataagttggagaaatactggctagatgcaagataaattggatggcttagaagccacttcatgattttacgcaaaggatgtcgattaatagaccagtgtcaggtggtgatggaagatctctggtgctatgtcacaagcttctgttctcaaccctgacacactggatgtttttgacagaacatgagtagaactacagagaggaggcccatcaaacttatgggtgataaaaagcagggagggcaggagtattttgggtgacagaagccaaatgggtgtctggacaggatgcgttttaaggcacttttggtacttgatgtctgaagaccaggatcaaacttataggcaatctgaacatttgccaaaataacaggttaattttgacagaagttattatttgtatgctgtctatttctttaatacacctagaaagtattgaaataacattttttgcagacactcattttgaaaattcagaaaaaaaattgttaactttcgtggaagagtaacagaaactcagtcattgacagctaaatacaatgtgttgcccagtaaaatagtccaccccttcactttcatggctaatataaaatttgatgaaagatacaaattccaaagattgaatatctgtacatttgcaaagcaaaacacaattttgggcacagaattgctcattctcatttttaaacatcttggttataactgaacaatagttttttataacaaagataatattttcaaattattatgaggttcaactgaaataatttatgtgaaagcaatgtctaaactctaaaattctatataaatataaattattattcaataaattcacatcaagaaaattttaagttttttaagaacaagagcctatggccttgtttttagaagctgtataccttatcggtagtaggtttattgactttaattaaatttattgagtatctattaaattgccaggaactgtggtgtgaatctttgccctcaaataatttacagtaagttgtggttgatgaatggtgatgacgatgatgaatatccagactatagtaagtggtatattcataagtcagaggattcttaaaaccagatgcaccctcagattcattcctttcatgttgtacttctaattgaaaaaaataaatcctaaattatgactgttctttataaattttaattgatcttataaaaggccatcaatacatttcaaagtatctaggtcttttaaatgcaatttttcaccctggtaattaaaagtacgaaagcaagaaactttaaatctttattttgataagttttaattagctcaagctacttgtaatcccacatcttgtcttgtaaatcatatctgagccattaaaataggtttacaattagaagggcaattcttttagaatctacttaaactaagtcacttcgacaaattaattcatcgttcagttggttttattaaaatgtatttatttcactgtaaaatgtctagtaaagcaatgtatgaagtattttattttcatgttagaaattttatgtaaaagatatcccaaaatacatagacattcagatactctctgtatcattaaccaacatttactaacttatcatttagagaaggccaaaattgtatgtactataactttgtataatttcataagaattaaaatattcgattaatgcctgtaatgccttctttctaaatcaaatcctcaagcttacctcgagttcaaagttcagtatttattgtaacacatctcatagatgacggatgaagatggtaagcaaaggaataataatttcttttctcttttcacacatatatacacacataccccataatcctaattcatataataataacagaaaacaaagggcttttgagaatagtgacatattaatatccattatatttacttcacagggagactggcaagtctaccttgagaggtaatgtcttatagtacagtggactagattgtttcaagatttgtcatttattttggcaactcacccagcttccctgaaagttaagttcctcatctataaactgttcatgataattacaacctgcctcattagcctcatcaagctatttaaaatatgaaaggaggtgctatctgtggatcctgtcaaaggagcttgaaaactgcagaacattattttagtgtaaaatactataacaatacatgttgaatataaaatggctttttcttaacttttattttaagttcaggagcacgtgtgcaggtttgttatataggtaaactcatgtcatgggggtttgttgtaccgattattttgttacccaggtattaagcgtagtacacattagatatttttcttgatcctctccctcctcccaccctccccactccagtaggcttccacgtctgttgttcctctctgtgtccatgtgttctcatcatttagctcccactaataagtgagaacatgcagtatttggttttctgttcctgcattagtttgctaaggacaatggcctgcagctccatccatgatctctgaagaatctccacactggttttcacaatgactgaaataacatacactataaccaacagtttataagcaatgctttttctccagaacctgttatttttgactatttagtgatagccattctgactggtatgtgatggtatctccttgtggttttgatttgcatttctccaatgatcagtgatgttgagctttttttcatatgcttgttggtcgcatgtatgttttcttttaaaaagtgtctgttcatgtgctttgctaaaagggccctttcaaatgtgtattattaaccacaagagagtactgagtaagagactaggtaataaaagtcacaaatatttcgatatcataattcagaatttagatcagcggttatgaaattgttcgtatttccaaattccactgacaggactctactataagtttatttcatctgttgatatgtttttagccacttctttcttttaaagtgaatctgttgtgtgtttgccatttgatattagaaaactgaacctgcctgctttgctgtcttctgaatattatgtatcaacaactaacaagctacagttagttgttttgttctgtttttctctaagttattgtggatgaggatatatataactgcacagtcttatcaggtttgtaagagatgatcttaggctcatcttttaaattggtttttatactattttaaacaaatccttttaggagagaagaaaagctgcttagtctatcaacattaggaaatatatctttaaagagtttatcactgcaagtaaccaaagccaacttaaaaattcgcattatacaaatcattgagaatttatttagaacagaaatgtgtccaactataggtcaacaccaattttaagtgtgtaattatctgggaagtagtgttaactgcatttttttctaaagatcccttacagttgtataaatgcccaaaaggatattttgagtctctgtatattaaccaaaccaaatgtaattcattactcccaacattatatttcaacctctccaaatagtaccttttcgtattgtatcagcagaaaaatataaaatgcagatcttaaagagtatcaatctctttaaaaattcaagaaagaaaaaaatatgtgtgtatagagacgtgtatttcatctgctcataacactgtgtacatttctttatcaactaatttttttcagtgatttatgagttgaaatacaaatcaaatgaaacgggtaatgcaaagtaaagtagaaaacacattttctactgctgtctcctaatgcaggtcttttcaggaaagtactaatggttttagggaaagtgtataattatggttgtttccctaatgataaattcgcaaatctctattttaaaaacattcataaggttaaaaaaatgagagatgaaatgtgtctttcaaaattccttacgtgattgataatgcctatactctcttactatctaaagtctaggtgatatgtatattttttttaaaaaataaaatgtctgtatcagtgaaggaagtttacacagatagcttcaaagctgtggtttatctttggaggattaatctatttctcatgccagtgtgttgctactgcacatgttaaaaagtcatcctgtggtgtctggggtgacaaaagatgggaatgagttttctgagaactaatcagcaatactttgggaacatttaggtcatggtttccaattaactctggagagtttgagtaatttagtaccagacctcaagagagaggggatgaaaacctcgttaattcatatgttggtgaacggcaaaccagcaaatttgcattaaaaatggatttttattttaaagcaaagagcagccagatcttttctgcaatagtttgggtaggagaatatctttgtatgtatgtgttcccttatgtgtaggtatttgtatgtttcaacgaccctgcatatggcaataacagaaaattaaatttgtgctctaaaatgaagaccaggattcagtgacataatcttccttgtgcctttctttcttttagtacaatgaatatatcagagaggagtgtattccaatatctgtcttcagagttacaaaaacttcttttctagaatgcaagacttgggctatacccccagctctgccacttaacttgtatacaaccttgggaacatcattacaattctctcagaatcaatctctccagccctaaaatgaaaccagcaaaagcctgtactgtatatctaaaaggttttttatttttatgaaaattagttaggcaaacttttgttaagcatccatcactctattttgagataaagccttgctggatgatctccacctcttttgatggaaagagtaaaacatgtttaagatacatttatcacttgtttggcaaattgagatagaagtttatgaaagcagattgatatatgttacatttgagctactgggaaggactccagatggtttatagccttaattacattgtaactctagttaaatgtttacctatctgtaccctctgttaaacttgaatatgttaaataccaaagtccatgtattattggattttctgtcaccatcatcaggcacagatcctggtacacaataggtacggaatggatgcatggatgaattattgaattagatgttggtaggcatgtggaaataagaatgaggttcagaattaaagataatctgtatcgagtgtaaagccattggcagagaatgaaatatccagctgagtatacatagaaaaagaaggtaggtagaaaaatggaaaatatcttatgaagtgatgatagaataactctgaatatgtttgaaaacatataaagagttatgtggatgttagctttaaaaattatcttccatgctgtacattagatctgccattcttcatgctgtggatgaaaagcaagcatcagaagttaaattaaaatgatgtcatatattcctcgccttacagtttcataacagaggagaaaagagaaacattctctcattgccaccacccttctccagtcatatttctaggtagatgttgcccaaaaacagataaaaccacagagttggttttgctaggaatggactactaatccaggcaatgttgacagcttttgcttctcattagtgcacgttactaatagaattgctagagattaaaaggaatcctttctacaaagtgctgtatatccataggtgacaaaattctagcttcccctcacaagtacaatataaagttatgttttaaaatcaaaatgcaatttactagcaaactagtaggaactgttatggttacaggaaatttgaatttcagattaactctggttctatgagtagcggttgatatggcaagaatcattttgatcttacatccaggtgctactaaggtctctctgacctatatctcaccaaaaaaaggaacaaaataatgatcctttaatctttctcctaaaatatcataggaaatgatagtggctaaattgcaaataaactaggaaggaaagattcagagtattttatgtgattactctataacaatgccaggccatagtgaaagtgttatttagcagaagactgagttctttgaatgttcctaatttatcacattttaaaaataacctgggcaaaataacctttcatatcagattgagcctttttctaaaaatactcaatatgtttctgtaattatacctacacacttacaattccacagtataatgcaccgataaagtatttttcatccatatatctaatagtagaatggtgtgtatacaataattaagctctttaggcttaccccggaaagcaacaagtttcccttcctttttcctttttatgtattatgttggccataagaaattgatgatattcaactcaatgcagtcttagagatttattcagaaataccatggtgtgtgtgtgtggcgggagtagggttctaatgacaggtcagaacttacttatttgatttcttcattgataatcaggtcttaaaaagaaaatgggtatgctgaaaacatgccttctgtgattctttaccttcatgtgcagttgtctctggataaacactttctttggcacgtatagggttgcactaagctttatagctccaacactccgccccttcagtagattcttgcttgtaactgatgataatgcaaacctgtattatctataggtctccttaaagggcaaccaaaagttcagtagcaattcaggcacaattactgcatgtgagaatcctccatcttgttccctttggagaccacatatatttcttaggcaagtatatttttaaaatccttgttcagcatgacaattcaggaggtcaagttctcccagaaagcagattctgagaaagtgattagcatgaaggaattttattggagagtgctctcaggattaacacctgtgagcggaggaaaggaaagggagcaggattgggcagaaggagaagctgggctaccatacagtcacaactacaacacaatcaaccctccgcctctccttcctagccttccccaggaggatctctgaagtctgaaggtagaatagcccttcagaattgtcctgagttgcagcaagggacccaggattttataccccacaactctcccatcaaccaatacgtgcagcccgtctcggggacatagtgggtaactttgggctaggcacctctctttagctgagtccagctctcagacaggaataacagctgaggactgtcagccagtagcactaccagcagctggggtcagaagtatttcagtcctgaaaaggggtccgggcagcccagcttagcatctactatgccagtcgttctcaaatctggttcctggcaactgtgattctcaagctttagcatatattggaaggcttgttaaaacacagcttgccggattttacccacagagtctctgattcagtagagctaggctgaggcctgggaatttgcatttctaataacttctcagacgttgctggtgctgctggtccatggactatgagaacactgtttcatgctgcccttatttacatactgagaatggtacacagtgctcttatgaatagaatgaaaaccttttgaaatcacattattccttactccatcaaattctcagctatttttgtgcaccataaagctggaatagctgattataaaactttgttatgtaaaaaagtacttaaccaatacagtagattctgtttgcaaagcattattacagtttctaatatctggtcattgttacttgtaaaattcagccaaattttctccagggcctgtagtttgataacttggacaaaggaatttaaaaaaaaatctaattcaagacctttggttttttttctgaacatatcttttttttctttatgattcttatttttacattttacttatcatataagccacttaaacccatatggttccggaaaatttaaaactatatgatacatttagagcatgttgaatgcacagatatggaaattaagtattcttgactcattctagactagacctggcacaattaaaatttagggattcaacgtacacacacatagattccgagagaaatgttgaagccgtaaaacccccacacaagcaggaaacaacagtcttacctattattcaagaggcacgtaaaggagctcatttgaggagattttctgctgttattgccatcgaatttttaacgtattttccaaattagaaaatattcagcctgatgttgtcaatatttcagaccacaagggtatcatttaggaaaatggtttcttactgtcctgaaagagttactgttcttccctaagggcctaatttacaaagcagcaaacttgctggtaggatttggctgaaaatcacattgtctcggtagaactctttcatctgatttatgtgcattgcattttgcaaataactcttggaaagttatttactagttactttctctggaagcagagggtaagcggcatttctagtttaaggatagaggagctaagatgcatcaagcgcagctcatcatgaagctgatgctgataaaatgcacaatattacattctctaagtttcactctgccatgggagaatttcatatttttaaattttgtttgaaattggactacattagaaaatatgtcaaatgtctaaccctgcatttatattctggaatgtgacagcttatttctgttccaaattttgcactggagatggagtaagtcttaatgcaaactgcatgaaactgccacttttataggtcacacccagtcaattgtcagcagttacacatggttcaaactgtaaggtgtatgcccaattgtagcattgagattcgtggagttgttgcagtggttctgaatttttcaagcatgatacataaaaagataaatgactcttttgatatttctccttgcattgatagtttgcctgaaaactagataagcagggagccggcagtccacgttagcccttgaactacatgaggtttaatttatttgcccaaccagaaccctacactacctttcagctgtgcagtattaaagtttatttaggagttgataaatagcttagtgcaatgcttccttttttccagtagctacatcctcataaacctattctaccctccaccagttaatgcagacagaagatttttatccagtatgagcactgaaactccactgtggaagactgtgtgctcagcaaaaacctcacccatgatgaataaacagctcttccgggggctttgctgccgctggctcggcaggagttgtttattgcctggtttgcacatcccatgataaagttgctgctgaaataaattgcagttttgcataattattgacaatcacatcttaacaagcaatgtgtatcatattcaagtgttcaattttttaaaatccatttttagcttatgtttaatcccagaaagtgtttgtgtagtaatagaaggcaaataagacatttaaatagagtactaatttcctcattgcagacaaagtttacctgaatctttttccataggactgttactgcctaaggcaattttcctttctaagctattattatatagatatttgctgagggcatatgtgtgtgtatccacaatacatgcattttatatatatatatatatatatatatatgatcaaaaatatgaatacatttttagagtttttgtcatgaaagagtttgtttcatctttttaaaatattacaggaatggggaaatgggatatgggtagaaggaactaatgtttttgagtaactgtaatgtataactgtataacgtggggcactcaacttcacaggaattttttattttaattctcatcacagcaatagatattgcagatgagaaactgagaatcagagagggaacttgccatatcacgtaagtggtaaagaacactgggaattgaactcagatctgcctagtttttaaaactctactctttttcattacacataacatttttattttggaaaatgttctcagttgtatgatcaagtagttaaatatgaaactaacacaataattataactgatgtcatgcaaaatgatagtttgcacaaaatgatagtttctatgaaatgttatttctttacttgttaagtctttcttcctttgccctccaatccccttctttttgtcttttcctctagtcttttccttttgattctaggtttgtattttcttgacttttctccttgcatatcaaatccttgttttctgcctcagagcagcatcaaagacaagcatggtacagggattttagggttttaactataaaggtttgtctcaaatttggcagtatattaaaaataagctttcaaaattgaccaacaaaaactacaaaattgaaaaaaaggtactttgaactttcacatgttcaaatatatgtatatatatttcacatatatatatgaaacctcctctgtggagaggggtttatagaaatctgtaattgtcattcttgcatgccttcccccatacaaacgcctttaagttaaataaaaatgaaagtaaatagactgcacaatattatagttgttgcttaaaggaagagctgtagcaacaactcaccccattgttggtatattacaatttagttcctccatctttctctttttatggagttcactaggtgcaccattctgatatttaataattgcatctgaacatttggtcctttgcag

SEQ ID NO: 771 - DNA, 50 bp, Homo sapiens
  source 1..50: mol_type = genomic DNA
gttagtatcaaagatacctttttaaaataaaatactggttacatttgata

SEQ ID NO: 772 - DNA, 44 bp, Homo sapiens
  source 1..44: mol_type = genomic DNA
atcacgttaaagctgaaatgaacagtagactttgtatatttatt

SEQ ID NO: 773 - DNA, 50 bp, Homo sapiens
  source 1..50: mol_type = genomic DNA
atgctgcatttgaaaagtttgtcctgaaaggtgggttaccttatactgtc

SEQ ID NO: 774 - DNA, 44 bp, Homo sapiens
  source 1..44: mol_type = genomic DNA
aagcaatctaatatatgtattctgacctgaggattcagaagctg

SEQ ID NO: 775 - DNA, 250 bp, Homo sapiens
  source 1..250: mol_type = genomic DNA
gtttatagaaatctgtaattgtcattcttgcatgccttcccccatacaaacgcctttaagttaaataaaaatgaaagtaaatagactgcacaatattatagttgttgcttaaaggaagagctgtagcaacaactcaccccattgttggtatattacaatttagttcctccatctttctctttttatggagttcactaggtgcaccattctgatatttaataattgcatctgaacatttggtcctttgcag

SEQ ID NO: 776 - DNA, 60 bp, Homo sapiens
  source 1..60: mol_type = genomic DNA
aattgcatctgaacatttggtcctttgcagcagttggccaaagacctccgccagtggcag

SEQ ID NO: 777 - DNA, 155 bp, Homo sapiens
  source 1..155: mol_type = genomic DNA
cagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaag

SEQ ID NO: 778 - protein, 7 aa, synthetic construct
  REGION 1..7: note = Synthetic
ASSLNIA

SEQ ID NO: 779 - protein, 12 aa, synthetic construct
  REGION 1..12: note = Synthetic
SKTFNTHPQSTP

SEQ ID NO: 780 - protein, 12 aa, synthetic construct
  REGION 1..12: note = Synthetic
TARGEHKEEELI

SEQ ID NO: 781 - protein, 9 aa, synthetic construct
  REGION 1..9: note = Synthetic
CQAQGQLVC

SEQ ID NO: 782 - protein, 9 aa, synthetic construct
  REGION 1..9: note = Synthetic
CSERSMNFC

SEQ ID NO: 783 - protein, 9 aa, synthetic construct
  REGION 1..9: note = Synthetic
CPKTRRVPC

SEQ ID NO: 784 - protein, 20 aa, synthetic construct
  REGION 1..20: note = Synthetic
WLSEAGPVVTVRALRGTGSW

SEQ ID NO: 785 - protein, 9 aa, synthetic construct
  REGION 1..9: note = Synthetic
CMQHSMRVC

SEQ ID NO: 786 - protein, 7 aa, synthetic construct
  REGION 1..7: note = Synthetic
DDTRHWG

SEQ ID NO: 787 - protein, 10 aa, synthetic construct
  REGION 1..10: note = Synthetic
GYTFTNYWMH

SEQ ID NO: 788 - protein, 17 aa, synthetic construct
  REGION 1..17: note = Synthetic
EINPINGRSNYGERFKT

SEQ ID NO: 789 - protein, 7 aa, synthetic construct
  REGION 1..7: note = Synthetic
GTRAMHY

SEQ ID NO: 790 - protein, 11 aa, synthetic construct
  REGION 1..11: note = Synthetic
RTSENIYNNLA

SEQ ID NO: 791 - protein, 7 aa, synthetic construct
  REGION 1..7: note = Synthetic
AATNLAD

SEQ ID NO: 792 - protein, 10 aa, synthetic construct
  REGION 1..10: note = Synthetic
QHFWGTPLTF

SEQ ID NO: 793 - protein, 116 aa, synthetic construct
  REGION 1..116: note = Synthetic
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 794 - protein, 107 aa, synthetic construct
  REGION 1..107: note = Synthetic
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 795 - protein, 9 aa, synthetic construct
  REGION 1..9: note = Synthetic
YTFTNYWMH

SEQ ID NO: 796 - protein, 17 aa, synthetic construct
  REGION 1..17: note = Synthetic
EINPINGRSNYAQKFQG

SEQ ID NO: 797 - protein, 17 aa, synthetic construct
  REGION 1..17: note = Synthetic
EINPINGRSNYAEKFQG

SEQ ID NO: 798 - protein, 17 aa, synthetic construct
  REGION 1..17: note = Synthetic
EINPIQGRSNYAEKFQG

SEQ ID NO: 799 - protein, 7 aa, synthetic construct
  REGION 1..7: note = Synthetic
AATNLAE

SEQ ID NO: 800 - protein, 11 aa, synthetic construct
  REGION 1..11: note = Synthetic
RTSENIYSNLA

SEQ ID NO: 801 - protein, 7 aa, synthetic construct
  REGION 1..7: note = Synthetic
AGTNLAD

SEQ ID NO: 802 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 803 - protein, 116 aa, synthetic construct
  REGION 1..116: note = Synthetic
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 804 - protein, 116 aa, synthetic construct
  REGION 1..116: note = Synthetic
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 805 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 806 - protein, 107 aa, synthetic construct
  REGION 1..107: note = Synthetic
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 807 - protein, 107 aa, synthetic construct
  REGION 1..107: note = Synthetic
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 808 - protein, 107 aa, synthetic construct
  REGION 1..107: note = Synthetic
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 809 - protein, 107 aa, synthetic construct
  REGION 1..107: note = Synthetic
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

(19) United States
(12) Patent Application Publication
(10) Pub. No.: US 2024/0368296 A1
(43) Pub. Date: Nov. 7, 2024

(54) MUSCLE TARGETING COMPLEXES AND USES THEREOF FOR TREATING DYSTROPHINOPATHIES

(71) Applicant: Dyne Therapeutics, Inc., Waltham, MA (US)

(72) Inventors: Cody A. Desjardins, Waltham, MA (US); Kim Tang, Waltham, MA (US); James McSwiggen, Arvada, CO (US); Romesh R. Subramanian, Framingham, MA (US); Timothy Weeden, Waltham, MA (US); Mohammed T. Qatanani, Waltham, MA (US); Brendan Quinn, Waltham, MA (US); John Najim, Waltham, MA (US)

(73) Assignee: Dyne Therapeutics, Inc., Waltham, MA (US)

(21) Appl. No.: 18/577,452

(22) PCT Filed: Jul. 8, 2022
(86) PCT No.: PCT/US2022/073541

§ 371 Date: Jan. 8, 2024

Related U.S. Application Data
(60) Provisional application No. 63/220,016, filed on Jul. 9, 2021.
(60) Provisional application No. 63/316,466, filed on Mar. 4, 2022.

(51) Int. Cl. C07K 16/28; A61K 39/00; A61K 47/68; C12N 15/113

(52) U.S. Cl. CPC C07K 16/2881; A61K 47/6807; A61K 47/6849; C12N 15/113; A61K 2039/505; C07K 2317/55; C07K 2317/77; C12N 2310/3233; C12N 2310/3513; C12N 2320/32; C12N 2320/33

(57) ABSTRACT

Aspects of the disclosure relate to complexes comprising a muscle-targeting agent covalently linked to a molecular payload. In some embodiments, the muscle-targeting agent specifically binds to an internalizing cell surface receptor on muscle cells. In some embodiments, the molecular payload promotes the expression or activity of a functional dystrophin protein. In some embodiments, the molecular payload is an oligonucleotide, such as an antisense oligonucleotide, e.g., an oligonucleotide that causes exon skipping in a mRNA expressed from a mutant DMD allele.

### RELATED APPLICATIONS

This application claims priority under 35 U.S.C. § 119(e) to U.S. Provisional Application Ser. No. 63/220,016, entitled “MUSCLE TARGETING COMPLEXES AND USES THEREOF FOR TREATING DYSTROPHINOPATHIES”, filed on Jul. 9, 2021, and to U.S. Provisional Application Ser. No. 63/316,466, entitled “MUSCLE TARGETING COMPLEXES AND USES THEREOF FOR TREATING DYSTROPHINOPATHIES”, filed on Mar. 4, 2022; the contents of each of which are incorporated herein by reference in their entirety.

### FIELD OF THE INVENTION

The present application relates to targeting complexes for delivering molecular payloads (e.g., oligonucleotides) to cells and uses thereof, particularly uses relating to treatment of disease.

### REFERENCE TO AN ELECTRONIC SEQUENCE LISTING

The contents of the electronic sequence listing (D082470055W000-SEQ-COB.xml; Size: 1,054,275 bytes; and Date of Creation: Jul. 7, 2022) are herein incorporated by reference in their entirety.

### BACKGROUND OF INVENTION

Dystrophinopathies are a group of distinct neuromuscular diseases that result from mutations in the gene encoding dystrophin. Dystrophinopathies include Duchenne muscular dystrophy, Becker muscular dystrophy, and X-linked dilated cardiomyopathy. The DMD gene (“DMD”), which encodes dystrophin, is a large gene, containing 79 exons and about 2.6 million total base pairs. Numerous mutations in DMD, including exonic frameshift, deletion, substitution, and duplicative mutations, are able to diminish the expression of functional dystrophin, leading to dystrophinopathies. Several agents that target exons of human DMD have been approved by the U.S. Food and Drug Administration (FDA), including casimersen, viltolarsen, golodirsen, and eteplirsen. Of these, viltolarsen and golodirsen target exon 53.

### SUMMARY OF INVENTION

According to some aspects, the disclosure provides complexes that target muscle cells for purposes of delivering molecular payloads to those cells, as well as molecular payloads that can be used therein. In some embodiments, complexes provided herein are particularly useful for delivering molecular payloads that increase or restore expression or activity of functional dystrophin protein. In some embodiments, complexes comprise oligonucleotide based molecular payloads that promote expression of functional dystrophin protein through an in-frame exon skipping mechanism or suppression of stop codons, such as by facilitating skipping of DMD exon 53. In some embodiments, molecular payloads provided herein are useful for facilitating exon skipping in a DMD sequence, such as skipping of DMD exon 53. Accordingly, in some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor mediated internalization, following which the molecular payload may be released to perform a function inside the cells. For example, complexes engineered to deliver oligonucleotides may release the oligonucleotides such that the oligonucleotides can promote expression of functional dystrophin protein (e.g., through an exon skipping mechanism, such as by facilitating skipping of DMD exon 53) in the muscle cells. In some embodiments, the oligonucleotides are released by endosomal cleavage of covalent linkers connecting oligonucleotides and muscle-targeting agents of the complexes. Complexes and molecular payloads provided herein can be used for treating subjects having a mutated DMD gene, such as a mutated DMD gene that is amenable to exon 53 skipping.

According to some aspects, complexes comprising an anti-transferrin receptor 1 (TfR1) antibody covalently linked to an oligonucleotide configured for inducing skipping of exon 53 in a DMD pre-mRNA are provided herein, wherein the oligonucleotide comprises a region of complementarity that is complementary with at least 8 consecutive nucleotides of any one of SEQ ID NOs: 224, 206, 209, 212, 277, 214, 207, 208, 205, 160-204, 210, 211, 213, 215-223, 225-276, and 278-334.

In some embodiments, the oligonucleotide comprises a region of complementarity that is complementary with at least 8 consecutive nucleotides of any one of SEQ ID NOs: 212, 224, and 209.

In some embodiments, the oligonucleotide comprises a region of complementarity that is complementary with at least 8 consecutive nucleotides of any one of SEQ ID NOs: 206, 277, and 205.

In some embodiments, the oligonucleotide comprises a region of complementarity that is complementary with at least 8 consecutive nucleotides of any one of SEQ ID NOs: 206, 224, and 209.

In some embodiments, the oligonucleotide comprises a region of complementarity that is complementary with at least 8 consecutive nucleotides of any one of SEQ ID NOs: 214, 207, and 208.

In some embodiments, the oligonucleotide comprises a region of complementarity that is complementary with at least 8 consecutive nucleotides of any one of SEQ ID NOs: 212, 206, and 209.

In some embodiments, the oligonucleotide comprises a region of complementarity that is complementary with at least 8 consecutive nucleotides of any one of SEQ ID NOs: 214, 207, and 205.

In some embodiments, the oligonucleotide comprises a region of complementarity that is complementary with at least 8 consecutive nucleotides of any one of SEQ ID NOs: 277, 214, and 208.

In some embodiments, the anti-TfR1 antibody comprises:
(i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.

In some embodiments, the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.

In some embodiments, the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.

In some embodiments, the anti-TfR1 antibody is a Fab fragment, a Fab′ fragment, a F(ab′)2 fragment, an scFv, an Fv, or a full-length IgG.

In some embodiments, the anti-TfR1 antibody is a Fab fragment.

In some embodiments, the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.

In some embodiments, the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.

In some embodiments, the anti-TfR1 antibody does not specifically bind to the transferrin binding site of the transferrin receptor 1 and/or the anti-TfR1 antibody does not inhibit binding of transferrin to the transferrin receptor 1.

In some embodiments, the oligonucleotide is complementary to at least 4 consecutive nucleotides of a splicing feature of the DMD pre-mRNA.

In some embodiments, the splicing feature is an exonic splicing enhancer (ESE) in exon 53 of the DMD pre-mRNA, optionally wherein the ESE comprises a sequence of any one of SEQ ID NOs: 689-715.

In some embodiments, the splicing feature is a branch point, a splice donor site, or a splice acceptor site, optionally wherein the splicing feature is across the junction of exon 52 and intron 52, in intron 52, across the junction of intron 52 and exon 53, across the junction of exon 53 and intron 53, in intron 53, or across the junction of intron 53 and exon 54 of the DMD pre-mRNA, and further optionally wherein the splicing feature comprises a sequence of any one of SEQ ID NOs: 685-688 and 716-718.

In some embodiments, the oligonucleotide comprises a sequence complementary to any one of SEQ ID NOs: 160-334 or comprises a sequence of any one of SEQ ID NOs: 335-684, wherein each thymine base (T) may independently and optionally be replaced with a uracil base (U), and each U may independently and optionally be replaced with a T.

In some embodiments, the oligonucleotide comprises a sequence of any one of SEQ ID NOs: 574, 556, 559, 562, 627, 564, 557, 558, and 555, wherein each thymine base (T) may independently and optionally be replaced with a uracil base (U), and each U may independently and optionally be replaced with a T.

In some embodiments, the oligonucleotide comprises a sequence of any one of SEQ ID NOs: 562, 574, and 559, wherein each T may independently and optionally be replaced with a U, and each U may independently and optionally be replaced with a T.

In some embodiments, the oligonucleotide comprises a sequence of any one of SEQ ID NOs: 556, 627, and 555, wherein each T may independently and optionally be replaced with a U, and each U may independently and optionally be replaced with a T.

In some embodiments, the oligonucleotide comprises a sequence of any one of SEQ ID NOs: 556, 574, and 559, wherein each T may independently and optionally be replaced with a U, and each U may independently and optionally be replaced with a T.

In some embodiments, the oligonucleotide comprises a sequence of any one of SEQ ID NOs: 564, 557, and 558, wherein each T may independently and optionally be replaced with a U, and each U may independently and optionally be replaced with a T.

In some embodiments, the oligonucleotide comprises a sequence of any one of SEQ ID NOs: 562, 556, and 559, wherein each T may independently and optionally be replaced with a U, and each U may independently and optionally be replaced with a T.

In some embodiments, the oligonucleotide comprises a sequence of any one of SEQ ID NOs: 564, 557, and 555, wherein each T may independently and optionally be replaced with a U, and each U may independently and optionally be replaced with a T.

In some embodiments, the oligonucleotide comprises a sequence of any one of SEQ ID NOs: 627, 564, and 558, wherein each T may independently and optionally be replaced with a U, and each U may independently and optionally be replaced with a T.

In some embodiments, the oligonucleotide comprises one or more phosphorodiamidate morpholinos, optionally wherein the oligonucleotide is a phosphorodiamidate morpholino oligomer (PMO).

In some embodiments, the anti-TfR1 antibody is covalently linked to the oligonucleotide via a cleavable linker, optionally wherein the cleavable linker comprises a valine-citrulline sequence.

In some embodiments, the anti-TfR1 antibody is covalently linked to the oligonucleotide via conjugation to a lysine residue or a cysteine residue of the antibody.

According to some aspects, oligonucleotides that target DMD are provided herein, wherein the oligonucleotide comprises a region of complementarity to any one of SEQ ID NOs: 160-334, optionally wherein the region of complementarity comprises at least 15 consecutive nucleosides complementary to any one of SEQ ID NOs: 160-334.

In some embodiments, the oligonucleotide comprises a region of complementarity to any one of SEQ ID NOs: 212, 224, and 209.

In some embodiments, the oligonucleotide comprises a region of complementarity to any one of SEQ ID NOs: 206, 277, and 205.

In some embodiments, the oligonucleotide comprises a region of complementarity to any one of SEQ ID NOs: 206, 224, and 209.

In some embodiments, the oligonucleotide comprises a region of complementarity to any one of SEQ ID NOs: 214, 207, and 208.

In some embodiments, the oligonucleotide comprises a region of complementarity to any one of SEQ ID NOs: 212, 206, and 209.

In some embodiments, the oligonucleotide comprises a region of complementarity to any one of SEQ ID NOs: 214, 207, and 205.

In some embodiments, the oligonucleotide comprises a region of complementarity to any one of SEQ ID NOs: 277, 214, and 208.

In some embodiments, the oligonucleotide comprises at least 15 consecutive nucleosides of any one of SEQ ID NOs: 335-684, optionally wherein the oligonucleotide comprises a sequence of any one of SEQ ID NOs: 335-684, wherein each thymine base (T) may independently and optionally be replaced with a uracil base (U), and each U may independently and optionally be replaced with a T.

In some embodiments, the oligonucleotide comprises a sequence of any one of SEQ ID NOs: 574, 556, 559, 562, 627, 564, 557, 558, and 555, wherein each thymine base (T) may independently and optionally be replaced with a uracil base (U), and each U may independently and optionally be replaced with a T.

In some embodiments, the oligonucleotide comprises a sequence of any one of SEQ ID NOs: 562, 574, and 559, wherein each T may independently and optionally be replaced with a U, and each U may independently and optionally be replaced with a T.

In some embodiments, the oligonucleotide comprises a sequence of any one of SEQ ID NOs: 556, 627, and 555, wherein each T may independently and optionally be replaced with a U, and each U may independently and optionally be replaced with a T.

In some embodiments, the oligonucleotide comprises a sequence of any one of SEQ ID NOs: 556, 574, and 559, wherein each T may independently and optionally be replaced with a U, and each U may independently and optionally be replaced with a T.

In some embodiments, the oligonucleotide comprises a sequence of any one of SEQ ID NOs: 564, 557, and 558, wherein each T may independently and optionally be replaced with a U, and each U may independently and optionally be replaced with a T.

In some embodiments, the oligonucleotide comprises a sequence of any one of SEQ ID NOs: 562, 556, and 559, wherein each T may independently and optionally be replaced with a U, and each U may independently and optionally be replaced with a T.

In some embodiments, the oligonucleotide comprises a sequence of any one of SEQ ID NOs: 564, 557, and 555, wherein each T may independently and optionally be replaced with a U, and each U may independently and optionally be replaced with a T.

In some embodiments, the oligonucleotide comprises a sequence of any one of SEQ ID NOs: 627, 564, and 558, wherein each T may independently and optionally be replaced with a U, and each U may independently and optionally be replaced with a T.

According to some aspects, methods of delivering an oligonucleotide to a cell are provided herein, the method comprising contacting the cell with a complex disclosed herein or with an oligonucleotide disclosed herein.

According to some aspects, methods of promoting the expression or activity of a dystrophin protein in a cell are provided herein, the method comprising contacting the cell with a complex disclosed herein or with an oligonucleotide disclosed herein in an amount effective for promoting internalization of the oligonucleotide to the cell, optionally wherein the cell is a muscle cell.

In some embodiments, the cell comprises a DMD gene that is amenable to skipping of exon 53.

In some embodiments, the dystrophin protein is a truncated dystrophin protein.

### BRIEF DESCRIPTION OF THE DRAWINGS

FIG. 1 shows data illustrating that conjugates containing anti-TfR1 Fab (3M12 VH4/Vκ3) conjugated to a DMD exon-skipping oligonucleotide resulted in enhanced exon skipping compared to the naked DMD exon skipping oligo in Duchenne muscular dystrophy patient myotubes.

FIG. 2 shows data illustrating that conjugates containing anti-TfR1 Fab (3M12 VH4/Vκ3) conjugated to DMD exon 53-skipping oligonucleotides facilitated skipping of exon 53 in Duchenne muscular dystrophy patient myotubes.

### DETAILED DESCRIPTION OF INVENTION

Aspects of the disclosure relate to a recognition that while certain molecular payloads (e.g., oligonucleotides, peptides, small molecules) can have beneficial effects in muscle cells, it has proven challenging to effectively target such cells. Accordingly, as described herein, the present disclosure provides complexes comprising muscle-targeting agents covalently linked to molecular payloads in order to overcome such challenges. In some embodiments, the complexes are particularly useful for delivering molecular payloads that modulate (e.g., promote) the expression or activity of dystrophin protein (e.g., a truncated dystrophin protein) or DMD (e.g., a mutated DMD allele). In some embodiments, complexes provided herein may comprise oligonucleotides that promote expression and activity of dystrophin protein or DMD, such as by facilitating in-frame exon skipping and/or suppression of premature stop codons. For example, complexes may comprise oligonucleotides that induce skipping of exon(s) of DMD RNA (e.g., pre-mRNA), such as oligonucleotides that induce skipping of exon 53. In some embodiments, synthetic nucleic acid payloads (e.g., DNA or RNA payloads) may be used that express one or more proteins that promote normal expression and activity of dystrophin protein or DMD.

Duchenne muscular dystrophy is an X-linked muscular disorder caused by one or more mutations in the DMD gene located on Xp21. Dystrophin protein typically forms the dystrophin-associated glycoprotein complex (DGC) at the sarcolemma, which links the muscle sarcomeric structure to the extracellular matrix and protects the sarcolemma from contraction-induced injury. In patients with Duchenne muscular dystrophy, the dystrophin protein is generally absent and muscle fibers typically become damaged due to mechanical overextension. Mutations in the DMD gene are associated with two types of muscular dystrophy, Duchenne muscular dystrophy and Becker muscular dystrophy, depending on whether the translational reading frame is lost or maintained. Becker muscular dystrophy is a clinically milder form of Duchenne muscular dystrophy, and is characterized by features similar to Duchenne muscular dystrophy. In some embodiments, exon skipping induced by oligonucleotides (e.g., delivered using complexes provided herein) can be used to restore the reading frame of a mutated DMD allele resulting in production of a truncated dystrophin protein that is sufficiently functional to improve muscle function. In some embodiments, such exon skipping converts a Duchenne muscular dystrophy phenotype into a milder Becker muscular dystrophy phenotype.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Branch point: As used herein, the term “branch point” or “branch site” refers to a nucleic acid sequence motif within an intron of a gene or pre-mRNA that is involved in splicing of pre-mRNA into mRNA (i.e., removing introns from the pre-mRNA), and can be referred to as a splicing feature. A branch point is typically located 18 to 40 nucleotides from the 3′ end of an intron, and contains an adenine but is otherwise relatively unrestricted in sequence. Common sequence motifs for branch points are YNYYRAY, YTRAC, and YNYTRAY, where Y is a pyrimidine, N is any nucleotide, R is any purine, and A is adenine. During splicing, the pre-mRNA is cleaved at the 5′ end of the intron, which then attaches to the branch point region downstream through transesterification bonding between guanines and adenines from the 5′ end and the branch point, respectively, to form a looped lariat structure.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.- P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene (DMD or DMD gene) may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2, NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 45, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Dystrophinopathy: As used herein, the term “dystrophinopathy” refers to a muscle disease results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs can be referred to as splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Splice acceptor site: As used herein, the term “splice acceptor site” or “splice acceptor” refers to a nucleic acid sequence motif at the 3′ end of an intron or across an intron/exon junction of a gene or pre-mRNA that is involved in splicing of pre-mRNA into mRNA (i.e., removing introns from the pre-mRNA), and can be referred to as a splicing feature. A splice acceptor site includes a terminal AG sequence at the 3′ end of an intron, which is typically preceded (5′-ward) by a region high in pyrimidines (C/U). Upstream from the splice acceptor site is the branch point. Formation of a lariat loop intermediate structure by a transesterification reaction between the branch point and the splice donor site releases a 3′-OH of the 5′ exon, which subsequently reacts with the first nucleotide of the 3′ exon, thereby joining the exons and releasing the intron lariat. The AG sequence at the 3′ end of the intron in the splice acceptor site is known to be critical for proper splicing, as changing one of these nucleotides results in inhibition of splicing. Rarely, alternative splice acceptor sites have an AC at the 3′ end of the intron, instead of the more common AG. A common splice acceptor site motif has a sequence of or similar to [Y-rich region]-NCAGG or YₓNYAGG, in which Y represents a pyrimidine, N represents any nucleotide, and x is a number from 4 to 20. The cut site follows the AG, which represent the 3′-terminal nucleotides of the excised intron.

Splice donor site: As used herein, the term “splice donor site” or “splice donor” refers to a nucleic acid sequence motif at the 5′ end of an intron or across an exon/intron junction of a gene or pre-mRNA that is involved in splicing of pre-mRNA into mRNA (i.e., removing introns from the pre-mRNA), and can be referred to as a splicing feature. A splice donor site includes a terminal GU sequence at the 5′ end of the intron, within a larger and fairly unconstrained sequence. During splicing, the 2′-OH of a nucleotide within the branch point initiates a transesterification reaction via a nucleophilic attack on the 5′ G of the intron within the splice donor site. The G is thereby cleaved from the pre-mRNA and bonds instead to the branch point nucleotide, forming a loop lariat structure. The 3′ nucleotide of the upstream exon subsequently binds the splice acceptor site, joining the exons and excising the intron. A typical splice donor site has a sequence of or similar to GGGURAGU or AGGURNG, in which R represents a purine and N represents any nucleotide. The cut site precedes the first GU (i.e., GG/GURAGU or AG/GURNG), which represent the 5′-terminal nucleotides of the excised intron.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy. In some embodiments, a subject is a patient that has a mutation of the DMD gene that is amenable to exon 53 skipping.

Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

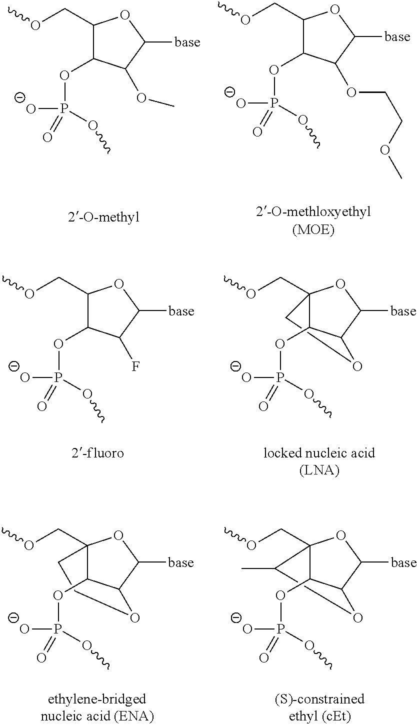

These examples are shown with phosphate groups, but any internucleoside linkages are contemplated between 2′-modified nucleosides.

### II. Complexes

Provided herein are complexes that comprise a targeting agent, e.g. an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present with a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell.

In some embodiments, a complex comprises a muscle-targeting agent, e.g., an anti-transferrin receptor antibody, covalently linked to a molecular payload, e.g., an antisense oligonucleotide that targets DMD to promote exon skipping, e.g., in a transcript encoded from a mutated DMD allele. In some embodiments, the complex targets a DMD pre-mRNA to promote skipping of exon 53 in the DMD pre-mRNA.

### A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure. It should also be appreciated that any muscle targets (e.g., muscle surface proteins) can be targeted by any type of muscle-targeting agent described herein. For example, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). A muscle-targeting agent may comprise, or consist of, a small molecule. Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-TfR1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor (TfR) Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor antibody, or an anti-TfR1 antibody. Antibodies that bind, e.g. specifically bind, to a transferrin receptor may be internalized into the cell, e.g. through receptor-mediated endocytosis, upon binding to a transferrin receptor.

It should be appreciated that anti-TfR1 antibodies may be produced, synthesized, and/or (e.g., and) derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-TfR1 antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor.

In some embodiments, the anti-TfR1 antibodies described herein (e.g., Anti-TfR clone 8 in Table 2 below) bind an epitope in TfR1, wherein the epitope comprises residues in amino acids 214-241 and/or amino acids 354-381 of SEQ ID NO: 105. In some embodiments, the anti-TfR1 antibodies described herein bind an epitope comprising residues in amino acids 214-241 and amino acids 354-381 of SEQ ID NO: 105. In some embodiments, the anti-TfR1 antibodies described herein bind an epitope comprising one or more of residues Y222, T227, K231, H234, T367, S368, S370, T376, and S378 of human TfR1 as set forth in SEQ ID NO: 105. In some embodiments, the anti-TfR1 antibodies described herein bind an epitope comprising residues Y222, T227, K231, H234, T367, S368, S370, T376, and S378 of human TfR1 as set forth in SEQ ID NO: 105.

In some embodiments, the anti-TfR1 antibody described herein (e.g., 3M12 in Table 2 below and its variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 2 below and its variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 2 below and its variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 2 below and its variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEE
ENADNNTKANVTKPKRCSGSICYGTIAVIVFFLIGFMIGYLGYCK
GVEPKTECERLAGTESPVREEPGEDFPAARRLYWDDLKRKLSEKL
DSTDFTGTIKLLNENSYVPREAGSQKDENLALYVENQFREFKLSK
VWRDQHFVKIQVKDSAQNSVIIVDKNGRLVYLVENPGGYVAYSKA
ATVTGKLVHANFGTKKDFEDLYTPVNGSIVIVRAGKITFAEKVAN
AESLNAIGVLIYMDQTKFPIVNAELSFFGHAHLGTGDPYTPGFPS
FNHTQFPPSRSSGLPNIPVQTISRAAAEKLFGNMEGDCPSDWKTD
STCRMVTSESKNVKLTVSNVLKEIKILNIFGVIKGFVEPDHYVVV
GAQRDAWGPGAAKSGVGTALLLKLAQMFSDMVLKDGFQPSRSIIF
ASWSAGDFGSVGATEWLEGYLSSLHLKAFTYINLDKAVLGTSNFK
VSASPLLYTLIEKTMQNVKHPVTGQFLYQDSNWASKVEKLTLDNA
AFPFLAYSGIPAVSFCFCEDTDYPYLGTTMDTYKELIERIPELNK
VARAAAEVAGQFVIKLTHDVELNLDYERYNSQLLSFVRDLNQYRA
DIKEMGLSLOWLYSARGDFFRATSRLTTDFGNAEKTDRFVMKKLN
DRVMRVEYHFLSPYVSPKESPFRHVFWGSGSHTLPALLENLKLRK
QNNGAFNETLFRNQLALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 106)
MMDQARSAFSNLFGGEPLSYTRFSLARQVDGDNSHVEMKLGVDEE
ENTDNNTKPNGTKPKRCGGNICYGTIAVIIFFLIGFMIGYLGYC
KGVEPKTECERLAGTESPAREEPEEDFPAAPRLYWDDLKRKLSEK
LDTTDFTSTIKLLNENLYVPREAGSQKDENLALYIENQFREFKLS
KVWRDQHFVKIQVKDSAQNSVIIVDKNGGLVYLVENPGGYVAYSK
AATVTGKLVHANFGTKKDFEDLDSPVNGSIVIVRAGKITFAEKVA
NAESLNAIGVLIYMDQTKFPIVKADLSFFGHAHLGTGDPYTPGFP
SFNHTQFPPSQSSGLPNIPVQTISRAAAEKLFGNMEGDCPSDWKT
DSTCKMVTSENKSVKLTVSNVLKETKILNIFGVIKGFVEPDHYVV
VGAQRDAWGPGAAKSSVGTALLLKLAQMFSDMVLKDGFQPSRSII
FASWSAGDFGSVGATEWLEGYLSSLHLKAFTYINLDKAVLGTSNF
KVSASPLLYTLIEKTMQDVKHPVTGRSLYQDSNWASKVEKLTLDN
AAFPFLAYSGIPAVSFCFCEDTDYPYLGTTMDTYKELVERIPEL
NKVARAAAEVAGQFVIKLTHDTELNLDYERYNSQLLLFLRDLNQY
RADVKEMGLSLOWLYSARGDFFRATSRLTTDFRNAEKRDKFVMKK
LNDRVMRVEYYFLSPYVSPKESPFRHVFWGSGSHTLSALLESLKL
RRQNNSAFNETLFRNQLALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEE
ENTDNNTKANGTKPKRCGGNICYGTIAVIIFFLIGFMIGYLGYC
KGVEPKTECERLAGTESPAREEPEEDFPAAPRLYWDDLKRKLSEK
LDTTDFTSTIKLLNENLYVPREAGSQKDENLALYIENQFREFKLS
KVWRDQHFVKIQVKDSAQNSVIIVDKNGGLVYLVENPGGYVAYSK
AATVTGKLVHANFGTKKDFEDLDSPVNGSIVIVRAGKITFAEKVA
NAESLNAIGVLIYMDQTKFPIVKADLSFFGHAHLGTGDPYTPGFP
SFNHTQFPPSQSSGLPNIPVQTISRAAAEKLFGNMEGDCPSDWKT
DSTCKMVTSENKSVKLTVSNVLKETKILNIFGVIKGFVEPDHYVV
VGAQRDAWGPGAAKSSVGTALLLKLAQMFSDMVLKDGFQPSRSII
FASWSAGDFGSVGATEWLEGYLSSLHLKAFTYINLDKAVLGTSNF
KVSASPLLYTLIEKTMQDVKHPVTGRSLYQDSNWASKVEKLTLDN
AAFPFLAYSGIPAVSFCFCEDTDYPYLGTTMDTYKELVERIPELN
KVARAAAEVAGQFVIKLTHDTELNLDYERYNSQLLLFLRDLNQYR
ADVKEMGLSLOWLYSARGDFFRATSRLTTDFRNAEKRDKFVMKKL
NDRVMRVEYYFLSPYVSPKESPFRHVFWGSGSHTLSALLESLKLR
RQNNSAFNETLFRNQLALATWTIQGAANALSGDVWDIDNEF.

An example mouse transferrin receptor amino acid sequence, corresponding to NCBI sequence NP_001344227.1 (transferrin receptor protein 1, Mus musculus) is as follows

(SEQ ID NO: 108)
MMDQARSAFSNLFGGEPLSYTRFSLARQVDGDNSHVEMKLAADEE
ENADNNMKASVRKPKRFNGRLCFAAIALVIFFLIGFMSGYLGYCK
RVEQKEECVKLAETEETDKSETMETEDVPTSSRLYWADLKTLLSE
KLNSIEFADTIKQLSQNTYTPREAGSQKDESLAYYIENQFHEFKF
SKVWRDEHYVKIQVKSSIGQNMVTIVQSNGNLDPVESPEGYVAFS
KPTEVSGKLVHANFGTKKDFEELSYSVNGSLVIVRAGEITFAEKV
ANAQSFNAIGVLIYMDKNKFPVVEADLALFGHAHLGTGDPYTPGF
PSFNHTQFPPSQSSGLPNIPVQTISRAAAEKLFGKMEGSCPARWN
IDSSCKLELSQNQNVKLIVKNVLKERRILNIFGVIKGYEEPDRYV
VVGAQRDALGAGVAAKSSVGTGLLLKLAQVESDMISKDGFRPSRS
IIFASWTAGDFGAVGATEWLEGYLSSLHLKAFTYINLDKVVLGTS
NFKVSASPLLYTLMGKIMQDVKHPVDGKSLYRDSNWISKVEKLSF
DNAAYPFLAYSGIPAVSFCFCEDADYPYLGTRLDTYEALTQKVPQ
LNQMVRTAAEVAGQLIIKLTHDVELNLDYEMYNSKLLSFMKDLNQ
FKTDIRDMGLSLOWLYSARGDYFRATSRLTTDFHNAEKTNRFVMR
EINDRIMKVEYHFLSPYVSPRESPFRHIFWGSGSHTLSALVENLK
LRQKNITAFNETLFRNQLALATWTIQGVANALSGDIWNIDNEF

In some embodiments, an anti-TfR1 antibody binds to an amino acid segment of the receptor as follows: FVKIQVKDSAQNSVIIVDKNGRLVYLVENPGGYVAYSKAATVTGKLVHANFGTKKDF EDLYTPVNGSIVIVRAGKITFAEKVANAESLNAIGVLIYMDQTKFPIVNAELSFFGHAH LGTGDPYTPGFPSFNHTQFPPSRSSGLPNIPVQTISRAAAEKLFGNMEGDCPSDWKTDS TCRMVTSESKNVKLTVSNVLKE (SEQ ID NO: 109) and does not inhibit the binding interactions between transferrin receptors and transferrin and/or (e.g., and) human hemochromatosis protein (also known as HFE). In some embodiments, the anti-TfR1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) a VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

Provided herein, in some aspects, are humanized antibodies that bind to transferrin receptor with high specificity and affinity. In some embodiments, the humanized anti-TfR1 antibody described herein specifically binds to any extracellular epitope of a transferrin receptor or an epitope that becomes exposed to an antibody. In some embodiments, the humanized anti-TfR1 antibodies provided herein bind specifically to transferrin receptor from human, non-human primates, mouse, rat, etc. In some embodiments, the humanized anti-TfR1 antibodies provided herein bind to human transferrin receptor. In some embodiments, the humanized anti-TfR1 antibody described herein binds to an amino acid segment of a human or non-human primate transferrin receptor, as provided in SEQ ID NOs: 105-108. In some embodiments, the humanized anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, an anti-TFR1 antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a KD of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibodies described herein does not complete with or inhibit transferrin binding to the TfR1. In some embodiments, binding of any one of the anti-TfR1 antibodies described herein does not complete with or inhibit HFE-beta-2-microglobulin binding to the TfR1.

Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY | DDYMY | GFNIKDD |
| | H1 | (SEQ ID NO: 1) | (SEQ ID NO: 7) | (SEQ ID NO: 12) |
| | CDR- | IDPENGDT | WIDPENGDTEYASKFQD | ENG |
| | H2 | (SEQ ID NO: 2) | (SEQ ID NO: 8) | (SEQ ID NO: 13) |
| | CDR- | TLWLRRGLDY | WLRRGLDY | LRRGLD |
| | H3 | (SEQ ID NO: 3) | (SEQ ID NO: 9) | (SEQ ID NO: 14) |
| | CDR- | KSLLHSNGYTY | RSSKSLLHSNGYTYLF | SKSLLHSNGYTY |
| | L1 | (SEQ ID NO: 4) | (SEQ ID NO: 10) | (SEQ ID NO: 15) |
| | CDR- | RMS | RMSNLAS | RMS |
| | L2 | (SEQ ID NO: 5) | (SEQ ID NO: 11) | (SEQ ID NO: 5) |
| | CDR- | MQHLEYPFT | MQHLEYPFT | HLEYPF |
| | L3 | (SEQ ID NO: 6) | (SEQ ID NO: 6) | (SEQ ID NO: 16) |
| | VH | EVQLQQSGAELVRPGASVKLSCTASGFNIKDDYMYWVKQR | | |
| | | PEQGLEWIGWIDPENGDTEYASKFQDKATVTADTSSNTAY | | |
| | | LQLSSLTSEDTAVYYCTLWLRRGLDYWGQGTSVTVSS | | |
| | | (SEQ ID NO: 17) | | |
| | VL | DIVMTQAAPSVPVTPGESVSISCRSSKSLLHSNGYTYLFW | | |
| | | FLQRPGQSPQLLIYRMSNLASGVPDRFSGSGSGTAFTLRI | | |
| | | SRVEAEDVGVYYCMQHLEYPFTFGGGTKLEIK | | |
| | | (SEQ ID NO: 18) | | |
| 3-A4 | CDR- | GFNIKDDY | DDYMY | GFNIKDD |
| N54T* | H1 | (SEQ ID NO: 1) | (SEQ ID NO: 7) | (SEQ ID NO: 12) |
| | CDR- | IDPETGDT | WIDPETGDTEYASKFQD | ETG |
| | H2 | (SEQ ID NO: 19) | (SEQ ID NO: 20) | (SEQ ID NO: 21) |
| | CDR- | TLWLRRGLDY | WLRRGLDY | LRRGLD |
| | H3 | (SEQ ID NO: 3) | (SEQ ID NO: 9) | (SEQ ID NO: 14) |
| | CDR- | KSLLHSNGYTY | RSSKSLLHSNGYTYLF | SKSLLHSNGYTY |
| | L1 | (SEQ ID NO: 4) | (SEQ ID NO: 10) | (SEQ ID NO: 15) |
| | CDR- | RMS | RMSNLAS | RMS |
| | L2 | (SEQ ID NO: 5) | (SEQ ID NO: 11) | (SEQ ID NO: 5) |
| | CDR- | MQHLEYPFT | MQHLEYPFT | HLEYPF |
| | L3 | (SEQ ID NO: 6) | (SEQ ID NO: 6) | (SEQ ID NO: 16) |
| | VH | EVQLQQSGAELVRPGASVKLSCTASGFNIKDDYMYWVKQR | | |
| | | PEQGLEWIGWIDPETGDTEYASKFQDKATVTADTSSNTAY | | |
| | | LQLSSLTSEDTAVYYCTLWLRRGLDYWGQGTSVTVSS | | |
| | | (SEQ ID NO: 22) | | |
| | VL | DIVMTQAAPSVPVTPGESVSISCRSSKSLLHSNGYTYLFW | | |
| | | FLQRPGQSPQLLIYRMSNLASGVPDRFSGSGSGTAFTLRI | | |
| | | SRVEAEDVGVYYCMQHLEYPFTFGGGTKLEIK | | |
| | | (SEQ ID NO: 18) | | |
| 3-A4 | CDR- | GFNIKDDY | DDYMY | GFNIKDD |
| N54S* | H1 | (SEQ ID NO: 1) | (SEQ ID NO: 7) | (SEQ ID NO: 12) |
| | CDR- | IDPESGDT | WIDPESGDTEYASKFQD | ESG |
| | H2 | (SEQ ID NO: 23) | (SEQ ID NO: 24) | (SEQ ID NO: 25) |
| | CDR- | TLWLRRGLDY | WLRRGLDY | LRRGLD |
| | H3 | (SEQ ID NO: 3) | (SEQ ID NO: 9) | (SEQ ID NO: 14) |
| | CDR- | KSLLHSNGYTY | RSSKSLLHSNGYTYLF | SKSLLHSNGYTY |
| | L1 | (SEQ ID NO: 4) | (SEQ ID NO: 10) | (SEQ ID NO: 15) |
| | CDR- | RMS | RMSNLAS | RMS |
| | L2 | (SEQ ID NO: 5) | (SEQ ID NO: 11) | (SEQ ID NO: 5) |
| | CDR- | MQHLEYPFT | MQHLEYPFT | HLEYPF |
| | L3 | (SEQ ID NO: 6) | (SEQ ID NO: 6) | (SEQ ID NO: 16) |
| | VH | EVQLQQSGAELVRPGASVKLSCTASGFNIKDDYMYWVKQR | | |
| | | PEQGLEWIGWIDPESGDTEYASKFQDKATVTADTSSNTAY | | |
| | | LQLSSLTSEDTAVYYCTLWLRRGLDYWGQGTSVTVSS | | |
| | | (SEQ ID NO: 26) | | |
| | VL | DIVMTQAAPSVPVTPGESVSISCRSSKSLLHSNGYTYLFW | | |
| | | FLQRPGQSPQLLIYRMSNLASGVPDRFSGSGSGTAFTLRI | | |
| | | SRVEAEDVGVYYCMQHLEYPFTFGGGTKLEIK | | |
| | | (SEQ ID NO: 18) | | |
| 3-M12 | CDR- | GYSITSGYY | SGYYWN | GYSITSGY |
| | H1 | (SEQ ID NO: 27) | (SEQ ID NO: 33) | (SEQ ID NO: 38) |
| | CDR- | ITFDGAN | YITFDGANNYNPSLKN | FDG |
| | H2 | (SEQ ID NO: 28) | (SEQ ID NO: 34) | (SEQ ID NO: 39) |
| | CDR- | TRSSYDYDVLDY | SSYDYDVLDY | SYDYDVLD |
| | H3 | (SEQ ID NO: 29) | (SEQ ID NO: 35) | (SEQ ID NO: 40) |
| | CDR- | QDISNF | RASQDISNFLN | SQDISNF |
| | L1 | (SEQ ID NO: 30) | (SEQ ID NO: 36) | (SEQ ID NO: 41) |
| | CDR- | YTS | YTSRLHS | YTS |
| | L2 | (SEQ ID NO: 31) | (SEQ ID NO: 37) | (SEQ ID NO: 31) |
| | CDR- | QQGHTLPYT | QQGHTLPYT | GHTLPY |
| | L3 | (SEQ ID NO: 32) | (SEQ ID NO: 32) | (SEQ ID NO: 42) |
| | VH | DVQLQESGPGLVKPSQSLSLTCSVTGYSITSGYYWNWIRQ | | |
| | | FPGNKLEWMGYITFDGANNYNPSLKNRISITRDTSKNQFF | | |
| | | LKLTSVTTEDTATYYCTRSSYDYDVLDYWGQGTTLTVSS | | |
| | | (SEQ ID NO: 43) | | |
| | VL | DIQMTQTTSSLSASLGDRVTISCRASQDISNFLNWYQQRPD | | |
| | | GTVKLLIYYTSRLHSGVPSRFSGSGSGTDFSLTVSNLEQE | | |
| | | DIATYFCQQGHTLPYTFGGGTKLEIK | | |
| | | (SEQ ID NO: 44) | | |
| 5-H12 | CDR- | GYSFTDYC | DYCIN | GYSFTDY |
| | H1 | (SEQ ID NO: 45) | (SEQ ID NO: 51) | (SEQ ID NO: 56) |
| | CDR- | IYPGSGNT | WIYPGSGNTRYSERFKG | GSG |
| | H2 | (SEQ ID NO: 46) | (SEQ ID NO: 52) | (SEQ ID NO: 57) |
| | CDR- | AREDYYPYHGMDY | EDYYPYHGMDY | DYYPYHGMD |
| | H3 | (SEQ ID NO: 47) | (SEQ ID NO: 53) | (SEQ ID NO: 58) |
| | CDR- | ESVDGYDNSF | RASESVDGYDNSFMH | SESVDGYDNSF |
| | L1 | (SEQ ID NO: 48) | (SEQ ID NO: 54) | (SEQ ID NO: 59) |
| | CDR- | RAS | RASNLES | RAS |
| | L2 | (SEQ ID NO: 49) | (SEQ ID NO: 55) | (SEQ ID NO: 49) |
| | CDR- | QQSSEDPWT | QQSSEDPWT | SSEDPW |
| | L3 | (SEQ ID NO: 50) | (SEQ ID NO: 50) | (SEQ ID NO: 60) |
| | VH | QIQLQQSGPELVRPGASVKISCKASGYSFTDYCINWVNQR | | |
| | | PGQGLEWIGWIYPGSGNTRYSERFKGKATLTVDTSSNTAY | | |
| | | MQLSSLTSEDSAVYFCAREDYYPYHGMDYWGQGTSVTVSS | | |
| | | (SEQ ID NO: 61) | | |
| | VL | DIVLTQSPTSLAVSLGQRATISCRASESVDGYDNSFMHWY | | |
| | | QQKPGQPPKLLIFRASNLESGIPARFSGSGSRTDFTLTIN | | |
| | | PVEAADVATYYCQQSSEDPWTFGGGTKLEIK | | |
| | | (SEQ ID NO: 62) | | |
| 5-H12 | CDR- | GYSFTDYY | DYYIN | GYSFTDY |
| C33Y* | H1 | (SEQ ID NO: 63) | (SEQ ID NO: 64) | (SEQ ID NO: 56) |
| | CDR- | IYPGSGNT | WIYPGSGNTRYSERFKG | GSG |
| | H2 | (SEQ ID NO: 46) | (SEQ ID NO: 52) | (SEQ ID NO: 57) |
| | CDR- | AREDYYPYHGMDY | EDYYPYHGMDY | DYYPYHGMD |
| | H3 | (SEQ ID NO: 47) | (SEQ ID NO: 53) | (SEQ ID NO: 58) |
| | CDR- | ESVDGYDNSF | RASESVDGYDNSFMH | SESVDGYDNSF |
| | L1 | (SEQ ID NO: 48) | (SEQ ID NO: 54) | (SEQ ID NO: 59) |
| | CDR- | RAS | RASNLES | RAS |
| | L2 | (SEQ ID NO: 49) | (SEQ ID NO: 55) | (SEQ ID NO: 49) |
| | CDR- | QQSSEDPWT | QQSSEDPWT | SSEDPW |
| | L3 | (SEQ ID NO: 50) | (SEQ ID NO: 50) | (SEQ ID NO: 60) |
| | VH | QIQLQQSGPELVRPGASVKISCKASGYSFTDYYINWVNQR | | |
| | | PGQGLEWIGWIYPGSGNTRYSERFKGKATLTVDTSSNTAY | | |
| | | MQLSSLTSEDSAVYFCAREDYYPYHGMDYWGQGTSVTVSS | | |
| | | (SEQ ID NO: 65) | | |
| | VL | DIVLTQSPTSLAVSLGQRATISCRASESVDGYDNSFMHWY | | |
| | | QQKPGQPPKLLIFRASNLESGIPARFSGSGSRTDFTLTIN | | |
| | | PVEAADVATYYCQQSSEDPWTFGGGTKLEIK | | |
| | | (SEQ ID NO: 62) | | |
| 5-H12 | CDR- | GYSFTDYD | DYDIN | GYSFTDY |
| C33D* | H1 | (SEQ ID NO: 66) | (SEQ ID NO: 67) | (SEQ ID NO: 56) |
| | CDR- | IYPGSGNT | WIYPGSGNTRYSERFKG | GSG |
| | H2 | (SEQ ID NO: 46) | (SEQ ID NO: 52) | (SEQ ID NO: 57) |
| | CDR- | AREDYYPYHGMDY | EDYYPYHGMDY | DYYPYHGMD |
| | H3 | (SEQ ID NO: 47) | (SEQ ID NO: 53) | (SEQ ID NO: 58) |
| | CDR- | ESVDGYDNSF | RASESVDGYDNSFMH | SESVDGYDNSF |
| | L1 | (SEQ ID NO: 48) | (SEQ ID NO: 54) | (SEQ ID NO: 59) |
| | CDR- | RAS | RASNLES | RAS |
| | L2 | (SEQ ID NO: 49) | (SEQ ID NO: 55) | (SEQ ID NO: 49) |
| | CDR- | QQSSEDPWT | QQSSEDPWT | SSEDPW |
| | L3 | (SEQ ID NO: 50) | (SEQ ID NO: 50) | (SEQ ID NO: 60) |
| | VH | QIQLQQSGPELVRPGASVKISCKASGYSFTDYDINWVNQR | | |
| | | PGQGLEWIGWIYPGSGNTRYSERFKGKATLTVDTSSNTAY | | |
| | | MQLSSLTSEDSAVYFCAREDYYPYHGMDYWGQGTSVTVSS | | |
| | | (SEQ ID NO: 68) | | |
| | VL | DIVLTQSPTSLAVSLGQRATISCRASESVDGYDNSFMHWY | | |
| | | QQKPGQPPKLLIFRASNLESGIPARFSGSGSRTDFTLTIN | | |
| | | PVEAADVATYYCQQSSEDPWTFGGGTKLEIK | | |
| | | (SEQ ID NO: 62) | | |
| Anti- | CDR- | GYSFTSYW | SYWIG | GYSFTSY |
| TfR | H1 | (SEQ ID NO: 138) | (SEQ ID NO: 144) | (SEQ ID NO: 149) |
| clone 8 | CDR- | IYPGDSDT | IIYPGDSDTRYSPSFQGQ | GDS |
| | H2 | (SEQ ID NO: 139) | (SEQ ID NO: 145) | (SEQ ID NO: 150) |
| | CDR- | ARFPYDSSGYYSFDY | FPYDSSGYYSFDY | PYDSSGYYSFD |
| | H3 | (SEQ ID NO: 140) | (SEQ ID NO: 146) | (SEQ ID NO: 151) |
| | CDR- | QSISSY | RASQSISSYLN | SQSISSY |
| | L1 | (SEQ ID NO: 141) | (SEQ ID NO: 147) | (SEQ ID NO: 152) |
| | CDR- | AAS | AASSLQS | AAS |
| | L2 | (SEQ ID NO: 142) | (SEQ ID NO: 148) | (SEQ ID NO: 142) |
| | CDR- | QQSYSTPLT | QQSYSTPLT | SYSTPL |
| | L3 | (SEQ ID NO: 143) | (SEQ ID NO: 143) | (SEQ ID NO: 153) |

* mutation positions are according to Kabat numbering of the respective VH sequences containing the mutations

In some embodiments, the anti-TfR1 antibody of the present disclosure is a humanized variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Examples of amino acid sequences of anti-TfR1 antibodies described herein are provided in Table 3.

TABLE 3
Variable Regions of Anti-TfR1 Antibodies

| Antibody | Variable Region Amino Acid Sequence** |
|---|---|
| 3A4 | V_{H}: |
| VH3 (N54T*)/Vκ4 | EVQLVQSGSELKKPGASVKVSCTASGFNIKDDYMYWVRQPPGKGLEWIGWID |
| | PETGDTEYASKFQDRVTVTADTSTNTAYMELSSLRSEDTAVYYCTLWLRRGL |
| | DYWGQGTLVTVSS (SEQ ID NO: 69) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGT |
| | KVEIK (SEQ ID NO: 70) |
| 3A4 | V_{H}: |
| VH3 (N54S*)/Vκ4 | EVQLVQSGSELKKPGASVKVSCTASGFNIKDDYMYWVRQPPGKGLEWIGWID |
| | PESGDTEYASKFQDRVTVTADTSTNTAYMELSSLRSEDTAVYYCTLWLRRGL |
| | DYWGQGTLVTVSS (SEQ ID NO: 71) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGT |
| | KVEIK (SEQ ID NO: 70) |
| 3A4 | V_{H}: |
| VH3 /Vκ4 | EVQLVQSGSELKKPGASVKVSCTASGFNIKDDYMYWVRQPPGKGLEWIGWID |
| | PENGDTEYASKFQDRVTVTADTSTNTAYMELSSLRSEDTAVYYCTLWLRRGL |
| | DYWGQGTLVTVSS (SEQ ID NO: 72) |
| | V_{L}: |
| | DIVMTQSPLSLPVTPGEPASISCRSSKSLLHSNGYTYLFWFQQRPGQSPRLLIYR |
| | MSNLASGVPDRFSGSGSGTDFTLKISRVEAEDVGVYYCMQHLEYPFTFGGGT |
| | KVEIK (SEQ ID NO: 70) |
| 3M12 | V_{H}: |
| VH3/Vκ2 | QVQLQESGPGLVKPSQTLSLTCSVTGYSITSGYYWNWIRQPPGKGLEWMGYIT |
| | FDGANNYNPSLKNRVSISRDTSKNQFSLKLSSVTAEDTATYYCTRSSYDYDVL |
| | DYWGQGTTVTVSS (SEQ ID NO: 73) |
| | V_{L}: |
| | DIQMTQSPSSLSASVGDRVTITCRASQDISNFLNWYQQKPGQPVKLLIYYTSRL |
| | HSGVPSRFSGSGSGTDFTLTISSLQPEDFATYFCQQGHTLPYTFGQGTKLEIK |
| | (SEQ ID NO: 74) |
| 3M12 | V_{H}: |
| VH3/Vκ3 | QVQLQESGPGLVKPSQTLSLTCSVTGYSITSGYYWNWIRQPPGKGLEWMGYIT |
| | FDGANNYNPSLKNRVSISRDTSKNQFSLKLSSVTAEDTATYYCTRSSYDYDVL |
| | DYWGQGTTVTVSS (SEQ ID NO: 73) |
| | V_{L}: |
| | DIQMTQSPSSLSASVGDRVTITCRASQDISNFLNWYQQKPGQPVKLLIYYTSRL |
| | HSGVPSRFSGSGSGTDFTLTISSLQPEDFATYYCQQGHTLPYTFGQGTKLEIK |
| | (SEQ ID NO: 75) |
| 3M12 | V_{H}: |
| VH4/Vκ2 | QVQLQESGPGLVKPSQTLSLTCTVTGYSITSGYYWNWIRQPPGKGLEWIGYITF |
| | DGANNYNPSLKNRVSISRDTSKNQFSLKLSSVTAEDTATYYCTRSSYDYDVLD |
| | YWGQGTTVTVSS (SEQ ID NO: 76) |
| | V_{L}: |
| | DIQMTQSPSSLSASVGDRVTITCRASQDISNFLNWYQQKPGQPVKLLIYYTSRL |
| | HSGVPSRFSGSGSGTDFTLTISSLQPEDFATYFCQQGHTLPYTFGQGTKLEIK |
| | (SEQ ID NO: 74) |
| 3M12 | V_{H}: |
| VH4/Vκ3 | QVQLQESGPGLVKPSQTLSLTCTVTGYSITSGYYWNWIRQPPGKGLEWIGYITF |
| | DGANNYNPSLKNRVSISRDTSKNQFSLKLSSVTAEDTATYYCTRSSYDYDVLD |
| | YWGQGTTVTVSS (SEQ ID NO: 76) |
| | V_{L}: |
| | DIQMTQSPSSLSASVGDRVTITCRASQDISNFLNWYQQKPGQPVKLLIYYTSRL |
| | HSGVPSRFSGSGSGTDFTLTISSLQPEDFATYYCQQGHTLPYTFGQGTKLEIK |
| | (SEQ ID NO: 75) |
| 5H12 | V_{H}: |
| VH5 (C33Y*)/Vκ3 | QVQLVQSGAEVKKPGASVKVSCKASGYSFTDYYINWVRQAPGQGLEWMGWI |
| | YPGSGNTRYSERFKGRVTITRDTSASTAYMELSSLRSEDTAVYYCAREDYYPY |
| | HGMDYWGQGTLVTVSS (SEQ ID NO: 77) |
| | V_{L}: |
| | DIVLTQSPDSLAVSLGERATINCRASESVDGYDNSFMHWYQQKPGQPPKLLIFR |
| | ASNLESGVPDRFSGSGSRTDFTLTISSLQAEDVAVYYCQQSSEDPWTFGQGTK |
| | LEIK (SEQ ID NO: 78) |
| 5H12 | V_{H}: |
| VH5 (C33D*)/Vκ4 | QVQLVQSGAEVKKPGASVKVSCKASGYSFTDYDINWVRQAPGQGLEWMGWI |
| | YPGSGNTRYSERFKGRVTITRDTSASTAYMELSSLRSEDTAVYYCAREDYYPY |
| | HGMDYWGQGTLVTVSS (SEQ ID NO: 79) |
| | V_{L}: |
| | DIVMTQSPDSLAVSLGERATINCRASESVDGYDNSFMHWYQQKPGQPPKLLIF |
| | RASNLESGVPDRFSGSGSGTDFTLTISSLQAEDVAVYYCQQSSEDPWTFGQGT |
| | KLEIK (SEQ ID NO: 80) |
| 5H12 | V_{H}: |
| VH5 (C33Y*)/Vκ4 | QVQLVQSGAEVKKPGASVKVSCKASGYSFTDYYINWVRQAPGQGLEWMGWI |
| | YPGSGNTRYSERFKGRVTITRDTSASTAYMELSSLRSEDTAVYYCAREDYYPY |
| | HGMDYWGQGTLVTVSS (SEQ ID NO: 77) |
| | V_{L}: |
| | DIVMTQSPDSLAVSLGERATINCRASESVDGYDNSFMHWYQQKPGQPPKLLIF |
| | RASNLESGVPDRFSGSGSGTDFTLTISSLQAEDVAVYYCQQSSEDPWTFGQGT |
| | KLEIK (SEQ ID NO: 80) |
| Anti-TfR clone 8 | V_{H}: |
| | QVQLVQSGAEVKKPGESLKISCKGSGYSFTSYWIGWVRQMPGKGLEWMGIIY |
| | PGDSDTRYSPSFQGQVTISADKSISTAYLQWSSLKASDTAMYYCARFPYDSSG |
| | YYSFDYWGQGTLVTVSS (SEQ ID NO: 154) |
| | V_{L}: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPLTFGGGTKVEIK |
| | (SEQ ID NO: 155) |

*mutation positions are according to Kabat numbering of the respective VH sequences containing the mutations
**CDRs according to the Kabat numbering system are bolded

In some embodiments, the anti-TfR1 antibody of the present disclosure comprises a VH comprising the CDR-H1, CDR-H2, and CDR-H3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the CDR-H1, CDR-H2, and CDR-H3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the amino acid sequence of SEQ ID NO: 69 and a VL comprising the amino acid sequence of SEQ ID NO: 70.

In some embodiments, the anti-TfR1 antibody of the present disclosure comprises a VH comprising the amino acid sequence of SEQ ID NO: 71 and a VL comprising the amino acid sequence of SEQ ID NO: 70.

In some embodiments, the anti-TfR1 antibody of the present disclosure comprises a VH comprising the amino acid sequence of SEQ ID NO: 72 and a VL comprising the amino acid sequence of SEQ ID NO: 70.

In some embodiments, the anti-TfR1 antibody of the present disclosure comprises a VH comprising the amino acid sequence of SEQ ID NO: 73 and a VL comprising the amino acid sequence of SEQ ID NO: 74.

In some embodiments, the anti-TfR1 antibody of the present disclosure comprises a VH comprising the amino acid sequence of SEQ ID NO: 73 and a VL comprising the amino acid sequence of SEQ ID NO: 75.

In some embodiments, the anti-TfR1 antibody of the present disclosure comprises a VH comprising the amino acid sequence of SEQ ID NO: 76 and a VL comprising the amino acid sequence of SEQ ID NO: 74.

In some embodiments, the anti-TfR1 antibody of the present disclosure comprises a VH comprising the amino acid sequence of SEQ ID NO: 76 and a VL comprising the amino acid sequence of SEQ ID NO: 75.

In some embodiments, the anti-TfR1 antibody of the present disclosure comprises a VH comprising the amino acid sequence of SEQ ID NO: 77 and a VL comprising the amino acid sequence of SEQ ID NO: 78.

In some embodiments, the anti-TfR1 antibody of the present disclosure comprises a VH comprising the amino acid sequence of SEQ ID NO: 79 and a VL comprising the amino acid sequence of SEQ ID NO: 80.

In some embodiments, the anti-TfR1 antibody of the present disclosure comprises a VH comprising the amino acid sequence of SEQ ID NO: 77 and a VL comprising the amino acid sequence of SEQ ID NO: 80.

In some embodiments, the anti-TfR1 antibody of the present disclosure comprises a VH comprising the amino acid sequence of SEQ ID NO: 154 and a VL comprising the amino acid sequence of SEQ ID NO: 155.

In some embodiments, the anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (www.imgt.org) or at www.vbase2.org/vbstat.php, both of which are incorporated by reference herein.

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region as set forth in SEQ ID NO: 81. In some embodiments, the anti-TfR1 antibody described herein comprises heavy chain comprising any one of the VH as listed in Table 3 or any variants thereof and a heavy chain constant region as set forth in SEQ ID NO: 82.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of examples of anti-TfR1 IgGs

| Antibody | IgG Heavy Chain/Light Chain Sequences** |
|---|---|
| 3A4 | Heavy Chain (with wild type human IgG1 constant region) |
| VH3 (N54T*)/Vκ4 | EVQLVQSGSELKKPGASVKVSCTASGFNIKDDYMYWVRQPPGKGLEWIGWIDP |
| | ETGDTEYASKFQDRVTVTADTSTNTAYMELSSLRSEDTAVYYCTLWLRRGLD |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEALH |
| | NHYTQKSLSLSPGK (SEQ ID NO: 84) |
| | Light Chain (with kappa light chain constant region) |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 85) |
| 3A4 | Heavy Chain (with wild type human IgG1 constant region) |
| VH3 (N54S*)/Vκ4 | EVQLVQSGSELKKPGASVKVSCTASGFNIKDDYMYWVRQPPGKGLEWIGWIDP |
| | ESGDTEYASKFQDRVTVTADTSTNTAYMELSSLRSEDTAVYYCTLWLRRGLDY |
| | WGQGTLVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK (SEQ ID NO: 86) |
| | Light Chain (with kappa light chain constant region) |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 85) |
| 3A4 | Heavy Chain (with wild type human IgG1 constant region) |
| VH3 /Vκ4 | EVQLVQSGSELKKPGASVKVSCTASGFNIKDDYMYWVRQPPGKGLEWIGWIDP |
| | ENGDTEYASKFQDRVTVTADTSTNTAYMELSSLRSEDTAVYYCTLWLRRGLD |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEALH |
| | NHYTQKSLSLSPGK (SEQ ID NO: 87) |
| | Light Chain (with kappa light chain constant region) |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 85) |
| 3M12 | Heavy Chain (with wild type human IgG1 constant region) |
| VH3/Vκ2 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 89) |
| 3M12 | Heavy Chain (with wild type human IgG1 constant region) |
| VH3/Vκ3 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALH |
| | NHYTQKSLSLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 90) |
| 3M12 | Heavy Chain (with wild type human IgG1 constant region) |
| VH4/Vκ2 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEW |
| | ESNGQPENNYKTTPPVLDSDGSFFLYSKLTVDKSRWQQGNVFSCSVMHEALHNH |
| | YTQKSLSLSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 89) |
| 3M12 | Heavy Chain (with wild type human IgG1 constant region) |
| VH4/Vκ3 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEP |
| | KSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEW |
| | ESNGQPENNYKTTPPVLDSDGSFFLYSKLTVDKSRWQQGNVFSCSVMHEALHNH |
| | YTQKSLSLSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 90) |
| 5H12 | Heavy Chain (with wild type human IgG1 constant region) |
| VH5 (C33Y*)/Vκ3 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMH |
| | EALHNHYTQKSLSLSPGK (SEQ ID NO: 92) |
| | Light Chain (with kappa light chain constant region) |
| | DIVLTQSPDSLAVSLGERATINCRASESVDGYDNSFMHWYQQKPGQPPKLLIFRA |
| | SNLESGVPDRFGSGSGSRTDFTLTISSLQAEDVAVYYCQQSSEDPWTFGQGTKLEI |
| | KRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 93) |
| 5H12 | Heavy Chain (with wild type human IgG1 constant region) |
| VH5 (C33D*)/Vκ4 | QVQLVQSGAEVKKPGASVKVSCKASGYSFTDYDINWVRQAPGQGLEWMGWIY |
| | PGSGNTRYSERFKGRVTITRDTSASTAYMELSSLRSEDTAVYYCAREDYYPYH |
| | GMDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMH |
| | EALHNHYTQKSLSLSPGK (SEQ ID NO: 94) |
| | Light Chain (with kappa light chain constant region) |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKLE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 95) |
| 5H12 | Heavy Chain (with wild type human IgG1 constant region) |
| VH5 (C33Y*)/Vκ4 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMH |
| | EALHNHYTQKSLSLSPGK (SEQ ID NO: 92) |
| | Light Chain (with kappa light chain constant region) |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKLE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 95) |
| Anti-TfR clone 8 | VH: |
| | QVQLVQSGAEVKKPGESLKISCKGSGYSFTSYWIGWVRQMPGKGLEWMGIIYP |
| | GDSDTRYSPSFQGQVTISADKSISTAYLQWSSLKASDTAMYYCARFPYDSSGYY |
| | SFDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK (SEQ ID NO: 156) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAA |
| | PSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 84, 86, 87, 88, 91, 92, 94, and 156. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, 94, and 156. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 84, 86, 87, 88, 91, 92, 94, and 156. Alternatively or in addition (e.g., in addition), the anti-TfR1 antibody described herein comprises a light chain comprising the amino acid sequence of any one of SEQ ID NOs: 85, 89, 90, 93, 95 and 157.

In some embodiments, the anti-TfR1 antibody of the present disclosure comprises a heavy chain comprising the amino acid sequence of SEQ ID NO: 84 and a light chain comprising the amino acid sequence of SEQ ID NO: 85.

In some embodiments, the anti-TfR1 antibody of the present disclosure comprises a heavy chain comprising the amino acid sequence of SEQ ID NO: 86 and a light chain comprising the amino acid sequence of SEQ ID NO: 85.

In some embodiments, the anti-TfR1 antibody of the present disclosure comprises a heavy chain comprising the amino acid sequence of SEQ ID NO: 87 and a light chain comprising the amino acid sequence of SEQ ID NO: 85.

In some embodiments, the anti-TfR1 antibody of the present disclosure comprises a heavy chain comprising the amino acid sequence of SEQ ID NO: 88 and a light chain comprising the amino acid sequence of SEQ ID NO: 89.

In some embodiments, the anti-TfR1 antibody of the present disclosure comprises a heavy chain comprising the amino acid sequence of SEQ ID NO: 88 and a light chain comprising the amino acid sequence of SEQ ID NO: 90.

In some embodiments, the anti-TfR1 antibody of the present disclosure comprises a heavy chain comprising the amino acid sequence of SEQ ID NO: 91 and a light chain comprising the amino acid sequence of SEQ ID NO: 89.

In some embodiments, the anti-TfR1 antibody of the present disclosure comprises a heavy chain comprising the amino acid sequence of SEQ ID NO: 91 and a light chain comprising the amino acid sequence of SEQ ID NO: 90.

In some embodiments, the anti-TfR1 antibody of the present disclosure comprises a heavy chain comprising the amino acid sequence of SEQ ID NO: 92 and a light chain comprising the amino acid sequence of SEQ ID NO: 93.

In some embodiments, the anti-TfR1 antibody of the present disclosure comprises a heavy chain comprising the amino acid sequence of SEQ ID NO: 94 and a light chain comprising the amino acid sequence of SEQ ID NO: 95.

In some embodiments, the anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody of the present disclosure comprises a heavy chain comprising the amino acid sequence of SEQ ID NO: 156 and a light chain comprising the amino acid sequence of SEQ ID NO: 157.

In some embodiments, the anti-TfR1 antibody is a Fab fragment, Fab′ fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full-length IgG using an enzyme such as papain). For example, F(ab′)₂ fragments can be produced by pepsin or papain digestion of an antibody molecule, and Fab fragments that can be generated by reducing the disulfide bridges of F(ab′)₂ fragments. In some embodiments, a heavy chain constant region in a Fab fragment of the anti-TfR1 antibody described herein comprises the amino acid sequence of:

(SEQ ID NO: 96)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHT

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the anti-TfR1 antibody described herein comprises a heavy chain comprising any one of the VH as listed in Table 3 or any variants thereof and a heavy chain constant region as set forth in SEQ ID NO: 96.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of examples of anti-TfR1 Fabs

| Antibody | Fab Heavy Chain/Light Chain Sequences* |
|---|---|
| 3A4 | Heavy Chain (with partial human IgG1 constant region) |
| VH3 (N54T*)/Vκ4 | EVQLVQSGSELKKPGASVKVSCTASGFNIKDDYMYWVRQPPGKGLEWIGWIDP |
| | ETGDTEYASKFQDRVTVTADTSTNTAYMELSSLRSEDTAVYYCTLWLRRGLD |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHT (SEQ ID NO: 97) |
| | Light Chain (with kappa light chain constant region) |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 85) |
| 3A4 | Heavy Chain (with partial human IgG1 constant region) |
| VH3 (N54S*)/Vκ4 | EVQLVQSGSELKKPGASVKVSCTASGFNIKDDYMYWVRQPPGKGLEWIGWIDP |
| | ESGDTEYASKFQDRVTVTADTSTNTAYMELSSLRSEDTAVYYCTLWLRRGLDY |
| | WGQGTLVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHT (SEQ ID NO: 98) |
| | Light Chain (with kappa light chain constant region) |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 85) |
| 3A4 | Heavy Chain (with partial human IgG1 constant region) |
| VH3 /Vκ4 | EVQLVQSGSELKKPGASVKVSCTASGFNIKDDYMYWVRQPPGKGLEWIGWIDP |
| | ENGDTEYASKFQDRVTVTADTSTNTAYMELSSLRSEDTAVYYCTLWLRRGLD |
| | YWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHT (SEQ ID NO: 99) |
| | Light Chain (with kappa light chain constant region) |
| | DIVMTQSPLSLPVTPGEPASISCRSSKSLLHSNGYTYLFWFQQRPGQSPRLLIYRM |
| | SNLASGVPDRFSGSGSGTDFTLKISRVEAEDVGVYYCMQHLEYPFTFGGGTKVE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 85) |
| 3M12 | Heavy Chain (with partial human IgG1 constant region) |
| VH3/Vκ2 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 89) |
| 3M12 | Heavy Chain (with partial human IgG1 constant region) |
| VH3/Vκ3 | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 90) |
| 3M12 | Heavy Chain (with partial human IgG1 constant region) |
| VH4/Vκ2 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEP |
| | KSCDKTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYFCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 89) |
| 3M12 | Heavy Chain (with partial human IgG1 constant region) |
| VH4/Vκ3 | QVQLQESGPGLVKPSQTLSLTCTVTGYSITSGYYWNWIRQPPGKGLEWIGYITFD |
| | GANNYNPSLKNRVSISRDTSKNQFSLKLSSVTAEDTATYYCTRSSYDYDVLDYW |
| | GQGTTVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEP |
| | KSCDKTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLH |
| | SGVPSRFSGSGSGTDFTLTISSLQPEDFATYYCQQGHTLPYTFGQGTKLEIKRTVA |
| | APSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC (SEQ ID |
| | NO: 90) |
| 5H12 | Heavy Chain (with partial human IgG1 constant region) |
| VH5 (C33Y*)/Vκ3 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTKV |
| | DKKVEPKSCDKTHT (SEQ ID NO: 102) |
| | Light Chain (with kappa light chain constant region) |
| | DIVLTQSPDSLAVSLGERATINCRASESVDGYDNSFMHWYQQKPGQPPKLLIFRA |
| | SNLESGVPDRFSGSGSRTDFTLTISSLQAEDVAVYYCQQSSEDPWTFGQGTKLEI |
| | KRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 93) |
| 5H12 | Heavy Chain (with partial human IgG1 constant region) |
| VH5 (C33D*)/Vκ4 | QVQLVQSGAEVKKPGASVKVSCKASGYSFTDYDINWVRQAPGQGLEWMGWIY |
| | PGSGNTRYSERFKGRVTITRDTSASTAYMELSSLRSEDTAVYYCAREDYYPYH |
| | GMDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTKV |
| | DKKVEPKSCDKTHT (SEQ ID NO: 103) |
| | Light Chain (with kappa light chain constant region) |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKLE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 95) |
| 5H12 | Heavy Chain (with partial human IgG1 constant region) |
| VH5 (C33Y*)/Vκ4 | QVQLVQSGAEVKKPGASVKVSCKASGYSFTDYYINWVRQAPGQGLEWMGWIY |
| | PGSGNTRYSERFKGRVTITRDTSASTAYMELSSLRSEDTAVYYCAREDYYPYH |
| | GMDYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNTKV |
| | DKKVEPKSCDKTHT (SEQ ID NO: 102) |
| | Light Chain (with kappa light chain constant region) |
| | DIVMTQSPDSLAVSLGERATINCRASESVDGYDNSFMHWYQQKPGQPPKLLIFR |
| | ASNLESGVPDRFSGSGSGTDFTLTISSLQAEDVAVYYCQQSSEDPWTFGQGTKLE |
| | IKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 95) |
| Anti-TfR clone 8 | VH: |
| Version 1 | QVQLVQSGAEVKKPGESLKISCKGSGYSFTSYWIGWVRQMPGKGLEWMGIIYP |
| | GDSDTRYSPSFQGQVTISADKSISTAYLQWSSLKASDTAMYYCARFPYDSSGYY |
| | SFDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVD |
| | KKVEPKSCDKTHTCP (SEQ ID NO: 158) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAA |
| | PSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 157) |
| Anti-TfR clone 8 | VH: |
| Version 2 | QVQLVQSGAEVKKPGESLKISCKGSGYSFTSYWIGWVRQMPGKGLEWMGIIYP |
| | GDSDTRYSPSFQGQVTISADKSISTAYLQWSSLKASDTAMYYCARFPYDSSGYY |
| | SFDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKVD |
| | KKVEPKSCDKTHT (SEQ ID NO: 159) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAA |
| | PSVFIFPPSDEQLKSGTASVVCLLNNFYPREAKVQWKVDNALQSGNSQESVTEQD |
| | SKDSTYSLSSTLTLSKADYEKHKVYACEVTHQGLSSPVTKSFNRGEC (SEQ ID |
| | NO: 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 97-103, 158 and 159. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103, 158 and 159. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 97-103, 158 and 159. Alternatively or in addition (e.g., in addition), the anti-TfR1 antibody described herein comprises a light chain comprising the amino acid sequence of any one of SEQ ID NOs: 85, 89, 90, 93, 95, and 157.

In some embodiments, the anti-TfR1 antibody of the present disclosure comprises a heavy chain comprising the amino acid sequence of SEQ ID NO: 97 and a light chain comprising the amino acid sequence of SEQ ID NO: 85.

In some embodiments, the anti-TfR1 antibody of the present disclosure comprises a heavy chain comprising the amino acid sequence of SEQ ID NO: 98 and a light chain comprising the amino acid sequence of SEQ ID NO: 85.

In some embodiments, the anti-TfR1 antibody of the present disclosure comprises a heavy chain comprising the amino acid sequence of SEQ ID NO: 99 and a light chain comprising the amino acid sequence of SEQ ID NO: 85.

In some embodiments, the anti-TfR1 antibody of the present disclosure comprises a heavy chain comprising the amino acid sequence of SEQ ID NO: 100 and a light chain comprising the amino acid sequence of SEQ ID NO: 89.

In some embodiments, the anti-TfR1 antibody of the present disclosure comprises a heavy chain comprising the amino acid sequence of SEQ ID NO: 100 and a light chain comprising the amino acid sequence of SEQ ID NO: 90.

In some embodiments, the anti-TfR1 antibody of the present disclosure comprises a heavy chain comprising the amino acid sequence of SEQ ID NO: 101 and a light chain comprising the amino acid sequence of SEQ ID NO: 89.

In some embodiments, the anti-TfR1 antibody of the present disclosure comprises a heavy chain comprising the amino acid sequence of SEQ ID NO: 101 and a light chain comprising the amino acid sequence of SEQ ID NO: 90.

In some embodiments, the anti-TfR1 antibody of the present disclosure comprises a heavy chain comprising the amino acid sequence of SEQ ID NO: 102 and a light chain comprising the amino acid sequence of SEQ ID NO: 93.

In some embodiments, the anti-TfR1 antibody of the present disclosure comprises a heavy chain comprising the amino acid sequence of SEQ ID NO: 103 and a light chain comprising the amino acid sequence of SEQ ID NO: 95.

In some embodiments, the anti-TfR1 antibody of the present disclosure comprises a heavy chain comprising the amino acid sequence of SEQ ID NO: 102 and a light chain comprising the amino acid sequence of SEQ ID NO: 95.

In some embodiments, the anti-TfR1 antibody of the present disclosure comprises a heavy chain comprising the amino acid sequence of SEQ ID NO: 158 and a light chain comprising the amino acid sequence of SEQ ID NO: 157.

In some embodiments, the anti-TfR1 antibody of the present disclosure comprises a heavy chain comprising the amino acid sequence of SEQ ID NO: 159 and a light chain comprising the amino acid sequence of SEQ ID NO: 157.

### Other Known Antd-TfR1 Antibodies

Any other appropriate anti-TfR1 antibodies known in the art may be used as the muscle-targeting agent in the complexes disclosed herein. Examples of known anti-TfR1 antibodies, including associated references and binding epitopes, are listed in Table 6. In some embodiments, the anti-TfR1 antibody comprises the complementarity determining regions (CDR-H1, CDR-H2, CDR-H3, CDR-L1, CDR-L2, and CDR-L3) of any of the anti-TfR1 antibodies provided herein, e.g., anti-TfR1 antibodies listed in Table 6.

TABLE 6
List of anti-TfR1 antibody clones, including associated references and binding
epitope information.

| Antibody Clone | | |
|---|---|---|
| Name | Reference(s) | Epitope/Notes |
| OKT9 | U.S. Pat. No.. 4,364,934, filed Dec. 4, 1979, | Apical domain of TfR1 |
| | entitled “MONOCLONAL ANTIBODY TO | (residues 305-366 of |
| | A HUMAN EARLY THYMOCYTE | human TfR1 sequence |
| | ANTIGEN AND METHODS FOR | XM_052730.3, available |
| | PREPARING SAME” | in GenBank) |
| | Schneider C. et al. “Structural features of the | |
| | cell surface receptor for transferrin that is | |
| | recognized by the monoclonal antibody | |
| | OKT9.” J Biol Chem. 1982, 257: 14, 8516- | |
| | 8522. | |
| (From JCR) | WO 2015/098989, filed Dec. 24, 2014, | Apical domain (residues |
| Clone M11 | “Novel anti-Transferrin receptor antibody | 230-244 and 326-347 of |
| Clone M23 | that passes through blood-brain barrier” | TfR1) and protease-like |
| Clone M27 | U.S. Pat. No. 9,994,641, filed | domain (residues 461- |
| Clone B84 | Dec. 24, 2014, “Novel anti-Transferrin | 473) |
| | receptor antibody that passes through | |
| | blood-brain barrier” | |
| (From | WO 2016/081643, filed May 26, 2016, | Apical domain and non- |
| Genentech) | entitled “ANTI-TRANSFERRIN | apical regions |
| 7A4, 8A2, 15D2, | RECEPTOR ANTIBODIES AND | |
| 10D11, 7B10, | METHODS OF USE” | |
| 15G11, 16G5, | U.S. Pat. No. 9,708,406, filed | |
| 13C3, 16G4, | May 20, 2014, “Anti-transferrin receptor | |
| 16F6, 7G7, 4C2, | antibodies and methods of use” | |
| 1B12, and 13D4 | | |
| (From Armagen) | Lee et al. “Targeting Rat Anti-Mouse | |
| 8D3 | Transferrin Receptor Monoclonal Antibodies | |
| | through Blood-Brain Barrier in Mouse” | |
| | 2000, J Pharmacol. Exp. Ther., 292: 1048- | |
| | 1052. | |
| | US Patent App. 2010/077498, filed | |
| | Sep. 11, 2008, entitled “COMPOSITIONS | |
| | AND METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of | |
| | the human transferrin receptor in plant cells: | |
| | effects of tyrphostin A23 and brefeldin A. | |
| | Plant J 48: 757-70 (2006). | |
| 1A1B2, 66IG10, | Commercially available anti- | Novus Biologicals |
| MEM-189, | transferrin receptor antibodies. | 8100 Southpark Way, A- |
| JF0956, 29806, | | 8 Littleton CO 80120 |
| 1A1B2, | | |
| TFRC/1818, | | |
| 1E6, 66Ig10, | | |
| TFRC/1059, | | |
| Q1/71, 23D10, | | |
| 13E4, | | |
| TFRC/1149, ER- | | |
| MP21, | | |
| YTA74.4, BU54, | | |
| 2B6, RI7 217 | | |
| (From INSERM) | US Patent App. 2011/0311544A1, | Does not compete with |
| BA120g | filed Jun. 15, 2005, entitled “ANTI-CD71 | OKT9 |
| | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I.S. et al. “Anti-transferrin | |
| B3/25 | receptor monoclonal antibody and toxin- | |
| T58/30 | antibody conjugates affect growth of | |
| | human tumour cells.” Nature, 1981, | |
| | volume 294, pages 171-173 | |
| R17 217.1.3, | Commercially available anti- | BioXcell |
| 5E9C11, | transferrin receptor antibodies. | 10 Technology Dr., Suite |
| OKT9 (BE0023 | | 2B |
| clone) | | West Lebanon, NH |
| | | 03784-1671 USA |
| BK19.9, B3/25, | Gatter, K.C. et al. “Transferrin receptors | |
| T56/14 and | in human tissues: their distribution and | |
| T58/1 | possible clinical relevance.” J Clin | |
| | Pathol. 1983 May; 36(5): 539-45. | |

| | Additional Anti-TfR1 antibody SEQ ID NOs | | | | |
|---|---|---|---|---|---|
| Anti-TfR1 antibody | | VH/VL | CDR1 | CDR2 | CDR3 |
| CDRH1 (SEQ ID NO: 787) | VH1 | 802 | 795 | 796 | 789 |
| CDRH2 (SEQ ID NO: 788) | VH2 | 803 | 795 | 797 | 789 |
| CDRH3 (SEQ ID NO: 789) | VH3 | 804 | 795 | 798 | 789 |
| CDRL1 (SEQ ID NO: 790) | VH4 | 805 | 795 | 797 | 789 |
| CDRL2 (SEQ ID NO: 791) | VL1 | 806 | 790 | 791 | 115 |
| CDRL3 (SEQ ID NO: 792) | VL2 | 807 | 790 | 791 | 115 |
| VH (SEQ ID NO: 793) | VL3 | 808 | 790 | 799 | 792 |
| VL (SEQ ID NO: 794) | VL4 | 809 | 800 | 801 | 792 |

In some embodiments, anti-TfR1 antibodies of the present disclosure include one or more of the CDR-H (e.g., CDR-H1, CDR-H2, and CDR-H3) amino acid sequences from any one of the anti-TfR1 antibodies selected from Table 6. In some embodiments, anti-TfR1 antibodies include the CDR-L1, CDR-L2, and CDR-L3 as provided for any one of the anti-TfR1 antibodies selected from Table 6. In some embodiments, anti-TfR1 antibodies include the CDR-H1, CDR-H2, CDR-H3, CDR-L1, CDR-L2, and CDR-L3 as provided for any one of the anti-TfR1 antibodies selected from Table 6.

In some embodiments, anti-TfR1 antibodies of the disclosure include any antibody that includes a heavy chain variable domain and/or (e.g., and) a light chain variable domain of any anti-TfR1 antibody, such as any one of the anti-TfR1 antibodies selected from Table 6. In some embodiments, anti-TfR1 antibodies of the disclosure include any antibody that includes the heavy chain variable and light chain variable pairs of any anti-TfR1 antibody, such as any one of the anti-TfR1 antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1 antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1 antibody, such as any one of the anti-TfR1 antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1 antibodies selected from Table 6.

An example of a transferrin receptor antibody that may be used in accordance with the present disclosure is described in International Application Publication WO 2016/081643, incorporated herein by reference. The amino acid sequences of this antibody are provided in Table 7.

TABLE 7
Heavy chain and light chain CDRs of an example of a known anti-TfR1 antibody

| Sequence Type | Kabat | Chothia | Contact |
|---|---|---|---|
| CDR-H1 | SYWMH (SEQ ID | GYTFTSY (SEQ ID NO: 116) | TSYWMH (SEQ ID NO: 118) |
| | NO: 110) | | |
| CDR-H2 | EINPTNGRTNYIE | NPTNGR (SEQ ID NO: 117) | WIGEINPTNGRTN (SEQ ID |
| | KFKS (SEQ ID | | NO: 119) |
| | NO: 111) | | |
| CDR-H3 | GTRAYHY (SEQ | GTRAYHY (SEQ ID NO: | ARGTRA (SEQ ID NO: 120) |
| | ID NO: 112) | 112) | |
| CDR-L1 | RASDNLYSNLA | RASDNLYSNLA (SEQ ID | YSNLAWY (SEQ ID NO: 121) |
| | (SEQ ID NO: 113) | NO: 113) | |
| CDR-L2 | DATNLAD (SEQ | DATNLAD (SEQ ID NO: | LLVYDATNLA (SEQ ID NO: |
| | ID NO: 114) | 114) | 122) |
| CDR-L3 | QHFWGTPLT | QHFWGTPLT (SEQ ID NO: | QHFWGTPL (SEQ ID NO: |
| | (SEQ ID NO: 115) | 115) | 123) |

| | |
|---|---|
| Murine VH | QVQLQQPGAELVKPGASVKLSCKASGYTFTSYWMHWVKQRPGQGLEWIGEINP |
| | TNGRTNYIEKFKSKATLTVDKSSSTAYMQLSSLTSEDSAVYYCARGTRAYHYW |
| | GQGTSVTVSS (SEQ ID NO: 124) |
| Murine VL | DIQMTQSPASLSVSVGETVTITCRASDNLYSNLAWYQQKQGKSPQLLVYDATNL |
| | ADGVPSRFSGSGSGTQYSLKINSLQSEDFGTYYCQHFWGTPLTFGAGTKLELK |
| | (SEQ ID NO: 125) |
| Humanized VH | EVQLVQSGAEVKKPGASVKVSCKASGYTFTSYWMHWVRQAPGQRLEWIGEIN |
| | PTNGRTNYIEKFKSRATLTVDKSASTAYMELSSLRSEDTAVYYCARGTRAYHY |
| | WGQGTMVTVSS (SEQ ID NO: 128) |
| Humanized VL | DIQMTQSPSSLSASVGDRVTITCRASDNLYSNLAWYQQKPGKSPKLLVYDATNL |
| | ADGVPSRFSGSGSGTDYTLTISSLQPEDFATYYCQHFWGTPLTFGQGTKVEIK |
| | (SEQ ID NO: 129) |
| HC of chimeric | QVQLQQPGAELVKPGASVKLSCKASGYTFTSYWMHWVKQRPGQGLEWIGEINP |
| full-length IgG1 | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEAL |
| | HNHYTQKSLSLSPGK (SEQ ID NO: 132) |
| LC of chimeric | DIQMTQSPASLSVSVGETVTITCRASDNLYSNLAWYQQKQGKSPQLLVYDATNL |
| full-length IgG1 | ADGVPSRFSGSGSGTQYSLKINSLQSEDFGTYYCQHFWGTPLTFGAGTKLELKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 133) |
| HC of fully human | EVQLVQSGAEVKKPGASVKVSCKASGYTFTSYWMHWVRQAPGQRLEWIGEIN |
| full-length IgG1 | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK (SEQ ID NO: 134) |
| LC of fully human | DIQMTQSPSSLSASVGDRVTITCRASDNLYSNLAWYQQKPGKSPKLLVYDATNL |
| full-length IgG1 | ADGVPSRFSGSGSGTDYTLTISSLQPEDFATYYCQHFWGTPLTFGQGTKVEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 135) |
| HC of chimeric | QVQLQQPGAELVKPGASVKLSCKASGYTFTSYWMHWVKQRPGQGLEWIGEINP |
| Fab | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCP (SEQ ID NO: 136) |
| HC of fully human | EVQLVQSGAEVKKPGASVKVSCKASGYTFTSYWMHWVRQAPGQRLEWIGEIN |
| Fab | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCP (SEQ ID NO: 137) |

In some embodiments, the anti-TfR1 antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1 antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1 antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1 antibody of the present disclosure comprises a CDR-L3 containing one amino acid variation as compared with the CDR-L3 as shown in Table 7. In some embodiments, the anti-TfR1 antibody of the present disclosure comprises a CDR-L3 of QHFAGTPLT (SEQ ID NO: 126) (according to the Kabat and Chothia definition system) or QHFAGTPL (SEQ ID NO: 127) (according to the Contact definition system). In some embodiments, the anti-TfR1 antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) (according to the Kabat and Chothia definition system) or QHFAGTPL (SEQ ID NO: 127) (according to the Contact definition system).

In some embodiments, the anti-TfR1 antibody of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1 antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1 antibody of the present disclosure comprises a VH comprising the amino acid sequence of SEQ ID NO: 124. Alternatively or in addition (e.g., in addition), the anti-TfR1 antibody of the present disclosure comprises a VL comprising the amino acid sequence of SEQ ID NO: 125.

In some embodiments, the anti-TfR1 antibody of the present disclosure comprises a VH comprising the amino acid sequence of SEQ ID NO: 128. Alternatively or in addition (e.g., in addition), the anti-TfR1 antibody of the present disclosure comprises a VL comprising the amino acid sequence of SEQ ID NO: 129.

In some embodiments, the anti-TfR1 antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1 antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the anti-TfR1 antibody of the present disclosure is a full-length IgG1 antibody, which can include a heavy constant region and a light constant region from a human antibody. In some embodiments, the heavy chain of any of the anti-TfR1 antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the anti-TfR1 antibody described herein is a chimeric antibody that comprises a heavy chain comprising the amino acid sequence of SEQ ID NO: 132. Alternatively or in addition (e.g., in addition), the anti-TfR1 antibody described herein comprises a light chain comprising the amino acid sequence of SEQ ID NO: 133.

In some embodiments, the anti-TfR1 antibody described herein is a fully human antibody that comprises a heavy chain comprising the amino acid sequence of SEQ ID NO: 134. Alternatively or in addition (e.g., in addition), the anti-TfR1 antibody described herein comprises a light chain comprising the amino acid sequence of SEQ ID NO: 135.

In some embodiments, the anti-TfR1 antibody is an antigen binding fragment (Fab) of an intact antibody (full-length antibody). In some embodiments, the anti-TfR1 Fab described herein comprises a heavy chain comprising the amino acid sequence of SEQ ID NO: 136. Alternatively or in addition (e.g., in addition), the anti-TfR1 Fab described herein comprises a light chain comprising the amino acid sequence of SEQ ID NO: 133. In some embodiments, the anti-TfR1 Fab described herein comprises a heavy chain comprising the amino acid sequence of SEQ ID NO: 137. Alternatively or in addition (e.g., in addition), the anti-TfR1 Fab described herein comprises a light chain comprising the amino acid sequence of SEQ ID NO: 135.

The anti-TfR1 antibodies described herein can be in any antibody form, including, but not limited to, intact (i.e., full-length) antibodies, antigen-binding fragments thereof (such as Fab, Fab′, F(ab′)₂, Fv), single chain antibodies, bi-specific antibodies, or nanobodies. In some embodiments, the anti-TfR1 antibody described herein is an scFv. In some embodiments, the anti-TfR1 antibody described herein is an scFv-Fab (e.g., scFv fused to a portion of a constant region). In some embodiments, the anti-TfR1 antibody described herein is an scFv fused to a constant region (e.g., human IgG1 constant region as set forth in SEQ ID NO: 81).

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

b. Other Muscle-Targeting Antibodies

In some embodiments, the muscle-targeting antibody is an antibody that specifically binds hemojuvelin, caveolin-3, Duchenne muscular dystrophy peptide, myosin IIb or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 778) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 778). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for Duchenne muscular dystrophy. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 779) was identified and this muscle-targeting peptide showed improved binding to C2C12 cells relative to the ASSLNIA (SEQ ID NO: 778) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 780) appeared most frequently. Accordingly, in some embodiments, the muscle-targeting agent comprises the amino acid sequence TARGEHKEEELI (SEQ ID NO: 780).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 781), CSERSMNFC (SEQ ID NO: 782), CPKTRRVPC (SEQ ID NO: 783), WLSEAGPVVTVRALRGTGSW (SEQ ID NO: 784), ASSLNIA (SEQ ID NO: 778), CMQHSMRVC (SEQ ID NO: 785), and DDTRHWG (SEQ ID NO: 786). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10⁻¹⁵ kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agent described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

### B. Molecular Payloads

Some aspects of the disclosure provide molecular payloads, e.g., for modulating a biological outcome, e.g., the transcription of a DNA sequence, the splicing and processing of a RNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of molecular payloads may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a mutated DMD allele. Exemplary molecular payloads are described in further detail herein, however, it should be appreciated that the exemplary molecular payloads provided herein are not meant to be limiting.

i. Oligonucleotides

Aspects of the disclosure relate to oligonucleotides configured to modulate (e.g., increase) expression of dystrophin, e.g., from a DMD allele. In some embodiments, oligonucleotides provided herein are configured to alter splicing of DMD pre-mRNA to promote expression of dystrophin protein (e.g., a functional truncated dystrophin protein). In some embodiments, oligonucleotides provided herein are configured to promote skipping of one or more exons in DMD, e.g., in a mutated DMD allele, in order to restore the reading frame. In some embodiments, the oligonucleotides allow for functional dystrophin protein expression (e.g., as described in Watanabe N, Nagata T, Satou Y, et al. NS-065/NCNP-01: an antisense oligonucleotide for potential treatment of exon 53 skipping in Duchenne muscular dystrophy. Mol Ther Nucleic Acids. 2018; 13:442-449). In some embodiments, oligonucleotides provided are configured to promote skipping of exon 53 to produce a shorter but functional version of dystrophin (e.g., containing an in-frame deletion). In some embodiments, oligonucleotides are provided that promote exon 53 skipping (e.g., which may be relevant in a substantial number of patients, including, for example, patients amenable to exon 53 skipping, such as those having deletions in DMD exons 3-52, 4-52, 5-52, 6-52, 9-52, 10-52, 11-52, 13-52, 14-52, 16-52, 17-52, 19-52, 21-52, 23-52, 24-52, 25-52, 26-52, 27-52, 28-52, 29-52, 30-52, 31-52, 32-52, 33-52, 34-52, 35-52, 36-52, 37-52, 38-52, 39-52, 40-52, 41-52, 42-52, 43-52, 45-52, 47-52, 48-52, 49-52, 50-52, 52, 54-58, 54-61, 54-63, 54-64, 54-66, 54-76, or 54-77).

Table 8 provides non-limiting examples of sequences of oligonucleotides that are useful for targeting DMD, e.g., for exon skipping, and for target sequences within DMD. In some embodiments, an oligonucleotide may comprise any antisense sequence provided in Table 8 or a sequence complementary to a target sequence provided in Table 8. Table 8. Oligonucleotide sequences for targeting DMD.

TABLE 8
Oligonucleotide sequences for targeting DMD.

| SEQ | | SEQ | Antisense | SEQ | Antisense | |
|---|---|---|---|---|---|---|
| ID | Target sequence^{†} | ID | Sequence* | ID | Sequence^{†} | |
| NO | (5′ to 3′) | NO | (5′ to 3′) | NO | (5′ to 3′) | Target Site |
| 160 | GAGUCAUGGAAGG | 335 | AUAGGGACCCUCC | 510 | ATAGGGACCCTCC | Exon 53 |
| | AGGGUCCCUAU | | UUCCAUGACUC | | TTCCATGACTC | |
| 161 | GGAGGGUCCCUAU | 336 | CAUCUACUGUAUA | 511 | CATCTACTGTATA | Exon 53 |
| | ACAGUAGAUG | | GGGACCCUCC | | GGGACCCTCC | |
| 162 | AGGAGGGUCCCUA | 337 | CAUCUACUGUAUA | 512 | CATCTACTGTATA | Exon 53 |
| | UACAGUAGAUG | | GGGACCCUCCU | | GGGACCCTCCT | |
| 163 | GCUUGAGUCAUGG | 338 | CCCUCCUUCCAUG | 513 | CCCTCCTTCCATG | Exon 53 |
| | AAGGAGGG | | ACUCAAGC | | ACTCAAGC | |
| 164 | AGCUUGAGUCAUG | 339 | CCCUCCUUCCAUG | 514 | CCCTCCTTCCATG | Exon 53 |
| | GAAGGAGGG | | ACUCAAGCU | | ACTCAAGCT | |
| 165 | GCUUGAGUCAUGG | 340 | CCUCCUUCCAUGA | 515 | CCTCCTTCCATGA | Exon 53 |
| | AAGGAGG | | CUCAAGC | | CTCAAGC | |
| 166 | AGCUUGAGUCAUG | 341 | CCUCCUUCCAUGA | 516 | CCTCCTTCCATGA | Exon 53 |
| | GAAGGAGG | | CUCAAGCU | | CTCAAGCT | |
| 167 | CAACACAAUGGCU | 342 | CCUUAGCUUCCAG | 517 | CCTTAGCTTCCAG | Exon 53 |
| | GGAAGCUAAGG | | CCAUUGUGUUG | | CCATTGTGTTG | |
| 168 | UCAACACAAUGGC | 343 | CCUUAGCUUCCAG | 518 | CCTTAGCTTCCAG | Exon 53 |
| | UGGAAGCUAAGG | | CCAUUGUGUUGA | | CCATTGTGTTGA | |
| 169 | AGCUUGAGUCAUG | 344 | CUCCUUCCAUGAC | 519 | CTCCTTCCATGAC | Exon 53 |
| | GAAGGAG | | UCAAGCU | | TCAAGCT | |
| 170 | AUCAGUGGGAUGA | 345 | CUUGUACUUCAUC | 520 | CTTGTACTTCATC | Exon 53 |
| | AGUACAAG | | CCACUGAU | | CCACTGAT | |
| 171 | GAAUCAGUGGGAU | 346 | CUUGUACUUCAUC | 521 | CTTGTACTTCATC | Exon 53 |
| | GAAGUACAAG | | CCACUGAUUC | | CCACTGATTC | |
| 172 | UGAGUCAUGGAAG | 347 | GACCCUCCUUCCA | 522 | GACCCTCCTTCCA | Exon 53 |
| | GAGGGUC | | UGACUCA | | TGACTCA | |
| 173 | UUGAGUCAUGGAA | 348 | GACCCUCCUUCCA | 523 | GACCCTCCTTCCA | Exon 53 |
| | GGAGGGUC | | UGACUCAA | | TGACTCAA | |
| 174 | CUUGAGUCAUGGA | 349 | GACCCUCCUUCCA | 524 | GACCCTCCTTCCA | Exon 53 |
| | AGGAGGGUC | | UGACUCAAG | | TGACTCAAG | |
| 175 | GCUUGAGUCAUGG | 350 | GACCCUCCUUCCA | 525 | GACCCTCCTTCCA | Exon 53 |
| | AAGGAGGGUC | | UGACUCAAGC | | TGACTCAAGC | |
| 176 | GGGUCCCUAUACA | 351 | GAUUGCAUCUACU | 526 | GATTGCATCTACT | Exon 53 |
| | GUAGAUGCAAUC | | GUAUAGGGACCC | | GTATAGGGACCC | |
| 177 | GGAGGGUCCCUAU | 352 | GCAUCUACUGUAU | 527 | GCATCTACTGTAT | Exon 53 |
| | ACAGUAGAUGC | | AGGGACCCUCC | | AGGGACCCTCC | |
| 178 | AAGCAUGGGACAC | 353 | GCUUUGUGUGUCC | 528 | GCTTTGTGTGTCC | Intron 52 |
| | ACAAAGC | | CAUGCUU | | CATGCTT | |
| 179 | CAAGCAUGGGACA | 354 | GCUUUGUGUGUCC | 529 | GCTTTGTGTGTCC | Intron 52 |
| | CACAAAGC | | CAUGCUUG | | CATGCTTG | |
| 180 | ACAAGCAUGGGAC | 355 | GCUUUGUGUGUCC | 530 | GCTTTGTGTGTCC | Intron 52 |
| | ACACAAAGC | | CAUGCUUGU | | CATGCTTGT | |
| 181 | AACAAGCAUGGGA | 356 | GCUUUGUGUGUCC | 531 | GCTTTGTGTGTCC | Intron 52 |
| | CACACAAAGC | | CAUGCUUGUU | | CATGCTTGTT | |
| 182 | UAACAAGCAUGGG | 357 | GCUUUGUGUGUCC | 532 | GCTTTGTGTGTCC | Intron 52 |
| | ACACACAAAGC | | CAUGCUUGUUA | | CATGCTTGTTA | |
| 183 | GAGUCAUGGAAGG | 358 | GGACCCUCCUUCC | 533 | GGACCCTCCTTCC | Exon 53 |
| | AGGGUCC | | AUGACUC | | ATGACTC | |
| 184 | GGUCCCUAUACAG | 359 | GGAUUGCAUCUAC | 534 | GGATTGCATCTAC | Exon 53 |
| | UAGAUGCAAUCC | | UGUAUAGGGACC | | TGTATAGGGACC | |
| 185 | UGAGUCAUGGAAG | 360 | GGGACCCUCCUUC | 535 | GGGACCCTCCTTC | Exon 53 |
| | GAGGGUCCC | | CAUGACUCA | | CATGACTCA | |
| 186 | AAGUUUGUCCUGA | 361 | GUAACCCACCUUU | 536 | GTAACCCACCTTT | Intron 53 |
| | AAGGUGGGUUAC | | CAGGACAAACUU | | CAGGACAAACTT | |
| 187 | GAAUCAGUGGGAU | 362 | GUACUUCAUCCCA | 537 | GTACTTCATCCCA | Exon 53 |
| | GAAGUAC | | CUGAUUC | | CTGATTC | |
| 188 | GUUCAUCAUCCUA | 363 | GUGUUAUGGCUAG | 538 | GTGTTATGGCTAG | Intron 52 |
| | GCCAUAACAC | | GAUGAUGAAC | | GATGATGAAC | |
| 189 | CAGUGGGAUGAAG | 364 | UCUUGUACUUCAU | 539 | TCTTGTACTTCAT | Exon 53 |
| | UACAAGA | | CCCACUG | | CCCACTG | |
| 190 | UCAGUGGGAUGAA | 365 | UCUUGUACUUCAU | 540 | TCTTGTACTTCAT | Exon 53 |
| | GUACAAGA | | CCCACUGA | | CCCACTGA | |
| 191 | AUCAGUGGGAUGA | 366 | UCUUGUACUUCAU | 541 | TCTTGTACTTCAT | Exon 53 |
| | AGUACAAGA | | CCCACUGAU | | CCCACTGAT | |
| 192 | AAUCAGUGGGAUG | 367 | UCUUGUACUUCAU | 542 | TCTTGTACTTCAT | Exon 53 |
| | AAGUACAAGA | | CCCACUGAUU | | CCCACTGATT | |
| 193 | GAAUCAGUGGGAU | 368 | UCUUGUACUUCAU | 543 | TCTTGTACTTCAT | Exon 53 |
| | GAAGUACAAGA | | CCCACUGAUUC | | CCCACTGATTC | |
| 194 | AGAACACCUUCAG | 369 | UGCCUCCGGUUCU | 544 | TGCCTCCGGTTCT | Exon 53 |
| | AACCGGAGGCA | | GAAGGUGUUCU | | GAAGGTGTTCT | |
| 195 | AAGAACACCUUCA | 370 | UGCCUCCGGUUCU | 545 | TGCCTCCGGTTCT | Exon 53 |
| | GAACCGGAGGCA | | GAAGGUGUUCUU | | GAAGGTGTTCTT | |
| 196 | AGCAUGGGACACA | 371 | UGCUUUGUGUGUC | 546 | TGCTTTGTGTGTC | Intron 52 |
| | CAAAGCA | | CCAUGCU | | CCATGCT | |
| 197 | AAGCAUGGGACAC | 372 | UGCUUUGUGUGUC | 547 | TGCTTTGTGTGTC | Intron 52 |
| | ACAAAGCA | | CCAUGCUU | | CCATGCTT | |
| 198 | CAAGCAUGGGACA | 373 | UGCUUUGUGUGUC | 548 | TGCTTTGTGTGTC | Intron 52 |
| | CACAAAGCA | | CCAUGCUUG | | CCATGCTTG | |
| 199 | ACAAGCAUGGGAC | 374 | UGCUUUGUGUGUC | 549 | TGCTTTGTGTGTC | Intron 52 |
| | ACACAAAGCA | | CCAUGCUUGU | | CCATGCTTGT | |
| 200 | AACAAGCAUGGGA | 375 | UGCUUUGUGUGUC | 550 | TGCTTTGTGTGTC | Intron 52 |
| | CACACAAAGCA | | CCAUGCUUGUU | | CCATGCTTGTT | |
| 201 | UAACAAGCAUGGG | 376 | UGCUUUGUGUGUC | 551 | TGCTTTGTGTGTC | Intron 52 |
| | ACACACAAAGCA | | CCAUGCUUGUUA | | CCATGCTTGTTA | |
| 202 | GAAUCAGUGGGAU | 377 | UGUACUUCAUCCC | 552 | TGTACTTCATCCC | Exon 53 |
| | GAAGUACA | | ACUGAUUC | | ACTGATTC | |
| 203 | UAACAAGCAUGGG | 378 | UGUGUGUCCCAUG | 553 | TGTGTGTCCCATG | Intron 52 |
| | ACACACA | | CUUGUUA | | CTTGTTA | |
| 204 | GUUCAUCAUCCUA | 379 | UGUGUUAUGGCUA | 554 | TGTGTTATGGCTA | Intron 52 |
| | GCCAUAACACA | | GGAUGAUGAAC | | GGATGATGAAC | |
| 205 | AGUGGGAUGAAGU | 380 | UGUUCUUGUACUU | 555 | TGTTCTTGTACTT | Exon 53 |
| | ACAAGAACA | | CAUCCCACU | | CATCCCACT | |
| 206 | CAGUGGGAUGAAG | 381 | UGUUCUUGUACUU | 556 | TGTTCTTGTACTT | Exon 53 |
| | UACAAGAACA | | CAUCCCACUG | | CATCCCACTG | |
| 207 | UCAGUGGGAUGAA | 382 | UGUUCUUGUACUU | 557 | TGTTCTTGTACTT | Exon 53 |
| | GUACAAGAACA | | CAUCCCACUGA | | CATCCCACTGA | |
| 208 | AUCAGUGGGAUGA | 383 | UGUUCUUGUACUU | 558 | TGTTCTTGTACTT | Exon 53 |
| | AGUACAAGAACA | | CAUCCCACUGAU | | CATCCCACTGAT | |
| 209 | CAGUGGGAUGAAG | 384 | UUCUUGUACUUCA | 559 | TTCTTGTACTTCA | Exon 53 |
| | UACAAGAA | | UCCCACUG | | TCCCACTG | |
| 210 | UCAGUGGGAUGAA | 385 | UUCUUGUACUUCA | 560 | TTCTTGTACTTCA | Exon 53 |
| | GUACAAGAA | | UCCCACUGA | | TCCCACTGA | |
| 211 | AUCAGUGGGAUGA | 386 | UUCUUGUACUUCA | 561 | TTCTTGTACTTCA | Exon 53 |
| | AGUACAAGAA | | UCCCACUGAU | | TCCCACTGAT | |
| 212 | AAUCAGUGGGAUG | 387 | UUCUUGUACUUCA | 562 | TTCTTGTACTTCA | Exon 53 |
| | AAGUACAAGAA | | UCCCACUGAUU | | TCCCACTGATT | |
| 213 | GAAUCAGUGGGAU | 388 | UUCUUGUACUUCA | 563 | TTCTTGTACTTCA | Exon 53 |
| | GAAGUACAAGAA | | UCCCACUGAUUC | | TCCCACTGATTC | |
| 214 | GAAUCAGUGGGAU | 389 | UUGUACUUCAUCC | 564 | TTGTACTTCATCC | Exon 53 |
| | GAAGUACAA | | CACUGAUUC | | CACTGATTC | |
| 215 | UAACAAGCAUGGG | 390 | UUGUGUGUCCCAU | 565 | TTGTGTGTCCCAT | Intron 52 |
| | ACACACAA | | GCUUGUUA | | GCTTGTTA | |
| 216 | GUUCAUCAUCCUA | 391 | UUGUGUUAUGGCU | 566 | TTGTGTTATGGCT | Intron 52 |
| | GCCAUAACACAA | | AGGAUGAUGAAC | | AGGATGATGAAC | |
| 217 | ACAAGCAUGGGAC | 392 | UUUGUGUGUCCCA | 567 | TTTGTGTGTCCCA | Intron 52 |
| | ACACAAA | | UGCUUGU | | TGCTTGT | |
| 218 | AACAAGCAUGGGA | 393 | UUUGUGUGUCCCA | 568 | TTTGTGTGTCCCA | Intron 52 |
| | CACACAAA | | UGCUUGUU | | TGCTTGTT | |
| 219 | UAACAAGCAUGGG | 394 | UUUGUGUGUCCCA | 569 | TTTGTGTGTCCCA | Intron 52 |
| | ACACACAAA | | UGCUUGUUA | | TGCTTGTTA | |
| 220 | AUGUCUCCUCCAG | 395 | AAAUGCUAGUCUG | 570 | AAATGCTAGTCTG | Intron 52 |
| | ACUAGCAUUU | | GAGGAGACAU | | GAGGAGACAT | |
| 221 | AAUGUCUCCUCCA | 396 | AAAUGCUAGUCUG | 571 | AAATGCTAGTCTG | Intron 52 |
| | GACUAGCAUUU | | GAGGAGACAUU | | GAGGAGACATT | |
| 222 | AAAUGUCUCCUCC | 397 | AAAUGCUAGUCUG | 572 | AAATGCTAGTCTG | Intron 52 |
| | AGACUAGCAUUU | | GAGGAGACAUUU | | GAGGAGACATTT | |
| 223 | AAAGUUUGUCCUG | 398 | AACCCACCUUUCA | 573 | AACCCACCTTTCA | Intron 53 |
| | AAAGGUGGGUU | | GGACAAACUUU | | GGACAAACTTT | |
| 224 | CCUUCAGAACCGG | 399 | AACUGUUGCCUCC | 574 | AACTGTTGCCTCC | Exon 53 |
| | AGGCAACAGUU | | GGUUCUGAAGG | | GGTTCTGAAGG | |
| 225 | GAGUCAUGGAAGG | 400 | AGGGACCCUCCUU | 575 | AGGGACCCTCCTT | Exon 53 |
| | AGGGUCCCU | | CCAUGACUC | | CCATGACTC | |
| 226 | AGUCAUGGAAGGA | 401 | AUAGGGACCCUCC | 576 | ATAGGGACCCTCC | Exon 53 |
| | GGGUCCCUAU | | UUCCAUGACU | | TTCCATGACT | |
| 227 | GGAGGGUCCCUAU | 402 | AUCUACUGUAUAG | 577 | ATCTACTGTATAG | Exon 53 |
| | ACAGUAGAU | | GGACCCUCC | | GGACCCTCC | |
| 228 | AGGAGGGUCCCUA | 403 | AUCUACUGUAUAG | 578 | ATCTACTGTATAG | Exon 53 |
| | UACAGUAGAU | | GGACCCUCCU | | GGACCCTCCT | |
| 229 | ACCAAGGUUAGUA | 404 | AUCUUUGAUACUA | 579 | ATCTTTGATACTA | Exon 53/intron 53 |
| | UCAAAGAU | | ACCUUGGU | | ACCTTGGT | junction |
| 230 | AACCAAGGUUAGU | 405 | AUCUUUGAUACUA | 580 | ATCTTTGATACTA | Exon 53/intron 53 |
| | AUCAAAGAU | | ACCUUGGUU | | ACCTTGGTT | junction |
| 231 | UCAUCAUCCUAGC | 406 | AUUGUGUUAUGGC | 581 | ATTGTGTTATGGC | Intron 52 |
| | CAUAACACAAU | | UAGGAUGAUGA | | TAGGATGATGA | |
| 232 | CCUUCAGAACCGG | 407 | CAACUGUUGCCUC | 582 | CAACTGTTGCCTC | Exon 53 |
| | AGGCAACAGUUG | | CGGUUCUGAAGG | | CGGTTCTGAAGG | |
| 233 | AGAACACCUUCAG | 408 | CCUCCGGUUCUGA | 583 | CCTCCGGTTCTGA | Exon 53 |
| | AACCGGAGG | | AGGUGUUCU | | AGGTGTTCT | |
| 234 | AAGAACACCUUCA | 409 | CCUCCGGUUCUGA | 584 | CCTCCGGTTCTGA | Exon 53 |
| | GAACCGGAGG | | AGGUGUUCUU | | AGGTGTTCTT | |
| 235 | CAAGAACACCUUC | 410 | CCUCCGGUUCUGA | 585 | CCTCCGGTTCTGA | Exon 53 |
| | AGAACCGGAGG | | AGGUGUUCUUG | | AGGTGTTCTTG | |
| 236 | ACACAAUGGCUGG | 411 | CCUUAGCUUCCAG | 586 | CCTTAGCTTCCAG | Exon 53 |
| | AAGCUAAGG | | CCAUUGUGU | | CCATTGTGT | |
| 237 | AACACAAUGGCUG | 412 | CCUUAGCUUCCAG | 587 | CCTTAGCTTCCAG | Exon 53 |
| | GAAGCUAAGG | | CCAUUGUGUU | | CCATTGTGTT | |
| 238 | CAAGAACACCUUC | 413 | CUCCGGUUCUGAA | 588 | CTCCGGTTCTGAA | Exon 53 |
| | AGAACCGGAG | | GGUGUUCUUG | | GGTGTTCTTG | |
| 239 | UACAAGAACACCU | 414 | CUCCGGUUCUGAA | 589 | CTCCGGTTCTGAA | Exon 53 |
| | UCAGAACCGGAG | | GGUGUUCUUGUA | | GGTGTTCTTGTA | |
| 240 | CAACACAAUGGCU | 415 | CUUAGCUUCCAGC | 590 | CTTAGCTTCCAGC | Exon 53 |
| | GGAAGCUAAG | | CAUUGUGUUG | | CATTGTGTTG | |
| 241 | UCAACACAAUGGC | 416 | CUUAGCUUCCAGC | 591 | CTTAGCTTCCAGC | Exon 53 |
| | UGGAAGCUAAG | | CAUUGUGUUGA | | CATTGTGTTGA | |
| 242 | UCAGUGGGAUGAA | 417 | CUUGUACUUCAUC | 592 | CTTGTACTTCATC | Exon 53 |
| | GUACAAG | | CCACUGA | | CCACTGA | |
| 243 | AAUCAGUGGGAUG | 418 | CUUGUACUUCAUC | 593 | CTTGTACTTCATC | Exon 53 |
| | AAGUACAAG | | CCACUGAUU | | CCACTGATT | |
| 244 | AUACAGUAGAUGC | 419 | CUUUUGGAUUGCA | 594 | CTTTTGGATTGCA | Exon 53 |
| | AAUCCAAAAG | | UCUACUGUAU | | TCTACTGTAT | |
| 245 | GGUCCCUAUACAG | 420 | GAUUGCAUCUACU | 595 | GATTGCATCTACT | Exon 53 |
| | UAGAUGCAAUC | | GUAUAGGGACC | | GTATAGGGACC | |
| 246 | GAGGGUCCCUAUA | 421 | GCAUCUACUGUAU | 596 | GCATCTACTGTAT | Exon 53 |
| | CAGUAGAUGC | | AGGGACCCUC | | AGGGACCCTC | |
| 247 | AAUGUCUCCUCCA | 422 | GCUAGUCUGGAGG | 597 | GCTAGTCTGGAGG | Intron 52 |
| | GACUAGC | | AGACAUU | | AGACATT | |
| 248 | AAAUGUCUCCUCC | 423 | GCUAGUCUGGAGG | 598 | GCTAGTCTGGAGG | Intron 52 |
| | AGACUAGC | | AGACAUUU | | AGACATTT | |
| 249 | AAAAUGUCUCCUC | 424 | GCUAGUCUGGAGG | 599 | GCTAGTCTGGAGG | Intron 52 |
| | CAGACUAGC | | AGACAUUUU | | AGACATTTT | |
| 250 | UAAAAUGUCUCCU | 425 | GCUAGUCUGGAGG | 600 | GCTAGTCTGGAGG | Intron 52 |
| | CCAGACUAGC | | AGACAUUUUA | | AGACATTTTA | |
| 251 | UGAGUCAUGGAAG | 426 | GGACCCUCCUUCC | 601 | GGACCCTCCTTCC | Exon 53 |
| | GAGGGUCC | | AUGACUCA | | ATGACTCA | |
| 252 | UUGAGUCAUGGAA | 427 | GGACCCUCCUUCC | 602 | GGACCCTCCTTCC | Exon 53 |
| | GGAGGGUCC | | AUGACUCAA | | ATGACTCAA | |
| 253 | GUCCCUAUACAGU | 428 | GGAUUGCAUCUAC | 603 | GGATTGCATCTAC | Exon 53 |
| | AGAUGCAAUCC | | UGUAUAGGGAC | | TGTATAGGGAC | |
| 254 | AGUCAUGGAAGGA | 429 | GGGACCCUCCUUC | 604 | GGGACCCTCCTTC | Exon 53 |
| | GGGUCCC | | CAUGACU | | CATGACT | |
| 255 | UUGAGUCAUGGAA | 430 | GGGACCCUCCUUC | 605 | GGGACCCTCCTTC | Exon 53 |
| | GGAGGGUCCC | | CAUGACUCAA | | CATGACTCAA | |
| 256 | AACUUAAGUUCAU | 431 | GGGAUAUAUGAAC | 606 | GGGATATATGAAC | Intron 52 |
| | AUAUCCC | | UUAAGUU | | TTAAGTT | |
| 257 | AGUUUGUCCUGAA | 432 | GGUAACCCACCUU | 607 | GGTAACCCACCTT | Intron 53 |
| | AGGUGGGUUACC | | UCAGGACAAACU | | TCAGGACAAACT | |
| 258 | AGUUUGUCCUGAA | 433 | GUAACCCACCUUU | 608 | GTAACCCACCTTT | Intron 53 |
| | AGGUGGGUUAC | | CAGGACAAACU | | CAGGACAAACT | |
| 259 | UCAUCAUCCUAGC | 434 | GUGUUAUGGCUAG | 609 | GTGTTATGGCTAG | Intron 52 |
| | CAUAACAC | | GAUGAUGA | | GATGATGA | |
| 260 | UUCAUCAUCCUAG | 435 | GUGUUAUGGCUAG | 610 | GTGTTATGGCTAG | Intron 52 |
| | CCAUAACAC | | GAUGAUGAA | | GATGATGAA | |
| 261 | GUUCAUCAUCCUA | 436 | GUUAUGGCUAGGA | 611 | GTTATGGCTAGGA | Intron 52 |
| | GCCAUAAC | | UGAUGAAC | | TGATGAAC | |
| 262 | AUGUCUCCUCCAG | 437 | UAAAUGCUAGUCU | 612 | TAAATGCTAGTCT | Intron 52 |
| | ACUAGCAUUUA | | GGAGGAGACAU | | GGAGGAGACAT | |
| 263 | AAUGUCUCCUCCA | 438 | UAAAUGCUAGUCU | 613 | TAAATGCTAGTCT | Intron 52 |
| | GACUAGCAUUUA | | GGAGGAGACAUU | | GGAGGAGACATT | |
| 264 | AAGUUUGUCCUGA | 439 | UAACCCACCUUUC | 614 | TAACCCACCTTTC | Intron 53 |
| | AAGGUGGGUUA | | AGGACAAACUU | | AGGACAAACTT | |
| 265 | AAAGUUUGUCCUG | 440 | UAACCCACCUUUC | 615 | TAACCCACCTTTC | Intron 53 |
| | AAAGGUGGGUUA | | AGGACAAACUUU | | AGGACAAACTTT | |
| 266 | GAGUCAUGGAAGG | 441 | UAGGGACCCUCCU | 616 | TAGGGACCCTCCT | Exon 53 |
| | AGGGUCCCUA | | UCCAUGACUC | | TCCATGACTC | |
| 267 | ACCAAGGUUAGUA | 442 | UAUCUUUGAUACU | 617 | TATCTTTGATACT | Exon 53/intron 53 |
| | UCAAAGAUA | | AACCUUGGU | | AACCTTGGT | junction |
| 268 | UACAGUAGAUGCA | 443 | UCUUUUGGAUUGC | 618 | TCTTTTGGATTGC | Exon 53 |
| | AUCCAAAAGA | | AUCUACUGUA | | ATCTACTGTA | |
| 269 | GUCCCUAUACAGU | 444 | UGGAUUGCAUCUA | 619 | TGGATTGCATCTA | Exon 53 |
| | AGAUGCAAUCCA | | CUGUAUAGGGAC | | CTGTATAGGGAC | |
| 270 | AAUCAGUGGGAUG | 445 | UGUACUUCAUCCC | 620 | TGTACTTCATCCC | Exon 53 |
| | AAGUACA | | ACUGAUU | | ACTGATT | |
| 271 | CAUCAUCCUAGCC | 446 | UGUGUUAUGGCUA | 621 | TGTGTTATGGCTA | Intron 52 |
| | AUAACACA | | GGAUGAUG | | GGATGATG | |
| 272 | UCAUCAUCCUAGC | 447 | UGUGUUAUGGCUA | 622 | TGTGTTATGGCTA | Intron 52 |
| | CAUAACACA | | GGAUGAUGA | | GGATGATGA | |
| 273 | UUCAUCAUCCUAG | 448 | UGUGUUAUGGCUA | 623 | TGTGTTATGGCTA | Intron 52 |
| | CCAUAACACA | | GGAUGAUGAA | | GGATGATGAA | |
| 274 | GUGGGAUGAAGUA | 449 | UGUUCUUGUACUU | 624 | TGTTCTTGTACTT | Exon 53 |
| | CAAGAACA | | CAUCCCAC | | CATCCCAC | |
| 275 | AGUGGGAUGAAGU | 450 | UUCUUGUACUUCA | 625 | TTCTTGTACTTCA | Exon 53 |
| | ACAAGAA | | UCCCACU | | TCCCACT | |
| 276 | AUCAGUGGGAUGA | 451 | UUGUACUUCAUCC | 626 | TTGTACTTCATCC | Exon 53 |
| | AGUACAA | | CACUGAU | | CACTGAT | |
| 277 | AAUCAGUGGGAUG | 452 | UUGUACUUCAUCC | 627 | TTGTACTTCATCC | Exon 53 |
| | AAGUACAA | | CACUGAUU | | CACTGATT | |
| 278 | AACAAGCAUGGGA | 453 | UUGUGUGUCCCAU | 628 | TTGTGTGTCCCAT | Intron 52 |
| | CACACAA | | GCUUGUU | | GCTTGTT | |
| 279 | UCAUCAUCCUAGC | 454 | UUGUGUUAUGGCU | 629 | TTGTGTTATGGCT | Intron 52 |
| | CAUAACACAA | | AGGAUGAUGA | | AGGATGATGA | |
| 280 | AAGUUUGUCCUGA | 455 | AACCCACCUUUCA | 630 | AACCCACCTTTCA | Intron 53 |
| | AAGGUGGGUU | | GGACAAACUU | | GGACAAACTT | |
| 281 | AAAAGUUUGUCCU | 456 | AACCCACCUUUCA | 631 | AACCCACCTTTCA | Intron 53 |
| | GAAAGGUGGGUU | | GGACAAACUUUU | | GGACAAACTTTT | |
| 282 | CCUAGCCAUAACA | 457 | AAUUAUUCAUUGU | 632 | AATTATTCATTGT | Intron 52 |
| | CAAUGAAUAAUU | | GUUAUGGCUAGG | | GTTATGGCTAGG | |
| 283 | AAGGAUUCAACAC | 458 | AGCCAUUGUGUUG | 633 | AGCCATTGTGTTG | Exon 53 |
| | AAUGGCU | | AAUCCUU | | AATCCTT | |
| 284 | UAAAGGAUUCAAC | 459 | AGCCAUUGUGUUG | 634 | AGCCATTGTGTTG | Exon 53 |
| | ACAAUGGCU | | AAUCCUUUA | | AATCCTTTA | |
| 285 | ACCAAGGUUAGUA | 460 | AGGUAUCUUUGAU | 635 | AGGTATCTTTGAT | Exon 53/intron 53 |
| | UCAAAGAUACCU | | ACUAACCUUGGU | | ACTAACCTTGGT | junction |
| 286 | GUCAUGGAAGGAG | 461 | AUAGGGACCCUCC | 636 | ATAGGGACCCTCC | Exon 53 |
| | GGUCCCUAU | | UUCCAUGAC | | TTCCATGAC | |
| 287 | AUAUAUGUAUUCU | 462 | AUCCUCAGGUCAG | 637 | ATCCTCAGGTCAG | Intron 53 |
| | GACCUGAGGAU | | AAUACAUAUAU | | AATACATATAT | |
| 288 | AAACCAAGGUUAG | 463 | AUCUUUGAUACUA | 638 | ATCTTTGATACTA | Exon 53/intron 53 |
| | UAUCAAAGAU | | ACCUUGGUUU | | ACCTTGGTTT | junction |
| 289 | CUAGCCAUAACAC | 464 | AUUAUUCAUUGUG | 639 | ATTATTCATTGTG | Intron 52 |
| | AAUGAAUAAU | | UUAUGGCUAG | | TTATGGCTAG | |
| 290 | CCUAGCCAUAACA | 465 | AUUAUUCAUUGUG | 640 | ATTATTCATTGTG | Intron 52 |
| | CAAUGAAUAAU | | UUAUGGCUAGG | | TTATGGCTAGG | |
| 291 | UCCUAGCCAUAAC | 466 | AUUAUUCAUUGUG | 641 | ATTATTCATTGTG | Intron 52 |
| | ACAAUGAAUAAU | | UUAUGGCUAGGA | | TTATGGCTAGGA | |
| 292 | UUCAUCAUCCUAG | 467 | AUUGUGUUAUGGC | 642 | ATTGTGTTATGGC | Intron 52 |
| | CCAUAACACAAU | | UAGGAUGAUGAA | | TAGGATGATGAA | |
| 293 | AGCUGAAAUGAAC | 468 | CAAAGUCUACUGU | 643 | CAAAGTCTACTGT | Intron 53 |
| | AGUAGACUUUG | | UCAUUUCAGCU | | TCATTTCAGCT | |
| 294 | UAAAGGAUUCAAC | 469 | CAGCCAUUGUGUU | 644 | CAGCCATTGTGTT | Exon 53 |
| | ACAAUGGCUG | | GAAUCCUUUA | | GAATCCTTTA | |
| 295 | GAGGGUCCCUAUA | 470 | CAUCUACUGUAUA | 645 | CATCTACTGTATA | Exon 53 |
| | CAGUAGAUG | | GGGACCCUC | | GGGACCCTC | |
| 296 | UGAAAAGUUUGUC | 471 | CCACCUUUCAGGA | 646 | CCACCTTTCAGGA | Intron 53 |
| | CUGAAAGGUGG | | CAAACUUUUCA | | CAAACTTTTCA | |
| 297 | AGGAUUCAACACA | 472 | CCAGCCAUUGUGU | 647 | CCAGCCATTGTGT | Exon 53 |
| | AUGGCUGG | | UGAAUCCU | | TGAATCCT | |
| 298 | AAGGAUUCAACAC | 473 | CCAGCCAUUGUGU | 648 | CCAGCCATTGTGT | Exon 53 |
| | AAUGGCUGG | | UGAAUCCUU | | TGAATCCTT | |
| 299 | AAAGGAUUCAACA | 474 | CCAGCCAUUGUGU | 649 | CCAGCCATTGTGT | Exon 53 |
| | CAAUGGCUGG | | UGAAUCCUUU | | TGAATCCTTT | |
| 300 | UAAAGGAUUCAAC | 475 | CCAGCCAUUGUGU | 650 | CCAGCCATTGTGT | Exon 53 |
| | ACAAUGGCUGG | | UGAAUCCUUUA | | TGAATCCTTTA | |
| 301 | GAAAAGUUUGUCC | 476 | CCCACCUUUCAGG | 651 | CCCACCTTTCAGG | Intron 53 |
| | UGAAAGGUGGG | | ACAAACUUUUC | | ACAAACTTTTC | |
| 302 | UGAAAAGUUUGUC | 477 | CCCACCUUUCAGG | 652 | CCCACCTTTCAGG | Intron 53 |
| | CUGAAAGGUGGG | | ACAAACUUUUCA | | ACAAACTTTTCA | |
| 303 | CUUGAGUCAUGGA | 478 | CCCUCCUUCCAUG | 653 | CCCTCCTTCCATG | Exon 53 |
| | AGGAGGG | | ACUCAAG | | ACTCAAG | |
| 304 | AGAACACCUUCAG | 479 | CUCCGGUUCUGAA | 654 | CTCCGGTTCTGAA | Exon 53 |
| | AACCGGAG | | GGUGUUCU | | GGTGTTCT | |
| 305 | AAGAACACCUUCA | 480 | CUCCGGUUCUGAA | 655 | CTCCGGTTCTGAA | Exon 53 |
| | GAACCGGAG | | GGUGUUCUU | | GGTGTTCTT | |
| 306 | GGAUUCAACACAA | 481 | CUUCCAGCCAUUG | 656 | CTTCCAGCCATTG | Exon 53 |
| | UGGCUGGAAG | | UGUUGAAUCC | | TGTTGAATCC | |
| 307 | AGGAUUCAACACA | 482 | CUUCCAGCCAUUG | 657 | CTTCCAGCCATTG | Exon 53 |
| | AUGGCUGGAAG | | UGUUGAAUCCU | | TGTTGAATCCT | |
| 308 | AAGGAUUCAACAC | 483 | CUUCCAGCCAUUG | 658 | CTTCCAGCCATTG | Exon 53 |
| | AAUGGCUGGAAG | | UGUUGAAUCCUU | | TGTTGAATCCTT | |
| 309 | AGCUUGAGUCAUG | 484 | GACCCUCCUUCCA | 659 | GACCCTCCTTCCA | Exon 53 |
| | GAAGGAGGGUC | | UGACUCAAGCU | | TGACTCAAGCT | |
| 310 | GGGUCCCUAUACA | 485 | GCAUCUACUGUAU | 660 | GCATCTACTGTAT | Exon 53 |
| | GUAGAUGC | | AGGGACCC | | AGGGACCC | |
| 311 | UAAAGGAUUCAAC | 486 | GCCAUUGUGUUGA | 661 | GCCATTGTGTTGA | Exon 53 |
| | ACAAUGGC | | AUCCUUUA | | ATCCTTTA | |
| 312 | UUAAAAUGUCUCC | 487 | GCUAGUCUGGAGG | 662 | GCTAGTCTGGAGG | Intron 52 |
| | UCCAGACUAGC | | AGACAUUUUAA | | AGACATTTTAA | |
| 313 | GAUUCAACACAAU | 488 | GCUUCCAGCCAUU | 663 | GCTTCCAGCCATT | Exon 53 |
| | GGCUGGAAGC | | GUGUUGAAUC | | GTGTTGAATC | |
| 314 | GGAUUCAACACAA | 489 | GCUUCCAGCCAUU | 664 | GCTTCCAGCCATT | Exon 53 |
| | UGGCUGGAAGC | | GUGUUGAAUCC | | GTGTTGAATCC | |
| 315 | AGGAUUCAACACA | 490 | GCUUCCAGCCAUU | 665 | GCTTCCAGCCATT | Exon 53 |
| | AUGGCUGGAAGC | | GUGUUGAAUCCU | | GTGTTGAATCCT | |
| 316 | CUUGAGUCAUGGA | 491 | GGACCCUCCUUCC | 666 | GGACCCTCCTTCC | Exon 53 |
| | AGGAGGGUCC | | AUGACUCAAG | | ATGACTCAAG | |
| 317 | GCUUGAGUCAUGG | 492 | GGACCCUCCUUCC | 667 | GGACCCTCCTTCC | Exon 53 |
| | AAGGAGGGUCC | | AUGACUCAAGC | | ATGACTCAAGC | |
| 318 | AGCUUGAGUCAUG | 493 | GGACCCUCCUUCC | 668 | GGACCCTCCTTCC | Exon 53 |
| | GAAGGAGGGUCC | | AUGACUCAAGCU | | ATGACTCAAGCT | |
| 319 | CUUGAGUCAUGGA | 494 | GGGACCCUCCUUC | 669 | GGGACCCTCCTTC | Exon 53 |
| | AGGAGGGUCCC | | CAUGACUCAAG | | CATGACTCAAG | |
| 320 | GCUUGAGUCAUGG | 495 | GGGACCCUCCUUC | 670 | GGGACCCTCCTTC | Exon 53 |
| | AAGGAGGGUCCC | | CAUGACUCAAGC | | CATGACTCAAGC | |
| 321 | GUCUCCUCCAGAC | 496 | GUAAAUGCUAGUC | 671 | GTAAATGCTAGTC | Intron 52 |
| | UAGCAUUUAC | | UGGAGGAGAC | | TGGAGGAGAC | |
| 322 | GUAAGUUUUUUAA | 497 | GUCCCAUGCUUGU | 672 | GTCCCATGCTTGT | Intron 52 |
| | CAAGCAUGGGAC | | UAAAAAACUUAC | | TAAAAAACTTAC | |
| 323 | AGCUGAAAUGAAC | 498 | GUCUACUGUUCAU | 673 | GTCTACTGTTCAT | Intron 53 |
| | AGUAGAC | | UUCAGCU | | TTCAGCT | |
| 324 | CAUCAUCCUAGCC | 499 | GUGUUAUGGCUAG | 674 | GTGTTATGGCTAG | Intron 52 |
| | AUAACAC | | GAUGAUG | | GATGATG | |
| 325 | GAUUCAACACAAU | 500 | UAGCUUCCAGCCA | 675 | TAGCTTCCAGCCA | Exon 53 |
| | GGCUGGAAGCUA | | UUGUGUUGAAUC | | TTGTGTTGAATC | |
| 326 | AACCAAGGUUAGU | 501 | UAUCUUUGAUACU | 676 | TATCTTTGATACT | Exon 53/intron 53 |
| | AUCAAAGAUA | | AACCUUGGUU | | AACCTTGGTT | junction |
| 327 | AGGAUUCAACACA | 502 | UCCAGCCAUUGUG | 677 | TCCAGCCATTGTG | Exon 53 |
| | AUGGCUGGA | | UUGAAUCCU | | TTGAATCCT | |
| 328 | AAGGAUUCAACAC | 503 | UCCAGCCAUUGUG | 678 | TCCAGCCATTGTG | Exon 53 |
| | AAUGGCUGGA | | UUGAAUCCUU | | TTGAATCCTT | |
| 329 | AAAGGAUUCAACA | 504 | UCCAGCCAUUGUG | 679 | TCCAGCCATTGTG | Exon 53 |
| | CAAUGGCUGGA | | UUGAAUCCUUU | | TTGAATCCTTT | |
| 330 | UAAAGGAUUCAAC | 505 | UCCAGCCAUUGUG | 680 | TCCAGCCATTGTG | Exon 53 |
| | ACAAUGGCUGGA | | UUGAAUCCUUUA | | TTGAATCCTTTA | |
| 331 | AUAUAUGUAUUCU | 506 | UCCUCAGGUCAGA | 681 | TCCTCAGGTCAGA | Intron 53 |
| | GACCUGAGGA | | AUACAUAUAU | | ATACATATAT | |
| 332 | GAACACCUUCAGA | 507 | UGCCUCCGGUUCU | 682 | TGCCTCCGGTTCT | Exon 53 |
| | ACCGGAGGCA | | GAAGGUGUUC | | GAAGGTGTTC | |
| 333 | GUUCAUCAUCCUA | 508 | UUAUGGCUAGGAU | 683 | TTATGGCTAGGAT | Intron 52 |
| | GCCAUAA | | GAUGAAC | | GATGAAC | |
| 334 | UUCAUCAUCCUAG | 509 | UUGUGUUAUGGCU | 684 | TTGTGTTATGGCT | Intron 52 |
| | CCAUAACACAA | | AGGAUGAUGAA | | AGGATGATGAA | |

^{†}Each thymine base (T) in any one of the oligonucleotides and/or target sequences provided in Table 8 may independently and optionally be replaced with a uracil base (U), and/or each U may independently and optionally be replaced with a T. Target sequences listed in Table 8 contain U's, but binding of a DMD-targeting oligonucleotide to RNA and/or DNA is contemplated.

In some embodiments, an oligonucleotide useful for targeting DMD (e.g., for exon skipping) targets a region of a DMD sequence. In some embodiments, an oligonucleotide useful for targeting DMD (e.g., for exon skipping) targets a region of a DMD RNA (e.g., the Dp427m transcript of SEQ ID NO: 130). In some embodiments, an oligonucleotide useful for targeting DMD (e.g., for exon skipping) comprises a region of complementarity to a DMD RNA (e.g., the Dp427m transcript of SEQ ID NO: 130). In some embodiments, an oligonucleotide useful for targeting DMD (e.g., for exon skipping) comprises a region of complementarity to an exon of a DMD RNA (e.g., SEQ ID NO: 131, 762, or 777). In some embodiments, an oligonucleotide useful for targeting DMD (e.g., for exon skipping) comprises a region of complementarity to an intron of a DMD RNA (e.g., SEQ ID NO: 754 or 770). In some embodiments, an oligonucleotide useful for targeting DMD (e.g., for exon skipping) comprises a region of complementarity to a portion of a DMD sequence (e.g., a sequence provided by any one of SEQ ID NOs: 753, 755-761, 763-769, and 771-776). Examples of DMD sequences are provided below. Each of the DMD sequences provided below include thymine nucleotides (T's), but it should be understood that each sequence can represent a DNA sequence or an RNA sequence in which any or all of the T's would be replaced with uracil nucleotides (U's).

Homo sapiens dystrophin (DMD), transcript variant Dp427m, mRNA (NCBI
Reference Sequence: NM_004006.2)
(SEQ ID NO: 130)
TCCTGGCATCAGTTACTGTGTTGACTCACTCAGTGTTGGGATCACTCACTTTCCCCCTACAGGACTCAGATCTGGG
AGGCAATTACCTTCGGAGAAAAACGAATAGGAAAAACTGAAGTGTTACTTTTTTTAAAGCTGCTGAAGTTTGTTGG
TTTCTCATTGTTTTTAAGCCTACTGGAGCAATAAAGTTTGAAGAACTTTTACCAGGTTTTTTTTATCGCTGCCTTG
ATATACACTTTTCAAAATGCTTTGGTGGGAAGAAGTAGAGGACTGTTATGAAAGAGAAGATGTTCAAAAGAAAACA
TTCACAAAATGGGTAAATGCACAATTTTCTAAGTTTGGGAAGCAGCATATTGAGAACCTCTTCAGTGACCTACAGG
ATGGGAGGCGCCTCCTAGACCTCCTCGAAGGCCTGACAGGGCAAAAACTGCCAAAAGAAAAAGGATCCACAAGAGT
TCATGCCCTGAACAATGTCAACAAGGCACTGCGGGTTTTGCAGAACAATAATGTTGATTTAGTGAATATTGGAAGT
ACTGACATCGTAGATGGAAATCATAAACTGACTCTTGGTTTGATTTGGAATATAATCCTCCACTGGCAGGTCAAAA
ATGTAATGAAAAATATCATGGCTGGATTGCAACAAACCAACAGTGAAAAGATTCTCCTGAGCTGGGTCCGACAATC
AACTCGTAATTATCCACAGGTTAATGTAATCAACTTCACCACCAGCTGGTCTGATGGCCTGGCTTTGAATGCTCTC
ATCCATAGTCATAGGCCAGACCTATTTGACTGGAATAGTGTGGTTTGCCAGCAGTCAGCCACACAACGACTGGAAC
ATGCATTCAACATCGCCAGATATCAATTAGGCATAGAGAAACTACTCGATCCTGAAGATGTTGATACCACCTATCC
AGATAAGAAGTCCATCTTAATGTACATCACATCACTCTTCCAAGTTTTGCCTCAACAAGTGAGCATTGAAGCCATC
CAGGAAGTGGAAATGTTGCCAAGGCCACCTAAAGTGACTAAAGAAGAACATTTTCAGTTACATCATCAAATGCACT
ATTCTCAACAGATCACGGTCAGTCTAGCACAGGGATATGAGAGAACTTCTTCCCCTAAGCCTCGATTCAAGAGCTA
TGCCTACACACAGGCTGCTTATGTCACCACCTCTGACCCTACACGGAGCCCATTTCCTTCACAGCATTTGGAAGCT
CCTGAAGACAAGTCATTTGGCAGTTCATTGATGGAGAGTGAAGTAAACCTGGACCGTTATCAAACAGCTTTAGAAG
AAGTATTATCGTGGCTTCTTTCTGCTGAGGACACATTGCAAGCACAAGGAGAGATTTCTAATGATGTGGAAGTGGT
GAAAGACCAGTTTCATACTCATGAGGGGTACATGATGGATTTGACAGCCCATCAGGGCCGGGTTGGTAATATTCTA
CAATTGGGAAGTAAGCTGATTGGAACAGGAAAATTATCAGAAGATGAAGAAACTGAAGTACAAGAGCAGATGAATC
TCCTAAATTCAAGATGGGAATGCCTCAGGGTAGCTAGCATGGAAAAACAAAGCAATTTACATAGAGTTTTAATGGA
TCTCCAGAATCAGAAACTGAAAGAGTTGAATGACTGGCTAACAAAAACAGAAGAAAGAACAAGGAAAATGGAGGAA
GAGCCTCTTGGACCTGATCTTGAAGACCTAAAACGCCAAGTACAACAACATAAGGTGCTTCAAGAAGATCTAGAAC
AAGAACAAGTCAGGGTCAATTCTCTCACTCACATGGTGGTGGTAGTTGATGAATCTAGTGGAGATCACGCAACTGC
TGCTTTGGAAGAACAACTTAAGGTATTGGGAGATCGATGGGCAAACATCTGTAGATGGACAGAAGACCGCTGGGTT
CTTTTACAAGACATCCTTCTCAAATGGCAACGTCTTACTGAAGAACAGTGCCTTTTTAGTGCATGGCTTTCAGAAA
AAGAAGATGCAGTGAACAAGATTCACACAACTGGCTTTAAAGATCAAAATGAAATGTTATCAAGTCTTCAAAAACT
GGCCGTTTTAAAAGCGGATCTAGAAAAGAAAAAGCAATCCATGGGCAAACTGTATTCACTCAAACAAGATCTTCTT
TCAACACTGAAGAATAAGTCAGTGACCCAGAAGACGGAAGCATGGCTGGATAACTTTGCCCGGTGTTGGGATAATT
TAGTCCAAAAACTTGAAAAGAGTACAGCACAGATTTCACAGGCTGTCACCACCACTCAGCCATCACTAACACAGAC
AACTGTAATGGAAACAGTAACTACGGTGACCACAAGGGAACAGATCCTGGTAAAGCATGCTCAAGAGGAACTTCCA
CCACCACCTCCCCAAAAGAAGAGGCAGATTACTGTGGATTCTGAAATTAGGAAAAGGTTGGATGTTGATATAACTG
AACTTCACAGCTGGATTACTCGCTCAGAAGCTGTGTTGCAGAGTCCTGAATTTGCAATCTTTCGGAAGGAAGGCAA
CTTCTCAGACTTAAAAGAAAAAGTCAATGCCATAGAGCGAGAAAAAGCTGAGAAGTTCAGAAAACTGCAAGATGCC
AGCAGATCAGCTCAGGCCCTGGTGGAACAGATGGTGAATGAGGGTGTTAATGCAGATAGCATCAAACAAGCCTCAG
AACAACTGAACAGCCGGTGGATCGAATTCTGCCAGTTGCTAAGTGAGAGACTTAACTGGCTGGAGTATCAGAACAA
CATCATCGCTTTCTATAATCAGCTACAACAATTGGAGCAGATGACAACTACTGCTGAAAACTGGTTGAAAATCCAA
CCCACCACCCCATCAGAGCCAACAGCAATTAAAAGTCAGTTAAAAATTTGTAAGGATGAAGTCAACCGGCTATCAG
GTCTTCAACCTCAAATTGAACGATTAAAAATTCAAAGCATAGCCCTGAAAGAGAAAGGACAAGGACCCATGTTCCT
GGATGCAGACTTTGTGGCCTTTACAAATCATTTTAAGCAAGTCTTTTCTGATGTGCAGGCCAGAGAGAAAGAGCTA
CAGACAATTTTTGACACTTTGCCACCAATGCGCTATCAGGAGACCATGAGTGCCATCAGGACATGGGTCCAGCAGT
CAGAAACCAAACTCTCCATACCTCAACTTAGTGTCACCGACTATGAAATCATGGAGCAGAGACTCGGGGAATTGCA
GGCTTTACAAAGTTCTCTGCAAGAGCAACAAAGTGGCCTATACTATCTCAGCACCACTGTGAAAGAGATGTCGAAG
AAAGCGCCCTCTGAAATTAGCCGGAAATATCAATCAGAATTTGAAGAAATTGAGGGACGCTGGAAGAAGCTCTCCT
CCCAGCTGGTTGAGCATTGTCAAAAGCTAGAGGAGCAAATGAATAAACTCCGAAAAATTCAGAATCACATACAAAC
CCTGAAGAAATGGATGGCTGAAGTTGATGTTTTTCTGAAGGAGGAATGGCCTGCCCTTGGGGATTCAGAAATTCTA
AAAAAGCAGCTGAAACAGTGCAGACTTTTAGTCAGTGATATTCAGACAATTCAGCCCAGTCTAAACAGTGTCAATG
AAGGTGGGCAGAAGATAAAGAATGAAGCAGAGCCAGAGTTTGCTTCGAGACTTGAGACAGAACTCAAAGAACTTAA
CACTCAGTGGGATCACATGTGCCAACAGGTCTATGCCAGAAAGGAGGCCTTGAAGGGAGGTTTGGAGAAAACTGTA
AGCCTCCAGAAAGATCTATCAGAGATGCACGAATGGATGACACAAGCTGAAGAAGAGTATCTTGAGAGAGATTTTG
AATATAAAACTCCAGATGAATTACAGAAAGCAGTTGAAGAGATGAAGAGAGCTAAAGAAGAGGCCCAACAAAAAGA
AGCGAAAGTGAAACTCCTTACTGAGTCTGTAAATAGTGTCATAGCTCAAGCTCCACCTGTAGCACAAGAGGCCTTA
AAAAAGGAACTTGAAACTCTAACCACCAACTACCAGTGGCTCTGCACTAGGCTGAATGGGAAATGCAAGACTTTGG
AAGAAGTTTGGGCATGTTGGCATGAGTTATTGTCATACTTGGAGAAAGCAAACAAGTGGCTAAATGAAGTAGAATT
TAAACTTAAAACCACTGAAAACATTCCTGGCGGAGCTGAGGAAATCTCTGAGGTGCTAGATTCACTTGAAAATTTG
ATGCGACATTCAGAGGATAACCCAAATCAGATTCGCATATTGGCACAGACCCTAACAGATGGCGGAGTCATGGATG
AGCTAATCAATGAGGAACTTGAGACATTTAATTCTCGTTGGAGGGAACTACATGAAGAGGCTGTAAGGAGGCAAAA
GTTGCTTGAACAGAGCATCCAGTCTGCCCAGGAGACTGAAAAATCCTTACACTTAATCCAGGAGTCCCTCACATTC
ATTGACAAGCAGTTGGCAGCTTATATTGCAGACAAGGTGGACGCAGCTCAAATGCCTCAGGAAGCCCAGAAAATCC
AATCTGATTTGACAAGTCATGAGATCAGTTTAGAAGAAATGAAGAAACATAATCAGGGGAAGGAGGCTGCCCAAAG
AGTCCTGTCTCAGATTGATGTTGCACAGAAAAAATTACAAGATGTCTCCATGAAGTTTCGATTATTCCAGAAACCA
GCCAATTTTGAGCAGCGTCTACAAGAAAGTAAGATGATTTTAGATGAAGTGAAGATGCACTTGCCTGCATTGGAAA
CAAAGAGTGTGGAACAGGAAGTAGTACAGTCACAGCTAAATCATTGTGTGAACTTGTATAAAAGTCTGAGTGAAGT
GAAGTCTGAAGTGGAAATGGTGATAAAGACTGGACGTCAGATTGTACAGAAAAAGCAGACGGAAAATCCCAAAGAA
CTTGATGAAAGAGTAACAGCTTTGAAATTGCATTATAATGAGCTGGGAGCAAAGGTAACAGAAAGAAAGCAACAGT
TGGAGAAATGCTTGAAATTGTCCCGTAAGATGCGAAAGGAAATGAATGTCTTGACAGAATGGCTGGCAGCTACAGA
TATGGAATTGACAAAGAGATCAGCAGTTGAAGGAATGCCTAGTAATTTGGATTCTGAAGTTGCCTGGGGAAAGGCT
ACTCAAAAAGAGATTGAGAAACAGAAGGTGCACCTGAAGAGTATCACAGAGGTAGGAGAGGCCTTGAAAACAGTTT
TGGGCAAGAAGGAGACGTTGGTGGAAGATAAACTCAGTCTTCTGAATAGTAACTGGATAGCTGTCACCTCCCGAGC
AGAAGAGTGGTTAAATCTTTTGTTGGAATACCAGAAACACATGGAAACTTTTGACCAGAATGTGGACCACATCACA
AAGTGGATCATTCAGGCTGACACACTTTTGGATGAATCAGAGAAAAAGAAACCCCAGCAAAAAGAAGACGTGCTTA
AGCGTTTAAAGGCAGAACTGAATGACATACGCCCAAAGGTGGACTCTACACGTGACCAAGCAGCAAACTTGATGGC
AAACCGCGGTGACCACTGCAGGAAATTAGTAGAGCCCCAAATCTCAGAGCTCAACCATCGATTTGCAGCCATTTCA
CACAGAATTAAGACTGGAAAGGCCTCCATTCCTTTGAAGGAATTGGAGCAGTTTAACTCAGATATACAAAAATTGC
TTGAACCACTGGAGGCTGAAATTCAGCAGGGGGTGAATCTGAAAGAGGAAGACTTCAATAAAGATATGAATGAAGA
CAATGAGGGTACTGTAAAAGAATTGTTGCAAAGAGGAGACAACTTACAACAAAGAATCACAGATGAGAGAAAGCGA
GAGGAAATAAAGATAAAACAGCAGCTGTTACAGACAAAACATAATGCTCTCAAGGATTTGAGGTCTCAAAGAAGAA
AAAAGGCTCTAGAAATTTCTCATCAGTGGTATCAGTACAAGAGGCAGGCTGATGATCTCCTGAAATGCTTGGATGA
CATTGAAAAAAAATTAGCCAGCCTACCTGAGCCCAGAGATGAAAGGAAAATAAAGGAAATTGATCGGGAATTGCAG
AAGAAGAAAGAGGAGCTGAATGCAGTGCGTAGGCAAGCTGAGGGCTTGTCTGAGGATGGGGCCGCAATGGCAGTGG
AGCCAACTCAGATCCAGCTCAGCAAGCGCTGGCGGGAAATTGAGAGCAAATTTGCTCAGTTTCGAAGACTCAACTT
TGCACAAATTCACACTGTCCGTGAAGAAACGATGATGGTGATGACTGAAGACATGCCTTTGGAAATTTCTTATGTG
CCTTCTACTTATTTGACTGAAATCACTCATGTCTCACAAGCCCTATTAGAAGTGGAACAACTTCTCAATGCTCCTG
ACCTCTGTGCTAAGGACTTTGAAGATCTCTTTAAGCAAGAGGAGTCTCTGAAGAATATAAAAGATAGTCTACAACA
AAGCTCAGGTCGGATTGACATTATTCATAGCAAGAAGACAGCAGCATTGCAAAGTGCAACGCCTGTGGAAAGGGTG
AAGCTACAGGAAGCTCTCTCCCAGCTTGATTTCCAATGGGAAAAAGTTAACAAAATGTACAAGGACCGACAAGGGC
GATTTGACAGATCTGTTGAGAAATGGCGGCGTTTTCATTATGATATAAAGATATTTAATCAGTGGCTAACAGAAGC
TGAACAGTTTCTCAGAAAGACACAAATTCCTGAGAATTGGGAACATGCTAAATACAAATGGTATCTTAAGGAACTC
CAGGATGGCATTGGGCAGCGGCAAACTGTTGTCAGAACATTGAATGCAACTGGGGAAGAAATAATTCAGCAATCCT
CAAAAACAGATGCCAGTATTCTACAGGAAAAATTGGGAAGCCTGAATCTGCGGTGGCAGGAGGTCTGCAAACAGCT
GTCAGACAGAAAAAAGAGGCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGCAGCAACTAAAAGAAAAGC
TTGAGCAAGTCAAGTTACTGGTGGAAGAGTTGCCCCTGCGCCAGGGAATTCTCAAACAATTAAATGAAACTGGAGG
ACCCGTGCTTGTAAGTGCTCCCATAAGCCCAGAAGAGCAAGATAAACTTGAAAATAAGCTCAAGCAGACAAATCTC
CAGTGGATAAAGGTTTCCAGAGCTTTACCTGAGAAACAAGGAGAAATTGAAGCTCAAATAAAAGACCTTGGGCAGC
TTGAAAAAAAGCTTGAAGACCTTGAAGAGCAGTTAAATCATCTGCTGCTGTGGTTATCTCCTATTAGGAATCAGTT
GGAAATTTATAACCAACCAAACCAAGAAGGACCATTTGACGTTCAGGAAACTGAAATAGCAGTTCAAGCTAAACAA
CCGGATGTGGAAGAGATTTTGTCTAAAGGGCAGCATTTGTACAAGGAAAAACCAGCCACTCAGCCAGTGAAGAGGA
AGTTAGAAGATCTGAGCTCTGAGTGGAAGGCGGTAAACCGTTTACTTCAAGAGCTGAGGGCAAAGCAGCCTGACCT
AGCTCCTGGACTGACCACTATTGGAGCCTCTCCTACTCAGACTGTTACTCTGGTGACACAACCTGTGGTTACTAAG
GAAACTGCCATCTCCAAACTAGAAATGCCATCTTCCTTGATGTTGGAGGTACCTGCTCTGGCAGATTTCAACCGGG
CTTGGACAGAACTTACCGACTGGCTTTCTCTGCTTGATCAAGTTATAAAATCACAGAGGGTGATGGTGGGTGACCT
TGAGGATATCAACGAGATGATCATCAAGCAGAAGGCAACAATGCAGGATTTGGAACAGAGGCGTCCCCAGTTGGAA
GAACTCATTACCGCTGCCCAAAATTTGAAAAACAAGACCAGCAATCAAGAGGCTAGAACAATCATTACGGATCGAA
TTGAAAGAATTCAGAATCAGTGGGATGAAGTACAAGAACACCTTCAGAACCGGAGGCAACAGTTGAATGAAATGTT
AAAGGATTCAACACAATGGCTGGAAGCTAAGGAAGAAGCTGAGCAGGTCTTAGGACAGGCCAGAGCCAAGCTTGAG
TCATGGAAGGAGGGTCCCTATACAGTAGATGCAATCCAAAAGAAAATCACAGAAACCAAGCAGTTGGCCAAAGACC
TCCGCCAGTGGCAGACAAATGTAGATGTGGCAAATGACTTGGCCCTGAAACTTCTCCGGGATTATTCTGCAGATGA
TACCAGAAAAGTCCACATGATAACAGAGAATATCAATGCCTCTTGGAGAAGCATTCATAAAAGGGTGAGTGAGCGA
GAGGCTGCTTTGGAAGAAACTCATAGATTACTGCAACAGTTCCCCCTGGACCTGGAAAAGTTTCTTGCCTGGCTTA
CAGAAGCTGAAACAACTGCCAATGTCCTACAGGATGCTACCCGTAAGGAAAGGCTCCTAGAAGACTCCAAGGGAGT
AAAAGAGCTGATGAAACAATGGCAAGACCTCCAAGGTGAAATTGAAGCTCACACAGATGTTTATCACAACCTGGAT
GAAAACAGCCAAAAAATCCTGAGATCCCTGGAAGGTTCCGATGATGCAGTCCTGTTACAAAGACGTTTGGATAACA
TGAACTTCAAGTGGAGTGAACTTCGGAAAAAGTCTCTCAACATTAGGTCCCATTTGGAAGCCAGTTCTGACCAGTG
GAAGCGTCTGCACCTTTCTCTGCAGGAACTTCTGGTGTGGCTACAGCTGAAAGATGATGAATTAAGCCGGCAGGCA
CCTATTGGAGGCGACTTTCCAGCAGTTCAGAAGCAGAACGATGTACATAGGGCCTTCAAGAGGGAATTGAAAACTA
AAGAACCTGTAATCATGAGTACTCTTGAGACTGTACGAATATTTCTGACAGAGCAGCCTTTGGAAGGACTAGAGAA
ACTCTACCAGGAGCCCAGAGAGCTGCCTCCTGAGGAGAGAGCCCAGAATGTCACTCGGCTTCTACGAAAGCAGGCT
GAGGAGGTCAATACTGAGTGGGAAAAATTGAACCTGCACTCCGCTGACTGGCAGAGAAAAATAGATGAGACCCTTG
AAAGACTCCAGGAACTTCAAGAGGCCACGGATGAGCTGGACCTCAAGCTGCGCCAAGCTGAGGTGATCAAGGGATC
CTGGCAGCCCGTGGGCGATCTCCTCATTGACTCTCTCCAAGATCACCTCGAGAAAGTCAAGGCACTTCGAGGAGAA
ATTGCGCCTCTGAAAGAGAACGTGAGCCACGTCAATGACCTTGCTCGCCAGCTTACCACTTTGGGCATTCAGCTCT
CACCGTATAACCTCAGCACTCTGGAAGACCTGAACACCAGATGGAAGCTTCTGCAGGTGGCCGTCGAGGACCGAGT
CAGGCAGCTGCATGAAGCCCACAGGGACTTTGGTCCAGCATCTCAGCACTTTCTTTCCACGTCTGTCCAGGGTCCC
TGGGAGAGAGCCATCTCGCCAAACAAAGTGCCCTACTATATCAACCACGAGACTCAAACAACTTGCTGGGACCATC
CCAAAATGACAGAGCTCTACCAGTCTTTAGCTGACCTGAATAATGTCAGATTCTCAGCTTATAGGACTGCCATGAA
ACTCCGAAGACTGCAGAAGGCCCTTTGCTTGGATCTCTTGAGCCTGTCAGCTGCATGTGATGCCTTGGACCAGCAC
AACCTCAAGCAAAATGACCAGCCCATGGATATCCTGCAGATTATTAATTGTTTGACCACTATTTATGACCGCCTGG
AGCAAGAGCACAACAATTTGGTCAACGTCCCTCTCTGCGTGGATATGTGTCTGAACTGGCTGCTGAATGTTTATGA
TACGGGACGAACAGGGAGGATCCGTGTCCTGTCTTTTAAAACTGGCATCATTTCCCTGTGTAAAGCACATTTGGAA
GACAAGTACAGATACCTTTTCAAGCAAGTGGCAAGTTCAACAGGATTTTGTGACCAGCGCAGGCTGGGCCTCCTTC
TGCATGATTCTATCCAAATTCCAAGACAGTTGGGTGAAGTTGCATCCTTTGGGGGCAGTAACATTGAGCCAAGTGT
CCGGAGCTGCTTCCAATTTGCTAATAATAAGCCAGAGATCGAAGCGGCCCTCTTCCTAGACTGGATGAGACTGGAA
CCCCAGTCCATGGTGTGGCTGCCCGTCCTGCACAGAGTGGCTGCTGCAGAAACTGCCAAGCATCAGGCCAAATGTA
ACATCTGCAAAGAGTGTCCAATCATTGGATTCAGGTACAGGAGTCTAAAGCACTTTAATTATGACATCTGCCAAAG
CTGCTTTTTTTCTGGTCGAGTTGCAAAAGGCCATAAAATGCACTATCCCATGGTGGAATATTGCACTCCGACTACA
TCAGGAGAAGATGTTCGAGACTTTGCCAAGGTACTAAAAAACAAATTTCGAACCAAAAGGTATTTTGCGAAGCATC
CCCGAATGGGCTACCTGCCAGTGCAGACTGTCTTAGAGGGGGACAACATGGAAACTCCCGTTACTCTGATCAACTT
CTGGCCAGTAGATTCTGCGCCTGCCTCGTCCCCTCAGCTTTCACACGATGATACTCATTCACGCATTGAACATTAT
GCTAGCAGGCTAGCAGAAATGGAAAACAGCAATGGATCTTATCTAAATGATAGCATCTCTCCTAATGAGAGCATAG
ATGATGAACATTTGTTAATCCAGCATTACTGCCAAAGTTTGAACCAGGACTCCCCCCTGAGCCAGCCTCGTAGTCC
TGCCCAGATCTTGATTTCCTTAGAGAGTGAGGAAAGAGGGGAGCTAGAGAGAATCCTAGCAGATCTTGAGGAAGAA
AACAGGAATCTGCAAGCAGAATATGACCGTCTAAAGCAGCAGCACGAACATAAAGGCCTGTCCCCACTGCCGTCCC
CTCCTGAAATGATGCCCACCTCTCCCCAGAGTCCCCGGGATGCTGAGCTCATTGCTGAGGCCAAGCTACTGCGTCA
ACACAAAGGCCGCCTGGAAGCCAGGATGCAAATCCTGGAAGACCACAATAAACAGCTGGAGTCACAGTTACACAGG
CTAAGGCAGCTGCTGGAGCAACCCCAGGCAGAGGCCAAAGTGAATGGCACAACGGTGTCCTCTCCTTCTACCTCTC
TACAGAGGTCCGACAGCAGTCAGCCTATGCTGCTCCGAGTGGTTGGCAGTCAAACTTCGGACTCCATGGGTGAGGA
AGATCTTCTCAGTCCTCCCCAGGACACAAGCACAGGGTTAGAGGAGGTGATGGAGCAACTCAACAACTCCTTCCCT
AGTTCAAGAGGAAGAAATACCCCTGGAAAGCCAATGAGAGAGGACACAATGTAGGAAGTCTTTTCCACATGGCAGA
TGATTTGGGCAGAGCGATGGAGTCCTTAGTATCAGTCATGACAGATGAAGAAGGAGCAGAATAAATGTTTTACAAC
TCCTGATTCCCGCATGGTTTTTATAATATTCATACAACAAAGAGGATTAGACAGTAAGAGTTTACAAGAAATAAAT
CTATATTTTTGTGAAGGGTAGTGGTATTATACTGTAGATTTCAGTAGTTTCTAAGTCTGTTATTGTTTTGTTAACA
ATGGCAGGTTTTACACGTCTATGCAATTGTACAAAAAAGTTATAAGAAAACTACATGTAAAATCTTGATAGCTAAA
TAACTTGCCATTTCTTTATATGGAACGCATTTTGGGTTGTTTAAAAATTTATAACAGTTATAAAGAAAGATTGTAA
ACTAAAGTGTGCTTTATAAAAAAAAGTTGTTTATAAAAACCCCTAAAAACAAAACAAACACACACACACACACATA
CACACACACACACAAAACTTTGAGGCAGCGCATTGTTTTGCATCCTTTTGGCGTGATATCCATATGAAATTCATGG
CTTTTTCTTTTTTTGCATATTAAAGATAAGACTTCCTCTACCACCACACCAAATGACTACTACACACTGCTCATTT
GAGAACTGTCAGCTGAGTGGGGCAGGCTTGAGTTTTCATTTCATATATCTATATGTCTATAAGTATATAAATACTA
TAGTTATATAGATAAAGAGATACGAATTTCTATAGACTGACTTTTTCCATTTTTTAAATGTTCATGTCACATCCTA
ATAGAAAGAAATTACTTCTAGTCAGTCATCCAGGCTTACCTGCTTGGTCTAGAATGGATTTTTCCCGGAGCCGGAA
GCCAGGAGGAAACTACACCACACTAAAACATTGTCTACAGCTCCAGATGTTTCTCATTTTAAACAACTTTCCACTG
ACAACGAAAGTAAAGTAAAGTATTGGATTTTTTTAAAGGGAACATGTGAATGAATACACAGGACTTATTATATCAG
AGTGAGTAATCGGTTGGTTGGTTGATTGATTGATTGATTGATACATTCAGCTTCCTGCTGCTAGCAATGCCACGAT
TTAGATTTAATGATGCTTCAGTGGAAATCAATCAGAAGGTATTCTGACCTTGTGAACATCAGAAGGTATTTTTTAA
CTCCCAAGCAGTAGCAGGACGATGATAGGGCTGGAGGGCTATGGATTCCCAGCCCATCCCTGTGAAGGAGTAGGCC
ACTCTTTAAGTGAAGGATTGGATGATTGTTCATAATACATAAAGTTCTCTGTAATTACAACTAAATTATTATGCCC
TCTTCTCACAGTCAAAAGGAACTGGGTGGTTTGGTTTTTGTTGCTTTTTTAGATTTATTGTCCCATGTGGGATGAG
TTTTTAAATGCCACAAGACATAATTTAAAATAAATAAACTTTGGGAAAAGGTGTAAAACAGTAGCCCCATCACATT
TGTGATACTGACAGGTATCAACCCAGAAGCCCATGAACTGTGTTTCCATCCTTTGCATTTCTCTGCGAGTAGTTCC
ACACAGGTTTGTAAGTAAGTAAGAAAGAAGGCAAATTGATTCAAATGTTACAAAAAAACCCTTCTTGGTGGATTAG
ACAGGTTAAATATATAAACAAACAAACAAAAATTGCTCAAAAAAGAGGAGAAAAGCTCAAGAGGAAAAGCTAAGGA
CTGGTAGGAAAAAGCTTTACTCTTTCATGCCATTTTATTTCTTTTTGATTTTTAAATCATTCATTCAATAGATACC
ACCGTGTGACCTATAATTTTGCAAATCTGTTACCTCTGACATCAAGTGTAATTAGCTTTTGGAGAGTGGGCTGACA
TCAAGTGTAATTAGCTTTTGGAGAGTGGGTTTTGTCCATTATTAATAATTAATTAATTAACATCAAACACGGCTTC
TCATGCTATTTCTACCTCACTTTGGTTTTGGGGTGTTCCTGATAATTGTGCACACCTGAGTTCACAGCTTCACCAC
TTGTCCATTGCGTTATTTTCTTTTTCCTTTATAATTCTTTCTTTTTCCTTCATAATTTTCAAAAGAAAACCCAAAG
CTCTAAGGTAACAAATTACCAAATTACATGAAGATTTGGTTTTTGTCTTGCATTTTTTTCCTTTATGTGACGCTGG
ACCTTTTCTTTACCCAAGGATTTTTAAAACTCAGATTTAAAACAAGGGGTTACTTTACATCCTACTAAGAAGTTTA
AGTAAGTAAGTTTCATTCTAAAATCAGAGGTAAATAGAGTGCATAAATAATTTTGTTTTAATCTTTTTGTTTTTCT
TTTAGACACATTAGCTCTGGAGTGAGTCTGTCATAATATTTGAACAAAAATTGAGAGCTTTATTGCTGCATTTTAA
GCATAATTAATTTGGACATTATTTCGTGTTGTGTTCTTTATAACCACCAAGTATTAAACTGTAAATCATAATGTAA
CTGAAGCATAAACATCACATGGCATGTTTTGTCATTGTTTTCAGGTACTGAGTTCTTACTTGAGTATCATAATATA
TTGTGTTTTAACACCAACACTGTAACATTTACGAATTATTTTTTTAAACTTCAGTTTTACTGCATTTTCACAACAT
ATCAGACTTCACCAAATATATGCCTTACTATTGTATTATAGTACTGCTTTACTGTGTATCTCAATAAAGCACGCAG
TTATGTTAC
Homo sapiens dystrophin (DMD), transcript variant Dp427m, exon 52
(nucleotide positions 7787-7904 of NCBI Reference Sequence: NM_004006.2; nucleotide
positions 1614862-1614979 of NCBI Reference Sequence: NG_012232.1)
(SEQ ID NO: 131)
GCAACAATGCAGGATTTGGAACAGAGGCGTCCCCAGTTGGAAGAACTCATTACCGCTGCCCAAAATTTGAAAAACA
AGACCAGCAATCAAGAGGCTAGAACAATCATTACGGATCGAA
Homo sapiens dystrophin (DMD) exon 52/intron 52 junction (nucleotide
positions 1614950-1615009 of NCBI Reference Sequence: NG_012232.1)
(SEQ ID NO: 753)
AAGAGGCTAGAACAATCATTACGGATCGAAGTAAGTTTTTTAACAAGCATGGGACACACA
Homo sapiens dystrophin (DMD), intron 52 (nucleotide positions 1614980-
1665023 of NCBI Reference Sequence: NG_012232.1)
(SEQ ID NO: 754)
GTAAGTTTTTTAACAAGCATGGGACACACAAAGCAAGATGCATGACAAGTTTCAATAAAAACTTAAGTTCATATAT
CCCCCTCACATTTATAAAAATAATGTGAAATAATTGTAAATGATAACAATTGTGCTGAGATTTTCAGTCCATAATG
TTACCTTTTAATAAATGAATGTAATTCCATTGAATAGAAGAAATACATTTTTAAATCAATTCAGGGCTTATATAGT
TGCAAAGCATGCATTGATGGGTGTGGTGACCACAGTGTGGCAGAACATTTGTGGCAGAACATTTGTTCTTTAGTTG
TCATCTGGGCTGGCATCCATGGAGATGCCAGTCTCTCCCTCATATCCTTGGCTGTTGGTCCAAGCAGGCAGTGGCT
TCTTCCTGGGCCATCTTTCATTCCCATGTGCAGTGACTTTCAGATCTGGATATCTCTCCGCTACTTTGATGCCCCC
ATTTTGTAATATCAAAAATCATCGTACTGTACCTTATGCCGTAGTAGGGTGGGCAGGAACTTTGGTAAGACCCATC
TGACTAGACGCTGTGCATATTCTTTTCTTCTGACATACACTCCTATCCATTTAATGGGGAGAGTGATTCGCAGTGA
TTGTGTGTTGTGTCAGTGAGTTTCCATGGGGTCAGGAAGAGTGACAGACGAAGGAGTAGGGGAAACTCGCCACCCG
GTTTCCCTCAGAGATTCTCCTCAGAAATGAGGTCCAAGTCAGCTCTGCTTTCAGGTTCTCTCAGATCTCTCTCGGT
TTCTTCACTTCTCTCTACCTTCCTTCCCTCCAGGGACACACACTGGTATTGAATTTTCTTGCTTCCTCTGCAATAT
CCCCTCATTTTCCTTCCCACAACCCGAAGAATCCTTTGTAGTGCAGGAAGGAGGAAAACCTTTCAGCCATCTTTTT
TTTTCTCTTTGAAATCTTTTGTCTTTTACCAGGCTTAGACTTTTCAAACTCGGAAACCATGAGAGTCTATATCTTC
ATAATTTATATTCTGCTATGTTAACCCTTCCCTAAGGAAATGACTAGTTGTCAATATGTTGGGGAAAGTGAAAGAG
TAACCAGAGTAAAAGGTTAATATTTTAAAATATTATTAGTCATACTTCCACATATTGGGTAAGTACTTATTGATAA
TAGCTAGTATTTATTCAGTACCTCATAAGCATTAGATGGTGTGTGCACATGTGTGTGTTTCTGTGTGTTTCTGTGT
GTGTGTACAAAATCTTTACAGAATCTTGTGAGCTATATTTTATAATCCCCATTTTATAGACGAGAAAACAGGTTCC
AAGAACAGTTACTCGGCCGGGCGCGGTGGCTCACGCCTGTAATCCCAGCACTTGGGGAGGCCGAGGCGGGCGGATC
ACGAGGTCAGGAGATCGAGACCATCCTGGCTAACACGGTGAAACCCCGTCTCTACTAAAAATACAAAAAAAAATTA
GCCGGGCGTGGTGGCGGGCGCCTGTAGTCCCAGCTACTTGGGAGGCTGAGGCAGGAGAATGGCGTGAACCCGGGAG
GCGGAGCTTGCAGTGAGCCGAGATTGCGCCACTGCACTCCAGCCTGGGCGACAGAGCGAGACTGCGTCGTTTACAA
ACAAGCAAAGAACAGAGCTTGGAATTTGTAAATGTCTCTTTCTGACTTGAAAATCTGCGCTCTTTCTAGTATGTTT
ACCATTTCCCATCTTGTTTTGTTGCTTTTGTTAATGACCTTAATCATTGTACTAAGACTAAATACTTCTTTTGTCT
GAAATTATGTATGTTTTGATTCACTTCCTAAAGACATGTCTTCTTTCAGTTGTAGTGATTGCTAATTAAAATAGGC
TGTTCTTGGTTTTGAAAGTTTAACTCTTTATTGTTGCTTAAACAATGAATGTGGATGTATGCTAATGTATTATTTC
AGTAACACTAGCCACTATAACAGGTAATCTCCCAAATCTTGGAGGCTTACTACAGCAGAAATTTACTTCTTATTTT
AGTGCAGTCCAAAATAAGCAGCCCTCTATGAAGTCATTCAGGAACCCAAGCTCCTTCCATCTTTTGTGTCCAGAGC
ATAAGACTCATATGCATTTAATGGGCAGATGAAGAATGAGTATTACTCATGAAAATTTGTCATGAGCCACGCCTGG
AAATGGTGTGTGTTAATTTTGTTCATATTCTGTTGGATGGATCTTGTCACATGGTCACACCTAACTGCGAGGGAAT
TAGGGAAATAGTGTACACTGTTGAGCCTAGGAAGAGGAACAGATTTGGTAAGATAGCCATACAATTGATTTAGCAG
AGCATTCCTCCACCATACTGGAACCTTGAGGGTTCTCCAACAAGTTGCAACACACTGACCCAAAAGAGTGAATCTT
AGTGAGTGATTTACTCAATATGAGACTGAATTCCCATTACACAGGTAAGTGATCAGTTTCTTGCCTGAGAAATATG
GAAATTTGGCAGTGAGGTTTATGCAAATCTGAACATACTATACAGAGAGTCATTTGTTATTTTACTAATGAAAAAT
CACTTCAATTTTTTCCCTAAGAGGAAGACAATATGAATGTATTATACAGTATTTGTTCAACATTGTCTGAACATTT
TCCATTATCCCCTGCATTTTTTTTTCATATTGCATTGACTTTTTCATGATAGAGATTAAAATTGAATGACCGGAGG
GCAAGTTTGTATCTCTCTGCCAATATTCAGTGATTTGAATAGTTCTTCTTTTCTAAGCTTTTGTCTTTTAGGAATG
AATACCTTTATGAATATTTGCAGCCTAGTGGAAAGGCTGTAATCCAAATGGTCCAAGAAAGCATTTTCTTAAAAGC
AAGTGTCTGTCGAGATGTGTCATAGCGCTTATTAAGAGTCTAAGCTGGAATCTTAGTTCCAAATATGCCTGGAGCC
TCTAAATGGTACCAGGATAATTCTGACAAGATATGTTTATCTAAACAATGTCATTTGCAGCCCTGCTACACTTCAG
TTTATCTCTCCCTTTGAAATCTATAAAATGGGATGGAAATTCAATACTCATAAACTTTTCGATCGTGTTCAAAATA
GAATTTTTCTTAGTAAAGATTTGGTTTTCAGGAAAAGGACAGAAATAAAATACTTGCTCATAAAATTGTATTTTCT
CATTTGATGATTTTGGTCTTCCTTTTTATTGCCATGAAACTTCTAGAAATGCTCAAAAAGAAATCAGCTAAATAAA
GAAAAAATAGTTAATATATGTATGTAATACTATATTGAAACATTTTTCTTTCTCTGGTAAATCCCATTTCATAACT
TTGAACAGTTGGGAAAATCTATACATAGTTATTGCAGTCTATCAAGAGAAAAGTTCAGTACAAAGCTATTTATGTC
TACTAGAAATATTCATGTTAAACTTCAAGTAATTGGGTGTGCAAGCCACCACCATGTTTTACTATATGAAACTATT
ACCGTGGTATCTGTTGTATTCAGGTAATTATATTGATGGAAATCATGCAGTAATAATCTAGGTAAGAGAGTAAATT
TTGTCTAAATCAGATCAAATGAAAAATTCTCCCTCTTTCTAATATTCGAATTGCTCATTTTTCTTTAACTCTTTGG
TGTCTGAATTTGTCAATCATTCCTGGCCATTTTCTTCTGCAAAAGGGCTGGGTCAGGGGACCAAAAGCAGATAAGA
TTAGAAGAATTTAAATTTTCTTCCTTGGAGGCGTCTGAATTACATGAAACTCTTGTTCGTGTCTGTTAATACTGCA
AGGCATAATACCATAATACCTTGCATAGCAGTGAAGAGGATTTGGAAAGATAAAACTGCTTCCTTTTATCATTCTG
TTTATTTCACAAACAATATTGGTGAATGTCGTTCCTGTAACATTTGGATTTAAGAGCCTTGTTTCTGTAGCTTCTC
CCTCCGTAACCCCCACCACTACCATTTCGGGGCTATACAGCAATAGCATGCATTACTTTAAAAGGCAGGCTGCCTA
GACTGGCCACTTGTTAGCTTTGTGGCCTTGAGCAAATGACTAATCTCAGTAAACTATCTGCTCTTAGTTTCCTTCT
CTGTAAAATAGGCTCACTTATAACTATCTCATGGGTTGGGAGGATTAGATGAAATAATTAATGTAGAGCCCTTAGA
TCAGGGCCATAGTAAAAGCTGAATGAATGTTAGCATTTGTTATTTTAATTATAATCTATTGGGGTGCTTTGAAGGC
TTAATGCAAAATACTTAATGAGCTTTTTGGTAGCTGTTTAGTTATTTCGCCCCCCACCACCACCCCAAAAGGAGAG
ATTTAAAAGACCGACAGGAGAAGGTTGCTTGGAAAAGATGGAATAAGATCTATAAATAGAATTAAACAAATATTCA
GGAAAGCCTTTTGTGGGAAATACTGCAAAATTTTTATTATCTATAAATTTAATAGGTAGATAAAATTACTACTCCC
ATTTTAGAGACAGAAAACCGAGACTCGGAGAGCTAACGTAACTTGTCTAGGGTCTTAGGAAGATGACAAGTGAGGA
AGTAGAATTCAAGCCCACGTCTATATGATTTTAAAGCCCGAGGCACATCAAATGGAAAAGGCTGGTTAGTCAGAAA
AATAGGAAGGTATATTTATCTGACAACTTAAAATATTAGGACTAACCTCAGGTAATTATAGTCTGGATATACATTT
TTGCTGCTCCTGTTTATACTTTTGACTTCTGTGTATTTGAGTGTCTAATCAAAGGATTGTCTTTTACATGTGTTGG
AGATGTACAGACTAGTGGACCCCAATGATCTATTAGCTGTGTAACCTTAGCCAAGTTAATTCTCTTTCCTAAACTG
TGGTTCTCTCATCTGTCATGTGGGGCTAATAATAGTACTTATGCTGGTAGGGTGATTAAGAAAGTAAAATAATTGG
TGTTTCTAAAGTAAATATGTGGCACATATTAGATGCTCATTAAATGGTACATATTGTTATGGTGAGATGGATTTGG
TACAGAGAGAACTGGAGATGGGAGAATATGAAGGGTGTATAATGTGGCCTTTTATTAGCTAAACCAAGGGAAGGAC
TTCTGAAACAGAATTCCAAGTTTTAAGAGGGAGTCGTTTATTTTGGAATTATTTTTTCAGCTAAGGATTTTTCAAC
CCAGTCCAGAATTCTTAGAGAAATTTAGTGATAGCTTATAAATTTTAAGAAAAGGAATTCACATTATATTGCATAA
AGAACTGGTATACAGGGCCATAGAAGGGGAGAATGTTCTTCTGTATGAGAATAAAAAAAAACATCTCTCACACGAT
TTTTGAATTAACTGACAGTTTTATAGCAGCTTTGTCAACCCATCATTCATTGCTGCAACCAAATCTATGAAATCCT
TCATGGCGAAATAAAAAGGCTCTGTTGTTCTCCACATTTGTATGAAATCTCTGTTGCTAATGAAATGCCAGCCAGT
ATCTTCCTCTCAGGTATTGTCTATTAGATGGTTGCTTATTTTAGAAGAAGTGGAGTCAACCATATAAATTTCCTTC
TTTTGACATCTAGCACCTGCTGTCAACCTGTTATAGCTACAAGCAGCTCTCAAAATTCACATCCACTAGGATGCCG
CTGGCAACCAAAGAGTTCAGTTCAGTTCAGCGAACGTTTGAATGCCTACTCTGTGCTATCTAATATCAGAGATGGT
AGAGGGGATACAGGAAAAAAGTAAGATTCAGCCTTTGTCTTTAAAGAGCTCACAATCAAATGTGGGTATTTGGACA
AGTATATTTAGGCAAGGCAGTTTAGGATAGGTGCTTCAGTAGAGCAGTATTACAAAATGTTGAGAGAAAACTAGAG
GAGGAGTTTTAAATGAGGGCTTAGTGGAATGCTTCCAGGAGGAGGCTGAATTTGACCTGGTCATGAATAAGACTTT
GAAAAGCAGAAGGAAGCTGGAGAGGGAAGGGTATTTCAGGAATTGATGACAGGAGAATACATAATTAGACCTGTTA
ACAGTGGGGTGGAAGACGAATATCAGCATGGGAGTGTGTGTGTGTGTGTGTGTGTGTGTGTGTGTGTGTGTGTGTG
TGTTGTGGAGGATAAGGGAGTAAATGAAGTCAGTAAGACCAGTTAGAAGGCAAGAGAGCCCAAGCTGGAGCAGTGA
CCATGGAGTAAAAAGAAAGAAGTGAAATTGAGACATAAGGTGGAGCTAGAATTAACAGGATTTTGACATTGATTGC
ATATATAAAACTGTCAATCTAAGAGAAGGCCTGCCTCTAAGAGTTGGAGATTGAATTCTTTGGAAGATTAATGTCA
TAACCAGAGACAAACTCAGGATAAGGAGTTGATTATGGGAGACAAAGTAATGTGCTTAACATGAAGCTAGATGACC
ATACTGAGATTTTAAAGAAATGGTTGAAAACATAGAGATTGCAGGAGTGATGTTACTTTTGGACAAGAATTCAAAA
GTTACCTGGAATGTCTGAGCTTGCACAGAGAGAGGGTACCTGGTGCAAAGAAAAGTGAACCTAGGAAAAAGCCTAG
AAGTTGGTTTACATTTAAGGAATTTGAGAAGAGGTAGCACCACCCACCCCCCACACAACCCCCAACCCTGCCAACT
TACAATATAGAAGCATTTAGACACACACTGAATAATAATTTTTTTTTTGAGACAGAGTCCTGCTCCATTGCCCAGG
CTGGAGTGCAGTGGTGCCATCTTTGCTCACTGCAACTCCGCCTCCCAGATTCAAGCTATTCTCTTGCCTCAGCCTA
CCGAGTAGCTGGCATTACAGGCTCCCACCATCATGCCCAGCTAATTTTTTTGTATTTTTAGTAGGGACGGGGTTTC
GTCATGTTGGCCAGGCTGGTCTCAAACTCTTGACTTCAGGTGATCCACCCGCCTCGGCCTCCCAAAGTGCTGGGAT
TACAGACCTGAGCCACCGCGCCCAGCCTAAATAATGAATTTATAGATGCTACACTGTATGGTTTCCTTTTTCTGCT
GCTGTACAACCATTCAAGTAACATAAGTTTCATCCTGGTTCTTAATGATACCATGAATAAAGTATAGAAACTCTTT
AGCTGAGGATTAAAGATTGTTGTGTTTAGGTAGACCAGGTTTCTGATGCAGCCCTCAGTATACCAGGGAAATTAGC
CATTACTGTTTGCCTTGTAGGTCTAATGAACCCTTTAGCTTTTTATTTTGTATATGTCTAAGTTACTCTACAAATT
CTTATGGAAGTATAAAGAGATAGTGAAAGACAATCTTATGAGAAATTTTAATAAGAATTGAAATACAGGCTGGGCA
CAGTGGCTCACTCCTGTAATTCCAGCACTTTGGGAGGCTGAAGCGATGGATCACCTGAGGTCAGGAGTTCGAAACC
AGCCTGGCTAACATGGTGAAGCCCCATCTCTACTAAAAATACAAAAAAATTAGCCAGGCATGGTGGCGGGCACCTG
TAATCCCAGCTACTCGGGAGGCTGAGGCAGGAGAATTGCTTGAACCCGGGAGGCGGAGGTTGCACTGAGCCGAGAT
CACGCCATTGCACTCTAACCTGGGCAACAAGAGTGAAACTCCATCGTAAATAAATAAATAAATAAATAATAAAAAA
GAAATACAACTTACTTTTTGTATCAAATAAATTTTGGTGCACAAACATCATAAATGACATAATATCTGTCACACAT
CCTTTAGCAAAACAGGATTGTTAAAATTCTTACAGCTAATATTATGTGGTGAAGTTCTTCCTGTTGAATAATTAAG
TGGATTAAAATATCATATTTCCTTTCTGTCATTAGAAATATATTGTGCATTAATCAGTCCTTGGGCCTGAGAACAT
TTATCTAGGTTTTCATCATCTGAAAACTCACAGTCCAATATTTCACCTGTATTATCAATTCACCATTATCATTAGA
GTTTAGAATGCTGCTGTCTCTTTCCATCTATCTTCTGTTGTATCTAATAATTAGGAGATATATATATATATATATA
TATATATAGAGAGAGAGAGAGAGAGAGAGAGAGAGAGAGAGTGAGAGAGAGAGAGAGATTGGTAATAATACTAATA
TATATAATAATATTATATATATACACACATACAGAGAGAGAGAGAGAGAGAGAGAGAGAGATTGATTTTAAGCCAT
TAACTCATATGATTGTGGGAGCTGAAAAGTCAAAAATCTGTAAAATCCAAAAGCCTGGCAGGATGGAGACCAAGGG
AAGAGTTGATGTTGCAATCTTGAGTCCAAAGGTAATCCGATAGCAGAATTCCTTCCTTTTCTGGGGACTTCAGTCT
TTTCTCTTAAGGACTTCAAAATTGATTGGAGAAGGCCCACCCACATTGTGGAGGGTAATCTGCTTTACTCAAAGTC
TGCTGATTGAAATGTTAATCACATCTAAAAAATACCTTCACAGCAACATCTAGATTGGTGTTTGACCAAACAACTG
GGAACCATAGCCCAGCCAAGTTAACACATAAAATTTACCATCATCAGCCATCATCATGAGGTTCATAATGTCATGT
GTTTCTTCACATATAAATCTCGTGGTTCTTTTATTTTTTTAGTAGTACCTAGTTAATAAGGTACCTGGAAATATAC
GTTAAATAATAGAAATGGTCTTTAATATCTTAATGAATTTCAGAGTTCTATCAGAGTAATTGTAACTTGTATATAA
AAACTGATATATAAAAAACAAGTATGTTTTTTCTTCCTATACGTTGTGTTAAATTAATACTAATTACCTATCATGA
AATGAATGTTTTCCATTTTTCATAAATGTATTTCATTTTTAAATTAAGAGGATTAATATCAAAACATATTTGAATA
GGATTTAAAATCCAAATTACCTGTTGCGATTATATTTAATTATAAATCTTTCAAGTGAGTTTATAATAGGAAAAAT
ATTTTTTATTAAAGTGATGTACCATAATTATGAACATAAATAGTGTTTTCTACTAATTGTTTTTGTCTTCATTATG
TTTCTTTTTAAGGGGAAAGATCTGGGTTAACAAATGTTTTATTCATCCACCTTGAAAATTAAACATAATAAACAAA
ACTATTAAATAAATAAACCAATTAAAGAAAATAGTTTTTCTTCCTTTTTTGTGATAAAGATGATTATGAGTAAATA
TATTAAAGAATTTTATACTCAGACAAAGTGAGCTAATAAGACAAATACAAACAGAGATGAGCCCAGAAAGAGATAC
TCTACATGTTTATAGGCTAGCATTTTTATCTTATGACCTACAGGGTCATTGTAACTAAAGTCTTGAATTTCTGTTA
TATTTTGGTACCTGTGGGCATAAATAGGAATATTACTCCCTGATGGCATCATGCATTTTAGATAAAACTAAGCCAA
GGGTTAAACACGTGCCATGGACTGTTCCTAAAAAATGCTCTCAGCTCTCAAGTTATAGAAAATTGTAGGGATTATT
ATTTTGTACCTACTACTTCTTTCTTTACCAAAATTTACTTATACATGATTTGAAAATTGCTTGCCACCTGTCTTTA
TTCTGTTGTACTTGTCTTAAGTAAAACATTTATAAAGTAAGTAGGAAGGATTAATAGATTGTGACCTTTCTACATG
AAAAGGGGAAAGCCGAGCTTGTTCTACTTTTGTCAGGAAAGAGTTTGAATAGTTACCTGTTACTGTAAAACAATCA
CCCCAAACTTAGTGTCCTAAAGTAGCAATAATCATTTATTATCACTCATAGGTTCTGTAGGTCAGGATTCATGCAT
TGCTTGGTTGAGGGGTTCTGTCTCACGATCACTCATGATGTTGCACTCAGATGTCAATTAGGCTGCAGTCATATGA
TGGCTTGACTGGGGCTGAAGGATTCACTTTCACAGTGAGTTACTTGTATAGCTTGTGAGTTGGTGCTGTCTAGTTG
GTTTCCCTCTAAACGAGTTCCTCCATGGGACTACTTGAGTGTTCTTATGATGTGGTGGCTGGCTTACCCCAGGACA
AGCAACCTATGAGTTTTGCCCTGAGTTTTCTGAAACATGATCCCATGTTGCTTTGAATATCCCCATTAGACACTCC
CACAAATGTCAATCATACTCTTTGCCCTAAAAGTTGCAGACTACTTGTACATGTCCTTTATCCTACATGTGTACAG
GAAACACGTGCAGAATAACATGTGTTTTTGTTTCTTGAAAGAAACAAGAACCCACCAAAACTTTATAGCTCCGGGA
CATGATGATAATTATCTCAGGTCCACTCAGCCTGGCTCCACATGGAAAATATGGGTATTGTAAAGAAAGCTTTTGA
ATTTTGGAGGCCTAACAAAAAGACCTAGGAAGCTAATAGTTGGTTATGAGATAATTATAACAATGGTGATGAAAGC
AATTGCGATTATTACTTTTATGTTAGTGAGATAAATACATAAGAATGTCCTTCTTTAATTTCATTCCTTAATTTCC
TTAATCCCCATTCTTGATTTCTGGATTAGAGGAAAGCTATACTATCTACCTGATTACACAAAAAGACTGGACTGAG
GGGTAACCATTAGGACTCTTGTTAGTTGAAGGTAATAAACTATATTACTTCAGTGGCTAAAACAAACAGGAGCTTT
TTTCTCATATAAAACTAGAAGTCAGGAAACAAATGGTTGCTGATGTTGGTTTATCCGTTTGACAGGTTCTACCTTT
ATCCTGGAGATTCTTTGGCCTTTATTTCTTTAGTAATATAGTAGTAACAGCACTGAATCTAAAATAAGAGCACTTG
GTCCAAGCACTGGCTCTGCCCCCTTAGTGACAGTGGGACCCATGGCAAATTATTTAATCTCTCCAACAGTTAATTT
TCTCATCTGTAAAACTGGGATCCTGATAACTTCCATACTAGTTTGGTGTTAGGATTCAATGAAATGACGCATGGAA
AAGTCCTTTTCAAATGGCTACTCACTATACAAATATTAATGTCTTAGTAAGCTCAGGCTGCTATAAAATAATGCCA
TAGACTGGGTGGCTTAAACAACAGGCAGTTGTTTTTCACGGTTCTGGAGGTTGAGAAGTACAAGATCATGGTGCTG
ACAGATGTGGTTCCTGGTGAGAACCTTCTTCTTGCTGTATCCTCGTATGTTGGGGAGAGAGGTGGTGCAGAGAGGT
ACTCTAGTCTCTCTTCTTGCTCTTCTTAAAAGGACACTAATCCAATTATGGAGGCCTCAACCTATGACCTCATCTA
ACCCTTATTATTTCTCAAAGGCCCCACCTCCAAATATCCATTGGGGGTTAGCACTTCAACATACAAATTTTAGCCG
GCGGGGGGGATGCCAGCATTCAGTCCATAACAATCAACTATCCTCATTATTGAGAGTTCCACTAGGCCTTCTCAAG
GGTTGACAATGATAGTGCCCTGCTAGCTATAAGAGTAAAAAAATTTTCACATCTAGAAAATGGGTGCACGAATATT
GGTAACTGTTCTAATGTCTATGCACAGAATTTTTAGAACTTGCTCGAGAAATTTGTCTTTCCTGACAGTGTTGCTT
CTCTTAAATTCCAGTCGAGGGCCTTGAAAAAAATAGGTTTTCCATCAAGATTCTCTGAATGAATAAATGAAATGCT
CTGATATTTTCTTTCAATATTAAGATAAAGCAAAATGTATACAGAATTTTCTATTTTCAGTGTTTCCAATTACTGC
ATGGTTTGGCTTATTACCATCTCTAAGTCTAACCCTGGGTCATAGGAGTAAAGCCATTGGGGGTCCCCTACAAAGG
ATACAAGGCAGTGGTAGATACAATAAGCCTGACAATTGGAGTCAGATTTTTCTCTTTCATACAGGAACCATGGAAC
TGTTGCCCTGGGCCACAGCTGGCTCTCAGAAAAACGTAGAACTGGTCGAGGAAAGGAAAGGCAGGGAGTCCTTGAT
TCACTTGTTATCAGAAAATTGTCTGTTCAAACAACAGAATGTTTGAAAAGATAAAATCAAGTCTCAAATGCACAGA
ATAAGAATGAGGGGAAAACACCTTTCTCTGTAATTAAACAGAAAAGTGTGCCATGAAGTGATAAAACCCAGTCAAC
CTGAAAATGTGAACATCAAGTGTAATTGTGCAGAACGATAAGGTAATATGAACAGCACAGGAATATATAGGCTCAC
GTTGGCATTTTAAAGCAATGGCACTTTACCTGTTCCTCATGAAGGATTTGGTTTAGTCTAATCCCTTGTAGGAGCC
ACTAAGAGGAGAAGGCACAGTCCTTTTGGGATGTAAAATGGGGAATTCTTTCTATGATTACAAGAATTTGTAAATT
GCTCAGAGTTTTAGGACAGCAGGTTATGTGTCTCTCAATGTTGGGTACCATGCCAAACGTACTTTAAGAGACATAG
CAATAAATAGAATGGGATTCATTTTTTTCTTAATGTCTGGCAGGGCAACCAAAATGCCCACGTTTCCCTTCAGTAG
CTTGGTATTTTGGTAACTAAAAACATGTTCCAGGGAACTCCAGAATATGAAACATTTCAGACAATTTGAAACTGTC
AAAATTTTCACTTCTTTATGGGACAAATAAAATCTAACTTTATTCAGATTTTAAAGTATCTCATAAAAGAGTAATA
CTTTAGATTTGTGCTGTGCTTTATACGAATTGGATGAGGAACTCTTATACATATATAAGTGGATTTTATTTTCACA
ACAGTCCCTATGGTAGATATTGTCCTTATTAAGTAAATGAGAGAACCAACTATCAAAGATATTAATATTTTGCATA
AGATCACACAGATAGCGGAACCAGGATTTAATCCAACTCCTCTGATTCTAAAAATAGGTGTTAGATGGGTATTCTT
TCCCCAAACCTATGCTGAAAGGAGGCTACATTTGGAGTCAGTTATTGAAAGTTTAAATATATGTGTATATATATAT
ATATATGTGTGTGTGTGTGTGTGTGTGTGTGTGTGTATTCTTATATACTCATGCATGTAGATAGTTATAAAGTACA
TGCTTATATGTCAATTTTATATATACGACTGATATATAAACTATCAATTTGTGATAGTTTATTTCAATTAAGTTCA
AACATATTGGGTATTTATTGACTCATGGGACTTAAAATTTCTCAGAGGCATTAATTTCTATGCCTTTAAGCAAAAT
AATGTTTAGCCCGTTTTAGAAAGATAAAAGGCTAACATATGTTTTGTTTTTGTTTGTTTTGTTTTGTTTTGTTTTG
TTTTGTTGAGATGGAGTTTTGCTCTTGTTGCCCAGGCTGGAGTGCAATGGTGCGATCTTGGCTCACAGCAACCTCC
GCCTCCTGGGTTCAAGCGATTCTCCTGCCTCAGCCATCTGAGTAGCTGGGATTACAGACACGTGCCACCATGCCCG
GCTAATTTTGTATTTTTAGTAGAGACAGAGTTTCTCCATGTTGGTCAGGCTGGTCTCGAGCTCCCAACCTCAGGTG
ATCCACCCGCCTCGGCCTCCCAAAGTGCTGGGATTACAGGCGTGAGCCACGGCTCCTGGCCGCTAACACATGTTTT
TAAAAAATAAACACCATTCGAGTAGGAAAAGTGGGAGAACTGGTGGTTCAGCTAGAGATACGGAGATGGGAGGGCA
AGTTGCTTTCTCATGTTGCCACATCTGTAAGTTACAAATGCCATAGATTAGGAATTTCAACGTTGTTGTGTCTCTC
TGAGGACATTCTCCATGTAGTATATGTTTCGCCTCCCTGATGGACAGTAAGCATCAAACGGTTACTCCTTTATATT
TGAATTATTTTAATTCTAACATCTTGTTCTGAAAATAACGGGAATGCCATAAATACTTACTGATTGATTGGACTGT
CTCGCAGGGGAGCTGGCAGCTAGTGTAGGCTACAAGCTGCCATCATCATCTCCATGACCATGAGTCATCACTGGAA
TTACCCTAAAGAGGTTAATAAAACGCCTTGTTTGAAAAGCAAACAAAAAGCCAGAAGAAACCCAAAGCAAACAGAC
CCAGACTTTGCAATGGGTAGTGAATTTTCAGTCTTAAGGGATTGAGAGTCCTTTTAGTACAATCTCTCATAAACAT
ATAACAGATTTTAAAAGCACCTTGGTCTGATTCCAGTTCACATACCTCATCCTTGCAGAATACAACATACTAGGCC
ATTTGAAATGTTCCTCAGTTTACTTTTGGAGGATCTGCTGATTTGAGCAAAATGGAAATGAAATGTATATAGAAAG
CTCTTAATTTTTTTTTTTTTTTTTTTTTGAGACGGAGTTTCGCTCTGTCGCCCAGGCTGGAGTGCAGTGGCGGGAT
CTCGGCTCACTGCAAGCTCCGCCTCCCGGGTTCACGCCATTCTCCTGCCTCAGCCTCCCGTGTAGCTGGGACTACA
GGCGCGCGCCACCATGCCCGGCTAATTTTTGTATTTTTAGTAGAGACGGGGTTTCACCGTGTTAGCCAGGATGGTC
TCGATCTCCTGACCTCGTGATCCGCCCGTCTCGGCCTCCCAAAGTGCTGGGATTACAGGCGTGAGCCACCGCGCCC
GGCCAGCTCTTAATCTTAAGAAGTTCAGTTTCGCAGCTCCCCCCCCACCAACCCCCAATCTTGAAAGCAGGAATTT
GGATTAGGGTTTTTTAATAAAACTTTCTTTTTCTTATTACCATGATGTTTTTTGAGAGGGTACAGAGAACTCATTA
AAATGTTATGTTATAACTTAAGACAAAATGGGAATGAGAATTTGCATTATATAAACAAAGTGTATGACTAAGTAAC
CTGTTACATATAAAGTGTTTGACTAAGTAACCTGTTGTCCCTAGGAGAAGTAGAATAAATATCAACATGTGGCAAC
TAACAGAATGTGTTGTGATCACTAAAGCAGCACATTCATTGACTTTACACTTCATTAAGTAGGTAACGAATTTTAC
AAATTTTAGGACTTAACCAGGCTGTCCATAATACTTTGCATCAGTAAGTACCAAAATCTACGATAGGGCACTTCGG
AGTTCCTTAATTTAATAATATTGATAATATTTTGTTTACATTTTGATTTAATTGTATCATTTCATTATTTTTGTGT
CCTGATTCTAAATATATACGTCAACAAGCTATATGGCCATTCAAAACAGGTAAACTTTAAATATGTTTTTGACATA
GAAATTATGTAACAGTCTTCATCATGAGTATAGGAGTCAGATTTCCAATTGAAAATTTCATCCTCTAAAATACTTG
GCATATGTATACAGTTGTCTAGTTCAGACAAAAAAGTATTGAATATAAAGTTGATTTTGTAAAACCAGAATTGGCA
TCCTTAAGGAATACAAATAAAGGGAATCTTAAGATAATATAATAATGCTGATTTGAAATGAAAATTAATAAAATTG
TTATTCTATTTTAGTATGCTAAATTGGACAGATCCTTATTTCATTGAAAAATTAGATTCTTCAGTATATCAGTGCA
CTGTGCTAATAACACAAGAAGATTAAATTATGTCCATATGTGATTTGGTAGAATTTAATTATAAATATAATAAATC
CTTCAATATGTACAGCAGTGAATATTCCAAATTTATCTCTCATATAAGTTGTGAGCAACTTAAGGATAGGCACCAT
CCCTTAACATACCTAGATTAAGAACAGTACCTAGAAAACAGTCATCAAATGCTTCCTAAATAAATTTTCACCTTCA
AGCTTATGGCAGCAAATTAAACATAGATTGACCCTTTTCTAATAGAGACAGAATAGATGAGACTGATGACTTTTTT
TTATAAATGTGTAAGTCTGCAAATATTTAGTAAGGATCTAATTTACACAAAGAAATCAACCTAGAGTGAGATTATT
TTATTCATTTCCCGGAGGCTGTGACTGAATTATATTTGCAACTTGCCCTAATTTTTATACTTAATAATTTGCATTA
GTATTTAAATTATGTGAAGGAAAAATATAAGTTTAAAATACTGAAAAGTATATTCACACATTCATCAAATGTTGAT
TATGTGCTTACAGTGTGCAAGGTATTGGGGCTAGGAATTATGTGGTGTTCCAAGGAAACCATGATACAGCCTCTTT
CATTACAGTGTTTATTATTAGGTAGAAAAAGTAAAATTAGTATAATATTAGATAGGATATATGTGTCATAGGAGTA
ATATAAACAATATTTTATGAACAATGTATGAAGGTATAATTTTGTTCATTTATCCATTTATCGCTCTGTACATCCA
TCCGTTTATTCATTGTTCATTACTTCATTTAATAATACTTTAGTGACCATTGGAAAAGGGATTTATAAAAGTGAGT
AAAATGTTAATCATTTTCTAAAGAAGTTGATAATCTAGGGAAAGACAAATGTGTACCAACAATAACTTGGATATAA
AGCATAAATTAGAGGTACAAGCAAAGAAAAATGAAAGTACAAAGGAGAAAAATATTTGGCTGCATGAGAGCAGGGA
CTGTGTCTTGTTTCATCATAGATTTTCCAGAACCTAGATCAGTGTTTGGCACAAAGTAGGTTCAGATGAGTTTGTG
TTGACTGACTGTCTACTGAAGTAATTGTGGAACATATCATAGAAGAAGTAGATCAGAGGCTGGATCTTAAAACTGG
GAGAAATACATTTCAGCTCCGTGAAACCCCAGAAGTGATATTTGACATTAGAAAGCACAAAGTCACACTCTAACAG
TGACAGTACTGAGTGAAGTAAGAATTTTCTGCACCCCTTCTTCTTCTTCCACTCCCATTAACCCTTGTTAGGGTGA
GAAATAGCAGCTGGTGAATGGTAGAGAAGACTAGCAAGACAGAAAGAGGAAGCCCACCATGCCCACTACCCCAGGT
AGTAAAGGACCTGTCCACTTCCATCCTCTTCTAATTGAGGAAGAAATCATATATGTGAGTTCAGATTGAAGTATTG
ATTAATATATTGAGCTGGATATTCTAATTTTAATGTCTCGATATGGAGCAGGACTTTGTTTCCTTAATGATGACCA
GAAAAGACATGAAACTTACCCATATTTTCACCCAAGAACAGAACAGAACAACCTACTTGAACAGATTTAAAGGGAG
AACCAAAGGCAGATAAAGTTGTCTTTATTCTTACACAGCATAAGTCCTGATTATTCATCAATTTATACATTTTATG
AATTTAAGAAAAAAGGAAAGGAAGACTATAAAGGGATTCATATCAGGATACACATGAAGGTAATGTAGTCAGTTTT
TTAGTGGAACCAAAATATTACAGTCATCACTGAACGAAAATATTACAATCATTACATCATTCACTAGATTAAGTGA
ATAAATTAAAAATATATTAAATAAAATTAACAAAGACATGAGCTTTTCAAAGTGTGTGGTAACCTGGAGCAATAAG
CAGTGTAGTTGGAATGTTACCTCTTAAGGATTATCAAAGAGGCTGGCTGTTCAGTAGGACAGTGGTATGGTAAAGC
AAGCTTGCTACATTGCAGAACTCCAGAGGGCAACATTCTAATTATCTTTAGCTATGGGGTCTGTTCCCATAGGATA
ACTATAACTTAGTAGGTTGACAGAGCCTCCCAAGAAACCACAGCAGTGCTCAATTGTGGCGAATTACTTCTCTATC
CCATCAAAATGTCGTGATTGGACCCACAACTGTGCATACATTTTTTTGGCATTTTCCTGCCAGAAGCATATTTACA
TTTCTTTAATATCCAGTCATATCCTTCCCTTATTTAAAAATAATTATTCAGAGAATTCCAGTTTTTTGTAATCCTC
TCTTCTATGAATTTTAGCAACACTTATATACAGATACTTGATATTCTGGAGAAATATGCTTCAAGACCATGTGGAT
TAACAGATTTTCAAATGCCGTTATAGTATATGTATATACTATATATGTATAAGTATGTGTAGTCCCCAAAATGCGT
ACTTTAAAATCCTCATAATAGATCTCTTACAGATGAGGAAAAGAAGATGTAGAAAGGTTACATAAAGTAGCTAACA
AGTATTCCAAATAATATTGAACTTCAGTCTGACTATTCCAAAATTGCATTCTTAACTCTGATTTCTATATTTGTTT
TCCATTCTAAACTGTGTATTGTGATATAAGTTCCTCACTAAGGCTCTTTTTCAGGGTCTTCCTAATACTAAAGTCA
CTCTTACAATGAGTATTTTCTTTACGTGTGAAATCCAATAGGCAAAAAAAAAAACTTGGCAATAAATTTTAGGCAT
TAACCTCATGCCAAGTAATTATCAGAAGGCTGTAATGCTTTGAAACTTCACAAGTCTGATTTTAAGATAATGGAAT
GAGGCTTGCATTGTGAACTTTCTTGATGCTTTACATTGCAATATGCTGTATAGATTGACTTCCTAAAAATAAAAAA
TAAAAAAAAAGATTTGTAGAGCATACTGGGAAGGTCTTGCCAATTAAAAACCGGAGATTGGCTGAAGGCTTGCAGC
AATTGAATTTTGAATACAGATGGTGTCAAATCGAGTGTTTCCATAGCAACAGACTCTTCCTTAATAACTTTTAGAT
GGGAGGGGGTACATAAAAGAGAAAACCATCTTTTAGCAGATGTATGTTTTCAGCATGTTTTCAGATTGATTTTGGA
AGCTAATTTGTACTTAACTAGTGATTGTTTTAAGTGGATCTAAATATTACTAAATTCTCCTGAGGAAACATTTTGA
GAATAACAGAAATAAACTCTAGGAACTTTATAAAGACATGAAAAGGGCACAATTTTACAAAACCTTTTTTTTTTTG
GTCTGGAGCCAATCAAACAGTATTTTATATTGAGTATGACCTATCAATAGTCAAAGAGTTCTTGATTCTTAATGCC
TGTTAATATTGAATGTTTAGAATATGGGTAAAATCAAGGAAAAAGTGCTATGTATCTCATGGATTTGGAATATCTT
AATATACTCTTTCTTCAGGTTAATGATTATTTTTAAAAAATGATGATATAAATCATTTCAGGAAGGACTGCATAAA
GAGGTCATTGATAGAGTTCATATCATATAGCAGAGTTGTCGTAGATTTAATGTCCAATCCTCAGTTTTGCATAGCC
AACAACTAAATTTTAGTAGTTTACCTAGAGATGATCAGGCTCTGGGAGACTGATTCATGCTACACAAACTCACAAG
GCACTGGTTTATAAACCATTTGAAAGAAGAAATAATAATTCAAACTATTATTTTACAACAAGACTGGCAGGAAAAT
CAATAATGGTAATGTGTTCGGGGTGTCCTCCCCAGTGAGGACTATCCAGAAGACCAAATGATAGATTATTGTTCAA
AGTGATAGAATTGGGAGAAGGGTAATAAAGCATTATGAAAGGATGCTTCCTTAAGTGAGAAAAGCTACATAAACTA
CCCATCTTTACTATTCTAGATGACCTTAAAAATATTAATAGAAACAGAGAAACAGACAATTCATCTCAGATGGTAC
AAAAAACATAATAGCACATGTTTGCTCCCTGGCTTCCTTTTAGATTCCCTGCAAGAGGTTTTCCCCCAACCACCAC
CATATACAGATATACAGCTTTACCTTCACTCTCCTATTATCTGTCACAGAACAATATAAAACTCAGATATGTTAGT
ATAAGTTAATAGTTGGTAGCTTTATGTTAATGACATTGCTACCTGTTACCCCCAGATTCATCTGGGTTACTTCATC
CCCAAAACCCTTTCAAAACGACATTTCCCACAAAGATTTCTAGTATTACTGGCCATTCCCGGACATCTTGCTTCCA
ACATAAGAAATTCTGACATAATAGGGATATAAGAAGGCCTTGGGAATCTGTATTTATAACAAGCTCCCCAGGCTAT
CTGAGGCTCAGCCAGTTTATTTTTGCAGATAGCTATTACTATCCACAATCTCTTTTATCCTATTGTAATTGCAGAA
GAAAATCTTTCTTTCTAGTTTCTTTGAAATTTCTGGAATGTTAGGACTTAGAGCCTCTAGGAACTTGTACATAAAA
AGAGAGACTTAAAGAGATATCAAAGTAGAGATAGAGATAGATACAGATAAGTGCATAGACATAGAAGTAGACATAT
ATAATAGAAACATATATGTATACATAGAAATAGAAATATGCCATTTAGAACTCTTAAGAAATATGTGCACTCTTAA
AATATAGTTTAAAAATGTAAAGAACTTGCTATTAATTGAAAATAGCATAATAAACTTAAATGAAATAAGGACCCCA
AACGCAAATATTTCTCTCTGACACACCACACACACACACACACACACACACACACACAGACAGAGAGAGAGAGAGA
GAGAGAGAGAGCTGTACAATAACAACCAATTCCTGACCCAGATAAAGAAAAAATTTTTATTTATCTTCTTTCTCAT
TCTATTATTATTTTTTCTAGATTAAATTAAAATGGTAATTATGTGTAAGAACAAGCATTATTGCCATATAAACAAT
GCAACAAAGGAATCAATTGAAAATTAAGTGAAAATAAAAGGTACAGCTTCGTTAAAGACCCAGTTCTTAGAGCTTA
GTCTCAAACTCTTTGTACTCTGCACTCTTTTCTTGATTTGACTCGATTGATTGATTGATTGATTGATTGAGTCAGA
GTCTCACTCTGTCACCCAGGCTAGAGTGTAGTGGCTCAATCTCGGCTCACTGCAACCTCCACCTCCTGTTTCGAGT
GATTCTCCTGCCTCAGCCTCCTGAGTAGCTGAGACTACAGACATGTACCACCATGCCCAGCTAATTTTTGTATTTT
TAGTAGAGATGGGACTTCACCATGTTGGTCAGGCTGGTCCTGAACTCCTGAATTCAAGTGATCTGTCTGCCTTGTT
CCCCCAAAGAGCTGGGATTACAGGTGTGAGCCACCACGCCCAGCCGATTTTACTCTCTTTAGAACTGCAAAAGTAG
GAATCTAGCTCATATGCAGACATTCTAGAAAGTTTGATTTCAAAAGTCTTCTCAAAAGAAAGAGAGCAAGAGCAAG
AAAGAAAGCAGAGAGAGAAAGCAGAAGATAAAATGGCATTGTTTGAACAGGGATGGAAACTGAGTAAGAAATTTGG
TCACTAAACACTTTAGTGTCTATCATTTAAGATTGTAATTTGGTTATTTATCACTGGAAAGTGATTAATAATCTAA
AATGCATTTTATAATACTAATACTATTAAAACATTAATTTTTGGAGAAAGTTTATTATAGATTGATTTATACTTAC
CACTGAATATTAAAATGTTTAATGGAAGTAGTTTCAAATAGTATTTAATGATATAGGGAATTATTACAACATTAAT
GCTCAGGAAAAAAAGTAGGATATGGAATTATTTATGCCATAGGATCCTAATTTTGTAAAAAACAGAGAAAACCAGC
AAAAGAAATTATAACTGGAAGGAAATACATCAAAGTGGTTTGCAGTTATCACTCATGAAATTGAGGTGAACTTGAT
TTTTTTCCTTTTATATCCATCCGTGTTTTAATACCACAGACATGCTTTAGATCTGCCCGTATATTGTGGCTTTCAG
AGGGTAATAAACCATTGTGACTCAGAAATAGCTAAGAATTTTTATCTCTCAAGAACAAATTTTCACTCCCTTGGGG
TGTCATTATCTGTTGAGAATGCATGCAATAGTTCAAGAGCCAAAAGACTCTGATTCAGTAAGTTTAGGGTAGAAAA
AAATTAAGTACATTTTTAAAATAGAGCTCTGGTGTTTCAAAGGCAATGTGCAAATATACCTACTTAATATTATTCT
AATTTTTTCAGGATAGCTGAAATATAAACATCTATTTTTAGTAATAACACAAATGATGGAATGCTTTTACATATTT
ATAAATCATTAAGGATTTGCTTTTTGTTTCTACTGTCTGGAACATATAAATTTGAACACAATTTAGAACAACATTC
AGGAAATATGATTTATTTATTACCTTCTTGACCTTTATTTTATTTTTACCATCTACTTTTATGTAAGTCTTTTTTT
TCAATCATTGTTTATTTCTTTACTTTTCCTTCCACATAGAATTAAAGGGAGATTCGGGCTTAGTGTCCTTAATTCA
TAGTTCCATTGTGGCTATTAAAAGGTGAACTGAAAGCTTGCAAACACGTGGTACCTTGTAGATAATTTTCTCCAGT
CAGACAGTTAGATAAAGGCCTCTGAGGTTTCTGGGGTCATTGTTGAAGCTATGTTTTAAAATCCTATATCCTTCTC
TCATTGTTGGTTCCTTTTCACAACACAGAAGTTTTCTTTTTTTTAATTTCAACTTTTAGATACAGAAGGTGCATGT
GCAGATTTGTCACGTGGGAGTATTGCATGATGCTGAGGTTTGGAGTACGGATCCCATCGCCATGTTAGTGAGCATA
GTAACTGATAGGTCGTTTTTTTAACCCACTCCCCTCCCTCCTCCCTCTAGTAGTCCCCAGGGTCTATTGTTCCCGT
ATTTATGTCCATGTGTGCTCAGTGCTTAGCTCCCACTTATAAGTAAGTGAGAACATGTGATATTTGGTAGAACTTT
TCATTTTTAAGTTAAAAAACAAAACAAAATGAGGATGAGTGGAAGAACTATTCAGCACAGCAGGTTATAAACCAAT
TAGGATGATGACGCCCTGAATGGAGATTTTCATGAACATCTCATATTAGCTATTTCAGCTTTGGTTTTTTTAATGT
TCAAAGTAAATAGAATAATGAAGAGGCTATTTAGGAAGGTTTAGACTGAGGGAAAAAAATCCTTTCATTAGGTTCC
AAATAACGGTTAGCTTATTAAACAGCAAGAGGCAGAGATTTAGCAGAGAAAAAATAAAAAGATTTAAAAAAAAACA
ACGAGATTAAAAGGTCAGTAATACCATTGGAACTGGCAGCATGGCAAGTTTATATCAGCTATCTTTTGTTTTGGAA
CACAACTATGCTAATTCTGTTCTGAACCTCTTGCTAATGCCTGTCTCAAGAAAATTTAACATACTTTATCTGTGTG
TACAAAAATACCTAAGGACAAAGCTATTACCCAAACTGTATTCAGATTGAAAGAATCCATATAGAAATTTGCAGCT
AACGTATTAGTCAGTGTATGTAATTTCTACTGCTTCACAGCACAACTCTTTCTAATTTTCAGGAGCAATATAGCAA
CTGCTTGCCAGCCAAGAGAAAACCATAGGAGCATTCTTATCATTGGAGCCAACATTAGTTCTGCCTACAGTGACTA
ACATAGATGCGTTTATTGCTAGCTGGAATTTTCCATTGGCACTAGTTACATGTAATAAGTTAGTGCTTTCAAATGG
ACCGTGGAATATAGGAAAACTAGAGTCTGACGTAACCAAAAAAAAATGTTGATAAACTCAGAGATTATGAAAGAGA
GGAGAGGAGTGGTTTTTGTGGAAATGATAGAAAAGCAAAAAAAGATGGCAGGATTTGGAAAAAAGAAAATCAGGTT
AGAGATTTAATTAGTAAAGGAGCTCCTTTTAATAATTATATAAGAGTATGAGTTTAGAGTAACTGCCTGGCTAATA
TGTACAATCTTCAAGTTCAGTCGTTTTCCAAAATTCTAACTTTTAGCATTTTTTTTTTGTAAATTTTAAGTGGAAA
TCTTCCACTTTTGTTGACTAACTTGGCTACCTGATATTTTACTCAACCTCCTACTTTCTTGTTCTCTACTTCTTCT
GAGTCTTTGCTTCCACAATGAGGTAGCTCATATTCCTTAAGTTTCCTGTCTGGTTTTGCTTTTTTTTTTTTTTTTA
ACAGCTAAAATAAACTCTACAAGCATTTCTATCATTTTCTTTACATCCAATCTCTTGTCAGCCATGTCCTAATCAC
TTTGGTAAAGTATCAGTGACCACCCAAGAGGTTCACTTTGCTTCGTCAAAGGACCACCTGTCATGCTTTATCTCCA
GAGCTTCTGCCTTGAAAGTAAGAATAATATAACCTTTCCTGAATGTCTTATTTCAATTTCATGCATTTGATCTGCC
TCTTCAGACCATTCCCAAGGCTTTGCCCTTGTTCTTTACCCTCATGGCTTCAGTTATTCATCACTATATGAATGAC
AGCTCTCAAACTGACAGCTCTGGCCTCTCACCTATTTCATCTATTCCATATGGCAAACTGTCTATATTACATTTCA
TCATGAGGGATAATTATAATTCCACATTCAACAAGAAATTGTGGGTCCGTGTTGATTTCCAACAGCAACCTTTATT
TTTTAGACTATGATAGAGAAAGGGTCAACTTTCTCTCTTACTTATTTATCTCAAATAACTCAAAGTCAAGCTAGAA
AAGCCAAGCAAGTAATATTTCCCAACAAAGCAGCTTTAAGCAAAATGATTTTCAGACACGACATGCAGCCTGTATT
GTGGAAGAAGCACAGTGACCTAGGTGTACTTTGCTGAAAAGCAGTGGTCACAGTCCATTAGTTTCATCTTTTTCCT
GCAAGAGAGAAAAGTATGGCAGGGCCAAGACTCCTAAGACCCCTTAAAAAATTGTGAGGTTTTTATAACCCTGTTC
ATTTCCCATCAAAAGCATTCATGAAGCACCTGCCATGTACCAGATGATGCATTCTAGGCAGAGGGAAGAACTTGGG
CAAAGTCCTGTACATTTTATCTTCTAAATCTTACTAATCTTTTCTTATCTTTCCATCTACCACCCACCATCAGGCT
GTGCCTGGACTCCTATGCTAACATCTTACTCTGTTTTTCTGCATCTTTTTTGCCCTCCCCTCCAATCTACTCTCCA
ATTAGCAACATGACTAATGTTTTCAAGCCCAGATTAGACGGTGTCACTTCCCTGCTTTAAACCTTTCAGTGGATTC
CCATTGCACTGAGGTTGAAGACCAAAATCTTTACCATAACTCACAAGGCCACTGGGAGCTATGAAAATTTTCTTTT
TTAGTTTTTCCTTTTAATTAGTGATGCATTCAATAAGTGGAGTCTATCGTGAAAGCATAGATTCCAAAGGCATGTG
AATACTCAATTATGTAATTTACTTACAGAATGTTTATTACTTCTTTTTTAGCTTTGATTTTGTATACATCCTAACT
CAATCTCAGTTATATAAGAGATTATAAAAAAGTTTGTGATGAATGCATACATAAAGGAACAAATAATTAACAAATA
TATAAAGACAAATCAGTATTGAATGACCAAATGGCTCTTTATTTATTAAAGAACTTAGAAAAATATAGTCTTGCTT
GAGACCAAGTTCTGGCAACGTATGTGTGTCTTTTTGTTTGGGTTAAAAAGCATTAGTTTTATATGTTTTAAAAGGA
TAGGATCTTCCCTTGGCTGCAAATAGTATAGGCAAAATGCTATTCTGTATCTTATTTTAAAGAAAATAACAATCAT
ACCTTATTATTTGCTGACAGTTTTGCAGTTTACAAAGCACTGTTCACATGGATTACCTTATTTTAAATTCCTCAAT
TTTTTTTCCTTTTACTATGGGAATTCTGTTTCAGGAGAATTTCCCTTTAAAATTAAGGTGATTATACTATTCTGTT
CCTGTATTTCGTAGCCATATGTTTCACCAAGGTATTGTCTCTCTCCAGGTCTTTCTTCTCTGGATGTATAGTTATG
ATTTTCCAGGAGGTAAATGAAAACAGTGGGAGCAATTGACAGAGTGTTTTTTGTTTCCCATAAACCCAAGCATTGC
ATTTAAGGAGCTGCTGTACTATTGTAAGAGTTCCTATACTAATTTTAAAAGTTCATTTACATATTTTGTAGATTTT
TAGGATAAGTCATCAGAAAAAAATTAAAAATAAATTTACATATTTTGTATTAAGCTGTTTTGTTACTTGAATGGGG
ATTATTGGAACGAGGAAAGAATTACTTTTATCTCCCCATTTTTCAAATCATTTACTATAACATTCATGAATTGCTG
AAGTTTAAACAATCAAAAATATCTGAAAATGGAGCCTAGGAAGGGTAACTTATGTCTTTCATACTCCTTCCTTTTT
GGTTTTTCTACCAGCATTTTAGTCTAAAATATATTTTATTTTCACTGATCCTGTGTTTGTCTTAATAATATAGTAT
GTATGTTAAATGAACCAGAATTCATTGATCTTTTTTTTTATTATTCCTCTTTTTGCTTTTCTGGAGAAGCATTTGA
AGAAATCAGTTCAGGCTACACTTCATCAATTCATTTCCTCATCTTCCCCTTAATACAAAAGCCCCATTTTCACTCT
TCAAAACATTTGATCACATACTTAATTTTCATAGCATTGACTAAATGTATGTGTTTATTTTGTTTATATATTGCAT
ATTTGTGATGATTTTCACTTTTACAGTTTAGGCATGGGTAGGAAGGACACTTACATGAGTACTTAAGTTCTTGGTT
ATGACTTGAGAAACTCACAAGGTAGCATGGAATGGCACGTAAACAAGGCTTTGATAGGGTGAAAGGGAATTATCAG
GACAAAACTGACTAGTGGCAGTGACTTTTGAGCAGAGTGCTGCAAACTGAAAAGGCAGAGAGCATTGTAGGCAGAG
AGAAAGCATAGACACATGTGAGATACATGTGAAATACTTAGACAAATGTGAGGTGTGTGGTAACATTGTACATACC
TAGGGCTCCTTCCTGTGTAGCTGTAAAGCTGGAGTATAGGCATCACAACAGGCAGCAGAAGCCAAGAAACTAGAAA
AGTAGACAAAGGCCACGTTATAAAAGATCTTGTAGGATAGGTAAAGAGTTTTAATACTATCCTGAAAGCAGTGTGG
GGTGGGAGGCATGACATTAAGGAAATTTAATAAGTGATGAAAGACCAAGATTTTCATCTTCAGAAGACTTCTCTGG
AGGTGGGTGGACCACTTGATGTCAGGAGTTCAAGACCAGCCTGGTCAACTTGATGAAACCCCATCTCTACTAAAAA
TACAAAAAAATAGCTGGGCATGGTGGTGCATGCCTGTAATCCCAGCTACTCAGGAGGCTGAGGCAGGAGAATAGCT
TGAACCCAAGAGGCAGAGGTGGCAGTGAGCCAAGATGGTGCCACTGCACTTCAGCCTGGATGACAGAGGGAGACTC
CCTATTAAAAAAAAAAAAAAAAAAGACTTCTCTGACTAGAGTTTTAAAGAAGGATGGGGAGTGCAAAAGGCTAACA
TTAAATCAGTGGTTCTCATCCCTGGCTGCACGTTAGAATCACTAGGGGGAGTTTAAAAAAAATGCCAATACTTGGA
CTCCACCCCAAACCAGTTAAATCAGAGCCTTAATAGGGCCCAGACATTCGTAGTTTTTAAAGCAGCCCTCTTGATT
CAAATGCACAGACAAGGTTGTGTTCCGCTGAACTAGAGAGACCATCAGGAGGCTTTTAACTAAAGCAATCTAGGGA
AGAGAGAGATGAATGATATAGAGGCCAGAAGAGCTATTAAGGAGAAGTGACTCGGCATCTTCTTGGTTAAAGTATA
AGGAAGAAAGAAGAATGAAGGATAATTTGCATGTTTCTCCTTCAGTGACTAAGTAGAGGAACAGATGTGTATCAGT
GATTAGGGGAGAGGAAGAATGAAGATGATGTAATCCAGTTTGGATATGTTGGCTATTTCATAGAGATTTAGTAGGC
GTTTGGCTCTATGGATACAGAGTTCAAGAGACAATTCTTAGCTTAAAACACAGATTTTACTCTGATTATTTTAGAG
ATAGTAAATGAAGCAATATACATACATGAACTCACCAGAGAGAATATGTAAAGTGAAAACAAAAAGATCAAAGGGA
GACCCCTAGGGAATAGCAACATTTAAGGAATGGGCCTAAGAGAGTGAGGAGTGGCCAAATAGGCAGAATAAAATCC
AAGAAGGAATCTTCCATATTAATCAAAAGAGAAGGTTTCAAAGAGGGTTCGTCAATTGGCAGGTACTGAAGGGATA
TCTGAAAACATAAAGACTTTAGACTCTCTAATACTGTAGCAACTTTAAGGTCACTGCTATCACCTACAAAAGTAAT
TTCAGTGGCAAAAGCCAAAACGTAATGGTTTAGAAGGGTATGTGAGGTGTGGATGTGGAAATAGTGGGTATAGACT
ATTGCTTCTCAAAATGTAATCACCTGAAAAATCTTATTTTAAAATGCAGATTTTGATTCATTAGCTCTAGAGTGGA
GCCTGAGATTCTGCATTTCTAACAAGTTATCAGGTGATGCTGATGCTGCTCATACACAAACCATCCTTTAAGTAGC
ACTGGTGTAAGCCACTCTCCCACAAAGGAGGAAGACATAGGGTTTCCATCAGGGGACGCAGGTGGGGTGTAGGACC
AAAGGAAATAATACTCTTTTGTCTTTTGTTTGGTTGGTTTTGGTTGTTTTCTTCTTAAAATAAACATGGTTGTAGA
CGGAGATTGAAGACATAGGAGGGGTAAAAGATGGAATCACTTCTCATAGAAGATGGAAGAGACTGGAACATTGAGT
ACAGCGAGGGAATTAGGCATGGGTAGGAGGGACACCTAAATCTGAAGAGAAGGAGGTAAGGAAGAATTGAAATACA
GAAAAGTTCTGTCAGTAAGCAATGTATGGAATTGTGCTTTTTAGCCATAGTTTCTGTTCAAGAAATTGTTTTCCAT
TTATTTTTATTTCTTAACTTGAATAGTTGGATGGTGGAGTATGCTTGTAAAAATAAGAGTGAGGCCACTTTGTTTT
TCGGGGTTAATTTCCGTTTTCTGCACTCATCAAGTAAAAGTTGAAGTGGCAAGGTGTAGATATGAAATTCAGTGTG
TGCTAAGGGAGAAAAAAATGCTTTTTATTCTACATGATTTTAAAAATATTTATATTCCAACAAATGCATCAAATTT
GATGTGCAAATTTACAGTGATGAATGAGTTTTATTGTGTGCATTGCATGCTGGTGACATGGTAATAAATCTGTGGT
GCTAGAATTATAATGGTCCCCTTTAGCTTCGCTTTAATGAACTCTTGCTGAACACTTTTGAGTTGTTAGTACTTTA
TTTGCTACATTTGGCACTTAATTAGTTAATGACTGAGATGCTGGACCGATGGATCATCTAATGATATGTTAGGCCT
ATTATCACATCTAGATAGTTTCTTTTCTGTGACTTGTAAGTGACCTAAGATGATAAACTGAAATATTTTTGCATAG
ATATACATCAAGCTTTTCTCCTAACTTCAGGCTTTCATCTTAAGCAATAGTTTCCAACACCCCCAAAAGAGAAGTC
ATTATGTTTTTAAAAAAATTATTCATTTTAATGTGATCAAATAATATCACATTTCAGCATTCACCTATTTAATTAA
TAAAACAACTTACATGTTTCATTATGACTGGATGTTGATATTTTTTCATAATCTATTATCCTCCAACCAGTGGTAA
AAACCCAATCCTCCTCTCACCCAGCTCATCTTTCCGTATGGGAAGCAATACATACTTCCCTATGTTTTATTACCAA
AACAGGAGAATGAGCTTTCTTTAGAAGGTTAACTCATTTTCTCTATTAGAATATTCAGCATACTTTTAAGGAGGTA
ATCTGGTCTTTGACAGTCTGTTGATTAGAAAATTAAGAGACCTGCCTAAATTCCATTTCCAACTCCTCTCCATACA
CATTGTGACTTTGAGCAAAACGTTTTGCCATTTCCACTCGTATAAGTTCTGTTTAGTATCTTTAAACTTCCATATC
CCACAGATGGTATTTTTTTCTTCATTGGAAAGTGGTGTTAGTGATTCAGAAAACTGCTTAAATAATACACTGCTTT
GTGTTTTCTGTGAGAGAATTTTTTTTTTTTTTTGAGACAGAGTCTCACTATGTTGCCTAGGCTGGAGTGCAGTGGC
GCAATCTCGGCTCACTGCAACCTCTGCCGCCCGGGTTCAAGCGATTCTCCTGCCTCAGCCTCCCGAGTAGCTGGGA
TTACAGGTGCCTGCCACTGCGCCTGGCTAATTTTTGTATTTTTAGTACAGACAGGGTTTCACCATCTTGGCCAGGT
TGATCTTAAGCTCCTGATCTCATGATCCGTTCACCTCGGCCTCCCAAAGTGCTGGGATTACAGGTGTGAGCCACCG
TGCCCAGCCGAGAGAACATTTTATCTAACATTCTATTTTAAAATTTTTCAAATACACAGAAAGCTGAAAGAATTGT
ACAGTGAGTGCTCATGAACCCACTCTCTAGATTTTGTTGTATCACTTTACTTCTTTCTCTACCCGCCAACCCCTAT
TATTTTCTGATGCCCTTCAAAGTAAGTTGGTGACATCGGTTCCCTTTACCCCTAAGTTCTTCAACATGCATGTCAT
TAACCAGAGCTCAATATTTGTTCACATTTCTTTTGTTTGTTTGTTTGTGGCAAAATTTGTATAGACTAGAATGTGT
AAATCTCATGTGTATTATGAAATGAGTTTTGATGAATGGGTATACCTGTGTGACACATGCCTCTCTTAAGATACAA
AAAAATAGCCTTGGCCCAGACAGGAGGGAACATTTTTTAGGTTGGCTTGAGTTTCCTTTAACTGACATGTAGCATG
ACTGAATATATGACCATAAGATTGCCAAGTTGAAATTTACCAAAGGTCCATCCAGGGGAACAGTATGGCTATTGAT
ACGTCATTTGTTCATCTAGGCACTGGGCTGGGTGAGTTCTTCACAAAAACCTTGTAAGGTGAGCTTTCAGCTCTGA
CAGAGGCATCAAGAAACGTATTTCACAAACTGTTAGAGCTGAAAACATCTCCAGGGATCATCCAATCTATCCTCCT
TTGCTTTATGACTCAGGATTTAAGAAGCCCCAGAAAAGTCCAACAGTGTTACCAAAGTACACGATTGCCTATTTAC
GCCCCTATAATTGAGAGCTGTCTAGGGGTCAGGTACTTTTCTCAGTGCCTTGTGTACATTATTTCCTTTTATCTTT
CTAGATTATTAATTTTCTTGTTATTATCCCCATATAATAGAGGAGTAAACAGTGTGAAAATAGCACTAAACTGTTG
ATCTTTTCCATGTTCCAAACTAACCTTAAATGTTAGGCTAATATTATAAAATTTTAAAATTTAGTCATTTGTTCTG
GGTCTGGCTTCCATGAACTTAACCAAAATGCTTGGCTGTTTCTGCATTCTAGCTTTTTGGGATTTAGAGAAAAAGC
AACTTGGTCATCATAGTGGCCAGGATTTATTTATGTGCCAATAACTCCCCTCTGTTTTTAGCTTTTTGATCTGTTA
CATCTCAGATACTTTATTCTCTATTTGTAAAACAACCACATTCCTCCTTTTCTATATCTAAGTGAAAAAAAATGCC
CGTTCATACTAGCTACATGGTTAGCATATATGTTTCTTCCTCTTTGTGGGTTTGAGTTGAAACTTCTTTTGCAAAG
CTTAACTATAGATAGGTCTTCCACAGAACCCTGTTTAAATGCCTTGTGGAAGTAGAAGGGACATATCTTAATATTC
CTTATAATCTGTGTTATTATTTGGACTTTCCTCAGTTCCTTATATGTCTTTGTATGTCAAACATTGAGTTATGCTT
ACATTACTCTAGGTATAGTTGTGTCCTAGCTTTGTACAAGGTTACAATAATGGTGGTTTCCTTGTTTTCAGCTTCA
TTCTTGATCATGCCTAACATTTTTTGATGGTTTTAGCCTCGGAGAACAGTCTACTGCTATTTCTCCCTTTTTCCTG
ATCCCAACAAATAGTTTAGAAATCTTTGTCTTAACTGCAATTGGGATCATTTTTCCTTTAGAGTTGCCCACAGTTA
GTCATGTCCGTCACTCTTCACTCACTCAAATGACCTCAAAAGATTTACCTGCAATTTAAGCCTTTCACCTTGGCAT
TTTCTACCTGGAACTCTGGCGTACTCTGTGGGATCAGTGAGATCCATAGTGTGCACTCCCTAGCAGATCGTGAATG
TTTGTTGATAACTATAATTCATTGTTTAGTCTTGTCTATACAGAGAAGCAGTAGCACCTTGCTTTAGTAGGATCCT
TTATGCTTTTATTCATTATTGAATTTTATCTTCACAATAATTCTGTGGTTCTGCAGTTGTCATTATTTCAGTTTGG
GAGTTGAGGAAACTGAGATCGTGGCAAATCAACTTTTCCCATTTAAATCCACTATTTCATCGCTAATCCAGGGTTA
TAACCCAAGACTAACTGCTAGTCAAAGTTGCTTTCTAAAAATCAGTTACACAGACTTCTAATTCTCGTGCTGTACA
GTCAGTGCAGAAATTGCAACAGAACTGTTCTTTGTTCTGTAGCAATGTGTATTCAAAAGGTAATCATTTCTTGTGT
ATAAATAACATTCCTCCCTAAGAGAGATGTTGTGGGAATTTATATCAGTCAAGAATGTATATCGATAATTAAACAT
AGCTAGTCTAAAAAACTTGAAGTGAAAGATTGGTTCGTATTCAATCAACTGAACTGTTTCTTTCTCTGCAATATTG
TGGTTCTTGACATGATTGCTGAGTTTCCAATTTGACACTTCTGGAGGTTCGTAAATCAGGACACATGCTGGGTCTC
AGTCTGTCATCCTGAGTCTTAGCATTGGTTATTTATTTCCCTTTATTACCCAACATCTATTTACTGCACAAATTAG
TCGAGAAGCTTTCCATGACGGACCTTTGTGAGAAAAAAAAAATGTGTACATTGGGTAGCTCTTCAATTACAAACAT
GCACAGATTGTCTGGGTTTATGTGCATATACTTTATTATTATTTCACTTTTGTATGTCAGATGAATTTGGTGTAAA
TGTAAATCTAACAATATGGGCTCTAGTGGCCAAATACATTGCCATAATATGTTTTTGATCTGTCAGTCTCCTGGGT
GAAGTGTCGCAAGTGTGGTAAAATTCTGTAGTATAAATTGATGCTCAGTTATGGACAATTACCAGGTCTATATGAT
GTCAGACTACCACACTGATCCACTTTTAGGATAACAGCTTCGCTCTGATGATCTTCAGATTTTAGATTTGCTTTCA
TTCCTTGACAAAATGAAATACATTTAAAACTTCTAAAATTGTCTTTTGTATATATTTCTGCAGGGTCTACTCTTGG
GACTACTATAACAATCAATTGTACTGCTTTGTTTCTTTTATTAAATAGCAGAATAGCTGCTTGATTCGTCTGTATG
TGCTGATGTGTGTCTGTGAATCTAGTCCAGTTCACTGTCCAATAAGAATTTCTGAAATGTTCTTTGTCACATCAAT
GTACAGTCAAAATCGTATTGTGTTTGTAGCAGAATGATTGCATCTTTTATTATTCACAGCAGCCAAAATGACACAT
ATTTCACACGTGACACCTTTTTTAAAAAAGATGAATTGTCCAAAGTTGTATGTAGAATATATTTCACAAATCAAAT
TCCCTATTTAATAAATGGTGCTGGGAGAACTGGCTAGCCATATGCAGAAGAGTGAATTTCTATAAATAGAATGCAA
CTGTGTAAATAGCATCCAAATCAAGAAACATAATATAATATAGCCAGAAGCTCAGGATCCCCCTTCATGTCTTCTC
CAGTCATGAACATTTCACAAGGGTGACTACTATCCTTACTTCAAAAGTTTTCTATTACTTTTTCCTGTTTTTGTGT
ATTATATGAAGGAATCGAACAGTCATATCTACTTGTCTGTGGCTGTATTTTTCCCCCCAACAATATGTTTGTGAGA
TTCATCCATATTATTGTATGTAGTTGTGGATTGTTCACTTTTCTTACTGAATAATATTCTATTGATACCACAGTGT
ATTTACACATGTTGATATAGATGAGAAATTGAGTAGTTTCCAACATGAGGAAATTGCTGTCAACAATTCTGCAATC
CATAGCCATGCTATCAACAGTCTAGTACATGGTTTTTGGTGAATATATTAATAACTATGCATTTCTGTTGGATATA
CACCTGGGAGTGGAATTACAACTCAAATAATATTCATTTTAAAAATTTTATTTCGTTTTTAGTCGACAACTATATT
ATGGGGTACATTGTGATGTTTCAATCCATATATATACATTGTGGAATAATCAAATCAGGCTAATAGCATATCTATC
ACCTCAAATACTTCTCATTTTTGTGGTGAGAATATTTAAAATCCTCCTTTTTAGCTATTTGGAAATATACAATATG
ACAATATTAGCTATAGTTCCTGTGCTGTGCAAAAGAACACCAGAACTTATTCCTCCTGTCTAACTGGAACTTTGTA
CCCATTGATAAACGTCTCTCATTTTTCCATCCACCCACCACTGCAGCTTTTGATCACCACCATAATACTCTCCATT
TCTATGAGTTCAACTTTTTTTAAACTGCACATACAAATGAGATTATATGATATGTGTCTCTCTGTGCCAAGTTTAT
TTCACTTAACCTAATGTCCTCCAGGCTCATCCTTATTATTCCAAATGACAGAATTTCCTAGGTTTTTAAAATTTTT
TTTTTATTTTTAATTTTTTGGGGTACATAGTAGGTATATATATTTACGGGGTACATGAGGTGTTTTGATATAGGCA
TGTAATGTGAAACAAGCACATCATGGAGAATGAGGCATCCATCCTGTCAAGCATTTATCCTTTGTGCTACAAACAA
TGTAATTGTACTTTTAGTTATTTTTTAATGTACAATTAAATTATTATTGACTATAGTCCCCCTGTTGCACTATCAA
ATACTAGGTCTTACTCATTCTTTCTAACTATTTTTTGTAGCCGCTAACAATCCCCACCTATCCCCTACCTCCACAC
TACCCTTTGTAGCCTCTGGTAACCATCCTTTTATTCTGTCTCCATGAGTTCAATAGTTTTAATTTTTAGATCCCAC
AAATAAGTGAGAACATGCAGTGATTTTCGTTCTGTGACTGGCTTATTTCATTTAATGTAATGACCTCCAGTTCCAT
CCAAGATGTTGCAAATGACAGGATATAATTCTTTTTTATTGCTAAATAGTACCGCATCATTTATATGAGCCACATT
TTCTGTATCCATTCGCATGTTGATGGACAGTTAGCTTGCTTCCAAATCTTGCCTGTTGTGAACAGTGCTACAACAA
TGTGAGAGTGCAGATAGCTCTTCAATACACTCCCTCCTTTTCTTTTGAGTATGTACCCAGAAGTGGGATTTCTGGA
ACATATGGTCATTCTCTTTTTATTATTTTGAGAAACATTCATACTGTCTTTATGGAGGCCGTTACTAATTCACAAT
ACTACCAATAGTGGATAAGGTTTCCTTATTCTCTGTATCCTCATGAACACTTGTTATCTTTCAACTTTTTGATAAT
AGCCAATCCAAAAGATATGAGGTGATATCTCATTGTGATTTTAATTTGCATTTTTTGATGATTAGAGATGTTGAGT
ATTATACATATATGTGTGTGTATATATATATATATATATATATATATATATATGCTGTTTGTCATCTTTTGAGAAT
GTCTATTCATATATTTGCCCATTTTTTTATTAGGGTTATTTGTTTTCTTGTTATTGAGTAGCTTGAGTTCCTTGTA
TATTTTGGATATTAGCACCTTATCTAATGTATGATTTGCAAATATCTTCTCCCAATCTGTGGGTTGTCTCTTTATT
CCATTAATTGTTTCCTTTGCTGTGCAAAGCTTTTTAGTTTGATGCAATCTTACTTACCTATTTTTGTGTTGATTGT
GTTTGGGGGTCATATGCAAGAAACCACTGCCCAGACCAATGTCATGGAGCTCTTCTCTTATGTTTTTGTAGTTTTT
AGTTTCAGGTATTACATTTAAGGCTTTAATCCATTTTGAGTTGATTCTTGTATAAGGGGTGAGATAAGGGTCCAGT
TTTATTCTGTATGTGAACATTCAGTTTTCCCAATACCATTTATTGAAGAGACTGTCCTCTCCCTATTGTGTGTTCT
TGCTACCTTTGTCAAAAATCAATTGATCAAAGGTGTGTAGGTTTATTTTAGTCCTCTTTGTCTTATTCCATTGGTC
TGTTTTTATGTACTTGCCATGCTGTTTTGATTATTATAGCTTTGTAATACATTTTGAAATCCAGTAGTGACATACT
TCCAATTTTATTCTTTTTAGTAAAGACAGCTTTGGCTATCCAGGGTCTTTTGTGGTTCCATGCAAATTTTAGGATT
TTTTAAAAAAAATTCTATAAAGAACAATATGCAGATTTTGTTAGTATTGTGTCGAATCTTTAGATTGCTTTATGTT
TAACAATATTAATTTTTCCAATTTATGAACACAGAAATCTTTCCATTTATTTGTGTCATCTTCAATTTCTTTCGTC
AGTGTCTTATAGTTTCAACACGCAGATCTTTCACTTTCTTGGTTAAATTCACTCCAAATATTTTTTCATGCTATTA
TAAGTGAGATTGTTTCCTTAATTTCTATTTTAGACAGTTTGTTGTTATTGTACAAACAATAACAATTGTTATCGCT
ACTGATTTTTGTAAGTTGATTTTGTACCCTGCAACTTTACTAAACTTGTGTATGAATTCTAACAGTTTTCAGTGGA
GTCCTTAGGATTTGCTGTATAAGATTATGTCATCAGCAAGAAGGGGCAATTTTACTTCATCCTTTTCAGTTTGGTT
GCCTTTTATTTCTTTCTCCTGCCTAATTGCTCTGGCTAGGACTCCCAGTACTAAGTTAAACAAGGGTGGGGAGAGT
GGGCATCTTTGTCTTGCTCCTGATCTTAGAGAAAAGCCTTCTACGTTTTACTGTTGTGCATGATGTTAGCTGTGGG
CTTGTAATTTATGGCTTTTATTCTTTTGGAGAACATTTCTTCTATACCTAATTTGCTAAGAGTTTTTCTCATAAAA
GGATGTTGAATTTTGTCAAATGCTCTTTCTGAGTTTATTAAAATGATCATACGGTTTTTGTACTTCATTCTGTTAT
ATGTTGAATCACATTTATTAATTTGCATATATTGAAACAACCTTCTATCCCAGGGATAAATCCCTCTTGGTCATGG
TGAATAATCCTTCTAATAAACTATTAAATATGGTTCACTAGTATTTCATTGAGAATTTTTGCATCTAATTTCATTC
GTGATATTGGCCTATAGTTTTCTTTCCTTGTAGTGTCTTTGCCTGGCTTTGGGATCAGGGTATTGCTGGCCTTGTA
AAATAAATTTGGACGAATCCCTTCCTCTTTAGTTTTCCAAAAGAGTTTGAGAAAGATTTGTGTTAGGTCTTCTTTA
AATGTTTGTAGAATTCTCCCATGAAGCCATCTGGTCTTGAGCTTTCCTTTGATGTGAGAACTTTTAAATACTGATG
CAATCTCCTTAACTCTTTCCTTAGCTGTTACTGGTCTTTTCAGATTTCCAATTTTCATTATTCAGTTTTGGTAGAT
TATGTATTTCTAAGAATTCATCCATTTCTGTTAGGATGTCCAATTTCTTGGTATATAATTGTTCATCGTAGTCTCT
TAGGATCCTTTGTATTTCTGTGTTATCAGTCATAATGTCTTCTCTTTGATTTCTGATTTGATTTATTTGAGCCTGC
TCTCTTCATTCTTAGTCTAGCTAAGGATTTGTCAATTGTGTTTAGCTTTTCAAAAAACCAACTTTTAGTTTTATTG
ACTTTTTTTCTATTGTTTCTCTAGTCTCTATTTCATTTATTTCTGCTCTGATCTTTGTTATTTTCTTCTTTCTGCT
AACTTTGGGCTTAATTCATTCTTCTTTTTGTAGTTTCCTGAGGTATAATGTTAGGTATTTCATTTGAGATATTTCT
TCTTTTTTGATGTAGGAATTTATTGATATAAACTTCCCTCTTAGCACTGCTTTTGCTACCCCCAGAAGTTTTTCTA
TGTTGTGTTTTCATTTCTGTTTGTCTCAAGACTTTTAAAAAATTTCCTCTTGAATTTCTTCTTTTGACCCAATAAT
TGTTTAGGAGCATATTGTTTAGTTTCCACATATTTCTTAATTTTATATGATTTCTCATGTAATTGATTTCTAATTT
TATATTGTGGTCAGAAAAGATACACGATGGGTTTTCTTAAATTTGTTGAGACTTGTCTGTGGCCTAACATATGATC
TATCCTGGAGAATGTTACATGTGTACTTGAGAAGAATCTGTATTTTCCTACTGTTCAGGGCACAATGTTCTGTATA
TGTCTGTTAGGTCCATTTGGTCTAAAATGTCATTCAAGTCCAATGTTTTCTTATGAATTTTTCTGTCTATTGCTTA
AAGTGGAATATTGAAATTGCCTGCTATTATTATGTTATAGGCTATGTTTCCCTTCAGATCCCTTAATGTTTGCTTT
ATATATTTAGGTGCTCTGATTTGGGATGCTTATATACTTGTTATGTCCTCTTGATGAAATAACCTTTATCAATATA
TAATGATGTTCTTTGTCACTTTGAACAGATTTGACCTAAAGATTATTTTGTCTGAAGTAAGTGTAACTACCCTGCT
CTCTTTTTGTTCTCATGTACATGGAGTATCTTTTTTCATCCCTTTACTTTCAGTCTATGCATGTCCTTTAAGGTGA
AATGAGCCACTTGTAGGCAGCACATATTTGGGTCTTGTTTTTTGTTGTTGTCGTTAATCCACTCAACCACTCTATG
CCTTTTGATTGGAGAGTTTAATCTATTTACATTCAAAATAATGATGGATGGGTAAGGACTTACTAGTGTCATTTTG
TTCATTGTTTCCTGGTTGTCTTACAGATTCTTTGTTCCTTTCTTCCTCTATTGCTGTCTTCCTTTGTGTTTTGATG
GTTTTGTGTAGTAGTATACTTTGGGTCTTTTGTTTTTATCATTCATGTATGTATTATAAGTTTGTGCTTTGTGGAT
ACTCCGAGGCTTACATAAAACATTTTATAAGCTGATAATAACTTAAATTTGATTGTGTGCATATACTCAACACTTT
GACTCTCCCTCCTCCCACATTTTATGTTTCTAACATCACAACTTACTTTTTTTTTTAATTATACTTTAAGTTCTAG
GGTACATGTGCACAACTTGCAGGTTTGTTACATATCTATACATGTGCCGTGTTGGTATGCTCCACCCATTAACTTG
TCATTTACATTAGGTATATCTCCCAATGCTATCCCTCCCCCGTCCCCTCACCCCACGACAGGCCCCGGTGTGTGAT
GTTCCCCTTCCTGCGTCCAGGTGTTCTCATTGTTCAATTCCCACCTATGAGTGAGAACATGCGGTGTTTGGTTTTC
TGTCCTTGTGATAGTTTGCTGAGAAAGATGATTTCCAGCTTCATCCATGTCCCTACAAAGGATGTGAACTCATCCT
TTTTTATGGTTGCATAGTATTCCATGGTGTATATGTGCCACATTTTCTTAATCCAGTCTATCATTGATGGACATTT
GGATTGGTTCCAGGTCTTTGCTATTGTGAATAGTGCAGCAATAAACATACCTGTGCATGTATCTTTACAGCAGCAG
GATTTATAATCCTTTGGGTATATACCCAGTAATGGGATAGCTGGGTCAAATGGTATTTCTAGTTCTAGATCCCTGA
GGAATCGCCACACTGACTTCCACAATGGTTGAACTAGTTTACAGTCTCACTAACAGTGTAAAGTGTTCCTATTTCT
CCACATCCTCTCCAGCACCTGTTGTTTCCTGACTTTTTAATGATTGCCATTCTAACTGGTGTGAGATGGTATCTCA
TTGTGGTTTTGATTTGCGTTTCTCTGATGGCCAGTGATGATGAGCATTTTTTCATGTGTCTTTTGGCTGCATAAAT
GTCTTCTTTTGAGAAGTGTCTGTTCATATCCTTCGCCCACTTTTTGATGGGGTTGTTTGTTTTTTCTTGTAAGTTT
GTTTGAGTTCTTTGTAGATTCTGGATATTAGCCCTTTGTCAGATGAGTAGATTGCAAAAATTTTCTCCCATTCTGT
AGGCTGCCTGTTCACTCTGATGGTAGTTTCTTTTGCTGTGCAGAAGCTCTTTAGTTTCATTAGATCCCATTTGTCC
GTTTTGGCTTTTGTTGCCATTGCTTTTGGTGTTTTAGACATGAAGTCCTTGCCCATGCCTACGTCCTGAATGGTAT
TGCCTAGGTTTTCTTCTAGGGTTTTTATGGTTTTAGGTCTAACATTAAGTCTTCAATCCATCTTGAATTAATTTTT
GTATATGGTATAAGGAAGGGATCCAGTTTCAGCTCTCTACATATGGCTAGCCAGTTTTCCCAGCACCATTTATTAA
ATAGGGAATCCTTTCCCCATTTCTTGTTTTTGTCAGGTTTGTCAAAGATCAGATGGTTGTAGATGTGTGGTATTAT
TTCTGAGAGCTCTGTTCTGTTCCATTGGTGTATATCTCTGTTTTGGTACCAGTACCATGCTGTTTTGGTTACCGTA
GCCTTGTAGTATAGTTTGAAGTCAGGTAGCGTGATGCCTCCAGCTTTGTTCTTTTGGCTCAGGATTGTCTTGGCAA
TGTGGGCTCTTTTTTGGTTCCATATGAACTTTAAAGTAGTTTTTTCCAATTCTGTGAAGAAAGTCATTGGTAGCTT
GATGGGGATGGCATTGAATCTATAAATTTCCTTGGGCAGTATGGCCATTTTCACGATATTGATTCTTCCTATCCAT
GAGCATGGAATGTTCTTCCATTTGTTTGTGTCCTCTTTTATTTCCTTGAGCAGTGGTTTGTAGTTCTCCTTGAAGA
GGTCCTTCACATCCCTTGTAAGTTGTATTTGTAGGTATTTTATTCTCTTTGAAGCAATTGTGAATGAGAGTTCACT
CATGATTTGGCTCTCTGTTTGTCTGTTATTGGTGTATAAGAATGCTTGTGATTTTTGCACATTGATTTTATATCCT
GAGACTTTGCTGAAGTTGCTTATCAGCTTAAGGAGATTTTGGGCTGAGACGATGGGGTTTTCTAAATATACAATCA
TGTCGTCTGCAAACAGGGACAATTTGACTTCCTCTTTTCCTAATTGAATACCCTTTATTTCTTTTTCCTGCCTGAT
CGCCCTGGCCAGAACTTCCAACACTATGTTGAATAGGAGTGGTGAGAGAGGGCATCCCTGTCTTGTGCCAGTTTTC
AAAGGGAATGCTTCCAGTTTTGCCCATTCAGTATGATATTGGCTGTGGGTTTGTCATAAATAGCTCTTACTATTTT
GAGATACGTCCCATCAATACCTAATTTATCGAGAGTTTTTAGCATGAAGGGCTGTTGAATTTGGTCAAGGGCCTTT
TCTGCATCTATTGAGATAACCATGTGGTTTTTGTCGTTGGTTCTGTTTATATGCTGGATTACATTTATTGATTTGC
GTATGTTGAACCAGCCTTGCATCCCAGGGATGAAGCCCACTTGATCATGGTGGATAAGCTTTTTGATGTGCTGCTG
GATTCGGTTTGCCAGTATTTTATTGAGGATTTTTGCATCGATGTTCATCAGGGATATTTGTCTAAAATTCTCTTTT
TTTGTTGTGTCTCTGCCAGGCTTTGGTATCAGGATGATGCTGGCCTCATAAAATGAGTTAGGGAGGATTCCCTCTT
TTTCTATTGATTGGAATAGTTTTAGAAGGAATGATACCAGTTCCTCTTTGCACCTCTGGTAGAATTCGGCTGTGAA
TCCGTCTGGTCCTGGACTTTTTTTGGTTTGTAGGCTATTAATTATTGCCTCAATTTCAGAGCCTGTTATTGGTCTA
TTCAGGGATTCAACTTCTTCCTGGTTTAGTCTTGGGAAGGTGTATGTGTGCAGGAATTTATCCATTTCTTCTAGAT
TTTCTAGTTTATTTGCGTAGAGGTGTTTATAGTATTCTCTGATGGTAGTTTGTATTTCTGTGGTGTCGGTGGTGAT
ATCCCCTTTATCATTTTTTATTGCATCTATTTGATTCTTCTCTCTTTTCTTCTTTATTAGTCTTACTAGCGGTCTA
TCAATTCTGTTGATCTTTTCAAAAAACTGGCTCCTGGATTCATTGATTTTTTTGAAGGGTTTTTTGTGTCTGGATC
TTGTAGGTATGCTTCATTGTTTCTTATTATTTTTTTCTTTTGTCTTCTCTGGCTGTGTATTTTCAAATAGGCTGTC
TGCCTACAAGATCCAGAAAATAGCCTCAAAAGGGTCAATCTAAGAGTTATTGGCCTTAAACAGGAGGTAGAGAAAG
AGATAGGGATAGAAAGTTGATACAAAGGGACAATATCAGAGAACTTCAGAAACAGAGAAAGATACCAACATTCAAG
TACGACAAAGTTATAGAACACCAAGCAGAATTATCTCAGAGACTACCTCAAGGCATGCAATAATCAAACTCCCACA
GGTCAAGGATAAAGAAAGAATCCTAAAAGCAGCAAGAGAAAGGAAACAAATAACATGCAGTGGAGCTTCAATACAT
CTGGCAGCAGATTTTTCGGTGGAAATCTTAGGCCCCGGGCATATCCAGAGATGCTGTCTGAGGGCCAGTCATTGGA
GTCAAAAACCTTAGCAGTTTACCTCATGTTCTATTCTATTGTGGCTAAGCTAGCACTCACACCACAATATAAAGTG
CTCCCTGCTCTTCCTTGCCCTTTTAAAAGGCAGAGGATCCTCTCCCTGTGGCCCTCACCACCATGAGGGTTCTGCT
TGGCCTCCACTGGTGTTCACTTAAAGCCCAAGGGCTCTTCCATCAGCTTGTGGTGAATGCTGAGAGAACTGGGACC
CATATTTTAGGGCCTTGGGCTCCCCTCTGGCCCAAGGCAGGACCAAAAATGCTGTCCAAGAACCTAGGCCAGGACT
CAGAAATCCCAGAAGCCTGCCTGCTTCTCTGCCTTTCTGTGGCTGAGCTGGTACCTAAGGGGCAAGACAAAGTCCC
CTTTACTTTTCTGTCTACTTTTCTCAAACAGAAGGGGTCTTTCACCATAACCACCACAGCTGGGAATTTGCTGGGT
GACCTATGAAGCCAGCACATCTCAGAGGCCAAGTCCCACAGTGTACTCCCTGGGTATTGCAACTGGTTATTCAACG
TTCAAGGCCTCTTTAGTTAGTAGCTGATGAATCCTGATAGGACTGAGTCCTTCCCTTTAAGGTAGCAGATTCCCTT
TTGGCCCAGGGTGTGTCTAGAAATGCTATCCAGGAACTAGGGCCTGGAATGGGGGCCTCATGACTCTGCCCATGCC
CCATCCTACTGTGGCTGAGCTGGTATCCAAGATGCAAGACAAAGTCTTCTTTACTTTTCGCTCTCTTCTCCTTAAC
GAGAAGTAAGGAGTCACTTCTGTTGCTGCAAGCTTCACTGCTGGGAGTAGGGGAGGTATGGTGCAACCACTCCCTT
AGCCATGCCAGCTGGTGTCTCCCTAGGTCATGTGGGAGACCCTAATCCACTGGCTTCAATATATAGAGGGACGTTT
CTAAATTATTTATCTGTAATTTGATTAAGAAGATAGACACAGGCAATAGGATATGCCAACAGATTCTCTCTAACCT
ATAGTTATTTTAAGAAAGTTAGGGAAAGAGAGGTCATTGATTAGTTTTGGCTTACTGAATTATTTGACCCTCCCAT
ATCTTTTAATTTATGGATTTTATATAAGGCACAGATATTCTACTAGTAAACATGACATTAAAGATGTTTTATACAA
ATGAATGTGGTTGATACAAAGGCATTAAATAAGAAACAAAGGAAATTCAGAGGACATTTGTTCGCCTGGAATAGAG
ATCATTAGCATAAGCATAAGAGGAAAATAAGGAAGGAAATGGGAAAGTCTTGAGTCCATTTTCAAATTATGAAAAC
TTGAAATGCAACAAACAAATAGTGGTTACTTAAGAAGAAAGACTGAAGATGCTGGGCAAAGGTTAACTTTAGAAAT
GGCATTTTTATATCATTATAAAGAGAAAGTGGGGATAATGGAAATAATCTACATGTACAACTCATTTGACAGACAT
TTATTGAAGGTATTCCAAATGCCAAACACTAGTTTGTGCACTAAGGATGTTTTTAATTTTTACTTTTTACTTTATT
TTTATTTTCTAGCTTCTTCCGCTTTGCCAGAGAAGGATTTTTTTAAATGGATAAAATACTAACTTAGGCTAGCTTC
TTTCAGAAACAGACCTTTAAACAATGATTAATGGTGAAAGTGGTTTGGTTGTAATGTGATCCCAGGAAGTACCAGT
AGGAGAGTGGGACAATGAGATAATAAATGAAAGAAGCCAATAATTAAGCCAGTTATGGAACTAGTTATTGCTTTGG
GCCACTGGGGTTTGATCCTGATGAAGGAGCTCTGGGAGATAGTAAACAAACAAACAAACAAAAAACATGCCTCAAA
GTTGTCAACCACAAGGGGGTAAAGGCAAGGAACCAGGGCTAACTATTCCAACTCTTATCCATCATTGGCTAAATGC
TTCCTGGTACATAAACTTTCCAGCACTTCTGGCCAGCGCACCTAGCAAGCTGAGGAAAATCTCTCAGGTTTGCAGT
AGGGCAAATGCTTGTACTAGGACACTGCTGGCATATACTTGAAGGATGAGTGCCAACGGCAGATAGATGGGCCCTG
ACAGCATCTTCTACAGATTCTGTCCTTGCCTTTGAGGCAAGGACACTGTCTAGAGTCAGAGACAGAAATGTAAAAT
GTAAAAATGTAAATCATAGTAGCACACAGTACAAGTTTTAAAATCTTAACAAAGTGTTATGGGAGAACCACGGTGA
GAGAAAATGACTGCACATATGAGAGCAGGGGAAGTCTTTATATGTGATGATGACATTTGAGTGGGTGTTCCGTTAT
CAAAGATCTACTTAAAAGGGAAAAAGAGTAGAATTATTTTAAACCATGGATGTAACCAGGCGTGGCCACCCTATAT
GTGAATGTCCTGGGATAATTTTGTCCCCAGATGTCTCTCTTTTTGAGTGTCATTTTCATTTATGTATTCATAATAT
AAAGAAATTGAATAGAAAGAGGTAAACATTGGTGGCAAAAATCCCTTCCTACCCATAAGGCTCACAGAATAATGTT
GTCTAAATGCCATAACATTTTTGGTAAGTTTTGGTTCTATGGGCTTTGGAAATTTTTTATGTAGGAACTCCTCCAA
GGTGATATAAGCATTCAGTGTATTTATCTAGATACCCTAGTTAAAGCTACTTTAAGCAGGATCAAAGTCCTCTTTT
AAACTTTTGTATTTGAAAGCTATTCCCATGTACAATTCCCATGATTCTTGTACAATTCCTAGGAGACCTTTGTTTA
CATCAAAAGAGTTTGTCTCTCTCTATAAGTTAGAACTTGTACATTGTAGTAATGGAAAATCCATTTCAAAGTAGCT
TAGACTTACTCAGACAAAATGTATTGATCAGTGTCATTAAACAGTTGAAAGATAGACGAGGCTTGATTCAGGAATT
AAATAGCATGACCAGATTCTAGTTTTCCTTCTCCATTGCATGGCTCTGCTCCCCAGTATTTGTTCCGTTTAATTCC
TCTTAATTGTTCTAAGATGGATATCAGGATACTGCAGAACTACGTGCTTTCTCATCCATCTTAAACAAGAAGCAAT
GGGTTTTCTCTTTTAGAATCACGAACAACAACAACAACAACAAAAATCCTAGAGTTGTTTCATTGGTTCTGATTGA
CCTTACTTGGAACCATAAGTCCATCTTTGAATTAACCACTAAAGCCAGGGAGATGAGTTAAAACTAAGTACACACC
ATCCACAGAGCAAAGAGAGGTCTATTGTGGGAAAACTGCACAAATGGAAAAATAACAGGGGAAACACAGGAGAAGG
AGGTAGTTAGTTAATGTTCAATACACGCACCTTCTCAGGGACTTTTCTTCCATCTGGCATCATCTTTGTCTTTTGA
CCATTTTCCCCAATCAATACCTATATTCATACTCAAGTTTTCCATATTTGGGAAAAAACTTCTCTTGACACCAAAC
TCCCCTCTGACTTCCTTCTTATAATGATCTATGGTTGGATAGTCTACAATTTACTGTCCTTACTTTTTCATAGGTA
TTTTTTAATCTCCTACAGTAAGATTTCTACCACTGCACTGAAATCATTTTGTGTATGGTCTTCAGGGAATTCCATG
TGGCCAAAGCCAATGGATACTTTTGGCATGGGCTTTGGAGGTCTAACTTCTTGAATCCAAGTGTCGGCTCTAACAA
TCACTATCTATGTGACCTTAGACCAGTTATTTAACGCTGTCTTTATTTCCTCATTTGTTAAATGAGGATAATTGCA
GCAATAGAAAAAGAACGTAAACAATTATACTTATAGGATTGTATGATGATTAAGTGATTAATGCAAGGAAAGAACA
ACACAGAGCTTCAGACAGTGTAGATATTCAAATAAATGTTAGTTATGATTTTTATTATGTCTTGTGACATCTATGA
GATTTGAAAGTGTTAATCACTCTTGTGTTTTTGAAAATCTTTCTTTTCCATGTTCTATAAAAGTACACTTTCTTGA
TATTCCTCCTCCTTCTCTAGTCTTTCATTCATCTTATTTGCTCTTTGTGTTTTATTTATTCTTTTTACGTTGTATT
TTCCAGGAGTCCATTCCCAGCCCGGTGTTGGGCTCAGTCTATGTGCCATATAAATGAGAATGCACTTGCATTTGAG
GCTTTGCTTTTTATTTCTCTGTAGTGACGGCCAATCCCAGATTTCTGTCTCTCCCAAAATACACAGCTATGCTATA
GATCCTTCAGTCCTCCTGACTAAAATGTTAGTTTTATGTGTGTGTTCCTCACAACCTCCCAGTGAATAGGTCCAAA
ACTGAACTCATTATCTTCTCCATGCTCACTCTGCATTCTTTGGATAGGCAGTGAATAGCACCACTATCTAACAGGC
ACCTAAACCAGAAGCCAGGTAATCTGTCTTAAACCCTTTTGTTTTCCAAGTTCTTATCATAATGGCTGGTGTCTAA
CAGATGGTAAATAAAATAGAGACTAGCTGGCTACATGGATGGATGTGTGGGGATGCACAGAGGGATAGATGGATTA
TTGAGTGAGTCTCTAGCCTATAAATATTTTTAATTAACTAATAAATTATGATAATGTACAGTTGAAGGTCAAGGGT
GAAAAAGCATACCCTCAGTGGGATGCACACCCCAAGGAGCCATTTTAACTTGCTACATAACACACATATGACCACT
TTTTTGCTGAAAGCCTTCTATTATTGAGCAAGCATTCAAATCCCATGTTTGCCTGAAATAATGGTTCAGGTTATAA
AAGTTTCCTTCTTTTTCCAGGATTAAAATTATCTTCCTACATAATAGGAAGAACTGCTTTATCTTTTCCTAATATC
TAGAGATGGCCTTTTAAAAATATAGACTGTTTTCCCTATTGAAATGAACTGTAGGATGTACAAAATATTTACTGGC
ATGAATCAAAAGAGCTTGCTATGTTTATGTGAAAACCACTAGGCATTCTAAAAAATATTGCTAGCATAGTAAAATG
TTAGTAATTAAGACTAACGAAAGCGAAGGCAAATTGGAATCAGAGACTATTTTTAAGGAATGTCAACTGTATTATT
TTCAAATACACATGGTACATAACAGTAGGATATGAGAAAAAGTCCCAAGTATGTGTACTAAAGTAGCCTGCTATGA
TAAGTTGAAAAAGGGTTTGTAATTGGAATATCCACAGAATATTTCAGAACACTTAAAGACATTTTCATTTACACTT
TATACAGCTTTCTTATAAGAGCATTTACACCATTTATTTTATAAACCAAAGATTAATTAGAAGACTAAACAATTAC
AAGGCCTCAACTACGAAAGCTGTTCCACTACCTAGTGGAACAACAACAATGAGACACACAAAACAATGGCGTTCAA
AGATTAGAGAGAGACTTACGGTTAAACAGAGGTTGACATGTTAACTGAAGTTGCAATATAATATGTCGACTAGTTT
TGCAATACATAGCAAACACCCAAACAGAAATAAACCTGATAAAAAAACAGTAGTCTATAATGTGTGCCACTTACTG
AGTTTTAATTATTCTGGGGACTATATTTTTGATTTCATGTTACAATCACTAGTTTTGTGGGGTCTTTCTAGTCCTG
ATGCTTATTTACAAAATATCTGAAGTATTTCTTTCTATGTATTTATTTTTGAGATGGAGTTTTGCTCTGTCACCCA
GGCTGGAGTGCAGCGGCATGATCTCGGCTCACTGCAACCTCTGCCTCCCGGGTTCAAGTGATTCTCCTGCCTCAGC
CTCCTGAGTAGCTGGGATTACAGGCGTGTGCTAATTTTTGTATTTTTAGTAGAGACTGGGTTTCAGCATGTCGGTC
AGGCTGGTCTCGAACTCCTGACCTCATGATCCACCCGCTTTGGCCTCCCAGAGACCTGGGATTACAGGCGTGAGCC
ACCGCACCTGGCCATCTCAAGTATTTCTTTAACTTATAACTTCACATAACTTTGTGGAGGCAACAGGGTTAATTAA
AAAGGACTTTACTTACATAACAAAATAAGAAGCATAGTTTTATATTCCTGTGCCATATACATTTTGTTTGTCCATC
TGTAGCCATTCTTTGACCTTCTCTCCCTTGCTCTTTATTCTGGGAGGCTGACCTCTGTCATCATTGGGCTCCCATG
CCCTTTGGCTTCCAGTTGGTTTAGGCACCCAAGAGCCCTAGAAGGAAATTGAAGACAGGAGGTAAAGTGAGGTCAA
GATATTTATTCTCCTAATTCCCTCCCTTTGAGGTTGCCACAGGCTGACTATGTCCTTTGACAAAAGGTTATTGCTC
TTCTCAGGGTGGTTTCTCATTCCAATTCTCTGCTTTTGGCCACTTTTCCCTCCCCTCATCCCTTGGGCCTAGATGT
AGTAACAGCTCTACTGTTGCAAGGTTCTTGTATTATCTGTGATGGTTTCTTATACCCTGCTTATCTTGTGATTTGT
TGCTTTGTAGATAAACCTCTCAGATTATCTAGGCAAGATCATAGAAGAACACGTATGTCCAGCTAAGATATTCCAG
AAGACAGTAGGGAAACATTGAAGGGTTTTATAAGAGGGAGTGCAGTGATCAGATTTATGTTAGTTTTAATCTTTAA
TTGGGGTAGAATTTACATTCTAAAACAGAGATTTGGGTCTGGGAGGATGATATAGAGCCTCTTATGGATGTGAGGG
CAAAAAATGATAGAGGTTTGCAGTGCCAATAGAAAGAGGAAAGAAGTTATATATGAGAGAAATTTGTAACTAATTA
TATGAGGTGTTGGGTGACGCTTAGGAAAGAATCTAGAATGTCTATCAGGTTTCATGCTTAAGGGATAAAGTAGATG
GCAGTTTTATTACTTGTTTTCTGTATTATTTTTATTTCATAAAACCAGCTTAGAGAAGTTGCATAGAAAAAATAAT
GTAGTCCTGTTTATTTTAATATTTGAAAAGAACATATTTCAGAGTAGAATCTATATAGTACCTCCCTCTTGGACTT
CCAATGATACCAGTGATAGCCTCAATATAAGCCAGTCTTACAAAATGCACCCAGCGTGAATTCTTAGGTATTGTTA
AAAGAAGTTGGCCAGGCGCGGTGGCTCACGCCTGTAATCCCAGCACTTTGGGAGGCCGAGGCAGGTGGATCACCTG
AGGTCAGGAGTTTGAGACCAGCCCGGCCAACATGGTGAAACCCTGTCTCTATTAAAAATACAAAAAAAAAAAATTA
GCTGGGCATGATGGCACGTGCCTGTAGTTCCTGCTACTCGGGAGGCTGAGACAGGAGGATAGCTTGAACCCGGGAG
GCGGAGGTTGCAGTGAGCGGAGATTGCACCATTGCACTCCAGCCTGGGTTACAAGAGCAAAACTCCATCAAAAAAA
AAAAAGAAAGAAAAAGAAGAAGTTTCTAATACACTTATCTTCCCTTGGGTTCACTCAGAAGACCCTTGGAAAAGGT
TTTAAGAGCAAGTGATTTATTTGGGGGGTAAATTAATCAGTAGAAGAGTGGAAAAATGAGACAGGTGAGGCAAGGC
AGCCAGTAAAGAGTGGTGCATTATCAAGCCAGCTGCTGTTGTGGGTCACTGGAGCTTTATCCCTTGGGAAACTCTG
GAACCCTTGTAAAATACATGCCTCAGAGTTATTTCCCCTAGCATCAAGGGAGCTAGTGTAACAATATCCCAATTCC
TACAATTAGTCATTATATACAGGCTGCCTCTGGGAGCTGGAGGGGAGGCATCAGTTGCCTGGTATGTCTAGCCTGT
CACATGGATGGCAAAGCAAACTCCTGTGGCAACAGAAAGCCTTCAGGCAATGAAATGCTGGCACTGGGAAATCAGG
CTGATGGGTGCTGAAGTGGCAAGGATGAGGGGATATGGATATTCTGCTGTAGTGCTTTTCTAACAGATGATTCATA
TTTGGTTCTAGGGATCAAGAATTGAGTTAAAATTTTATATATATGTTGATGTTCTATGTCACCTTCAGGAAAATAA
TTTAACAGAAACTAATATTTGCCATCAAAAAAGCAAAGAATCCTGTTGTTCATCATCCTAGCCATAACACAATGAA
TAATTTTTTAAATAAGCAACATAAATGTGAGATAACGTTTGGAAGTTACATTTAAAATGTCTCCTCCAGACTAGCA
TTTACTACTATATATTTATTTTTCCTTTTATTCTAG
Homo sapiens dystrophin (DMD), intron 52 target sequence 1 (nucleotide
positions 1614980-1615029 of NCBI Reference Sequence: NG_012232.1)
(SEQ ID NO: 755)
GTAAGTTTTTTAACAAGCATGGGACACACAAAGCAAGATGCATGACAAGT
Homo sapiens dystrophin (DMD), intron 52 target sequence 2 (nucleotide
positions 1614980-1615024 of NCBI Reference Sequence: NG_012232.1)
(SEQ ID NO: 756)
GTAAGTTTTTTAACAAGCATGGGACACACAAAGCAAGATGCATGA
Homo sapiens dystrophin (DMD), intron 52 target sequence 3 (nucleotide
positions 1615029-1615068 of NCBI Reference Sequence: NG_012232.1)
(SEQ ID NO: 757)
TTTCAATAAAAACTTAAGTTCATATATCCCCCTCACATTT
Homo sapiens dystrophin (DMD), intron 52 target sequence 4 (nucleotide
positions 1664873-1664926 of NCBI Reference Sequence: NG_012232.1)
(SEQ ID NO: 758)
GAATCCTGTTGTTCATCATCCTAGCCATAACACAATGAATAATTTTTTAAATAA
Homo sapiens dystrophin (DMD), intron 52 target sequence 5 (nucleotide
positions 1664953-1665002 of NCBI Reference Sequence: NG_012232.1)
(SEQ ID NO: 759)
GAAGTTACATTTAAAATGTCTCCTCCAGACTAGCATTTACTACTATATAT
Homo sapiens dystrophin (DMD), intron 52 target sequence 6 (nucleotide
positions 1664774-1665023 of NCBI Reference Sequence: NG_012232.1)
(SEQ ID NO: 760)
TCAAGAATTGAGTTAAAATTTTATATATATGTTGATGTTCTATGTCACCTTCAGGAAAATAATTTAACAGAAACTA
ATATTTGCCATCAAAAAAGCAAAGAATCCTGTTGTTCATCATCCTAGCCATAACACAATGAATAATTTTTTAAATA
AGCAACATAAATGTGAGATAACGTTTGGAAGTTACATTTAAAATGTCTCCTCCAGACTAGCATTTACTACTATATA
TTTATTTTTCCTTTTATTCTAG
Homo sapiens dystrophin (DMD) intron 52/exon 53 junction (nucleotide
positions 1664994-1665053 of NCBI Reference Sequence: NG_012232.1)
(SEQ ID NO: 761)
ACTATATATTTATTTTTCCTTTTATTCTAGTTGAAAGAATTCAGAATCAGTGGGATGAAG
Homo sapiens dystrophin (DMD), transcript variant Dp427m, exon 53
(nucleotide positions 7905-8116 of NCBI Reference Sequence: NM_004006.2; nucleotide
positions 1665024-1665235 of NCBI Reference Sequence: NG_012232.1)
(SEQ ID NO: 762)
TTGAAAGAATTCAGAATCAGTGGGATGAAGTACAAGAACACCTTCAGAACCGGAGGCAACAGTTGAATGAAATGTT
AAAGGATTCAACACAATGGCTGGAAGCTAAGGAAGAAGCTGAGCAGGTCTTAGGACAGGCCAGAGCCAAGCTTGAG
TCATGGAAGGAGGGTCCCTATACAGTAGATGCAATCCAAAAGAAAATCACAGAAACCAAG
Homo sapiens dystrophin (DMD), exon 53 target sequence 1 (nucleotide
positions 1665027-1665073 of NCBI Reference Sequence: NG_012232.1)
(SEQ ID NO: 763)
AAAGAATTCAGAATCAGTGGGATGAAGTACAAGAACACCTTCAGAAC
Homo sapiens dystrophin (DMD), exon 53 target sequence 2 (nucleotide
positions 1665044-1665098 of NCBI Reference Sequence: NG_012232.1)
(SEQ ID NO: 764)
TGGGATGAAGTACAAGAACACCTTCAGAACCGGAGGCAACAGTTGAATGAAATGT
Homo sapiens dystrophin (DMD), exon 53 target sequence 3 (nucleotide
positions 1665089-1665141 of NCBI Reference Sequence: NG_012232.1)
(SEQ ID NO: 765)
AATGAAATGTTAAAGGATTCAACACAATGGCTGGAAGCTAAGGAAGAAGCTGA
Homo sapiens dystrophin (DMD), exon 53 target sequence 4 (nucleotide
positions 1665158-1665206 of NCBI Reference Sequence: NG_012232.1)
(SEQ ID NO: 766)
GCCAGAGCCAAGCTTGAGTCATGGAAGGAGGGTCCCTATACAGTAGATG
Homo sapiens dystrophin (DMD), exon 53 target sequence 5 (nucleotide
positions 1665173-1665228 of NCBI Reference Sequence: NG_012232.1)
(SEQ ID NO: 767)
GAGTCATGGAAGGAGGGTCCCTATACAGTAGATGCAATCCAAAAGAAAATCACAGA
Homo sapiens dystrophin (DMD) exon 53/intron 53 junction (nucleotide
positions 1665206-1665265 of NCBI Reference Sequence: NG_012232.1)
(SEQ ID NO: 768)
GCAATCCAAAAGAAAATCACAGAAACCAAGGTTAGTATCAAAGATACCTTTTTAAAATAA
Homo sapiens dystrophin (DMD) exon 53/intron 53 junction target sequence 1
(nucleotide positions 1665218-1665264 of NCBI Reference Sequence: NG_012232.1)
(SEQ ID NO: 769)
AAAATCACAGAAACCAAGGTTAGTATCAAAGATACCTTTTTAAAATA
Homo sapiens dystrophin (DMD), intron 53 (nucleotide positions 1665236-
1716747 of NCBI Reference Sequence: NG_012232.1)
(SEQ ID NO: 770)
GTTAGTATCAAAGATACCTTTTTAAAATAAAATACTGGTTACATTTGATAAAATTATACCATAGATTGTAATTTAA
TGATGTTTAATGTAAAGTTATTAACAGAAAATCACGTTAAAGCTGAAATGAACAGTAGACTTTGTATATTTATTTT
CTTAGAGACAGAGTCTCACTGTCACCCAGGCTAAAGTGCAGTGGCACAATCATAGCTCACTGAGCCTTGAACTCTG
GGGCTCAAGCAGTCCTCCTGCCTCAGCCTCCCTAGTAGCTGGGACTACTAGCCAGGCGTGTACCACCACGCCTGGC
TAATTTTTTAAAAATTTTTGTTTTCTGTAGAGATGGGTTCTTGAACTCTTGGCCTCAAGCAATTCTCCTTCCTTGG
CCTCCCAAAGCACTAGGATTACAGGCATGAGTTAGCATGCCTAGCCAGTAGACTTTTGAGTCAAGGTAGAGAATAG
AGGAAAATTGACAGCTAATGTCAATGTTAAAGTTAATTTTGTTTAGTAATCTGGATATAGTTGGCTGGTTTTCTGT
TGCACCATCTTTAAGATCACTTCAAAATTGGTATACGTATTTATGTAGTTGCATGAAGTCAACCATTCGGGCTTAA
TTCCTTCCTAGAAAAAGTAACAGGTCTACTCTTTCATATCTTATTTGGAGGCCCTGGATCAAGAAATGGGGGTGAG
TGGGTATCAGGATTGGGGAGGAATAAGGAGTACTCTCACTTTCTCCCCCCATTCTCTTACCTCCAGCCCAGCTCAT
TCTTCTCTTCTTTCTCTTTCCTCTGAAATTTCCTCAAATATCTGTCTGCTTAGTAGTCTATAATTCCTCAGGATAA
AAGAAAAAGGAAAAGGAAGAAAATTGCTTAATAACTATGTTTTATCAAGCAGCATGCATAGTGCTTTGCATGCGTT
TAATCCCTTCAAACTTAGTATTTACTAGTCAGTTTGAAAAGCCCTTAAATGTTAAACAATGGTTAAGCTTTAGGGT
ATTTTGAGCAGGGAAGTAACATGGTAGGAGGTTGATCAGATTGGGCACTTGATCAAAACCCTCTATAGATATTTTA
TCTGACAGAAAAGGCCTTTTGCTAAACCAGTATCTGATTCTCAAATTGGTTATGTCATCTAAGAAGGAAACCGTAT
CATTTTCTTGGATGAAATGGCTTCCATTTCCAAATTAGTCAGGTTAGGCTATGCTAAGCTATAGTAAGAAATGAAC
TCTGAAATCTCAATAGCTTAACACAATAAAGACTGCTTTCTTGGTTATATCATAGTCCAGTAGAGGTTGACAAGCC
ATTCTTAGGAGCTGTCCCCCAACATGGTCACTTGGAAACCCAGACTTTTCCAATTTTGTGACACTTGTCATTTTCA
ACACACTGCCAAAAGCCACTGCAGAAAAGGAAGACAGAATGTGGTCAGTCAATCCATGGTGTTTTATGGCCAGGCC
TGGAAATTATGTATATCACTTCCACCACATCCCATCACACAGAACTTGTTCTCGATTGAATTTCAGGAAGAGTTAA
TGGAATTAGTGAGCTTTTAGCCAATCTCTGTCATAGCCATTTTATCGAAGGGACCTGAGCTGCTATGAGGACAAGG
TTTTAGTTTATATGATGAGGATGTTAGAAAGCCACATAGAATTTTACAGTCTTATCATTAGCTCTGTTGGAGTCAA
AATCTAGGTGAATAAAAATTCTTTCAAGAGACACATGTTACCACTAATGGTTTTTTCAATGGATGAGCATAGATGA
ATTAGGAATCTCCAATGGTCATTTTCCTAATTCCTGGTCTGACTATGTAATATATAGAATCATGTGGTTATTCTTG
AAAGCTCTAGTCATATTCGTGAGGTAGTCAGCATGTTATGCTACTTCTTTTTCTTCTCTATAAGTTTCGGATACTA
TGAATCTATTGGATCACAAACCAAAGTAGTTAACTGGTAAGAAGGTATAAGCAGAAAATACGAAGTTTGGACTGGA
TCCTTGAGTTAGATGAGAAGAAGGAGAGATGAGAGGCATACAGTGATAGATTTGACTAGGGCAAGAGGACTCTGGT
AGTTAGCAAATGAGAAATAAATTTAACCTAAGAAAATCCTAAAGTGATGGTAAGAATGGTTAGGGTTTAATTGCTC
TTAGAAGAGCAAGTAGCTTTAAATAAGAGACAAAAATATCTGCAGAAGGAAATATGAAAGTAGAAAACAAAACAAT
TGCATTGTCAAAACATGTAAGATGTTTTAATCACCTAAAATAAAACCAGAATTGTATTATAATTGCTATCCTTTGT
CAAACTTTTGAAATTAAAAAAAAAAAACGCTTTGGAATGGTCTAAAACTACATCATAAAAATGGCTCAGCCACTAT
CTATAGAGATTGACATTTTTTGTTTGTGCTCTGTGTTTAGGAATTTAATGATGTGTATTGCTGCAGATTCAATGTA
AGTTCCCGATACAGATAAAGATGGCCAAAGCTGTAATTTTTTCATCCATTTCTTGAATGATGTATGCTAAAATTAA
AGTAATCCTAATGTTAATAATAACTTTTAGTGAATGCTTGCTGTGTGTCAAAAAGTATGCTAGTCACTTTATATAT
ATTGGTTTATTTCATCCTTTCAAAAACACTCTTTGAAAAGTACTCTTATTATTCTTAGTTTGCAGAGAAGAAAACT
GAAGGCTAGAGAAGTTCAGAATCTTCTTTTGAAAGTAGTAGAGTTGTGATGCGAATGCATAGCAATTACTCTTGTA
GATGACCTCACAGGCTTCCTCTTCCAATTGTCTACGTGGCTAAAACAAAGCAAGAAAACCCCATAAGAATCTGAAA
AAGACTCATGTAATTATTAATTGACAAATAAGATTATATTACATTACTGATAAACCAACAGGTAGAAAATGAAAGC
AATGAAATAGTATACTGCGAAAAAAAATTTCATATTATATTCTTTGATGAAAAAATAAAACAATAATTGCAAACTG
CATTTCATTTTTAATGAAACGACAATGTTTAATTCTTCTAAAAGGTAGGGAGCAAATAAAATGGTTATCTGGTCTT
TACATCGAATGTGTCATCACTACTAAGTGATATTAGCTGGCTTATTACTTAAAATTTTCTTTTAGGAAAAAAGTGT
GAAAATAGACAGGAGGAGACATTAAATGGCTAAAGCAACACCCCCTTGCTCCGTAAGTTCTTTCTGAGTCTGTGTG
CCTAAGATTAACAAGAGAGCCTGTGAGCCTCACAAGGCAATCACAAAGCTATGTCTACAGCCTTGGAGAGCTGTGA
TTACAGCAGCCAGCCTCCTTTGCAGCACCTCAGTGATGACTACACTAACTCTCACTACTGAGCAACAATGAAATGT
TTCAAACCCAACCACCAACACTGTTATAACTCTTAAAATTTGAGCCAGTCACCATACCTGAACGTAAAAAGAAAAT
TACCAAAAATGCTTGATACCAAAAAGCAATTAATGATTCATAATTGCTAATCTTTATTGACAGTCTCTTCTAGTTC
TTATCTACATATATTTTAATTGTCTACATAATGTAAATTAAATTTTAAATTCTGATTTGTAAACTTAATATTGCAT
AAATATTTATCAATGTTGTTAAACATCTTTTAATAATTATTTTAACTTCTTTATATTATTTCATTAATTTCCATCT
TTATGTCCTTGCTGGTCTGACATTTAGAAAAAACTCCTCCTTTTATTTAGAAAAAAAGAATGGTGAGGCCGGGCGC
GGTGGCTCATACCTGTAATCCCAGCACTTTGGGAGGCCGAGGCAGGCAGATCACGAGATCAGGAGTTTGAGACCGG
CCTGACCAACATGGTGAAACCCTGTCTCTACTAAAAATACAAAAACTAGCCTGGCGTGGTGCTGCATGCCTGTAGT
CCCAGCTACTTGGGAGGCTGAGGCAGGGGAATCGCTTGAACCCGGGAGGCGGAGGTTGCAGTGAGCCAAGATCACG
CCACTGCACTCCAGCCTGGGTGACAGAGCAAGACTCTGTCTCAAAAATACATAAATAAGGAGAATAAATGGGGACA
ATAACAACCTATTCACAGAACAGAAATAAAACCGGAGATGTCATGTGGAAAAAGCTTATCTTGAAAGCGTGGTCAG
AGTGGAAAGAAACAGCAACAGGAGAGTCTTGATTTGGTGAAAAAACAGAATGCAGATGGCATAGAGTATCAACAAT
TAATTAGCAGGCCATCAAGAGACGGCCATAACATATTGGTAATTGTTTTCTTTCACCCTTAATTTTTTCTAAAACA
AAAACATGAAGTTAGAAAGTAGTTAATTTTGTTAGCCACATGGCTTCATGGTTTAATTTTATCTTCTCTATGGGCC
TGGTACAACAATAAAGTTCTTTTGAGATAGAAAGACAGATGTTAGGCAAAAACATACAAGATTTTCATTCTAATCA
AAATCTTTGCCAATAGGCTATGACAGAATCCTATGAGAGATGATGCCATTTAGAACTTTCACAAGAGGCAAAAATA
GTATGCCATTGGCACCTAAAACTGCCAACAGTGATCATCATAGGATTTATTTCATTTGGCGGATTCTTGGTTGAGT
TCATAACCTGTTCCCTAGGCTTTTAAGATTGGTCCAAACTTGGCAATGGATTCTTTGGAAAAATTATTTCTTGATC
GTTTTAAACTTTCTTTTCCAGTGTCAAGAGAGATTTTCCCAGGCTCCGAGTTTTCATTTATTTTTTCCCCTGTCAG
TTTTTGAAAGAGAAATCCCCTTTTTGTCGGCTTTGTGATTGCTTTTGTGTCAAATTCAGCGTAAAAGCTTTTCAAA
CGTGACAGCATTAGCTGTTTTGTGCTTGGCTGAGCTAAGAGTACATGAATTCAATAGAGAAGTGAAATCTTGTTTG
AGCCTGGCTGATTTGGCTATATAATATTGAAAACTATCTGAAGCAAGAAATTCAATACTTTTCAAATTATTGAATA
AACATTTCTAAGGCTGCAAAATCTCTGTGTTTTTGAAGGCATTCTTTAGGAAACTTGAAACACAGACATTCTATAA
TCACATAATTAATTTTAAAAGTTGAACTTATTGTATGTAGTAACTAAGGCAGCTATTTGAGTGTCTCAAATTATTT
TACTAAAAAGGGAGACATTTTTCAAATATGGAAGTAGTGCCTTACATTTTTAGTTATCTGTGGAAGGATCGATGGC
ATCATATATCTCATCCACCATAAAAATATGTACGGTAGTGGAAACTGAATTTTTCTTTTATATATTTTGTGTATTT
TTTGGAGTTGTACAAAATTAAATCAGCAAATTGACCCACACTAGTAGTTTGAGGTTTGGGTTTCTGGTACAATAAT
GAAAAGCATTATTAGAAATCTTCTTGCCAGGACCTACCATTTCTTCATGTATTTTCTTTATTCCCTTTTGTTCTCC
CCTAAAGCTTCTATGTGATTAAAGAGAAAAGATCAATAACCCAATAGTACTTTTCCTTCTAAAGTATGCATGAAAC
TCTTAAAGTTCTCAAAGCATACAAACAGAGTGCTCTCTACATTGCTGATAGAACACAGGTAGTAATGGGCTAGTTT
CCCCAGGTAACTATTATATTATATAAATATTTAATGCAGAGTCACCAAGCCTTGAATAACTTTATGCTTTGATTGT
ACCATATTTTCTCTAGTTTGCTTTTCACAATTTAACTTATCCCGTGCTTGAAAATATAATGTGGTAACTGTGTAAA
TCTGCTCCTCAAAAAATTTTCGAAATCATCTTTTCTTTTTTCTTTTCTTTCTTTTTTTCCTTTTTTTGAGATGGAG
TCTCACTCTGTCACCCAGGCTGGAGTGCAGTGGCATGATCTTGGCTTAGTGCAACCTCCACCTCCTGTGATCAAGT
GAGTCTCTCAGCATCCCAAGTAGATGGAACTACAGGCACATTCCACCACGCCCAACTATTTCTCGTGTTTTTAGTA
GAGACAGGGTTTCACTATGTTGGCCAGGCTGGTCTCAAACTCCTGACCTCGAGTGTTCCGCCCACTTCAGCCTCCC
AAAGTGCTGGGATACAGGCGTGAGCCAACATGCCTGGCCCCACATCGCCTTTTCTGAACCCTGGGAATTAACCAAA
GGCTGGCAACAATTTTTGAAATGCTGACTCTCAATAAAAATGGCAAAGTTTGAGGTTTTTTAAGTTGGCCTTTCCC
GTCACTTTCTCCCAGCTCCACAGTAGCCTTGAAAATCAGTAGCCTTGCAACCATAATAGCTGTGAAAACTAGCAGC
CTTTTAGCCACCAGAGAGTGTAGACCAGGATTGGAATTCTTCAAAAAGGCCAATTTTATTCCAAGAACCTTGCCAC
TGTTTGACCTATCTTGCAGCTCCCTGGAATAAACCTATATACATGGCCTTGCCTTTATTTGGCCCGACTTAGAGAT
AACTTTCTAGGAAAAGCCCTAGCTCCAGGGTGTTGGTCCAAACCAATTAGCAGCAATTCTTTAACATAACAGGTTC
CTAAGGCTGTGATGCTAGTTGAGATAAACATAGGTCTGGAGAAAAACTTCCAGTGAAGCTCTGGAGAATGAGGTGT
CCATAGGGGATTTTGGCAACCTCTGACATGTTCTTGCGGGATCTAGAAGCCCATGTGCTTGTCTTAACAAATAGAG
AGTATCAATAAAGAGATAGAAATTGTAAATAAGAACTCAGTGGAATATCTGGAGTTGGAAAGCACAATAACTAAAA
TTAAATTTTCACTTGACGTCAGATTCAAGCTGCAAAAGAAAGAATCAGCAAACTTGAAAATAGGTAAATGGGATAA
TCCAATCCAAAGAAAAGAAAGAAAAAATATTAAGGAAATACAGAAAGCAAAAACTGACTGAACTGAAAGAAGAATT
AGGTAATTCAACAAAAATAGTTGGAGACTTCAACATCTCACTTTTAATAACAGGTAAAAGAACTAGGCAAATTAAC
AAGGAAATAGACACTTGAAAAAGACTATAAAGACTATAAACTTACAAGACCTAACAGATATCTATACAGCGTTTCA
TCCAATAATAGCAGAATACTCATTCTTTTCAACTGTACACGAAATATTCTCTAGGGTAGGCCATATGCTAGGTCAT
AAAATAAGTCTCACTACATTCAAAGGGATTAAAATCATACAATGCAGATTTTATGACCATAATGGAGTACAATTAA
AAATCAGTAACAGATGTAAATTTGGGAGTTCATAAATATGAGAAAATTATACAACACACTCTTAAATAGCCAATGG
GACAAAGAAGAAATAAGAAAATACAAAAATATCTTGAGATAAATGGAAATAAAAATACAATATACCAAAACTTATG
GGAGGAGGCTGAAGCAGTGCTTAGAGTAAAATGTATAACTATAAATACCTATATTTAAAAAGGAAAAAGATCTGAA
ATCAGTAGCCTAAGCTTCTACCTTAAGAAACTAGAAAAAGAAGAGAAAAGTAAATCTACAGTAAGCAGCAGGAAGT
AATAACAATTAGTGTGAAAAAAATCAGATAGAGGAAGAAAATATAATAGAAAAAATCAACAAAACCAAAATCTAGA
AATTTAGTTATTTGAAAAGGTCAACAAAATTGACAAAGTTTTAGCTGATTGGTTAGGAAAGAAATGGTCAAATTAC
TAATATTAGAGAAAAAGAGGGTGCATTACTACCAACATTACAGAAATAAAAAGAAGTAAAAGAGGATATTATGAAC
AACTAAACAAATAGATAACCTGGATAGACCTATAACAAGAAATTGAATTGGTATTAAAAACTTCTCCCAATGAAAG
GCTCAGGCCCAGAAGTCTTCCCCCGATGAATTCTACCAAACATATAAACAACTCTTCCAAAAAATAGAAGAGAAAG
GAACACTTCCTAACCAGACAAAGATATCACAAGAAAACAAGCTACAGAGCAATATGCCTTATGAATATAGACTTGA
AACGCCTCAAAAAATTATCAAACCTAATACAACAACATATTAACATGATTGCTACCATAACCAGGTAAGATTTATC
ACAGGAATGTAAGGTTGGTTTAACATCCAAAATTTAATCAATGTATTATACCATATCAATAGAATAAAGGACAAAA
ATCGTATGGTCATCACAATAAATATAGAAAATTCATGTGACAAAGTACAAAACCCTTTCAAGCTTGAAAAATAAAG
AACACTTAACAAATGAAGAATAGAAGGTATTGGCCAGGTGTGGTGGCTCACGCCTGTAATCCCAGCACTTTGGGAG
GCCGAGGCAGTTGGATCACGAGGTCAGGAGATTGAGACCATCCTGGCTAACACGGTAAAACCCCGTCTCTGCTAAA
AATACAAAAAATTAGCTGGGCACGGTGGCGGGCGCCTGTAGTCCCAGCTACTCAGGAGGCTGAGGCAGGAGAATGG
CGCGAACCCTGGAGGCAGAGTTTGCAGTGAGCCGAGACAGCAGCACTGCAGTCCGGCCTGGGCGAAAGAGCGAGAC
TCCGTCTAAAAAAAAAAAAAAAAAGAAGGTTTTGTGGATTGAATAGTGTCCCCCGCTCCCAAAAGACATACTAAAA
GCCTGAGCCCAGGTATCTGCAAATGTGACCTTATGTAGAAATAGAGTCTTTGCAGATGCAGTCAAGTTAAGATGGT
ATTTTTATAAGAAGAGGAAAAGAGGCATACACAGAGAGAAGAATGCCACATAAAGGCACTGACCACGGGAAAAACA
CCATGTGATGTTACAGTTAGAGATTAAAGTGCTACAGTTGCAAGACAAGTAAAACTAATGATTAACGGCCATCATT
AGAAGCAAGGAAGAGGCAAGGAAAAATTCTCCCCTACAGGTTTCAGCAGGAGTATGGGCCTGCTGGAATCTTGATT
TTGGATCTAGCCTCCAGAAGTCTGAGACAATGAGTTTTGTCTACCCAGCTACGCAAATTGTGACACTTAATTACAG
AAGCCCGAGGAAACGAATACAGAAGAGGATTTCCTCAACCTGATAAAAGGCAGCTACAAAAATTCCACAGCTAATG
TTATTGTGAAACACTGAATGCTTTCCCCTAAGATCAGGAAATAGTGAAGGATGCCTGGTCTCACCACTTCTATTCA
GTATTGTACTGGAAGTTCAGGCCAGGGAAATTAGGCAAGAAAAATAAATAAAAGACATCCAGATTAGAAAGGAATA
AGTAAAATTATCTCTACTTGCAGACAACATGACCTTGTAATTAGAAAATTTTGAAGAATCTATGAAAAAATATTAG
AAATAATAAAATAATTCAGCAAATTTGTAGGATACAAGATCAATATACAAAATGAATTATTTTATACATTAAAAAT
GAATAATCTAAAAGTGAAATTAAGAAAATATTTACAATGGCATCAAAAAATGTTAAAAACTTAGGAATAAAAGAGA
TGCAAGGCTTGTATACTGAAGAATACAAAGCAGTGTTGAAAGAAATTAAAGATAAAAAAATAGAAAAATATCACAT
GTTCTCAGATCAAAAGACTTAATATTATTAAGATATCAATATCCCCCCAATTGATCTACAGACTCTATGGAATCAT
TATCAAAATCACAACTGGCTTTTTTTTTTTTTTCAGAAATTGACAAGTGGATCCTACCATTTCTATTGAAATGCAA
GGGATGTAGAATAGTCAAAACAACCTTGAAAAAAGAACAACATTGGAGGAGTTAACTTTCCAATTTCAAAAACTAC
TACAAAGCTACAATTATTAAGTCAATGTGGTACTAGAATGTGAATAGACATATAGATCAATGAAATAGAATTGAAA
GTTCAAAAGTAAACCTTTGCATTTATGGTCAACTGATTTTTGACAAGGGTACCAAGAGAATTCAATGGAAAAAGAA
TAGTCTTTCACAACCTGGTGCTAGAATAACTGGATATACACATGAAAAATAATGAAATTTGGCCCCACCTCACACC
ATACACAAAAATTAGCTTAAATGTATCATAAAAGAAAACAAATAATCTGATTAAAATGGGCAAAACACTTAAACAT
TTCTCAAAAGAAGATGTACAAATAGCTAACGGGTTTGTGAAAAATGCTCAACATCACTAATCATTGGGGAAATGCA
TATTACAATCGCAGTGAGATGTCACCTTACACCTGTTAGAATGGCTGTTATAAAAAGAGAAGAATGATAACAAGTG
TTGATCAAGGATATGGAAAAAAGGTAACACTTGTATATCATTGGTGGGAATGTATATTAGTACAACCATTATGGAG
AACAGTATGGTAGCTCCTCAAAAAACTGAAAACAGAATTACCATAGGATCCAGCAATCCCACTTCCGGGTATATAT
CCAAAAGAATTTAAACCAGTATGTCAAAGAGATATCTTCACTCCTGTGTTTATTGCAGCATTAGTCACAAAAGCCA
TGATATGGAATCAACCTAAGTTTCCATCAGTGGATGAATGGATAAAGAAAATGTGGTACATATACACAATGGAGTA
TTATTCAGCCTTAAATAAAGAATATCCTTTTGTTTATGACAGCGTGGATGAACCTGGAGGACATTATACTAAATGA
AATAAGCCAGGCACAGAAAGTCAAATACTGCGTGATCTCACTTATGTGTGGAATCTAAAAAAGTCAAACTCATTGA
AGTAGAGAGTAGAATGGTGGTTACCAGAGGCTGTGGATTTAGGGGTGGAAAATGGGGAGATGTTGATCAAAAGGGG
TTAAAAACTTTAGTCAGGAGGAATAAGTTTTTGAGACTTTATGACACAACCTGGTGACCATAGTTAATAATATTAT
ATTGTATATTTCAAAATTGCTAAAATAACAGATTTTAAATGTTCTTGCTATAAAATATGATAAATATATGCAGTGA
TAGATACGTTAAATTGCTCGATATAATCATTCCACAGTGTATACATATATCAGAACATACATGTACCCCATAAATG
TATATAATTATTTGTCAAAGCAAAATAAAATTAAAAATAAAATGTTTTAAAACTTTTGTAAAATAAACTTTATAAT
AATAATATATTTTTAATGAATCATAGAGCTAAATATATTAATAAGAACTAAAACTTTAAAACTCTTCTAAGAAAAT
GTAGGAATACAATGGATTATGCAGTGTTTTCTTAGATATGACACCAAAAGCAAAAGTAACCGAAAAATACACAATT
TGGACTTTAACAAAATTAAAACTTTTGTACTGCAAGCAATATTGTAAAGAAAGGGAAAAGACAATCCTCAGAATGG
GTAAAAATATTTGTAAATCATATATTTGATAAGATACTTGTATCAAGAATATATAAAAACTCATAATTCAACAACA
AAAAGGATAAATGACTTAATTACAAGTGAAGAAACAGTTTGAATAGGCATTTCTCCAAAGTAGATGTATAGATCTC
CATAAGCACATGAAAAGATGGTTAACATCATTGATTGTTTAAAAAATGCAAATGAAAACCACAAGGAGATAGTACT
TTTTACCCACTAGGATGGCTAATACAAAAAATACAGACAATAACAAGTGTTGAGAAGATGAAGAATGCTTGGAATT
TTCACATTACTGGTGGAAATGTAAAATGGTGAAACAACTTTGAAAACAGTTTGGAAGTTCCTGGAAATATTTTTTT
AATTTTTAATTTTTGTGGGTACACAGTAGGTATATATATTTATGGGATACAGGAAATATTTTGATATAGGCATACA
ATGTGTAACAGTCACATTGGGGTACCTGGCCTATCCCTCACCTCAAGCAATTATCTTTTCTGTTACAAACGACAAT
CTAATAATAATCAGTTATTTTTAAATTCACAATAAATTTTTTGACTGTAGTCATCCTCTTGTGCTATCAAATACTA
GATCTTATTCATTCTATCAAACTATATTTTTGTATTATAAATTTCACTACCCATTAGCCATCCCCCCCTTTCCCCC
TACCACTACCCTTCCCAGTCTCCTGGAAATATTAAACATGAAGCTACCATATGACAAAACCATTCTACTCCCAGGT
GTATGCTGAACAGAAATAAAAATACATATGTACACAGAAAACCTTTATACAGATGTTCATAGCAGCATTATTTATA
ATAGCCAAAAAGTAGAACCAACCCAAATGTCCATCAGCTAATGAATAAATTTTAAAATGTCCTCTACATCCAAAAA
TGGAATATTATTTAGCAATAAAAAGAAATGAAATACTGATATGCACTGTAACATGAGTGAACCTCAAAAATATGCT
AAGTGATGGAATCCAGTCACAAAAGACCCCAAATTGTATCAATACATTCATATAAAACATCCAGTAAAGACTGAAG
GAGGAGGAGAATAGACAATAGAAAGGGACTGTTAATTGGTATGGGTTTCATTGGGGGCAAATAAAATGTTCTCAAA
TTAGATCATAATAATGGTTGCACAACCCTGAATACATTAAGAACCAGAAAATTATGCACTTTAAGTGGGAGAATTT
TATGGTATGTTAAAAAGTATATCTGCATGTCATTGACTACAGATCTTTTCTTATTTTTTCAAGTTTTATGAACACA
GTTTTAGAAACATAGTCTCCATTTTTCTCTTTCATGAAGCTGCTTGCCAGCTATAGAAATGAAAAAATAGCCATGT
CTGTGGAACATAACTGCTACCTAGATGGGAGCCTCCATCACACAATGGATAGGGAGCTATTTTCTTTTATGGTCAA
GATAGGCAGGTTCTGTCCTATAGAATATATTCTGAGATGTTACCTGTAGTTCAGAAAAACAGGGATATTGTTGTTC
TAAACATTTCCTCAACATTTATGTGGCTTCCTATTTATTAATAAGAGAACCTGTTCTTTATGTGTCATTATACTAG
TTTATTCTAGCCCTGGTCCTGGTCCAGGACTGTCTGGAGGTATTAGAATTGCCCTACTCTATGTGAAATAAGGTCA
TATCTGAATAGGTATGTCAGTCAGCATAGGCTAAGCTACAGTAATAAGCAACCTCAAATCTAGAGGCTTAATAGTC
TATATTTTACTCATGTTACATGTTCGTTGTAGACTGATGAGAGCATTCTACTTAGCTTAGTCACTCGGGGACCATG
GCTGATGGTATCTCCATCTTGGTACATTCTTTCATGATCATAGAGGCAAGGAACATGGTTCTTGAGCCTTCTGCCT
GGAAGTGACCAGTAGACAAAACCAGTCACATGGCCATTCCTGAGTTCAATGGGAATGCCGATGTATAATCTTCTGG
CGTGAAAAGGCACTGGATATTTGAGAACAATAATGCAGTCTACCACAATATGCTCATGGGGTCTGAAAAGGAGCTG
AAAAAACGCCATTTTCTGGGGTTCTGTCCTCTGGCATTGTTTCTGGAATCGTTTCCACCCATCCATTTCTACACCC
TCTCAACTGACTTTTCTCAGATGCCTGGGGTAGCTGGGTGATTTGTTAATTACAGTATTGCTTAAGCCTTTTAATA
AAGTCAGCAAATTAAAGAGAGAGCCCAGATAAAGAGAATGCTTTCAAAAATGTCATAATGAAATTAAGGTAACGAA
GCACTTGGAGACCTCTCCTCCCTTCTTTTTGTATGAAACTTATTCCAAAGCAGCTGGAAGAGAAATGGGGCCTGAA
AGTAAAGAGGGGAAAAATATATTGTATGCCATTAGTTTAATTATAAAAATTCATTACATGAAGCCATGTAAGACAG
AAAATAATGGAACTCTAATGGGTTGGCCGATAACACATTTACTAGTCTGCTCTCAGCTTGTAGTCATTGTTGAAAG
GACAGAAAAGTAACGACGTTGGTATGTAGTCTTCAAAGCTATTGTAAAGATTAATCATTTTCAGTAAGCCGGAAAA
TGAGGGCCATTTTCTTAAATAGTATGCCCTGTATAGAGTGTTACCTTCTGAAACAGAGTTGTTTGGTAATGTCAGA
GGTTTAATAGATGAGTCTTGAAGTTAATGGTGAGAGGCTTTACTTTTGAAATGGCTTTCTTACAGCTATTTTAACA
TTTCTATTATAAATCCATAAAGAGGTTTTATAGGTCGTTTTAATTCACACAAGGAGGGAAGAAATTGTGCTTCTCT
TTTCCCAATAAGGATTTATAGAACATTAATTCATTCTTGGGACCAAATTGTTTAGTGATTTTCAAAAATAAACTAT
GTATTTTTCTGCTGTCTTTTTTTCAGAAATGCCTTACTTTATTATACATAATAATTTTCCAGTCATGCTATAATCA
TCAAGATTGTTTTATATTCTTCAAAGCTCAGTTAAAATAAACCCAGTCCTTTTCCTTAAATTTCTTTATATGGATG
CATAACTTCTTTAACTGAATTGAAATGAAACCTGAATTGAGAGTCCAGAAACCTGCATTTTTATGATGACTCTTTC
ACTGACTAGCTATGTCACCTTGAACAAGTCACTTAACCTTTCAGAGCATTTATATTCTCATGTGGACAATTATGGA
ATTGCTCAAAAAGGAAGAAGATATCAAAAGATGAAAGTGAATATGTAGGTAGGGGTCATATATTCGAGGGCCTTGT
AATCCAAAGAGAGAGGTTAGGACTTTGTCTTGCAGGTAATAGCAAACCATTGAAAGGTTTTACACAGAGGAGAAAT
GAGGTTCTATTTGCATTACAAAAAGATCATTTACGCAGTTGTATGTAGGTGGAAGGGTGGTTTGAGAGTGGAGGGA
GGGAGCCAATAAAAAAGATTATTTTAGTACCTGAGCAAGAGATGATAGGGACGTAAATTAAGGTAGTGTCAATGGA
AGTAGAAGATTTGGATAAAAGTTAAAAAAAAAAAAAAAAAAACTTATGATACAACTACCCAGGGAGTATGTTCCCT
GCCTCCTGCTCCAGTTGAGGACCAACATATCAGATTGATCCAGAGACAGAATTTTGGAGAACATTAATATTTAAAG
GACAGGCAAAAAAAAAAAAAAAAAAGCTTGAACACTCAGAGAATACAGAGGCGACCAAAAGAGTGGAAGCCAAGAA
AGCAGAGAACTTCGAGAAGAGAATAGTCAGCAGCACCAGTTGCTGCAAATAAATCAAGTAAATAAGAACTTAAATC
TTTCCATAGGCATTGGCTTTTGGTAACTGAATCAGTTAACTATTTTCACAAATGTGAGTGGCATATAGCAATAAGC
CCATATTGCCTTTCATGAATTTTCAGCTTGGGTGGGGTGGCACTTCTTCATGTTGCAGGTTGATTGGGGTCGCTCT
GCTCCATATGTCACTCATTCTCCTCCTGAGATGAGTAGGCTAGTCCAGAGCATGTTTGTCTTAAGGTAATGGTGAA
GTGCAAAAGGGATAGCCACACTGCATAAATACATTTCAAGCCTCTGCTTGTGTTACTTCTAGTAATATCCCACTGG
GATTAGGCAAAGCAAGGCGCATGACCAAGCTCAATGTCAAGGGCATGCTGTCTTTCGTAGGAAGAATTTCAGAGTT
ACTTAGCAAAGGGCATGGATACAGGGAGAGGTAAAGAATTGGGGCCAGTAATTCAATTTGCCATAGTGACTTCTGT
CAGAATAGTTTTAGTGGAACGATGATGGCAGAAGCCTGATTTTCATGAATTGGAGAGTGGCACGAATGTAGTCAAG
TTGAGATAATGAGTGAATATCTGCCTTCTAAAGAGTGTGACTGTGAAAGGAAAGAAAATGATAGAGTGCAGATTAT
TGTTGTTGTTGTTGTTTAAAGATGAGAGAGACACTCATGTTTGAAGTCTCTAGAGAAGGTCCCCGCAGAGAGGCAA
GAAGTTGGAACACACAAAGAGAGAGGGAGTATGTGGAGGAGGATCCCAGAATAGATGGGTGCCCAGATGGTGAAAT
CAACTTCCAATAGCAGAAAAGTCGCCTCTTCCTCTGAGACTAGAGAGAAGGATGCAAGATGTAAAGTCTGATATTT
CATGAAATGTATGTTTGATATATTAAATTTCCCCCTGTGGAATTGTAGGTAGGATCATTTACTGTGAGATTTCTTT
CAGTTCGTGTTGTCAGGAATTAGTCTGTAAACCACATTCGATATTTCAAACAGAGGAAATTCAATACAGAGGACTG
GCTCCTCCATAGGAATGAAGGAGCTGACAAACCCAACAGAGGACAGTGAGGCAACCCAGAGTTTAGCAACTTCAGG
AAACTATTACCACCTCAAGGGCTGGGGAACCGACAAGTTTTACCAAAGCCCAGAAACCAGAACCATCGGGTGCTAA
TTCACCCAATGTAAGGTCTGCACAGCAGGTAATGGAATTACAGAGGGAGCAGCCGTCCAGTGGGAAACGGAGACAA
AGGAGATACTGCCAAGACAGAGAGAGAAGGGGGATAATTATCTTGTTTCACCCTTTCTCATGCTCTTCTGTCTCCC
ACTAGTGTGTCTCATGACTTAACCTAGTTAGAAGCCAGCTAGCAAGAAATCCCAGGAAATTCTGACCTCCTATGGG
AGTTAGTACATCTCAATACTGAGTGGAGTAGGAAATGGGTAGAGAATGGATCTAAGAGCAAACGACCTCTTGGTGG
TTAGCTAACAGAGTTAAATTTTTCCATGAATCCCTTGGTTCATGAATGTAAATACAGGATCCAAGGAAAAATAACA
AATCCAACATGTAGAACTTTTTGTTCATAAGCATATTAGGATCCTGTTTTACAATTTTCCGAGCAATTGCTTGATT
TTGACACTTCAGGACAAGCCGGAGGCATGGTCAGAGCAAGTAACCCCATCCTAGTTTACAGATGACAAAACTGGGA
CTTACAAGGTTAATGACTTCCTTAAGATCAAACTGCAGAACAATGGACAAGCAAAACTTCCTGGGAAGATGTCCTT
TCGTTACTCTGCACTGCCTTTCTTGAAGTTCCTTTGAGACACAGTCATTAAAAATTTAAGTAATAGTTCACCCGGA
ACAAACATTTATTTTACTTGTAGGTGAATATCACAAAACACATTTTAAAAGGAAATTTAACCAATATTCAACCTTA
AAATTTTAATGATAGAGCTATGCACTTGCTTTTATCTTCCAAGTTAAAGGGAGAATTTTAGAGTTGCAGCATTTCT
AGACAATGTCCATAAAACAGCGAGCTACTTGCTTTGCATTCAGAAGGGGCTCACACTGTGGGGAATATAGTTTGAC
TCATTAAAATTAGTAGTAGATGTGCTAGCAGTAGTAATGATGATGATGATGCTGATGCTGATGATGATTAATATTT
ATTTAATCCTTCGGGCACGCCAGGCACCTTTGTAAATGTTCTCCCTGAATTCTCTAAGTTAACCCACCTAATCATA
TTTCCACATAAATACTGTTAATATTCCCATTTAGCAAAGGAGGAAACTGAGGCACAGAAAAGATAATTAACTCTCC
CATTTGCCACTAAGCGATCACGCCAGGATTCAAACCCAGCAGGCTCATTGTATTGCTCAGGCTTTTAGCTACTCTT
TCTCTTCCTCCTCTCTTTTCTACCATGTAGGGTCAATATCACTAAAATAAGTTCTTACACATAAAAGAAATGAGAG
AAGACAAACAATTAAAGTAGAAAAAAATACTCAAAATTCTTGAAATTAATTCAGGTTGTCTCATAGTCCACAATCA
CTTTTTCTACCTAAGATACTGTGAATATATATCAAGCACATAGTGCAGTAGCAGCATAATGGGTTTGAATGTGAAA
CATTTCTTTCCTGGTTGGGGAAACAATCTTTTGGGGATTTAGCATTTTATATTCTTCATAGAAAACACACACACAT
ACACACACACACACATACACACACACACATATACAAAGGCAATCTTTTTTTATAAGCTAGTGCTCACAGTATTTTA
AATTTCTTCACACACCATGATCACCACACAAGCAATTCCCTGTGGTGTACACCATTGTCAATGGAATTCAGAAAGA
GAAAAGGGTGTGTGTGAAGTCTGTTGTATTTTTGAATTTACACTCCTTCTGAGGTCTGGAGTTATCCCAGTAGTAA
ATTTCACAGCAGTAGGCAAAGCTGGTCAAGGTCATGGTTCCTAGTGTGAATGACACTTGGGTAAAACCCAAGGTGG
GCGGGCAGAATATTACCGTAGGAGAAAGGGAAAACATTCTGTGTGGGGAATGTCTTTAAGAAGGGAGAAAAAAAAA
AAAGGCTACCTTTGAGAACCTTTGTGTTTATATAAGGGCCTGAGCCCCCTCAATGTCAGTCAAGGTCAGTGGGAGT
TTTACTTTTTAATCCTACATCCTCACAAAACCTGGGCAGTATTCAGTGAGGGTTAGCACTCAGTCAAGATTTCCTT
AGGTATAACAGTCCTAACTTTCGCTGTGAATACAATTTTGCAGAAAAATATATGGCACGCGTATTTCTACCTTTCA
AGCATTAAAGTACATCAAAAAGTTCTGGTTTGACGTCAAGCCTCACTCTTTACTACCTTGTTTAGTAGGAAATCTG
TCATTTAACTCTAGCTTACTATTTCTTTCAGAGATTTTGGAAAATTCTAGGATATGTTATAATTTATAATTTTAGG
CATTTTTATGATCTATTGATTATGTTTTTAGATTCATTGGCACCTTCTGCATTTTATGTTTTTTAATTTTAACAGC
TTTATTGAAGTGTAATTGATATACAAAAACTGCATAAAATTATATGGGCTAGAACATGTGCATATACCCATGAAAC
CATCCAAATGGTATAACCTCTTATATCTAGACTTTTCAAACAGGAACCTATGAGTGATGGGTGGCTTCCAAGGTTA
TTTAACTTGCTTGAGTAAAATAAATGATTTTAATTATATATTTACTAAAAATTTTGTTTTTATTTTGTATTCCTGA
GCTTTTACATATATGCTCTGATACTGGTCCATCATAAGTATTTTATTAACAATTCAGTGTTTTATAAAAGGAATTC
ATTTGAAAAAAAGGTAACCAAGTTATTGGTCTCAGTTTTGTGGATGTTCATGCTATTAATTAGCAATTTAATAGAT
GAGAGATAATAAGCTCTTCTTTTAAAATGCAAATAGCACCCTCTGGATACTAAGGGCCAATTACATGTTCTTTCGT
ATCTTGGAATACATTTCTTTTATCACTTTAGGTTTGGCTTAAGTGAGGAGGAGAAAGAAAGAAGTGGTAATTTTGT
ACCCTAGGATATTTCTAGAAAAGGCAAGCCTTTGGCAGAAGTTCCCTCAGAAGGGCTTTCAAATAGTGCGAGGATC
TGAAAAGTGGAGAATAGAAACTGTAATGCTTGATAAAAGGAGTATGGAAGAAAAGGCAGGCATTCCACGCGGATAA
ATGTTAATTGTTTTGTAATAGCAGCAACTATGCTCTGTGCCTTTAGACACTGTCCTCTGCTTTCATAGCAAAAGTC
AGTGATATTGGCATTAGCTTCTTGATAATAGTTTGGTAATCTGGGTCTATTTGCTATATGCAGTAAATACTATTAC
TTGGTTTGATTCTAGGATGTTTTAGTTAACTGAGAAATTACATTATATATAGATTGTATATGTTCAATAGGAGTTC
ACCAAACACGCTGAAATTATGCTAAGAACACTGTTTGATTTTTATTCTGTAAATATCATACTAACGCAAAAGAACA
AATATTAAAACAAAAATGATTTTTCTCCTCAGAAGCATTATTTTCAATTTCTTCCATTTCGTGTTCAGCTCATGAT
TATGTATTCTTAGAGATAAACACAGTGTAGTTAAAAATTGTGTTCTGTTTTTTACACAAGAAAATTGTGCTCTTTT
TACAGTGATTACTGTGCACATTTGCTACATATTGGACCATTGGAATGATGTATTATGTTCTTTTAATGTCCCTGCA
TTAAACATCGACATTGTTTCTCTTTTTTTTTTTTTTTTTTTTTTTTTTTTTCCGAGATGGAGTCTCACTCTGTCGC
CCAGGCTGGAGTGCAGTGGCGTGATCTTGGCTCACTGCAACTTCCATCTCCTGGGTTCAAGCAATTCTCTGCCTCA
GCCTCCCGAGTAGCTGGGATTACAGGAGCCTACCAGCACACCCGGCTAATTTTTATATTTTTAGTAGAGACGGTGT
TTCACCATCTTGGCCAGACTGGTCTTGAACTCCTGACCTCGTGATCCACCCGCCTTGGCCTCCCTAAGTGCTGGGA
TTACAGGCATGAGCCACCGCACCCGGCCTGTTTCTCATTTTTTTACTGCTTTGGTTAATGAAGATTTTAAGTCATA
TAGTATATTGATCTTCTTTTGAAGATTTTATCAGGATAAAGTACCAAAAGTGAGATTATTGAGTCAATGGGAATAA
GCATTTGTATGTCACTTGGTATCTATTGCCAAATTGTCCTAATAGATAACGGTCCCCAGCAGTAATACGTGAATGT
GCTAGTTTTACTGGTCTTTTCAGCTTTGGATTTTATTTTTTGTTAATTTAATTGGTGTCCAATTGTACCTTTTCAA
TTTAATACTCTATTTTTTATTACTTGAAAGTTAACTTTTTTCTTTTTAAATAAAATCATCCCACTCTTTCTCTTGT
TTGAACTACCTGTTCCTATTCCTGTGTCCATTTATCAATGACTTAACCCATCCTATTGAATTTGATATAACTATCT
TAACAATTAGTATTATCTTAATGCTTAGAAGCCTCCTCTCTGCACTTGCAGAAATGTTTAATAGAAATTTCAGGTT
GATATGCTTCGAGATAAAACCTACCATTAATTGACTTCTTTCTTTGAAATATGTTTCAAGTGATGAGATAGCAAGT
TTGAAAACACTGAATTGCCAAAGACAAACAACGAAGAAAATGTAGGTGATTTGGTTTAGTAAGATTCTTATATTTT
GACTTTTTTTCTAATTTTAAAGTAAGGTATTCTAAAAGTATTTTGATCCTTCAAAAAATTCGTGACTAGAATAACA
GTACTATTCTTTTGTTGTTCAATATTTTCTGTATGCAGGTTTAGTTGAACGCCAGTAGATGGCACTAATTACATAA
ACAATTCAACAAGTTAATGAAGAGGGAAAGAAATGTATGAGGTTTTTTTCGTTCAAATGTTGTTATATGTCACATA
TTCAACAATTATATATGAGCTTATTTTTGTAGTTTTTTTCTCTTGTGATAAAAACAATTAAGCCCACTTTATTGCC
AATTAATTGCTACTAAGTTGAAATACTTGATACTGGTTATTGCTCAAGATGCTGCATTTGAAAAGTTTGTCCTGAA
AGGTGGGTTACCTTATACTGTCATGATTGACTAAATCATATGGTAGGTTAAAAGCAATCTAATATATGTATTCTGA
CCTGAGGATTCAGAAGCTGTTTACGAAGTATTTTAAGACACTCCAACTAGAGATTTCATAAAAAAAACTGACATTC
ATTCTCTTTCTCATAAAAATCTATAGCAGTTGGCCAAAGACCTCCGCCAGTGGCAGACAAATGTAGATGTGGCAAA
TGACTTGGCCCTGAAACTTCTCCGGGATTATTCTGCAGATGATACCAGAAAAGTCCACATGATAACAGAGAATATC
AATGCCTCTTGGAGAAGCATTCATAAAAGGTATGAATTACATTATTTCTAAAACTACTGTTGGCTGTAATAATGGG
GTGGTGAAACTGGATGGACCATGAGGATTTGTTTTTCCAATCCAGCTAAACTGGAGCTTGGGAGGGTTCAAGACGA
TAAATACCAACTAAACTCACGGACTTGGCTCAGACTTCTATTTTAAAAACGAGGAACATAAGATCTCATTTGCCCG
CTGTCACAAAAGTAGTGACATAACCAAGAGATTAAACAAAAAGCAAAATACTGATTTATAGCTAGAAGAGCCATTT
ATCAGTCTACTTTGATAACTCTATCCAAAGGAATATCTTTCTATCTCATCATGGCGCACACTGCCTTACCTGTTAT
CTGATAAATAAGTCACTTTGGGATTCATGATAGAGTTATAGCTGTACATGGTCTCATCCTAGTATCTCACTCCACA
CACCCAATGGGAAAATTTGTGGAGGGCAATATGACTCGTCACTTCATTTCCCATTATATATGAATGGAAATTAACA
GCGCTTATAGACAGTATCTCCTCAAACTAAGCCTTGTATCCTTATTATACCTCTCTTGATCTCTAGTGCTTTTTTC
ACTAGCATTTATTCCAATCATAAATAAAAATATAAATTATGTAACTAATTGTTAAATATTTGTCCTTTAAATTAAT
CTAAATGCCATGAGGGCAGAGATTTTGTCTTTCTCATTTGATACATCCCCAGGTCCTGAACCACGTGATATAATAG
GGAGCTAGTAAATGTTTTTTGAATGATGACTCCCTTTGCAGAATGTACAATTACCTTGTGCAAGCTGAAAAAATAG
CACCTGTACAATATGAGGAAGACCACGGTGAAAAATAATTGAGTTCCAAAATATGACATCAATTACTGAAAAAATA
AGCTCGGTGATTTTTAACAAGAAGTAAAAGTCACCACTGGGGCCAAAACAGATTTTGAACTAAGAGTAGGAAGTCT
TAGGAGAAATGAGATAATGATATATGGAAATTAAGCGGCCAACTAAATTTTGAAACTGAGCTAGACATTAGAGAGT
AAAAACTCCTGTGAAGCTGAATTTAAGCTGGTCACCCTGGGGAATAGAGCAACTCTAATCCTGAATTCCAGACAGT
AGGTGTATAGATGGAAAAGACCATGGAAAAGAAGATTCAACCTAAAGTTGGGAAGTTTTAATTGGAGCCCTATGAA
AAAGACCCTGGTGGAGAAAGGGCAAACTTGAATATGGAGCTGATATTTGGAAAAATTCTCATAGTAACTACTTTTT
CTCAATGGCAAGGCTTGGACTTTCTTCTCAAAATACAGATCTTATATGTGTTCAATTAAACAGGGACAGATTAGGT
TCAGGAAGAATTATTCACATGGAATCAATTGGTATCAGAGAGTCAACCATTAGATCTTAGTGGGAAATATCTGCTT
CTCAAAGAGAAGTCTTTTGGGGAAAGCAAATTAAAGTCAGAGATTAATTTGATGAGTTTAGGTAATATAAACTAAG
GGGCCAAGAAAAAAGCTTGCTCATGGTATGAAACTAGAGCTTGAGGACACTGATCTAGTCTATCTATACTACTCTT
TCTGACAGACCCCTCTCTTCATTCTCATGCTCCTTGATGGCCCAAGCCACTCTCTCAGTTTTTTAAAAAATTGTTT
TATCAAGGTCTCTGGATTCTTCATGGGAATGACTTCCAGTTTATATTTTTTGGCTTGGTTCCAAAAAGCTATCAGC
TAAGGAATGCATATACTTACTTCCCCTATGGGTAAAGTAAATGAGAATTTTAGAAGCCAACTCACATTTTTAGCCT
GTACAGAATCTGCAATTCACCAAGCTACTTCTGACTCATGTCTATAAAGTTCTTCCCTGTTCTTTTCTCACTTCAC
ATGTACTCTTTGCAAGAATTCATCCACTTGTGTAGTTTCAGTCTGTTGATGACTACCCATCTATAATTCCAGCTGA
GAATGATCTTTTGAGTTTTAGACATGTAGATCCTGCTGCTTTCTTTCGATGTTAATGTCCCACAGGAACTTCACAT
TGAAGAGGTCCAAAGCTAAACTCATCTTTGCCTTCTTCCAATCTCTTTCTCCAAATGCAACCTACTTCTGTTGTCC
TTGTCTTAGTCCTTTTCGTGCTTCCGTAACAAAATACCACAGACTGGGTAATTTATAATGAACAGGGATTTGTTGG
CTCATAGTTCTGGAGGCTGCGAAGTCCAAGATCAAGGGGCTGGAATCTGGTAAGGGCCTTCTTGTTGTGTCATGAT
TCCATGATGGAAGGTGGAAGACCAAAAGAGAGAAAAAATGGGGCCAAACTTGTCCTTATATGAAACTCACTCCCAC
AATAATGATGCTAATCCGTTCATGAAGGCAGAGCCTTCATGTCCTAATCACCTCTTCAAGGTCACATTTACTACTG
TTGCAATGGCAATTAAATTTTACCATAAGTTTGGGAAGGGAAAAACATTAAACCATAGCATTCTGCCCCCTTTTCC
CCAAAATTCTTGTTCTTCTCAAAGACAAAATACATTCATTTCATCCCCAAAGCCCCAAAAATCTTATTTCAGCATA
AACTCAAAAGTGCAATCTAATATAAATTAGATATGGGTGAGACTCAAGGCACAATTCATCGTGAGGCAAATTCCCT
TCCATCTCTGAGCCTGCAAAATCGAATCAAGTTCATCCCCTCACCCCCTACCCTTCCCAGCATCAGGTAACCACCA
ATCACAGAAAGTTTTACTGATAGTCCTGCTCTAGATCATCTTTGTCTATGTTCACTTTAGCTATTTATCCTAGTGT
TCCATTATTGGAATACTAAGCATGTGGGAATTATTTATATTCTACTGTTCAAGGTCCTCACCAAGGTCTGATTGCA
AAAATTCAAAAAATTGCAACCTTAGGCATAAATGGGTTAAGCAGTTTAGGGTACATTTATAATAATTATTTACTGT
GCTACTTCAAAAATCTTATTGCCTCTATTTATAAATAAAAAGTGTTGTCTCTACACAGTGGCTTGTTGTAATGCAT
TTACTTGTTTCTGCCTGATTTTTTCTATTTATACATTTTCTTTTTTATTTTTATTTTTATTTTTTCACTTTTAAGT
TCAGGGGTACATGTGCAGGTTTGTTACATAGGTAAACTTGTGTCATGGGGGTCTGTTGTACAGATTATTTCATCAC
CTAGGTATTAATCCTGGTACCCGTTAGTTGACTTTCCTGATCCTCTCGCTCCTCCCACCCTCCACACTCTAATAGT
CCCTAGCATGTGTTGTTCCCCTCTACGTGTCCATGTGTTCTCATCATTTAGCTCCCACTTATAAATGAGAACATGG
GGTATTTGGTTTTTTGTTCCTGTATTAGTTTGATAAGGACAATGGCCTCCAGATCCATCTATGTCCCTGCAAAGGA
CATGATCTCATTCTTTTTTTATGGCTACGTAGTATTCCATGGTATTTGTGTTGGTCTCAAAAACTACAACTATGAC
AGGATGGCATTTTCACTTTTGTTGTTATATTAAACTCATCTTAAAAAGGAAAGATTAATAATGTCAATATTTGGGT
TATGGAGAAAAAGTATCTCATATCTTTGAAAAAGTTCTGTAACTATAGCTTTTTAGGTAGGAGGGATTCTGTGGAA
AGTTTTCTGATTACATCATTTCTCACAGTTCAGGTTAGACACCATTTTACTATGAAACACTAATGCATTGCCTGCA
CTGAGACTTTCAGTCACATGGAGAAACCTAGGCAAAATTTTTGTACACTTGGAAGAATATTTAAATTAGTAATAAA
ATCTTTAGTTTTAAACTGTTGAATGTTAAATAAGATATAAAATGTACTTGAAAGAAATTTGCTTTGATATCAGACA
CTGCCATGTTGCAGTTTCAAGACATAATAAAAAAGTAAACTAATGTTTATATTTTGCTGTTTAAGTTTATTAATAC
ATCAGATGAGTCTTCAAATTCTACAGTGGCTTTTGATATGATCATTTTTACTTGCCATTTTATATAGAATAAATAT
AAATAGGCATTTATGCTTAAAAGGAACTAATCTATCTATGGAAAAAAGAGAAGGCTGCTTCTCAACTAAATTGTAC
AGTTTAGAAACCCAGATCTGAACATAGATTATTGTTGTGACCTATGTAGGAAAATATGTTGTTTTCCTTATCGTAG
TCCTTACAGAGTCCATGATAACATATAAAGCCAGAAATGTGAGCCTCTGCAAGTTCATTTCTTTGTCTTCAATCTC
TGTGAATAGATATGAGTTTGTGAATAAGATAATATTAGATGTGATATTACAAATTATTGTGAGAAGCCTCTAAGGA
TTAGATTTCAAGGACTGCCATCTGGCTGATGACTTTATGATGACACTGTCATGAGATTTCATTTCCTTATTTCTGT
TCCAGGATCACTCTTTAAACAAGAAATAAGCATTAACTCTGAATTGTCTGCTTGTAGCTGTATGAGGGCTTCCACA
ACTGCCAACTAGCCAGGTACAAACTCATCAAGCAGAGGAGATGGTCCTTGCATCAGAGGGTTAAACATGCCTAGAA
GTTCCTTAGCTAAGCTCCCAGATACTAAAAAATCCCTCTAGGTTCTAAGAAAGATTCAGCATGTACATGTGTGTAC
ATGTATGTGTGTACATATATACATATACGTGTATATGCATATGCATGCATATACATACAAACACATTTTCTTCCAT
AACATCTCAGTATTCTCTGTTCTTTATAATACTGTTTTGTATTTTAATGATCAAAATTAATAGTTGATCATCTGAA
AACATTTTGACCTGTTTTCTCCGTCTTTGACAACCTTGAAGGCACTTGTAAGTCACTCTTTGCTTCTCTATTCCTA
GGTCCTTTCTCATCTTCATTGCAACAAGAAAAGAGAAAACAATTGAGCCCTATTTTGTGTGTAGCAAGGAGCTACT
CTAGTTAAACACTAGATCTCTTTTACATTCTCCAACATGTTGTTTTAGTAATTATTCTACTTTCCTTTTTTTGGGA
TATTCAATTTCTTCTTTCTTTTTGCTCCTCCCCTTTAGCAGGCCAACATACTCAAGTCTCCCTCATCCTAAGAGAA
CTTTTTTAGTATATCATTTTTTTTCTATCCAGCTGTACTTGCTTCTGCTTACTATATCATTTTTAAGCAGTAGTTG
GCATTACTGTTTCCTGTTCTTTAGCTACTAGTTGTACTTTGACCCACTCCAGTCTCACTTCCCCAGCACCACCACT
TTATGAAAACAAGGACTTACTAAGATCATCAGTGACTTTGTAATAGCTAATTAGTGTATTTTAATTCGTCCATCTT
CTTGACTATATTTTAACATTGATCCTGTTGGTCAACTCTGCTAATCAAAACTTTATCCTCCTTGGTTCCCAGAACA
ATATTATCTTGAATATCTCATTTCTCTAATCATATAATAATTGTGAGGTGCTTGGCACAATGCCTAGTGCGTAGTA
AGAACTCAGTAAAATATCATCTGCCATCGACACCATAAAAATTAATTTACTTACTCAACAAATACTTTTGTATGAA
GTTTGTGCTAGGTAGGCCCAGTAATTGGTACTTGGTATAGAGCAATGAAAAGCCCTACCCTCATAAAGCTTATATT
CTTGGAAGCAGAAGTTGGAAGACAGACATTGACAAATAAAAATTAAATACATGATGTGTCAGATGGTCATACACAC
AGTGTGGAAGAACAAAGAGGAAAACAAGTGGAGAGAGAGAGGGAGGTGGAAGAGGAGTGCTGCCATGAAAATGTGG
TAATCAAAAAAGGTCTTACTGAAAAGGTGGCATTTAAGCAAATTCTAAAAGACCTGAGGATGTGGGCCATATGTAT
AATTGGGGGGGAAAAAGTAGTCCAGGAGAGTCCTAATAAGTTAAAATGCCCCAAAGCAGGAATATTCTTGGCATGT
TGAAGGAACCTTAAAAGGGAGATCAGTTAGGCAGAAAAGGATCAAGCGAGCAGGAAGGTAGTTGACAATAAATTTA
GAGGGGTAACTGGCATCTGATTATATTGGCCTTTTAGGCCTGTGGACTTTAGCTTTTAATCTGAATGAGATGGGAG
TTATTGGAGGGTTTTGAATGGAGGAGTGACATGTTTTGTCTTATCTGGCTCCTCTGTTACAATAGACTAAACAGAA
GTAGTGAGACCATTAGGAAACTGTTGTCATAATTCAGTCAAGAGATGACTGTGGCTGGGATCAGAATGGGAGAGGT
GAATGTGGTGAGGAGTGGTTGGATTCTACTATATTTTGGGTACAGAGCACAACAGATTTTATAATGGAATAAATTT
AGGTGTGAGAGAAAGAGTCAAGAAGACTCAAGAATTTTTAGCCTGAGCAACGGAAAGATGGGGTCATCATTTACTG
AGATGGGGAAGGCTCCAGGAGTAACATATTTTGGGAGGAAGATGTGGATATGTTACATTTGAAATGCCTATTATAC
ATCTAGGAGATGTGTGGAGTAGATAGCTGGATATATGAATCTTAAGTTATGGGGAGTAGCTCAAGATACAAAGTTG
GGAGTTGTAACAATGATCAGTGCAAGTTCTCTGTCTTCAATGCAATTTTAAATGTTGATGTTCCATTCTTAATTGT
CTCTCTTCTTTCTCTCTGCACATTTTGAGTAGCTTTGTCTGTTGGCTTCAGTTAACATTAAGACTCCTCAGTGTCA
ACTTCCATCTTACACTCTTCTCCTGATCTCCAGAACTGTACTTTCTGCCACCTAACCTACATTACCACCTGGATAT
GCTACAGGCTGCAAAATGTGTCAAGTAGAATGCATTATCTTGCCCCTAAAAGAAAGTTAAATTTTCTGTGTTTTCA
GTGTAGTGTAATTGTCTAACTTAATTGTCTCTAAAACTGGAAACCTAAGAATTACCTTCTACCTTTCTCTTGATCT
CTCTTTCCCAATCTACTGACACATGTATTAAACTGGCTTCCAAATTCTGTGAATTCTACTTCAAAAATTGCTCTAG
AAACAATTCCCTCTCTTTATCCCTATTGTCACCTCATCCTAAAGCCTCTTCATCCTTTGTAGATTTCTGGGAGATT
GTAACCAACTTTTCTCTATTCTGCCAGTTATCAAGTCTTTACGCTCATTTGACATTCACAACAGCCTTGGATCTGT
CTTCCTTGAAATGAATCTTCTTGCTTCCCTTTGATTCCAGTGCTTTTTTTTTACCCTCCTGAGACTTGATGCATGA
TATTTACATGTATGACATGTTTCCAAAAGCATTCTCAAATTTTTCTGAAAGTAAAAACAAATGAAAAAGTAAAACA
TTTTCCTGGGAAGAAAAGCAAATAGTGTTATACATTTTTGCTTGTTCATTTGTTTGTTTATTTAGGAGAGGGACAA
GCATTAGAACTTCATAAGAGTCTTATATGCTGTATCTACAAATACCGTCCCTTGGCAATATAATTTTAGAGTTCCT
TTTCTGGAACTACTTAAGGACTGTTTTATGATCCTCAGCAGACTGTTATATTATTTTATAGCCATACCTTTTATTT
GCTGAGTAATTGTACTCAATAATTGTTTGTAATTGAATGAAACAATTCATCAGATGTTGGGCACTGAATGGCTTTG
GATTATTTCCAAAAATTTAAAGGATAAAGATTTGCTGCCTTCAAAGCTATGTACAAAAATATGATAGAATGCTAGC
GGGATATTTGTTTAAAATACAACCTTTATTACATTGGGGCCTGCTCATAATATATATGTGGCACATTTTATTTAAA
ATATTAAAGTTCCTGGTGGGACATGTCCCCATAATCCCAGCACTTTGGGAGGCCGAGGTGGGGGTGGGAGGATCAC
TAGAGGCCAAGAGTTTGAGACCAGCCTGGGCAACATAGTGAGATACCATTTCTACAAAACATAAAAAAAAAAAAAA
AAAAGCCAAGTTTGTAGTCCCAGCTACTTGGGAAGCTGAGGCAAGAGGATTTCTTGAACCTAGGAGTTCAGTTCAA
GGCTGCAGTGAGCTATGATCATGCCAGTGTACTCCAGCCTGGGTGACGTAGTGAGACTCCATCTCTTAAAATTAAA
TTAAATTTAAAGCTACAAATGACCCCAAAGCCACCAGTTCAACCCTCTCAATTTTGAATACCCTATTTTAAATTCC
TCTTATGCGAAATGTACCTTGTAGTCCATTTTAAGGACTGAGAGGATTTGGTATGTTAAAAAATTCAATCCATTAT
CAACTCCTTTAGGTACACTTAGCAGTATGAAAATGTGTCTTTCGGCTCTTCAGGAGAGAGTCATATGTATAGTTAC
AAGACAATCCCATTTTTATATTGCTGAGACCCAAATCTTCCCAACTGATTATGAAGCATAAGAACTCTTCGGAGGT
TTAAGTGAGCTGAGATTGTGCCACTGCACTACAGCCTGGGCGACAGAGCAAGACTTTGTCTCAAAAAAAAAAAAAA
ATTCTCTGCATTCTACAGTAGGGTAATATAACATCTATGATGTGAAATCTTGGGGCTCCGGGCCAGAGAGTGTCAT
GATCCATATGGATCTAAAAGGTTCATAGTGGTAACAGCCTGCTTCATTTTATGTCATCTCCTTTCAAGTAATTAGA
ATGTTTCTAGCTTGCAGGGATTGCACACAAAGGGAGACATTTGGAACCATGTCATTGGTGATTTACTGGTGTGGAA
AATTACCTGGTGATGTAGCCAAGTAGCCATTTTCATTCTAACCCAGTCCTACAGTCCTGAACTGGGCTGAACCAAC
GCACCAAAATATATGCTTAGAAATGCTCCTATGTATCAGTTTTCCCAGGAAAAACAATAGTATTATCGAAAACTTA
CCATTGTTTCCTAATAAAAAATTATAGGATACCAACAGACTGTTTTTTGTTCATAAATTTAATATTACAGTATCAA
ATATTAAAGCAAATGGGAGAAAGTTTTTCTTATTTGGTTTAATTGAACCATTAATGTTAGCTACAATACCCATCAT
GTTACTTTTCAATTATATTTATATTTTCATTTTATTTCTATCTGTATCATTCTCAGAAAGACTTCTTTAAAACATT
CAATAAAAATAGAATTTAGGTAGATTTATTTTTAGAAAGTTGAGTTTTTTTAATAAATGAATATAATCATCACTTG
ACTTAATTTTTTTCTGCACAATTCTAGAAATCTTATAGTTTTGGGATCCTTTGGCTTTATTCAGTATGTAACAGGG
ATCTGTTTCCTTTCTCTAAATCATTAATTCAAATGATTTCTTATATTAAAAATGTTTGGACATATAGGTATTAATG
AGTTTTATGAAATCTAATCTTTCCAATTTCCCCCTAAAAAGGGATGTCATTTAATCAGTTCTAGGTTGTGATCAAT
AGCAGATTTTTTTTTTTTTTTTTTTTTTTTTTTTTTTTTTTTTTTTTTTTTTTGCAGCTCCCTTTCACCCCGTAGG
GAAACCTGATATCATCCTTGACTAATTGCAGCAAAGAGCCTGGCTCAGGTCCTTTGTCTTATACCGAGTGTTTATA
GATTCTTGAGCCCAGCAGAATCTGAACTCCTGGCTACTGCTACCTACTTCCCAGCCCAGGCCCCAAAAGCCCTATG
TCTGCAGCCCCGTGCACCACTGTGTGTTTTTGTGGCATTTCTGAAACACAGAGCTACTTAACTTGTTTCTAAGCCC
AGATTGTGCCTTTTTGATTTTCTATTTTGGTATTTTATCTACCATTTTTCTGTGTTTGGATGTTTCTTCTATATTT
TGAAATAACTTCTTTCCTTTAGTACAAGTGATTCTTATTGTAGAAACTATCAAAAATTTACAAATAAAGAATCATT
CTCAACATTCTTAGCAATTCCTTCTATCATATTTTTGCAAATATATTTTTGCCTATTTTTATTTTACTTACTCCCT
GTTTATTAACAGTTAAAAGCATTTTCAGATAGTTTTATTTTTTCATTTAAAAAAATCTTACCACATTTTTATTAGG
AAGGAAATGGACAGGTGTTTATCTTTTCAATAAAAAACATGGGGGAAATAATTTCTTGAAGTACATAGTGACATTC
TTCCAGCCAATGTTTTATGCTGTGGTCATTCCGTCTGTCATCAGTATTCATAGAAAGAGATGAAAATTATTTAAAT
TAACTAGGAAATCAATTCCCCATTCAAAGCAGTAGTTGTGTGTTTCAAATATCTTCTAATAGTCAGTTTCACACTT
AGCTTTATCAAATTCCTAATTATGATACTCATTACATCACTCTGTGTCCAGTCAGTGTGTTTATGCCACAGAGCAA
TTAAAGCAAATCAGGTGAACCAAATTCAATCACCTTTGTAGATAATAACCTACGTTGCTTAAACTTATGGCCGCTC
ATACAATTACTGATGGATTGCCTTTTTCTTTTATATTGCCAGTATTTTAAATGTCCTAGTGAAGTTGGGGTAGCTG
TTGAACTTCAACTTTATCACAACCTCTTTTTTAAAATGTGTAAACGAAAAAACCCTCCATGAAATGACCAAATACA
GTTTTCATGCTGGGACAAATTAGATGAATAATAATCATAAATTCATAATGATTATTTATGATTTTATGTTTTTATA
GTGAGATATGTTTTGTTGAAATGTGTTATATAAGTGATACTTAAGTTTCCTATTAAAATAGAAATGCTAAAATGGC
ATTGTTCTCTTTAGCTGTGAGTCTAGCTTTTGACCTCTGCTTAAACGGAACTGTTGTTCCATCCCAAATCTGCAAC
TCTGAGGCCTATGCTCCCTTCACTGCTGTCTAATGGATACCTATCAATTTGGAAGGAGGTTTCAGGCAGCTATTCC
CGGTAATCTAATCTCAGCTCTGTCCTTTTCAATATTTTCATCAGTGGCTTGGATGAAGACATAGATAACATTCTTA
TCAAATCAATGCCACAAAGCAGGGAGAAATAGCAAATATAGCAGACAAGAGTATCAGGAGCCAAAAAGTTTTCAAC
AAGTTGGACTGGTAGGCTGAATACTGAAAGATGTAATGTAAATGCAAGGTGCTACATGTGGGTTCAAAAGAAACAT
GAAACAAAAAACCCATCTAACTTAGACTGGGCTCCCTGGAAATAGACTAAGATAGAGAGTTGTGTGCATAAGGTTT
GTTGAGGAGTGTTCCCATGAGATACATGTGTAAGGTTGTAAGATAGGCAAGATTGCACAGACGAAGAAGTGCAGTG
AAGCCTGCAGTGCGTTGCGGCCTCATCAGATTTTCAGGGGAGTTCTGGAAATTGCATGGCCCTTTAGAGACACGCT
GAATTGAAGCAAGGGATCTGGACCTTTGAACCCAATACTAGAGAGTTAATGGTCCTGGGTCACCCCATGGGAAAGA
GCAGACTGGAGTAAGATTGTTACCTACAGCTGAAGGCAATTTCCAGGGAGGGAGGCAGCTGTGAGCTGTTAGTAGT
CAATATTCCAACCAGCTAGGGCATGAGGTCTTGGCAGAGCAACAGTGTACCCAAGACCGCAGTGTTACCCAAAGTA
TGGTCCTCTGACTGGCAGCATTGGTATCACCTATGAGCTCACTAGAAATTTAAATTTGTAGGTCCTACCCCATCCA
ACTAAATCAGAATCTCTGGGGATGGGACTTGGGGAACTTTTAACAAGCTTTCAGGCCTCCAAGTTATTTCTATGCA
TATTAAAATTTGAGAACCACTGCCTACACCAACCAAAAACATTCCAAATATGGAGATAACATAGAGTTTTTAGCAA
CAATAATCTCCTTCTGTTTCACTTCTCTCTTTACACACACACACACACACACACACACACAACACACAACACACAA
TGTGATAGAACAGTGGGAAAGGAAAGCCAAAGGGGATCTTAGGCCGAATAAATTTAAGCATATAACCTAGTCCTAA
GAACGTATATTTCAGCTTAATAGAGAGAGGAATATTGTTATAAAGCTGTCCAAAGATGGAACAGGCTGCCTTGTAA
AGTTGTAGAAGTATTCAGGAACAGGTTGGTGATACCTTGGTGGTTGTATGGTATAACATCCTGATCTTCACATACT
CATCATCTAGAGTGGGAGTTTTCTTTTTCCAAATGGGGTTTTGGCAGAACTAGTTCCACTGTATCTTAATAAGTAA
TAACTCAAGAAAGGGTTCTATGGATGAAAAAATGATTAGGTAATATCAAGTTAAATCAAAGCGAACAGACTTCTTT
CCCATAGGAGTAATCAGACCCTTATTACAGTGCATGCTTGGTGAATCAACAAAGTATGTGTATTTATGAAAGTATG
GGGGGAAGGGATAATCTATACAGTATGCATCCCTTCTAAAAGTTTGACCATGAAAACAATTTCTCAAGAATCTTAT
ACAACACTACAGTATCTGGTCCAATACTATGCATAGAACATGCACTCAGTAAGTGTTTGTAAGATAGATAGCATAG
CATATAGGCCAGGCCACTGAAGGGAAATCATCTCACCGTGAGTTACCTGAATAGTATTCTCTAGTGCCATTAGCTC
AATTCTTCACGTAGGCATAAGCCTATACATTTGCCATGCTAACCAAGGGAATTTGTGTTACGTGAATTTTGACTCT
ATTCAGACATTTTTTTCTATGACTCCTCCAAGGCTGTTATTCTTACCTCATATTCTGGTAGAAGTTTAAGGACTTT
TTTCTGGGAATATTGATTAATTAGCTAGCTAGCTAGAGACAGAGAGAGGATAGAGATTGATTCTCTGGCAGAGCCT
ATTTGAATCATATTGAATCTTTTTTTTTCCTGAGACTTCCCACAAGGAGGATGGAGGAGAAATTTTTTAGAAATCC
ACCGAAGTAATCAGGGATATCTTCAGTAAAAGAAGCTATACTTAATAAAGTCTCTATTTTAGCAGATGGCAATCAA
CAATAGAGGCAATAGACAATAGAGTCTATTAAAATTGCTGGGATCTGCTAATAACGTTTTTCTTTTCCCTGAAACA
AATGCCATTAACCCTCCTTGACACTCTGTCTTCATCAACATTCTAATAGAATGGAAGTAACTCATAATTTTGAGGA
TTTTTTTCCCACACAAAACCTATAAACCACACCACGCTAGTGATTACTTTTAGCCTAGTTGCTAGGTTGCTGCTGG
TAACAGTAAAACTTATCCTGACAGGTAGGCAATTCCAGAAGCCCAGCCAAGCACTTGGTGTGTGTGAGTAAACCCC
CATACACTTCTCATGTAGAGTAACCCTGGCCAACCCATAACTCTTAGCAACTATTCCTGGTGGACGGACCTGGTCT
ACTCTAAGAAGAGGCCAAGGTTCTTTAATAGTGCAGTTGCAAGAACCAGAATTGAAAGTCAAAGTTCTAGCAAGAT
TTTGCAGACTCCTTGGCAAACCAGTGGCTTGGGACTCATTCTTGACTTCAAGCCCTTAATTGATAATGGTAGGACA
GCTTGCTTGCGCTGGGTTCTGCTCCCTGGGATATGCACTGTTTGCCAAATGAGTAGCAGGTGGACAGACATCTTTA
CAATTTGCTGTCCCATATTCTAAATGAACGTGACATTCTATAGGTCTGAGTTAACCTATGAAGTCACCAATTTCAA
TATCAAAATATTTATGACAGAGAAAAGGATACTGAGGCACAGAGAGTCTGTGACTTTCCTAAGCTCAAAACACCAG
TTTGTGTTAATTCTGACACAGAAATTCTTGTATTTGCTATCAGTCTCCTTTTTCTGTGTGTGTGTGTGTTTTTACA
TTGCAGCATCACCTATATGATGTTAGGTTCTGTAACTTTTTGAGAATTTTCTCACATACAGTGATGTGTTACTTTT
TGATATTTCAAATAGTTCTAGTAAGTCTTTTCTACTTTTATTAGCGTATTAACATACTGGCTCTAAGAGGGCATCT
CACCACATCTTTGCCATTCTTCCTGGAAAGGCAAGTTTCTCTCCATCTTCTTTTTTGTATTCCAAAGTTTTGCCAA
AGTTTGCTTTTGAAAATGGGTTACCTGGCAGAGCTTTATTATTCTAACTTTGAAAGTACAAGTCAGAATCAGACAG
TGGCAGTTATATATGCACTACTGTGATTACTATATAATGAAAGTATCTATGGTGAAAATACTGATACTGACATATA
TTTGCCATTTTCTAATTAAGTGCTTCAGTAAAAATTAAGCACTCACTCTTTGCCAGATACTGCAATAGATATTGAG
CACATTGAACAAAATTCTCCATATACATATATATGAGTCCACATTCTATGAAAGTATAATGTTTTTCTGAGAAAAG
GCATAATATTCTATTAATATCAGCTTTTGCTTCTTCCACCATATATTGAAAGAATTCTGAATACTGTTATAATTTA
ATGGGAGAATCTAGAGAATTCTGTATTTGCTTTCACTGCATTGATGAACTAAGATTTTTAAAAAATGTATTCTTCA
TAGAACTACTTTTCCATATTTACCTAATATTATTCTTATATCATTTGAGCACATATTTCACTAACAAAACAAATGT
GCAATGTTATTAGTTCTAACATCAAAATTACACTGATACTTTAATTTTTATCCTATTATTTTTCATGCAGATTAAA
ATAATTATAGCTACATCACATGTTGCAAGTTTTAAGAGCTACTTTAAAAATATATGCTTCAGGAAAGACATGATTA
GATGGGGAAATGGATGATGTTCATATTTTCAAATGAAAAGTTTTAAAAAAGTGCCTATCACAAACACTAAATTTTT
ACATAAATTATCAACTACTAATATATCTACAAGAAATACCATTTTTCCCTACAAAAACTCTTAACAATAATTGTTA
AACTTAGTCCTGGAACCTGCTAATATAATCGGACAAATGTTGTCAATAAGAAGGTGAAAAAGAAAGCATATATAGT
TTATCAAACTATAAAATATAGTTTATCAAAACCAATTTTTCCTATTGACATTTATTCAGGAAGGAAAATGGATGAG
TGAAATGAACAATGGTCTCTAAGAGAGGTGGGAGATAGCAATAAATTCAGACCACGTTTCCTGTCATTACAGCAGG
GAAGTAAAAGAGCTACAGTCAACTCTCGAAAGTACTTGGGGGAACTAATGATTCCCTGTAGACCTGTGATGTTTTT
GAAATTTAATTCAACAATTTGATATACACCGCAAAGCGAACAGATAGTCAGATCAAAATCGGAAGAACGATTGTCT
GAATGGCATCCATTTTTCCTAGATGTGCTGTCCCATCCTGTGTCAATTAAACTTTCAGGTGATCTTCAAACATATT
TCCAAGTAAAAGGTATTGCAGTTATCCTATAAACTGGCCTCTTCCCCAGCACTGCTTTTGCTGTGGTCAACTTTAT
TTCTTTGGGCTCACAAAACTGATAGAGCAAAATAAGGAAAACGGAACATTGGATTAAAATAAATTAATTCCCATTC
TGTGACTCACTAAAAAAAAAATGATAACTATGCTTCTGTGAGCATTAATAAGGAAATGAATAAGGAAATGACCAAA
TTGTTCAGTGGACAACTTGTATGGGATTTTTAAGTATTGTGTCATCATCAATGTTGTCAATTAGCATATACTTTGA
AATCAACTAAAGCAAATCAGTTGACTAATCATTAAGGGTCTTTTTAAATGACAACATCTAAACAGCAAATGTTTTA
TTTTGGAAAATCATGACAGCACAAGAATGAGCCAGATGTTTTACAACATGATATCCATAATTTAAAGTATGTAGTA
GTCACTCAAAGGATTTCTATTTCAGTTTCCTTATGATTTGGCTAAGCTAGAATTTGGAAAAACACTTTAAGGTAAT
GTGAGAAACAGCAAAATTCAACATGTGGATTTTTTCACTAAAGCTTATTTCTGATTATTTTTTACAAACTTTACTA
GGTATATGTTAACTTCATGACACTTATAGCAGTGGACCGTAGTTTTAATAAAATGTGAATGTATACTCTTTTCTCA
ATAATATTAAAGAATGTTGACTTTCGTGAGGATATTTTTATTTTTCTCAACATTAAGAACTGTCAAAGATTTAATT
CTACAACAGAAGACGTGAATTTTGTTTTCTAAAGGAGAACAGAATCTATAGAAGAAGTGTTGCTCATAGTACTCAG
ATTGTTGACCAATCTTAAAGGAGAAACCGTCAATTAATTTACCGAGAAGTAATAACATTATCTTTTTCTTCAATTA
TGCACATCCACAAAGATTTGGGGCAAAATCCACTTAAATGATATTATACATAATAGATGAGTATTCATATGTTGTA
AGAGTCCTGGCTTCTTTCCTGCAAAATGATTAAAACTTGGATCAGAAACCAATTAAAAATCCATTCTAATTCCCAA
ATGTATGTAACTGTACTATAAGAAAAATAAATATTTCTTCTTGAGGGATATCCATTAGTTAAGGATATTCATAACA
TGGTGTCTTGTAGGAAATGTTAATCTTTGGGTGAATAGGGATGTTTGGGAATAACAAGACTCAAAGAGATGTTGCA
CTTACTCACTTTTCTCTGAGTTGTTATTTCTGTCATTTCCCCAGTGCGCCTGTCCTCAACTTTGCCTCTCTCCTTA
TTCCTTTTTTTTTTTTTTTTTTTTTGAGACGGAGTCTCGCTCTCTTGCCCAGGCTGTAGTGCAGTGGTGCGATCTT
GGCTCACTGCAACCTCTCCCTCCTGGGTTCAAGCAATTCTCTGTCTCAGCCTCCCGAGTGGCTGGGATTACAGGCA
CCCACCACCACGCCCTGCTAATTTTTTTTGTATTTTTAGTAGAGACAGGGTTTCACCATCTTGGCCAGGCTGGTCT
TGAACTCCTGACCTCGTGATCCACCCACCTTGGCCTCCCAAAGTGCTGGGATTACAGGCGTGAGCCACCATGGCCG
ATCCCTCCTTATTTCTTTTTATCTCTACCTCTGCCTCAATGGTATTTCTCTATTACTGTTAGCATTTGCTTTCTGT
GAGCTCTTGCACACTGTCAGCTTATATACATGTTCCTGTTCACATGTTTTCCTGTCCCCAGTGGTTACAACATGTC
TTCTATCTCAGCCCACTCTAGAATTGTCTTACTTTTCCAGGTCTCCTGCTCCTCAGTATTTTTCCCACTTTTCTAG
ATTCATGTTTTCCCATCTGCATATTTCTCTTCCATGTCTGCACTGTCATCCGCTTAGAAGACAGCGCATAAGGACA
CTGTTATCTGAGCAAATCTTCAGCACAGCCACCATGAAGCATGGTTACCTTGTCACTTTCCATTTTTCCCATAGTG
TGTGCAAACTGCCCTGATCTGCATAGAAAGGTATCATAATTGAGGAAACAAAATGCACAAAAATGTCCTTGGTTAT
TCCACCCCTCAGAAATATAGGAGAGAAGTAATTTACAGAATTACACAGAATAACGCTATGTCACATGGACATGGAG
TTATCGGGTTAGCATATAATTGGAAAATATTTCCTAGGACCTTGACATTTACTCACTTTTTGTTTTCAAATTACAT
GTCCCTATCTATTAGTTGCAAATTATTTTAATGCACCGTTTACCAAAGAAAGGCTGTTTCTTCTGAAAGCTTTCAT
TTGACAAGTAACTTGTAAAAATATTCACATTGTGTATCTGTTTTCCCCTTCTAGTCCAAACTCTAGTTATCTTAAA
CTTTGCGCAGTTATAAAAAATCATAACAAAAAAAGCTTCCTCGTTGTCATTCTTGTCAAAACAGGTTTACCAGACT
TAGGTAAACTTAAAATAGTTAGTGTAAAAGTTAAAAAGCTGATTTGCTCCTTCCAGCGTGTTTGTTGCCTTTTTGC
CACAGCAAAATTGTAAATGTAAACGTATTCCCTAGGAGATGAGCTGGGCTGCAATTTTCAGCTAATTGGGAGAAGC
AGCCCTGAGTTGAGCACTGTCAGGCTGATTTGAGTCTTAAGATATGATGATGATTATTGTGTCAAATGTAATCAAG
AACGTGGGCTCTGAACTGACTCAAGGGCTGGCTGTTTTTAATTCAGGTTCGTATATGAAGTAGACCTCCGGTTCAC
CGATAGTCACAGCTGGTTGTAGAAGAGAGCAATTTTTAAAATGCTATTTCATTCTCTATGGAGCTCTAGGGATCAG
AGATTGGATGCACAGGGAGGGGACACATCCTCATTCTCTCCTGAAAAATTCTATTAATTTTCAGTATAATAAACTT
TCTCTTGAGATTCCCCAGTGGCTCTGTATCGGTGGTTTTCAAACTTCTCAGACCCAATGCCACCCCTCTTTTCTTT
TTTAAATAACAAATACTTTGTAATACCTTCTTTACGATTATAAGCCAAAATATGTAGACAACATACCCTACTTATA
CAGGCAATAGTTTAAATGATGCCGTAACTCTATTTTAAAGAGAAATAAGAGTCATTTATAATAAAATAATATGTGT
TGTAGTATGCAGTTATTCAGGCAGGATCACACTGGAACACAAGTGAAGTTTTTAGATCACGAGACTATCAATGCAG
TATAAACAAATGCAGAATGACACCATTGTGTTGTATGGAGACTCAAATACCATGAGGGGCATTGGTCATCCATAGC
GTAATTTTCCAAAATGCTGAACAACTCTTGGCAAAATTCCTAACACCATGAAATAAATTTTTTCTTGGATCGTTAT
GGCAGTTAGTTGCATGGCTGAAAAATTCAATGTCTTAAAATCATGAGGAAAATATCTTATGTTTACGTGTAAAATT
GAGTTACGTTCCAGGTTTAGGTGTTTATAAACAGGGTTTCCACATACATGCATGTCCAGTGGGATATTCCAAAGTG
CTGTCAGACTTGGGAGAGTTCTTTGTTGTATAAGAAGTCTACCATCTTCATTCCCTCTCCACAGAATGCTATTATA
GTAACACTCTTCAATCACTGTGATAGTCAAATGTCCTCCCTCAATTTCTAGGATGCCTCTTTTTTTTGTGGTCTGT
ATAATTTGGTTAAATATCTTTCCAGACAAATACTGATTTGTGAATTAATGAAATAGCAGTATTTTCGGAGCACCTA
ACCTATTTCTGAGTGATACAGTTGCCATTTTTACAAGACTAAATGAAATTACCATTTCAGACCTGCCAGATTGTCT
AGCCCAGTCTTTTACAATTCTGTGATTATCACTGCAATTATAATCTATTTTCACCACTTGAATGGCATGATCTCTA
TAAAAGGGTGGTGATAACACTCATCTATTCTCCTTCCCCTCACATAGCTATATCAATCGCCCCCTAACCAGTTGTT
GATAAATGCAGTTGAATTTTATGTAAAAATTATAAGAGATATTATTGTAGCTGTCCAAGACATTTAAAATGCTAAA
TGCAACTTACGTGGAGGCTATAAGAGAAATATGAACCCATTTATTGAAGAGATTAGCTAATTTAGTAAAACAACAC
AGATATACCTGCATACAGGGATAAATCCCTATTGTCTAAATTATTGAGATAAAATAATGTTTTACAATGAAAAACT
TTTAGACAAGTAGGTAAGTAAAATGCAGCAGTCTATTTGCATTTCATCTGGGCATTTGACAAAGTCTTTCGTTATA
CTCTTGTGAATAAGTTGGAGAAATACTGGCTAGATGCAAGATAAATTGGATGGCTTAGAAGCCACTTCATGATTTT
ACGCAAAGGATGTCGATTAATAGACCAGTGTCAGGTGGTGATGGAAGATCTCTGGTGCTATGTCACAAGCTTCTGT
TCTCAACCCTGACACACTGGATGTTTTTGACAGAACATGAGTAGAACTACAGAGAGGAGGCCCATCAAACTTATGG
GTGATAAAAAGCAGGGAGGGCAGGAGTATTTTGGGTGACAGAAGCCAAATGGGTGTCTGGACAGGATGCGTTTTAA
GGCACTTTTGGTACTTGATGTCTGAAGACCAGGATCAAACTTATAGGCAATCTGAACATTTGCCAAAATAACAGGT
TAATTTTGACAGAAGTTATTATTTGTATGCTGTCTATTTCTTTAATACACCTAGAAAGTATTGAAATAACATTTTT
TGCAGACACTCATTTTGAAAATTCAGAAAAAAAATTGTTAACTTTCGTGGAAGAGTAACAGAAACTCAGTCATTGA
CAGCTAAATACAATGTGTTGCCCAGTAAAATAGTCCACCCCTTCACTTTCATGGCTAATATAAAATTTGATGAAAG
ATACAAATTCCAAAGATTGAATATCTGTACATTTGCAAAGCAAAACACAATTTTGGGCACAGAATTGCTCATTCTC
ATTTTTAAACATCTTGGTTATAACTGAACAATAGTTTTTTATAACAAAGATAATATTTTCAAATTATTATGAGGTT
CAACTGAAATAATTTATGTGAAAGCAATGTCTAAACTCTAAAATTCTATATAAATATAAATTATTATTCAATAAAT
TCACATCAAGAAAATTTTAAGTTTTTTAAGAACAAGAGCCTATGGCCTTGTTTTTAGAAGCTGTATACCTTATCGG
TAGTAGGTTTATTGACTTTAATTAAATTTATTGAGTATCTATTAAATTGCCAGGAACTGTGGTGTGAATCTTTGCC
CTCAAATAATTTACAGTAAGTTGTGGTTGATGAATGGTGATGACGATGATGAATATCCAGACTATAGTAAGTGGTA
TATTCATAAGTCAGAGGATTCTTAAAACCAGATGCACCCTCAGATTCATTCCTTTCATGTTGTACTTCTAATTGAA
AAAAATAAATCCTAAATTATGACTGTTCTTTATAAATTTTAATTGATCTTATAAAAGGCCATCAATACATTTCAAA
GTATCTAGGTCTTTTAAATGCAATTTTTCACCCTGGTAATTAAAAGTACGAAAGCAAGAAACTTTAAATCTTTATT
TTGATAAGTTTTAATTAGCTCAAGCTACTTGTAATCCCACATCTTGTCTTGTAAATCATATCTGAGCCATTAAAAT
AGGTTTACAATTAGAAGGGCAATTCTTTTAGAATCTACTTAAACTAAGTCACTTCGACAAATTAATTCATCGTTCA
GTTGGTTTTATTAAAATGTATTTATTTCACTGTAAAATGTCTAGTAAAGCAATGTATGAAGTATTTTATTTTCATG
TTAGAAATTTTATGTAAAAGATATCCCAAAATACATAGACATTCAGATACTCTCTGTATCATTAACCAACATTTAC
TAACTTATCATTTAGAGAAGGCCAAAATTGTATGTACTATAACTTTGTATAATTTCATAAGAATTAAAATATTCGA
TTAATGCCTGTAATGCCTTCTTTCTAAATCAAATCCTCAAGCTTACCTCGAGTTCAAAGTTCAGTATTTATTGTAA
CACATCTCATAGATGACGGATGAAGATGGTAAGCAAAGGAATAATAATTTCTTTTCTCTTTTCACACATATATACA
CACATACCCCATAATCCTAATTCATATAATAATAACAGAAAACAAAGGGCTTTTGAGAATAGTGACATATTAATAT
CCATTATATTTACTTCACAGGGAGACTGGCAAGTCTACCTTGAGAGGTAATGTCTTATAGTACAGTGGACTAGATT
GTTTCAAGATTTGTCATTTATTTTGGCAACTCACCCAGCTTCCCTGAAAGTTAAGTTCCTCATCTATAAACTGTTC
ATGATAATTACAACCTGCCTCATTAGCCTCATCAAGCTATTTAAAATATGAAAGGAGGTGCTATCTGTGGATCCTG
TCAAAGGAGCTTGAAAACTGCAGAACATTATTTTAGTGTAAAATACTATAACAATACATGTTGAATATAAAATGGC
TTTTTCTTAACTTTTATTTTAAGTTCAGGAGCACGTGTGCAGGTTTGTTATATAGGTAAACTCATGTCATGGGGGT
TTGTTGTACCGATTATTTTGTTACCCAGGTATTAAGCGTAGTACACATTAGATATTTTTCTTGATCCTCTCCCTCC
TCCCACCCTCCCCACTCCAGTAGGCTTCCACGTCTGTTGTTCCTCTCTGTGTCCATGTGTTCTCATCATTTAGCTC
CCACTAATAAGTGAGAACATGCAGTATTTGGTTTTCTGTTCCTGCATTAGTTTGCTAAGGACAATGGCCTGCAGCT
CCATCCATGATCTCTGAAGAATCTCCACACTGGTTTTCACAATGACTGAAATAACATACACTATAACCAACAGTTT
ATAAGCAATGCTTTTTCTCCAGAACCTGTTATTTTTGACTATTTAGTGATAGCCATTCTGACTGGTATGTGATGGT
ATCTCCTTGTGGTTTTGATTTGCATTTCTCCAATGATCAGTGATGTTGAGCTTTTTTTCATATGCTTGTTGGTCGC
ATGTATGTTTTCTTTTAAAAAGTGTCTGTTCATGTGCTTTGCTAAAAGGGCCCTTTCAAATGTGTATTATTAACCA
CAAGAGAGTACTGAGTAAGAGACTAGGTAATAAAAGTCACAAATATTTCGATATCATAATTCAGAATTTAGATCAG
CGGTTATGAAATTGTTCGTATTTCCAAATTCCACTGACAGGACTCTACTATAAGTTTATTTCATCTGTTGATATGT
TTTTAGCCACTTCTTTCTTTTAAAGTGAATCTGTTGTGTGTTTGCCATTTGATATTAGAAAACTGAACCTGCCTGC
TTTGCTGTCTTCTGAATATTATGTATCAACAACTAACAAGCTACAGTTAGTTGTTTTGTTCTGTTTTTCTCTAAGT
TATTGTGGATGAGGATATATATAACTGCACAGTCTTATCAGGTTTGTAAGAGATGATCTTAGGCTCATCTTTTAAA
TTGGTTTTTATACTATTTTAAACAAATCCTTTTAGGAGAGAAGAAAAGCTGCTTAGTCTATCAACATTAGGAAATA
TATCTTTAAAGAGTTTATCACTGCAAGTAACCAAAGCCAACTTAAAAATTCGCATTATACAAATCATTGAGAATTT
ATTTAGAACAGAAATGTGTCCAACTATAGGTCAACACCAATTTTAAGTGTGTAATTATCTGGGAAGTAGTGTTAAC
TGCATTTTTTTCTAAAGATCCCTTACAGTTGTATAAATGCCCAAAAGGATATTTTGAGTCTCTGTATATTAACCAA
ACCAAATGTAATTCATTACTCCCAACATTATATTTCAACCTCTCCAAATAGTACCTTTTCGTATTGTATCAGCAGA
AAAATATAAAATGCAGATCTTAAAGAGTATCAATCTCTTTAAAAATTCAAGAAAGAAAAAAATATGTGTGTATAGA
GACGTGTATTTCATCTGCTCATAACACTGTGTACATTTCTTTATCAACTAATTTTTTTCAGTGATTTATGAGTTGA
AATACAAATCAAATGAAACGGGTAATGCAAAGTAAAGTAGAAAACACATTTTCTACTGCTGTCTCCTAATGCAGGT
CTTTTCAGGAAAGTACTAATGGTTTTAGGGAAAGTGTATAATTATGGTTGTTTCCCTAATGATAAATTCGCAAATC
TCTATTTTAAAAACATTCATAAGGTTAAAAAAATGAGAGATGAAATGTGTCTTTCAAAATTCCTTACGTGATTGAT
AATGCCTATACTCTCTTACTATCTAAAGTCTAGGTGATATGTATATTTTTTTTAAAAAATAAAATGTCTGTATCAG
TGAAGGAAGTTTACACAGATAGCTTCAAAGCTGTGGTTTATCTTTGGAGGATTAATCTATTTCTCATGCCAGTGTG
TTGCTACTGCACATGTTAAAAAGTCATCCTGTGGTGTCTGGGGTGACAAAAGATGGGAATGAGTTTTCTGAGAACT
AATCAGCAATACTTTGGGAACATTTAGGTCATGGTTTCCAATTAACTCTGGAGAGTTTGAGTAATTTAGTACCAGA
CCTCAAGAGAGAGGGGATGAAAACCTCGTTAATTCATATGTTGGTGAACGGCAAACCAGCAAATTTGCATTAAAAA
TGGATTTTTATTTTAAAGCAAAGAGCAGCCAGATCTTTTCTGCAATAGTTTGGGTAGGAGAATATCTTTGTATGTA
TGTGTTCCCTTATGTGTAGGTATTTGTATGTTTCAACGACCCTGCATATGGCAATAACAGAAAATTAAATTTGTGC
TCTAAAATGAAGACCAGGATTCAGTGACATAATCTTCCTTGTGCCTTTCTTTCTTTTAGTACAATGAATATATCAG
AGAGGAGTGTATTCCAATATCTGTCTTCAGAGTTACAAAAACTTCTTTTCTAGAATGCAAGACTTGGGCTATACCC
CCAGCTCTGCCACTTAACTTGTATACAACCTTGGGAACATCATTACAATTCTCTCAGAATCAATCTCTCCAGCCCT
AAAATGAAACCAGCAAAAGCCTGTACTGTATATCTAAAAGGTTTTTTATTTTTATGAAAATTAGTTAGGCAAACTT
TTGTTAAGCATCCATCACTCTATTTTGAGATAAAGCCTTGCTGGATGATCTCCACCTCTTTTGATGGAAAGAGTAA
AACATGTTTAAGATACATTTATCACTTGTTTGGCAAATTGAGATAGAAGTTTATGAAAGCAGATTGATATATGTTA
CATTTGAGCTACTGGGAAGGACTCCAGATGGTTTATAGCCTTAATTACATTGTAACTCTAGTTAAATGTTTACCTA
TCTGTACCCTCTGTTAAACTTGAATATGTTAAATACCAAAGTCCATGTATTATTGGATTTTCTGTCACCATCATCA
GGCACAGATCCTGGTACACAATAGGTACGGAATGGATGCATGGATGAATTATTGAATTAGATGTTGGTAGGCATGT
GGAAATAAGAATGAGGTTCAGAATTAAAGATAATCTGTATCGAGTGTAAAGCCATTGGCAGAGAATGAAATATCCA
GCTGAGTATACATAGAAAAAGAAGGTAGGTAGAAAAATGGAAAATATCTTATGAAGTGATGATAGAATAACTCTGA
ATATGTTTGAAAACATATAAAGAGTTATGTGGATGTTAGCTTTAAAAATTATCTTCCATGCTGTACATTAGATCTG
CCATTCTTCATGCTGTGGATGAAAAGCAAGCATCAGAAGTTAAATTAAAATGATGTCATATATTCCTCGCCTTACA
GTTTCATAACAGAGGAGAAAAGAGAAACATTCTCTCATTGCCACCACCCTTCTCCAGTCATATTTCTAGGTAGATG
TTGCCCAAAAACAGATAAAACCACAGAGTTGGTTTTGCTAGGAATGGACTACTAATCCAGGCAATGTTGACAGCTT
TTGCTTCTCATTAGTGCACGTTACTAATAGAATTGCTAGAGATTAAAAGGAATCCTTTCTACAAAGTGCTGTATAT
CCATAGGTGACAAAATTCTAGCTTCCCCTCACAAGTACAATATAAAGTTATGTTTTAAAATCAAAATGCAATTTAC
TAGCAAACTAGTAGGAACTGTTATGGTTACAGGAAATTTGAATTTCAGATTAACTCTGGTTCTATGAGTAGCGGTT
GATATGGCAAGAATCATTTTGATCTTACATCCAGGTGCTACTAAGGTCTCTCTGACCTATATCTCACCAAAAAAAG
GAACAAAATAATGATCCTTTAATCTTTCTCCTAAAATATCATAGGAAATGATAGTGGCTAAATTGCAAATAAACTA
GGAAGGAAAGATTCAGAGTATTTTATGTGATTACTCTATAACAATGCCAGGCCATAGTGAAAGTGTTATTTAGCAG
AAGACTGAGTTCTTTGAATGTTCCTAATTTATCACATTTTAAAAATAACCTGGGCAAAATAACCTTTCATATCAGA
TTGAGCCTTTTTCTAAAAATACTCAATATGTTTCTGTAATTATACCTACACACTTACAATTCCACAGTATAATGCA
CCGATAAAGTATTTTTCATCCATATATCTAATAGTAGAATGGTGTGTATACAATAATTAAGCTCTTTAGGCTTACC
CCGGAAAGCAACAAGTTTCCCTTCCTTTTTCCTTTTTATGTATTATGTTGGCCATAAGAAATTGATGATATTCAAC
TCAATGCAGTCTTAGAGATTTATTCAGAAATACCATGGTGTGTGTGTGTGGCGGGAGTAGGGTTCTAATGACAGGT
CAGAACTTACTTATTTGATTTCTTCATTGATAATCAGGTCTTAAAAAGAAAATGGGTATGCTGAAAACATGCCTTC
TGTGATTCTTTACCTTCATGTGCAGTTGTCTCTGGATAAACACTTTCTTTGGCACGTATAGGGTTGCACTAAGCTT
TATAGCTCCAACACTCCGCCCCTTCAGTAGATTCTTGCTTGTAACTGATGATAATGCAAACCTGTATTATCTATAG
GTCTCCTTAAAGGGCAACCAAAAGTTCAGTAGCAATTCAGGCACAATTACTGCATGTGAGAATCCTCCATCTTGTT
CCCTTTGGAGACCACATATATTTCTTAGGCAAGTATATTTTTAAAATCCTTGTTCAGCATGACAATTCAGGAGGTC
AAGTTCTCCCAGAAAGCAGATTCTGAGAAAGTGATTAGCATGAAGGAATTTTATTGGAGAGTGCTCTCAGGATTAA
CACCTGTGAGCGGAGGAAAGGAAAGGGAGCAGGATTGGGCAGAAGGAGAAGCTGGGCTACCATACAGTCACAACTA
CAACACAATCAACCCTCCGCCTCTCCTTCCTAGCCTTCCCCAGGAGGATCTCTGAAGTCTGAAGGTAGAATAGCCC
TTCAGAATTGTCCTGAGTTGCAGCAAGGGACCCAGGATTTTATACCCCACAACTCTCCCATCAACCAATACGTGCA
GCCCGTCTCGGGGACATAGTGGGTAACTTTGGGCTAGGCACCTCTCTTTAGCTGAGTCCAGCTCTCAGACAGGAAT
AACAGCTGAGGACTGTCAGCCAGTAGCACTACCAGCAGCTGGGGTCAGAAGTATTTCAGTCCTGAAAAGGGGTCCG
GGCAGCCCAGCTTAGCATCTACTATGCCAGTCGTTCTCAAATCTGGTTCCTGGCAACTGTGATTCTCAAGCTTTAG
CATATATTGGAAGGCTTGTTAAAACACAGCTTGCCGGATTTTACCCACAGAGTCTCTGATTCAGTAGAGCTAGGCT
GAGGCCTGGGAATTTGCATTTCTAATAACTTCTCAGACGTTGCTGGTGCTGCTGGTCCATGGACTATGAGAACACT
GTTTCATGCTGCCCTTATTTACATACTGAGAATGGTACACAGTGCTCTTATGAATAGAATGAAAACCTTTTGAAAT
CACATTATTCCTTACTCCATCAAATTCTCAGCTATTTTTGTGCACCATAAAGCTGGAATAGCTGATTATAAAACTT
TGTTATGTAAAAAAGTACTTAACCAATACAGTAGATTCTGTTTGCAAAGCATTATTACAGTTTCTAATATCTGGTC
ATTGTTACTTGTAAAATTCAGCCAAATTTTCTCCAGGGCCTGTAGTTTGATAACTTGGACAAAGGAATTTAAAAAA
AAATCTAATTCAAGACCTTTGGTTTTTTTTCTGAACATATCTTTTTTTTCTTTATGATTCTTATTTTTACATTTTA
CTTATCATATAAGCCACTTAAACCCATATGGTTCCGGAAAATTTAAAACTATATGATACATTTAGAGCATGTTGAA
TGCACAGATATGGAAATTAAGTATTCTTGACTCATTCTAGACTAGACCTGGCACAATTAAAATTTAGGGATTCAAC
GTACACACACATAGATTCCGAGAGAAATGTTGAAGCCGTAAAACCCCCACACAAGCAGGAAACAACAGTCTTACCT
ATTATTCAAGAGGCACGTAAAGGAGCTCATTTGAGGAGATTTTCTGCTGTTATTGCCATCGAATTTTTAACGTATT
TTCCAAATTAGAAAATATTCAGCCTGATGTTGTCAATATTTCAGACCACAAGGGTATCATTTAGGAAAATGGTTTC
TTACTGTCCTGAAAGAGTTACTGTTCTTCCCTAAGGGCCTAATTTACAAAGCAGCAAACTTGCTGGTAGGATTTGG
CTGAAAATCACATTGTCTCGGTAGAACTCTTTCATCTGATTTATGTGCATTGCATTTTGCAAATAACTCTTGGAAA
GTTATTTACTAGTTACTTTCTCTGGAAGCAGAGGGTAAGCGGCATTTCTAGTTTAAGGATAGAGGAGCTAAGATGC
ATCAAGCGCAGCTCATCATGAAGCTGATGCTGATAAAATGCACAATATTACATTCTCTAAGTTTCACTCTGCCATG
GGAGAATTTCATATTTTTAAATTTTGTTTGAAATTGGACTACATTAGAAAATATGTCAAATGTCTAACCCTGCATT
TATATTCTGGAATGTGACAGCTTATTTCTGTTCCAAATTTTGCACTGGAGATGGAGTAAGTCTTAATGCAAACTGC
ATGAAACTGCCACTTTTATAGGTCACACCCAGTCAATTGTCAGCAGTTACACATGGTTCAAACTGTAAGGTGTATG
CCCAATTGTAGCATTGAGATTCGTGGAGTTGTTGCAGTGGTTCTGAATTTTTCAAGCATGATACATAAAAAGATAA
ATGACTCTTTTGATATTTCTCCTTGCATTGATAGTTTGCCTGAAAACTAGATAAGCAGGGAGCCGGCAGTCCACGT
TAGCCCTTGAACTACATGAGGTTTAATTTATTTGCCCAACCAGAACCCTACACTACCTTTCAGCTGTGCAGTATTA
AAGTTTATTTAGGAGTTGATAAATAGCTTAGTGCAATGCTTCCTTTTTTCCAGTAGCTACATCCTCATAAACCTAT
TCTACCCTCCACCAGTTAATGCAGACAGAAGATTTTTATCCAGTATGAGCACTGAAACTCCACTGTGGAAGACTGT
GTGCTCAGCAAAAACCTCACCCATGATGAATAAACAGCTCTTCCGGGGGCTTTGCTGCCGCTGGCTCGGCAGGAGT
TGTTTATTGCCTGGTTTGCACATCCCATGATAAAGTTGCTGCTGAAATAAATTGCAGTTTTGCATAATTATTGACA
ATCACATCTTAACAAGCAATGTGTATCATATTCAAGTGTTCAATTTTTTAAAATCCATTTTTAGCTTATGTTTAAT
CCCAGAAAGTGTTTGTGTAGTAATAGAAGGCAAATAAGACATTTAAATAGAGTACTAATTTCCTCATTGCAGACAA
AGTTTACCTGAATCTTTTTCCATAGGACTGTTACTGCCTAAGGCAATTTTCCTTTCTAAGCTATTATTATATAGAT
ATTTGCTGAGGGCATATGTGTGTGTATCCACAATACATGCATTTTATATATATATATATATATATATATATGATCA
AAAATATGAATACATTTTTAGAGTTTTTGTCATGAAAGAGTTTGTTTCATCTTTTTAAAATATTACAGGAATGGGG
AAATGGGATATGGGTAGAAGGAACTAATGTTTTTGAGTAACTGTAATGTATAACTGTATAACGTGGGGCACTCAAC
TTCACAGGAATTTTTTATTTTAATTCTCATCACAGCAATAGATATTGCAGATGAGAAACTGAGAATCAGAGAGGGA
ACTTGCCATATCACGTAAGTGGTAAAGAACACTGGGAATTGAACTCAGATCTGCCTAGTTTTTAAAACTCTACTCT
TTTTCATTACACATAACATTTTTATTTTGGAAAATGTTCTCAGTTGTATGATCAAGTAGTTAAATATGAAACTAAC
ACAATAATTATAACTGATGTCATGCAAAATGATAGTTTGCACAAAATGATAGTTTCTATGAAATGTTATTTCTTTA
CTTGTTAAGTCTTTCTTCCTTTGCCCTCCAATCCCCTTCTTTTTGTCTTTTCCTCTAGTCTTTTCCTTTTGATTCT
AGGTTTGTATTTTCTTGACTTTTCTCCTTGCATATCAAATCCTTGTTTTCTGCCTCAGAGCAGCATCAAAGACAAG
CATGGTACAGGGATTTTAGGGTTTTAACTATAAAGGTTTGTCTCAAATTTGGCAGTATATTAAAAATAAGCTTTCA
AAATTGACCAACAAAAACTACAAAATTGAAAAAAAGGTACTTTGAACTTTCACATGTTCAAATATATGTATATATA
TTTCACATATATATATGAAACCTCCTCTGTGGAGAGGGGTTTATAGAAATCTGTAATTGTCATTCTTGCATGCCTT
CCCCCATACAAACGCCTTTAAGTTAAATAAAAATGAAAGTAAATAGACTGCACAATATTATAGTTGTTGCTTAAAG
GAAGAGCTGTAGCAACAACTCACCCCATTGTTGGTATATTACAATTTAGTTCCTCCATCTTTCTCTTTTTATGGAG
TTCACTAGGTGCACCATTCTGATATTTAATAATTGCATCTGAACATTTGGTCCTTTGCAG
Homo sapiens dystrophin (DMD), intron 53 target sequence 1 (nucleotide
positions 1665236-1665285 of NCBI Reference Sequence: NG_012232.1)
(SEQ ID NO: 771)
GTTAGTATCAAAGATACCTTTTTAAAATAAAATACTGGTTACATTTGATA
Homo sapiens dystrophin (DMD), intron 53 target sequence 2 (nucleotide
positions 1665342-1665385 of NCBI Reference Sequence: NG_012232.1)
(SEQ ID NO: 772)
ATCACGTTAAAGCTGAAATGAACAGTAGACTTTGTATATTTATT
Homo sapiens dystrophin (DMD), intron 53 target sequence 3 (nucleotide
positions 1686260-1686309 of NCBI Reference Sequence: NG_012232.1)
(SEQ ID NO: 773)
ATGCTGCATTTGAAAAGTTTGTCCTGAAAGGTGGGTTACCTTATACTGTC
Homo sapiens dystrophin (DMD), intron 53 target sequence 4 (nucleotide
positions 1686339-1686382 of NCBI Reference Sequence: NG_012232.1)
(SEQ ID NO: 774)
AAGCAATCTAATATATGTATTCTGACCTGAGGATTCAGAAGCTG
Homo sapiens dystrophin (DMD), intron 53 target sequence 5 (nucleotide
positions 1716498-1716747 of NCBI Reference Sequence: NG_012232.1)
(SEQ ID NO: 775)
GTTTATAGAAATCTGTAATTGTCATTCTTGCATGCCTTCCCCCATACAAACGCCTTTAAGTTAAATAAAAATGAAA
GTAAATAGACTGCACAATATTATAGTTGTTGCTTAAAGGAAGAGCTGTAGCAACAACTCACCCCATTGTTGGTATA
TTACAATTTAGTTCCTCCATCTTTCTCTTTTTATGGAGTTCACTAGGTGCACCATTCTGATATTTAATAATTGCAT
CTGAACATTTGGTCCTTTGCAG
Homo sapiens dystrophin (DMD), intron 53/exon 54 junction (nucleotide
positions 1686464-1686495 of NCBI Reference Sequence: NG_012232.1)
(SEQ ID NO: 776)
AATTGCATCTGAACATTTGGTCCTTTGCAGCAGTTGGCCAAAGACCTCCGCCAGTGGCAG
Homo sapiens dystrophin (DMD), transcript variant Dp427m, exon 54
(nucleotide positions 8117-8271 of NCBI Reference Sequence: NM_004006.2; nucleotide
positions 1686466-1686620 of NCBI Reference Sequence: NG_012232.1)
(SEQ ID NO: 777)
CAGTTGGCCAAAGACCTCCGCCAGTGGCAGACAAATGTAGATGTGGCAAATGACTTGGCCCTGAAACTTCTCCGGG
ATTATTCTGCAGATGATACCAGAAAAGTCCACATGATAACAGAGAATATCAATGCCTCTTGGAGAAGCATTCATAA
AAG

In some embodiments, an oligonucleotide useful for targeting DMD (e.g., for exon skipping) targets a splicing feature in a DMD sequence (e.g., a DMD pre-mRNA). In some embodiments, a splicing feature in a DMD sequence is an exonic splicing enhancer (ESE), a branch point, a splice donor site, or a splice acceptor site in a DMD sequence. In some embodiments, an ESE is in exon 53 of a DMD sequence (e.g., a DMD pre-mRNA). In some embodiments, a branch point is in intron 52 or intron 53 of a DMD sequence (e.g., a DMD pre-mRNA). In some embodiments, a splice donor site is across the junction of exon 52 and intron 52, in intron 52, across the junction of exon 53 and intron 53, or in intron 53 of a DMD sequence (e.g., a DMD pre-mRNA). In some embodiments, a splice acceptor site is in intron 52, across the junction of intron 52 and exon 53, in intron 53, or across the junction of intron 53 and exon 54 of a DMD sequence (e.g., a DMD pre-mRNA). In some embodiments, the oligonucleotide useful for targeting DMD promotes skipping of exon 53, such as by targeting a splicing feature (e.g., an ESE, a branch point, a splice donor site, or a splice acceptor site) in a DMD sequence (e.g., a DMD pre-mRNA). Examples of ESEs, branch points, splice donor sites, and splice acceptor sites are provided in Table 9.

In some embodiments, an oligonucleotide useful for targeting DMD (e.g., for exon skipping) targets an exonic splicing enhancer (ESE) in a DMD sequence. In some embodiments, an oligonucleotide useful for targeting DMD (e.g., for exon skipping) targets an ESE in DMD exon 53 (e.g., an ESE listed in Table 9).

In some embodiments, an oligonucleotide useful for targeting DMD (e.g., for exon skipping, such as for skipping exon 53) comprises a region of complementarity to a target sequence comprising one or more full or partial ESEs of a DMD transcript (e.g., one or more full or partial ESEs listed in Table 9). In some embodiments, the oligonucleotide comprises a region of complementarity to a target sequence comprising one or more full or partial ESEs of DMD exon 53. In some embodiments, the oligonucleotide comprises a region of complementarity to a target sequence comprising one or more full or partial ESEs as set forth in any one of SEQ ID NOs: 689-715. In some embodiments, the oligonucleotide comprises a region of complementarity to a target sequence comprising at least 4 (e.g., 4, 5, 6, 7, or 8) consecutive nucleotides of an ESE as set forth in any one of SEQ ID NOs: 689-715. In some embodiments, the oligonucleotide comprises at least 4 (e.g., 4, 5, 6, 7, or 8) consecutive nucleotides of an ESE antisense sequence as set forth in any one of SEQ ID NOs: 723-749.

In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) of DMD exon 53. In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) as set forth in any one of SEQ ID NOs: 689-715. In some embodiments, the oligonucleotide comprises at least 6 (e.g., 6, 7, 8, 9, 10, 11, 12, 13, 14, 15, 16, 17, 18, 19, 20, or more) nucleotides of one or more ESE antisense sequences (e.g., antisense sequences of 2, 3, 4, or more adjacent ESEs) as set forth in any one of SEQ ID NOs: 723-749.

In some embodiments, an oligonucleotide useful for targeting DMD (e.g., for exon skipping, such as for skipping exon 53) is 18-35 nucleotides in length, and comprises a region of complementarity to a target sequence comprising at least 4 (e.g., 4, 5, 6, 7, or 8) consecutive nucleotides of an ESE as set forth in any one of SEQ ID NOs: 689-715. In some embodiments, an oligonucleotide useful for targeting DMD (e.g., for exon skipping, such as for skipping exon 53) is 20-30 (e.g., 20, 25, 30) nucleotides in length, and comprises a region of complementarity to a target sequence comprising at least 4 (e.g., 4, 5, 6, 7, or 8) consecutive nucleotides of an ESE as set forth in any one of SEQ ID NOs: 689-715. In some embodiments, an oligonucleotide useful for targeting DMD (e.g., for exon skipping, such as for skipping exon 53) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of an ESE as set forth in any one of SEQ ID NOs: 689-715. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, or 8) consecutive nucleotides of an ESE as set forth in any one of SEQ ID NOs: 689-715.

In some embodiments, an oligonucleotide useful for targeting DMD (e.g., for exon skipping) targets a branch point in a DMD sequence. In some embodiments, an oligonucleotide useful for targeting DMD (e.g., for exon skipping) targets a branch point in DMD intron 52 or intron 53 (e.g., a branch point listed in Table 9).

In some embodiments, an oligonucleotide useful for targeting DMD (e.g., for exon skipping, such as for skipping exon 53) comprises a region of complementarity to a target sequence comprising a full or partial branch point of a DMD transcript (e.g., a full or partial branch point listed in Table 9). In some embodiments, the oligonucleotide comprises a region of complementarity to a target sequence comprising a full or partial branch point of DMD intron 52 or intron 53. In some embodiments, the oligonucleotide comprises a region of complementarity to a target sequence comprising a full or partial branch point as set forth in SEQ ID NO: 686, 687, or 717. In some embodiments, the oligonucleotide comprises a region of complementarity to a target sequence comprising at least 4 (e.g., 4, 5, 6, or 7) consecutive nucleotides of a branch point as set forth in SEQ ID NO: 686, 687, or 717. In some embodiments, the oligonucleotide comprises at least 4 (e.g., 4, 5, 6, or 7) consecutive nucleotides of a branch point antisense sequence as set forth in SEQ ID NO: 720, 721, or 751.

In some embodiments, an oligonucleotide useful for targeting DMD (e.g., for exon skipping, such as for skipping exon 53) is 18-35 nucleotides in length, and comprises a region of complementarity to a target sequence comprising at least 4 (e.g., 4, 5, 6, or 7) consecutive nucleotides of a branch point as set forth in SEQ ID NO: 686, 687, or 717. In some embodiments, an oligonucleotide useful for targeting DMD (e.g., for exon skipping, such as for skipping exon 53) is 20-30 (e.g., 20, 25, 30) nucleotides in length, and comprises a region of complementarity to a target sequence comprising at least 4 (e.g., 4, 5, 6, or 7) consecutive nucleotides of a branch point as set forth in SEQ ID NO: 686, 687, or 717. In some embodiments, an oligonucleotide useful for targeting DMD (e.g., for exon skipping, such as for skipping exon 53) is 20-25 (i.e., 20, 21, 22, 23, 24, or 25) nucleotides in length, and comprises a region of complementarity to a target sequence comprising at least 4 (e.g., 4, 5, 6, or 7) consecutive nucleotides of a branch point as set forth in SEQ ID NO: 686, 687, or 717. In some embodiments, an oligonucleotide useful for targeting DMD (e.g., for exon skipping) is 30 nucleotides in length, and comprises a region of complementarity to a target sequence comprising at least 4 (e.g., 4, 5, 6, or 7) consecutive nucleotides of a branch point as set forth in SEQ ID NO: 686, 687, or 717.

In some embodiments, an oligonucleotide useful for targeting DMD (e.g., for exon skipping) targets a splice donor site in a DMD sequence. In some embodiments, an oligonucleotide useful for targeting DMD (e.g., for exon skipping) targets a splice donor site across the junction of exon 52 and intron 52, in intron 52, across the junction of exon 53 and intron 53, or in intron 53 (e.g., a splice donor site listed in Table 9).

In some embodiments, an oligonucleotide useful for targeting DMD (e.g., for exon skipping, such as for skipping exon 53) comprises a region of complementarity to a target sequence comprising a full or partial splice donor site of a DMD transcript (e.g., a full or partial splice donor site listed in Table 9). In some embodiments, the oligonucleotide comprises a region of complementarity to a target sequence comprising a full or partial splice donor site across the junction of exon 52 and intron 52, in intron 52, across the junction of exon 53 and intron 53, or in intron 53 of DMD. In some embodiments, the oligonucleotide comprises a region of complementarity to a target sequence comprising a full or partial splice donor site as set forth in SEQ ID NO: 685 or 716. In some embodiments, the oligonucleotide comprises a region of complementarity to a target sequence comprising at least 4 (e.g., 4, 5, 6, 7, or 8) consecutive nucleotides of a splice donor site as set forth in SEQ ID NO: 685 or 716. In some embodiments, the oligonucleotide comprises at least 4 (e.g., 4, 5, 6, 7, or 8) consecutive nucleotides of a splice donor site antisense sequence as set forth in SEQ ID NO: 719 or 750.

In some embodiments, an oligonucleotide useful for targeting DMD (e.g., for exon skipping, such as for skipping exon 53) is 18-35 nucleotides in length, and comprises a region of complementarity to a target sequence comprising at least 4 (e.g., 4, 5, 6, 7, or 8) consecutive nucleotides of a splice donor site as set forth in SEQ ID NO: 685 or 716. In some embodiments, an oligonucleotide useful for targeting DMD (e.g., for exon skipping, such as for skipping exon 53) is 20-30 (e.g., 20, 25, 30) nucleotides in length, and comprises a region of complementarity to a target sequence comprising at least 4 (e.g., 4, 5, 6, 7, or 8) consecutive nucleotides of a splice donor site as set forth in SEQ ID NO: 685 or 716. In some embodiments, an oligonucleotide useful for targeting DMD (e.g., for exon skipping, such as for skipping exon 53) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of a splice donor site as set forth in SEQ ID NO: 685 or 716. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, or 8) consecutive nucleotides of a splice donor site as set forth in SEQ ID NO: 685 or 716.

In some embodiments, an oligonucleotide useful for targeting DMD (e.g., for exon skipping) targets a splice acceptor site in a DMD sequence. In some embodiments, an oligonucleotide useful for targeting DMD (e.g., for exon skipping) targets a splice acceptor site in intron 52, across the junction of intron 52 and exon 53, in intron 53, or across the junction of intron 53 and exon 54 (e.g., a splice acceptor site listed in Table 9).

In some embodiments, an oligonucleotide useful for targeting DMD (e.g., for exon skipping, such as for skipping exon 53) comprises a region of complementarity to a target sequence comprising a full or partial splice acceptor site of a DMD transcript (e.g., a full or partial splice acceptor site listed in Table 9). In some embodiments, the oligonucleotide comprises a region of complementarity to a target sequence comprising a full or partial splice acceptor site in intron 52, across the junction of intron 52 and exon 53, in intron 53, or across the junction of intron 53 and exon 54 of DMD. In some embodiments, the oligonucleotide comprises a region of complementarity to a target sequence comprising a full or partial splice acceptor site as set forth in SEQ ID NO: 688 or 718. In some embodiments, the oligonucleotide comprises a region of complementarity to a target sequence comprising at least 4 (e.g., 4, 5, 6, 7, 8, or 9) consecutive nucleotides of a splice acceptor site as set forth in SEQ ID NO: 688 or 718. In some embodiments, the oligonucleotide comprises at least 4 (e.g., 4, 5, 6, 7, 8, or 9) consecutive nucleotides of a splice acceptor site antisense sequence as set forth in SEQ ID NO: 722 or 752.

In some embodiments, an oligonucleotide useful for targeting DMD (e.g., for exon skipping, such as for skipping exon 53) is 18-35 nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 688 or 718. In some embodiments, an oligonucleotide useful for targeting DMD (e.g., for exon skipping, such as for skipping exon 53) is 20-30 (e.g., 20, 25, 30) nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 688 or 718. In some embodiments, an oligonucleotide useful for targeting DMD (e.g., for exon skipping, such as for skipping exon 53) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of a splice acceptor site as set forth in SEQ ID NO: 688 or 718. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 688 or 718.

In some embodiments, an oligonucleotide useful for targeting DMD (e.g., for exon skipping) comprises a region of complementarity to a junction of an exon and an intron of a DMD RNA (e.g., any one of the exon/intron junctions provided by SEQ ID NOs: 753, 761, 768, and 776). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a junction of an exon and an intron of a DMD RNA (e.g., any one of the exon/intron junctions provided by SEQ ID NOs: 753, 761, 768, and 776). In some embodiments, an oligonucleotide useful for targeting DMD (e.g., for exon skipping) is complementary to any one of SEQ ID NOs: 753, 761, 768, and 776.

In some embodiments, an oligonucleotide useful for targeting DMD (e.g., for exon skipping) comprises a region of complementarity to a target sequence of a DMD RNA (e.g., a target sequence provided by any one of SEQ ID NOs: 755-760, 763-767, 771-775, and 769). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 755-760, 763-767, 771-775, and 769). In some embodiments, an oligonucleotide useful for targeting DMD (e.g., for exon skipping) is complementary to any one of SEQ ID NOs: 755-760, 763-767, 771-775, and 769.

TABLE 9
Example target sequence motifs

| | | SEQ | | SEQ | Motif |
|---|---|---|---|---|---|
| Location | | ID | Motif | ID | Antisense |
| in DMD | Type | NO: | Sequence^{†} | NO: | Sequence^{†} |
| Across | Splice | 685 | AAGTAAGT | 719 | ACTTACTT |
| exon 52/ | Donor | | | | |
| intron 52 | | | | | |
| junction | | | | | |
| Intron 52 | Branch | 686 | TTAAC | 720 | GTTAA |
| | Point | | | | |
| Intron 52 | Branch | 687 | TGTTGAT | 721 | ATCAACA |
| | Point | | | | |
| Across | Splice | 688 | TATTCTAG | 722 | ACTAGAAT |
| intron 52/ | Acceptor | | T | | A |
| exon 53 | | | | | |
| junction | | | | | |
| Exon 53 | ESE | 689 | GAATTCAG | 723 | CTGAATTC |
| Exon 53 | ESE | 690 | TCAGTGG | 724 | CCACTGA |
| Exon 53 | ESE | 691 | CAGTGGG | 725 | CCCACTG |
| Exon 53 | ESE | 692 | GTACAAG | 726 | CTTGTAC |
| Exon 53 | ESE | 693 | TCAGAAC | 727 | GTTCTGA |
| Exon 53 | ESE | 694 | AACCGGA | 728 | TCCGGTT |
| Exon 53 | ESE | 695 | CGGAGGC | 729 | GCCTCCG |
| Exon 53 | ESE | 696 | TTAAAGG | 730 | CCTTTAA |
| Exon 53 | ESE | 697 | GGATTCAA | 731 | TTGAATCC |
| Exon 53 | ESE | 698 | ACAATGG | 732 | CCATTGT |
| Exon 53 | ESE | 699 | GGCTGGAA | 733 | TTCCAGCC |
| Exon 53 | ESE | 700 | CTAAGGA | 734 | TCCTTAG |
| Exon 53 | ESE | 701 | CTGAGCA | 735 | TGCTCAG |
| Exon 53 | ESE | 702 | AGCAGGT | 736 | ACCTGCT |
| Exon 53 | ESE | 703 | TCTTAGG | 737 | CCTAAGA |
| Exon 53 | ESE | 704 | CTTAGGA | 738 | TCCTAAG |
| Exon 53 | ESE | 705 | GGACAGG | 739 | CCTGTCC |
| Exon 53 | ESE | 706 | GACAGGC | 740 | GCCTGTC |
| Exon 53 | ESE | 707 | GGCCAGAG | 741 | CTCTGGCC |
| Exon 53 | ESE | 708 | CCAGAGC | 742 | GCTCTGG |
| Exon 53 | ESE | 709 | TGAGTC | 743 | GACTCA |
| Exon 53 | ESE | 710 | AGGAGGG | 744 | CCCTCCT |
| Exon 53 | ESE | 711 | GGTCCCTA | 745 | TAGGGACC |
| Exon 53 | ESE | 712 | ACAGTAG | 746 | CTACTGT |
| Exon 53 | ESE | 713 | CCAAAAG | 747 | CTTTTGG |
| Exon 53 | ESE | 714 | TCACAGA | 748 | TCTGTGA |
| Exon 53 | ESE | 715 | CACAGA | 749 | TCTGTG |
| Across | Splice | 716 | AGGTTAGT | 750 | ACTAACCT |
| exon 53/ | Donor | | | | |
| intron 53 | | | | | |
| junction | | | | | |
| Intron 53 | Branch | 717 | TTCTGAT | 751 | ATCAGAA |
| | Point | | | | |
| Across | Splice | 718 | TCCTTTGC | 752 | GCTGCAAA |
| intron 53/ | Acceptor | | AGC | | GGA |
| exon 54 | | | | | |
| junction | | | | | |

^{†}Each thymine base (T) in any one of the sequences provided in Table 9 may independently and optionally be replaced with a uracil base (U). Motif sequences and antisense sequences listed in Table 9 contain T's, but binding of a motif sequence in RNA and/or DNA is contemplated.

In some embodiments, any one of the oligonucleotides useful for targeting DMD (e.g., for exon skipping) is a phosphorodiamidate morpholino oligomer (PMO).

In some embodiments, the oligonucleotide may have region of complementarity to a mutant DMD allele, for example, a DMD allele with at least one mutation in any of exons 1-79 of DMD in humans that leads to a frameshift and improper RNA splicing/processing.

In some embodiments, any one of the oligonucleotides can be in salt form, e.g., as sodium, potassium, or magnesium salts.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., an mRNA or pre-mRNA molecule) results in modulation of activity or expression of the target (e.g., decreased mRNA translation, altered pre-mRNA splicing, exon skipping, target mRNA degradation, etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, 15 to 20, 20 to 25, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of the any one of the oligonucleotides described herein (e.g., the oligonucleotides listed in Table 8). In some embodiments, the oligonucleotide is complementary (e.g., at least 85% at least 90%, at least 95%, or 100%) to a target sequence of the any one of the oligonucleotides provided by SEQ ID NO: 335-684. In some embodiments, such target sequence is 100% complementary to an oligonucleotide listed in Table 8. In some embodiments, such target sequence is 100% complementary to an oligonucleotide provided by SEQ ID NO: 335-684. In some embodiments, the oligonucleotide is complementary (e.g., at least 85% at least 90%, at least 95%, or 100%) to a target sequence provided herein (e.g., a target sequence listed in Table 8). In some embodiments, the oligonucleotide is complementary (e.g., at least 85% at least 90%, at least 95%, or 100%) to any one of SEQ ID NO: 160-334.

In some embodiments, an oligonucleotide useful for targeting DMD (e.g., for exon skipping) comprises a region of complementarity to a target sequence of a DMD RNA (e.g., a target sequence provided by any one of SEQ ID NOs: 160-334). In some embodiments, an oligonucleotide useful for targeting DMD (e.g., for exon skipping) comprises a region of complementarity to at least 8 (e.g., 8, 9, 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 160-334). In some embodiments, an oligonucleotide useful for targeting DMD (e.g., for exon skipping) is complementary to any one of SEQ ID NOs: 160-334.

In some embodiments, an oligonucleotide useful for targeting DMD (e.g., for exon skipping) comprises a sequence comprising at least 8 (e.g., 8, 9, 10, 11, 12, 13, 14, 15, 16, 17, 18, 19, 20, 21, 22, 23, 24, 25, or more) consecutive nucleobases of a DMD-targeting sequence provided herein (e.g., an antisense sequence listed in Table 8). In some embodiments, the oligonucleotide comprises a sequence comprising at least 8 (e.g., 8, 9, 10, 11, 12, 13, 14, 15, 16, 17, 18, 19, 20, 21, 22, 23, 24, 25, or more) consecutive nucleobases of any one of SEQ ID NOs: 335-684. In some embodiments, the oligonucleotide comprises the sequence of any one of SEQ ID NOs: 335-684.

In some embodiments, an oligonucleotide useful for targeting DMD (e.g., for exon skipping) comprises a region of complementarity to a target sequence of a DMD RNA (e.g., a target sequence provided by any one of SEQ ID NOs: 212, 206, 224, 277, 214, 209, 207, 208, and 205). In some embodiments, an oligonucleotide useful for targeting DMD (e.g., for exon skipping) comprises a region of complementarity to at least 8 (e.g., 8, 9, 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 212, 206, 224, 277, 214, 209, 207, 208, and 205). In some embodiments, an oligonucleotide useful for targeting DMD (e.g., for exon skipping) is complementary to any one of SEQ ID NOs: 212, 206, 224, 277, 214, 209, 207, 208, and 205.

In some embodiments, an oligonucleotide useful for targeting DMD (e.g., for exon skipping) comprises a region of complementarity to a target sequence of a DMD RNA provided by any one of SEQ ID NOs: 212, 224, and 209.

In some embodiments, an oligonucleotide useful for targeting DMD (e.g., for exon skipping) comprises a region of complementarity to a target sequence of a DMD RNA provided by any one of SEQ ID NOs: 206, 277, and 205.

In some embodiments, an oligonucleotide useful for targeting DMD (e.g., for exon skipping) comprises a region of complementarity to a target sequence of a DMD RNA provided by any one of SEQ ID NOs: 206, 224, and 209.

In some embodiments, an oligonucleotide useful for targeting DMD (e.g., for exon skipping) comprises a region of complementarity to a target sequence of a DMD RNA provided by any one of SEQ ID NOs: 214, 207, and 208.

In some embodiments, an oligonucleotide useful for targeting DMD (e.g., for exon skipping) comprises a region of complementarity to a target sequence of a DMD RNA provided by any one of SEQ ID NOs: 212, 206, and 209.

In some embodiments, an oligonucleotide useful for targeting DMD (e.g., for exon skipping) comprises a region of complementarity to a target sequence of a DMD RNA provided by any one of SEQ ID NOs: 214, 207, and 205.

In some embodiments, an oligonucleotide useful for targeting DMD (e.g., for exon skipping) comprises a region of complementarity to a target sequence of a DMD RNA provided by any one of SEQ ID NOs: 277, 214, and 208.

In some embodiments, an oligonucleotide useful for targeting DMD (e.g., for exon skipping) comprises a sequence comprising at least 8 (e.g., 8, 9, 10, 11, 12, 13, 14, 15, 16, 17, 18, 19, 20, 21, 22, 23, 24, 25, or more) contiguous nucleobases of a DMD-targeting sequence provided herein (e.g., a sequence of any one of SEQ ID NOs: 562, 556, 574, 627, 564, 559, 557, 558, and 555). In some embodiments, the oligonucleotide comprises at least 8 (e.g., 8, 9, 10, 11, 12, 13, 14, 15, 16, 17, 18, 19, 20, 21, 22, 23, 24, 25, or more) consecutive nucleosides of a DMD-targeting sequence provided herein (e.g., a sequence of any one of SEQ ID NOs: 562, 556, 574, 627, 564, 559, 557, 558, and 555). In some embodiments, the oligonucleotide comprises the sequence of any one of SEQ ID NOs: 562, 556, 574, 627, 564, 559, 557, 558, and 555.

In some embodiments, an oligonucleotide useful for targeting DMD (e.g., for exon skipping) comprises a sequence of any one of SEQ ID NOs: 562, 574, and 559.

In some embodiments, an oligonucleotide useful for targeting DMD (e.g., for exon skipping) comprises a sequence of any one of SEQ ID NOs: 556, 627, and 555.

In some embodiments, an oligonucleotide useful for targeting DMD (e.g., for exon skipping) comprises a sequence of any one of SEQ ID NOs: 556, 574, and 559.

In some embodiments, an oligonucleotide useful for targeting DMD (e.g., for exon skipping) comprises a sequence of any one of SEQ ID NOs: 564, 557, and 558.

In some embodiments, an oligonucleotide useful for targeting DMD (e.g., for exon skipping) comprises a sequence of any one of SEQ ID NOs: 562, 556, and 559.

In some embodiments, an oligonucleotide useful for targeting DMD (e.g., for exon skipping) comprises a sequence of any one of SEQ ID NOs: 564, 557, and 555.

In some embodiments, an oligonucleotide useful for targeting DMD (e.g., for exon skipping) comprises a sequence of any one of SEQ ID NOs: 627, 564, and 558.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in Table 8) may independently and optionally be uracil bases (U's), and/or any one or more of the U's in the oligonucleotides provided herein may independently and optionally be T's. In some embodiments, any one or more of the thymine bases (T's) in any one of the oligonucleotides provided by SEQ ID NOs: 510-684 or in an oligonucleotide complementary to any one of SEQ ID NOs: 160-334 may optionally be uracil bases (U's), and/or any one or more of the U's in the oligonucleotides may optionally be T's. In some embodiments, any one or more of the uracil bases (U's) in any one of the oligonucleotides provided by SEQ ID NOs: 335-509 or in an oligonucleotide complementary to any one of SEQ ID NOs: 160-334 may optionally be thymine bases (T's), and/or any one or more of the T's in the oligonucleotides may optionally be U's.

b. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States patent or patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleosides, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)—Uridine Phosphoramidite, and Exon Skipping Using MNA/2′-O-Methyl Mixmer Antisense Oligonucleotide, Molecules 2016, 21, 1582, the contents of each which are incorporated herein by reference.

i. Multimers

In some embodiments, molecular payloads may comprise multimers (e.g., concatemers) of 2 or more oligonucleotides connected by a linker. In this way, in some embodiments, the oligonucleotide loading of a complex can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

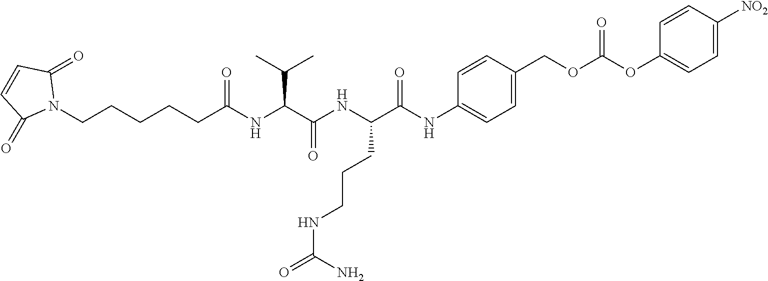 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

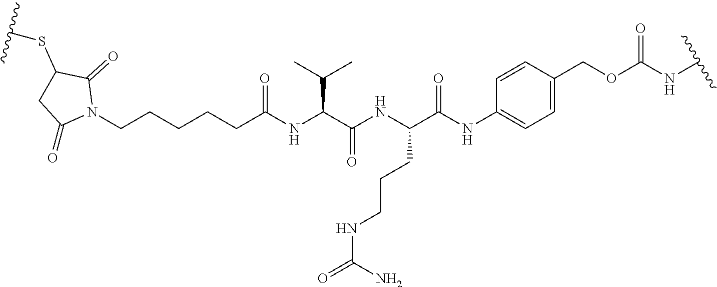 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1]

In some embodiments, before conjugation, a linker comprises a structure of:

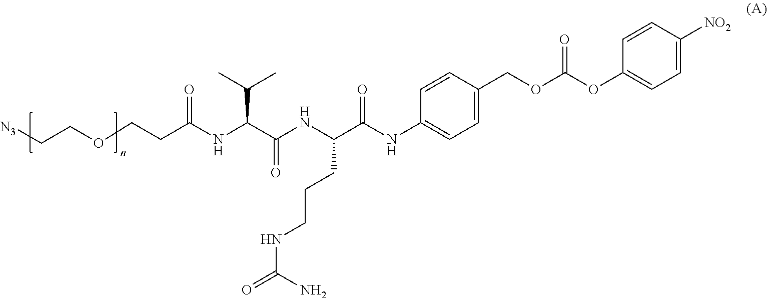

wherein n is any number from 0-10. In some embodiments, n is 3.

In some embodiments, a linker comprises a structure of:

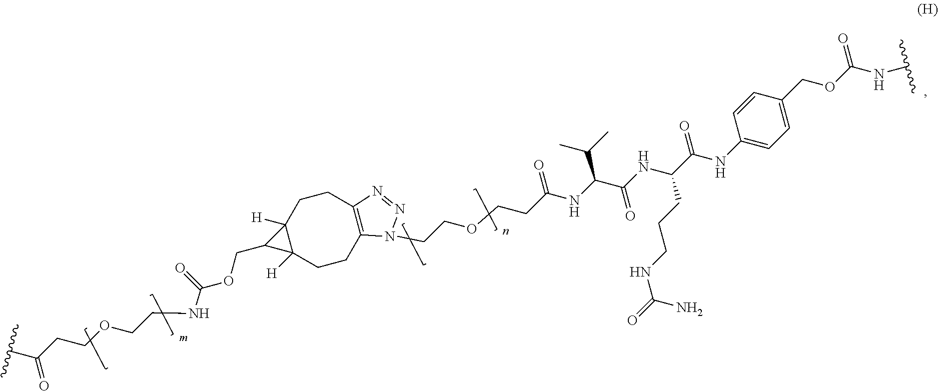

wherein n is any number from 0-10, wherein m is any number from 0-10. In some embodiments, n is 3 and/or (e.g., and) m is 4.

In some embodiments, a linker comprises a structure of:

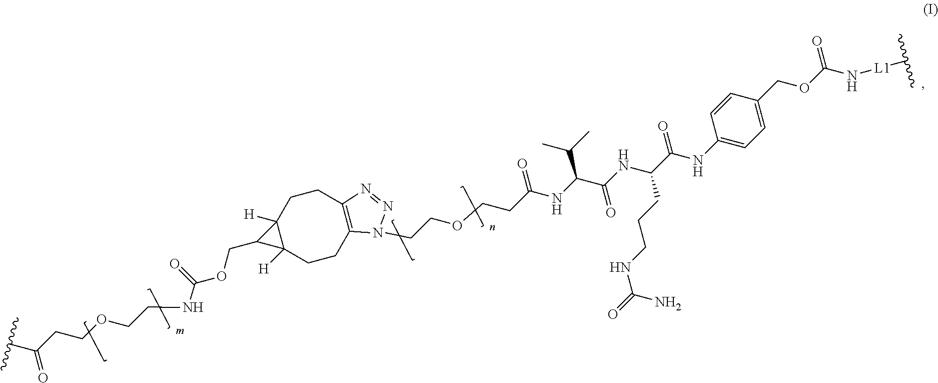

wherein n is any number from 0-10, wherein m is any number from 0-10. In some embodiments, n is 3 and/or (e.g., and) m is 4.
ii. Non-cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S, an imino, an optionally substituted nitrogen species, an optionally substituted oxygen species 0, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody—Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reaction between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

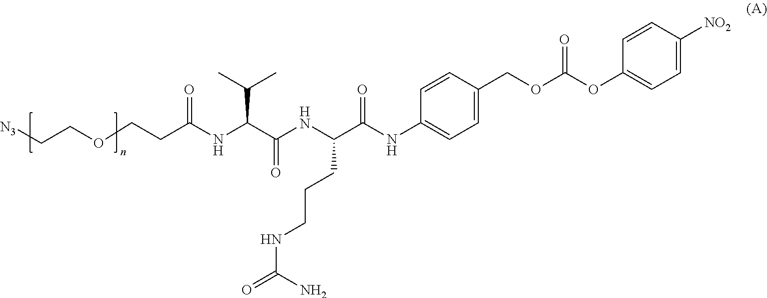

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of:

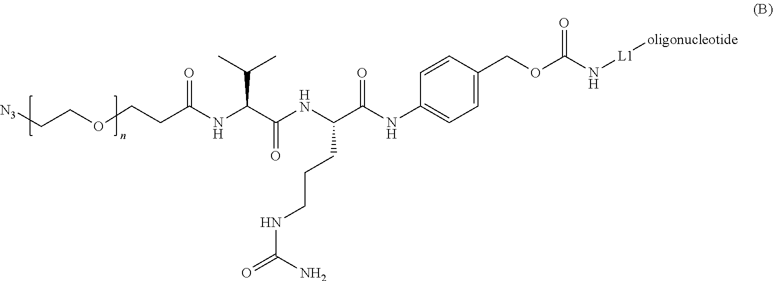

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

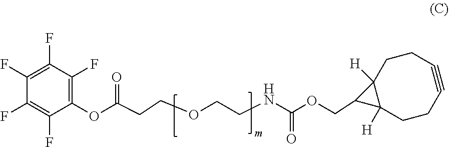

wherein m is any number from 0-10. In some embodiments, m is 4.

In some embodiments, the azide of the compound of structure (B) forms a triazole via a click reaction with the alkyne of the compound of structure (C), forming a compound comprising a structure of:

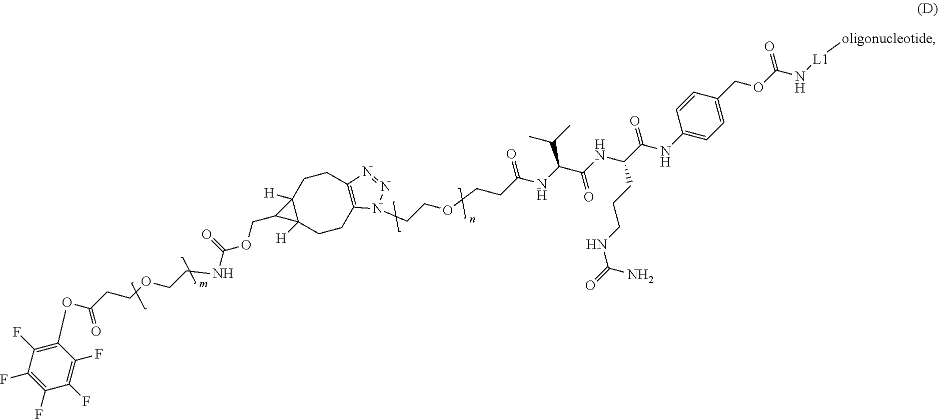

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

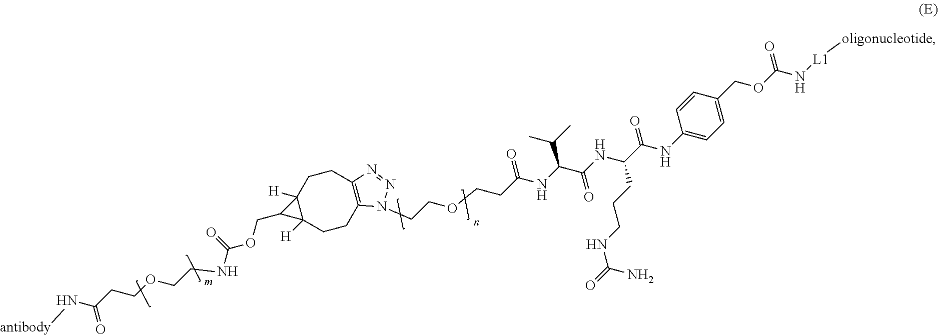

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

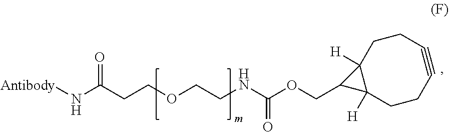

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

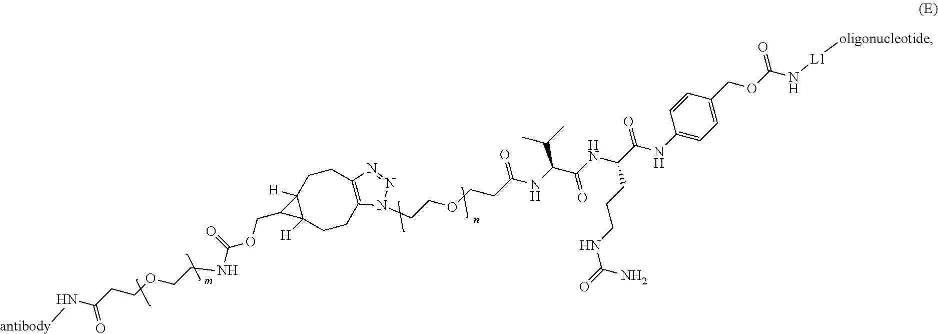

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising a structure of:

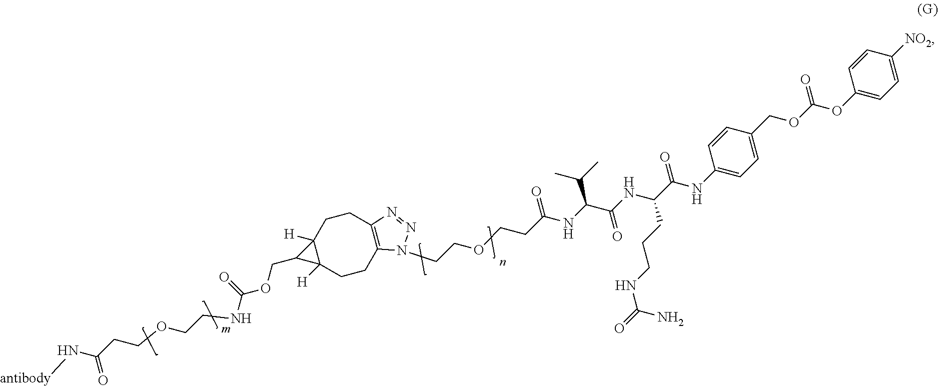

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

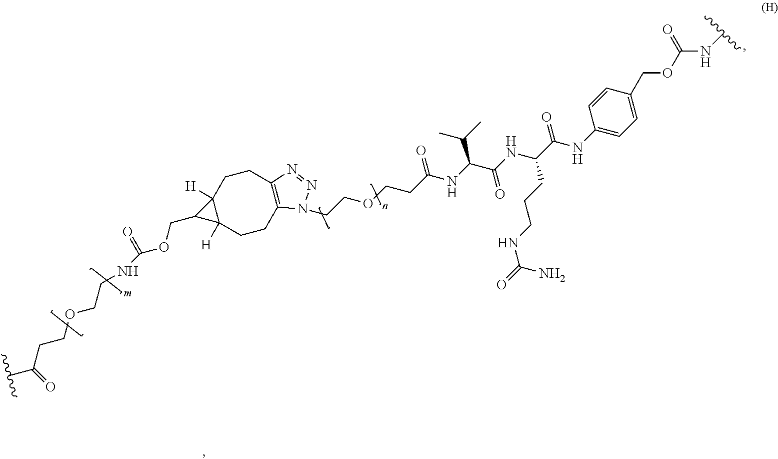

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

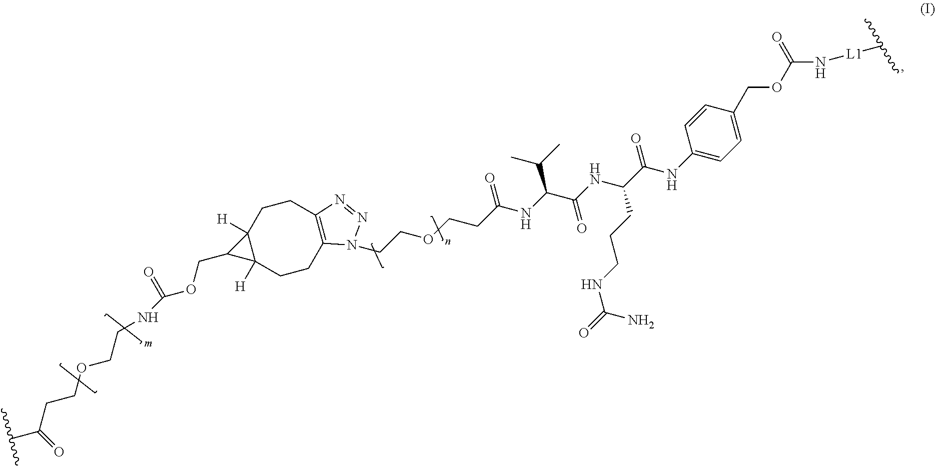

wherein n is any number from 0-10, wherein m is any number from 0-10. In some embodiments, n is 3 and/or (e.g., and) m is 4.

In some embodiments, in formulae (B), (D), (E), and (I), L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

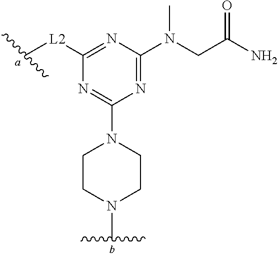

wherein L2 is

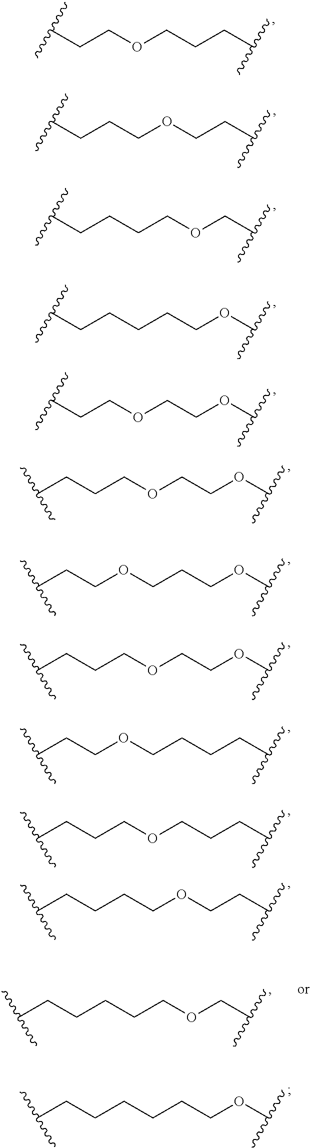

wherein a labels the site directly linked to the carbamate moiety of formulae (B), (D), (E), and (I); and b labels the site covalently linked (directly or via additional chemical moieties) to the oligonucleotide.

In some embodiments, L1 is:

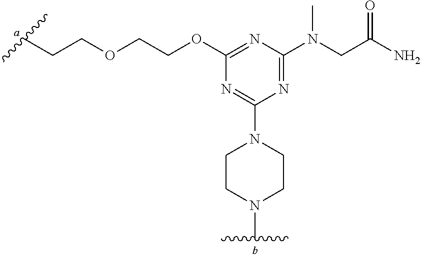 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

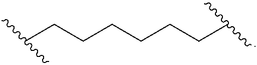 [SMILES: *CCCCCC*]

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

In some embodiments, any one of the complexes described herein has a structure of:

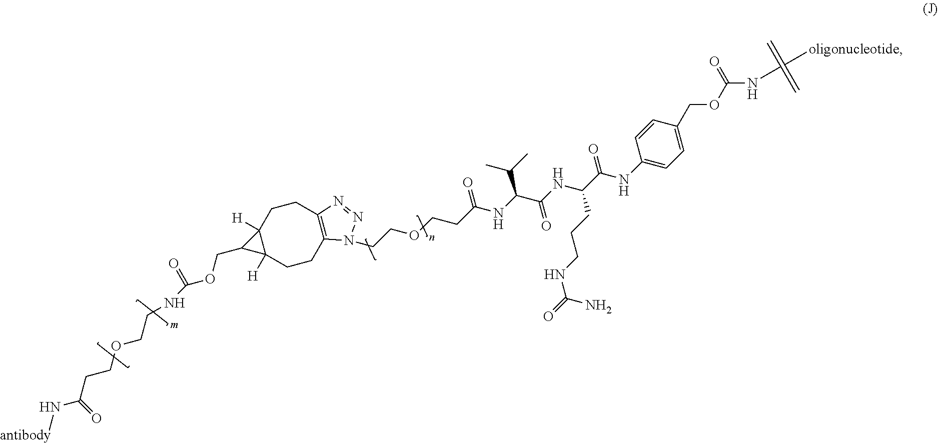

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

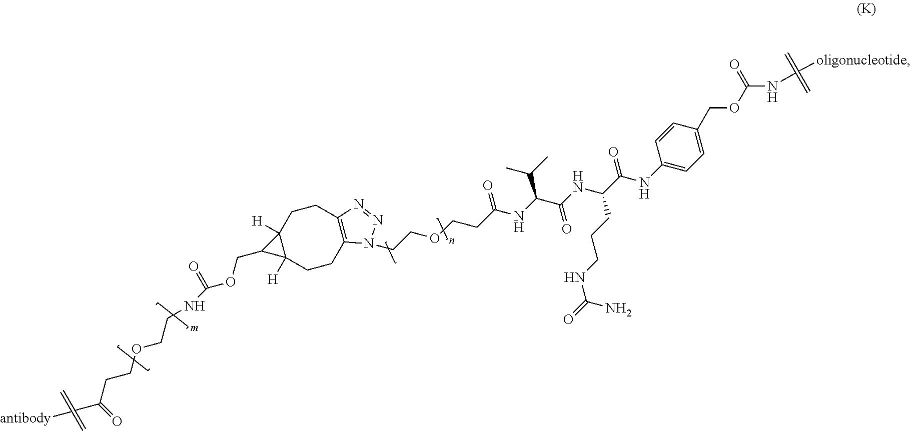

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Further provided herein are non-limiting examples of complexes comprising any one the anti-TfR1 antibodies described herein covalently linked to any of the molecular payloads (e.g., an oligonucleotide) described herein. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 8) via a linker. Any of the linkers described herein may be used. In some embodiments, if the molecular payload is an oligonucleotide, the linker is linked to the 5′ end of the oligonucleotide, the 3′ end of the oligonucleotide, or to an internal site of the oligonucleotide. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

An example of a structure of a complex comprising an anti-TfR1 antibody covalently linked to a molecular payload via a linker is provided below:

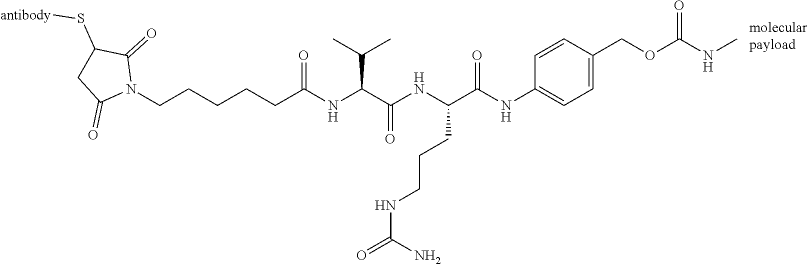

wherein the linker is linked to the antibody via a thiol-reactive linkage (e.g., via a cysteine in the antibody). In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

Another example of a structure of a complex comprising an anti-TfR1 antibody covalently linked to a molecular payload via a linker is provided below:

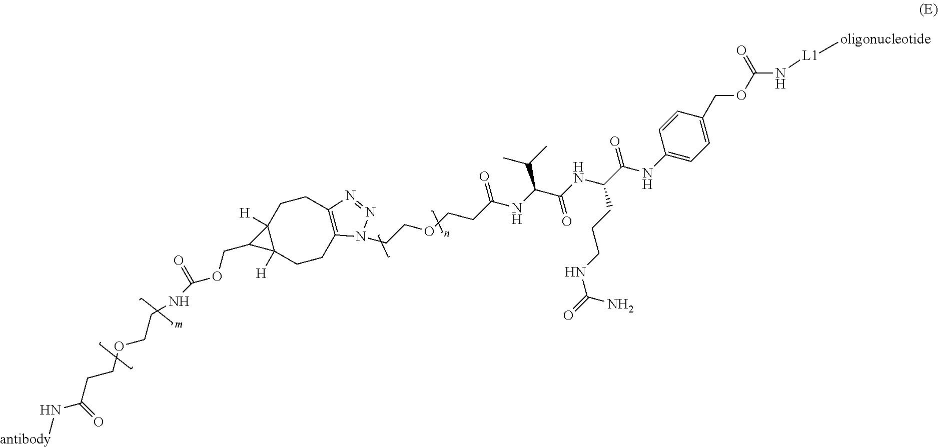

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334). It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. An average DAR of complexes in a mixture need not be an integer value. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., the antibodies provided in Tables 2-7) covalently linked to a molecular payload. In some embodiments, the complex described herein comprises an anti-TfR1 antibody described herein (e.g., the antibodies provided in Tables 2-7) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 of any one of the antibodies listed in Table 2. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a VH comprising the amino acid sequence of SEQ ID NO: 69, SEQ ID NO: 71, or SEQ ID NO: 72, and a VL comprising the amino acid sequence of SEQ ID NO: 70. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 74. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 75. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a VH comprising the amino acid sequence of SEQ ID NO: 77, and a VL comprising the amino acid sequence of SEQ ID NO: 78. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a VH comprising the amino acid sequence of SEQ ID NO: 77 or SEQ ID NO: 79, and a VL comprising the amino acid sequence of SEQ ID NO: 80. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a VH comprising the amino acid sequence of SEQ ID NO: 154, and a VL comprising the amino acid sequence of SEQ ID NO: 155. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 84, SEQ ID NO: 86 or SEQ ID NO: 87 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92 or SEQ ID NO: 94, and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92, and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 156, and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 97, SEQ ID NO: 98, or SEQ ID NO: 99 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 or SEQ ID NO: 103 and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In some embodiments, the complex described herein comprises an anti-TfR1 antibody covalently linked to a molecular payload, wherein the anti-TfR1 antibody comprises a heavy chain comprising the amino acid sequence of SEQ ID NO: 158 or SEQ ID NO: 159 and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 335-684, or complementary to any one of SEQ ID NO: 160-334).

In any of the example complexes described herein, in some embodiments, the anti-TfR1 antibody is covalently linked to the molecular payload via a linker comprising a structure of:

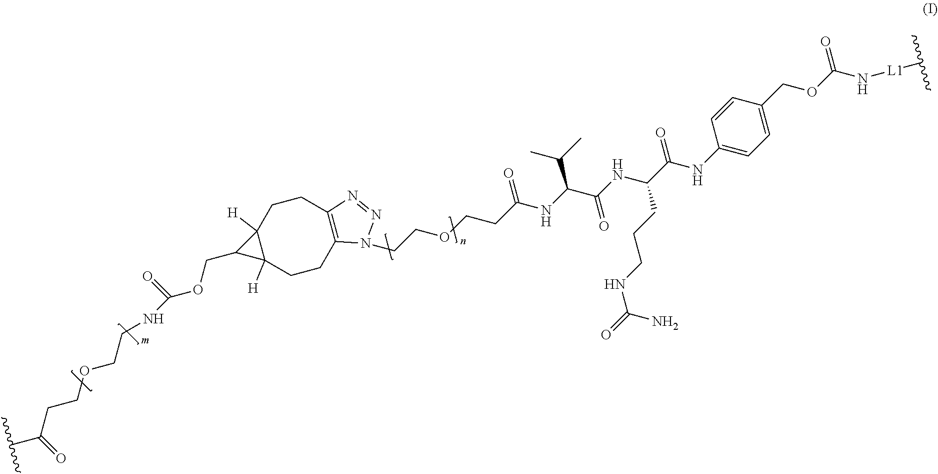

wherein n is 3, m is 4.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 335-684, or complementary to any one of SEQ ID NO: 160-334) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of:

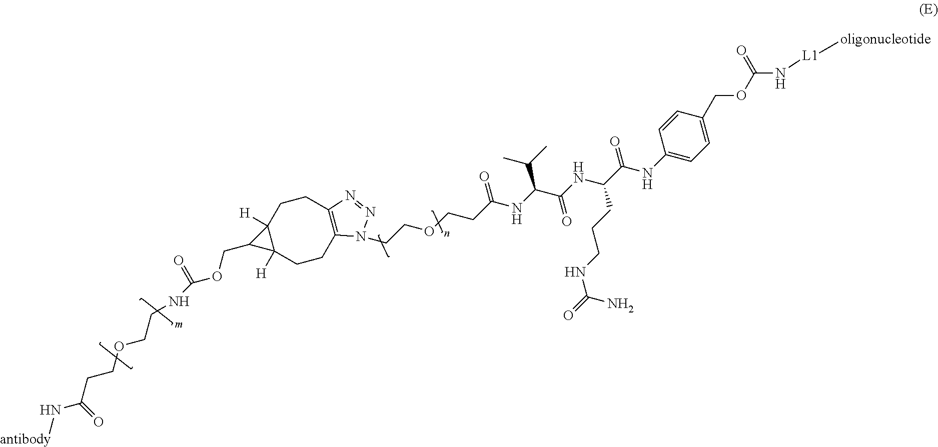

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 335-684, or complementary to any one of SEQ ID NO: 160-334) via a lysine in the anti-TfR1 antibody, wherein the anti-TfR1 antibody comprises a VH and VL of any one of the antibodies listed in Table 3, wherein the complex has a structure of:

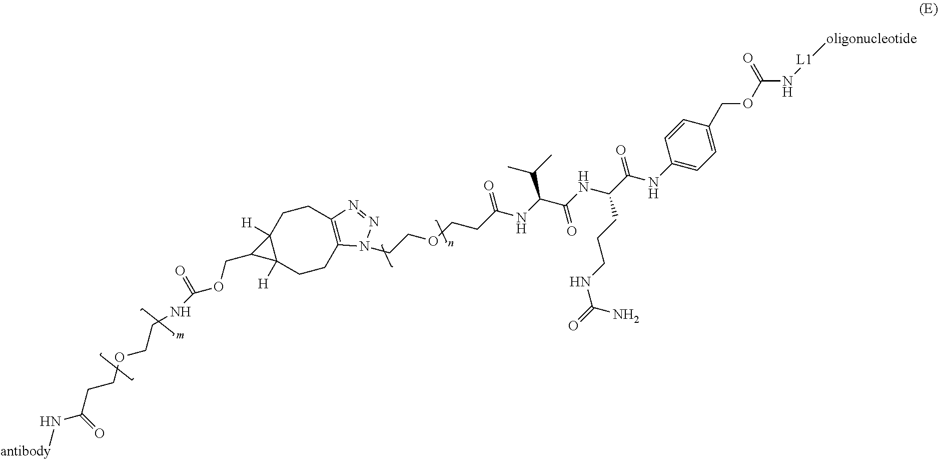

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 335-684, or complementary to any one of SEQ ID NO: 160-334) via a lysine in the anti-TfR1 antibody, wherein the anti-TfR1 antibody comprises a heavy chain and light chain of any one of the antibodies listed in Table 4, wherein the complex has a structure of:

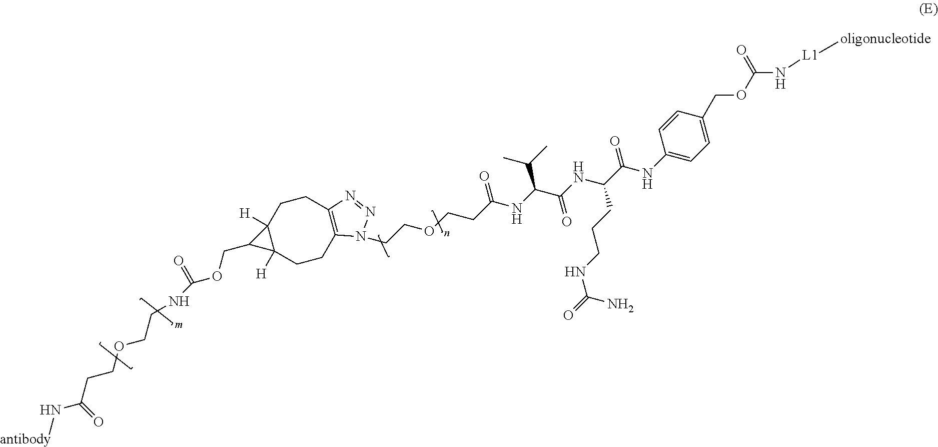

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 Fab covalently linked to the 5′ end of a DMD-targeting oligonucleotide (e.g., a DMD-targeting oligonucleotide listed in Table 8, provided by any one of SEQ ID NO: 335-684, or complementary to any one of SEQ ID NO: 160-334) via a lysine in the anti-TfR1 antibody, wherein the anti-TfR1 Fab comprises a heavy chain and light chain of any one of the antibodies listed in Table 5, wherein the complex has a structure of:

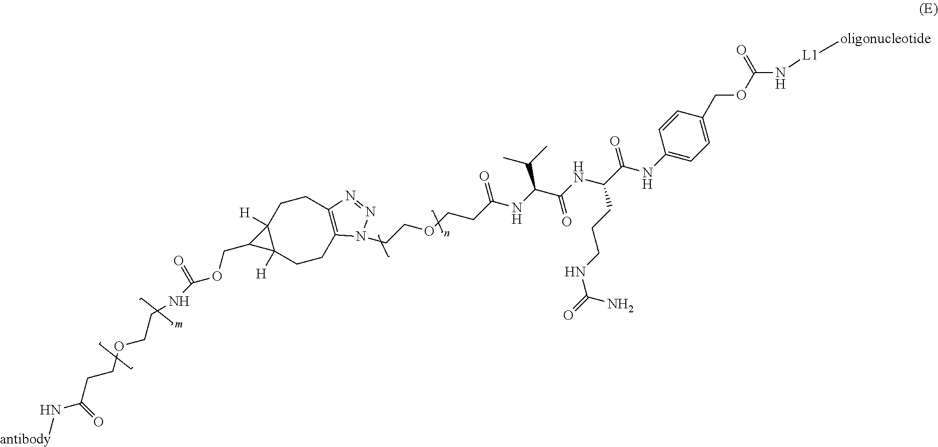

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, in any one of the examples of complexes described herein, L1 is:

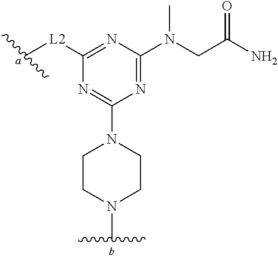

wherein L2 is

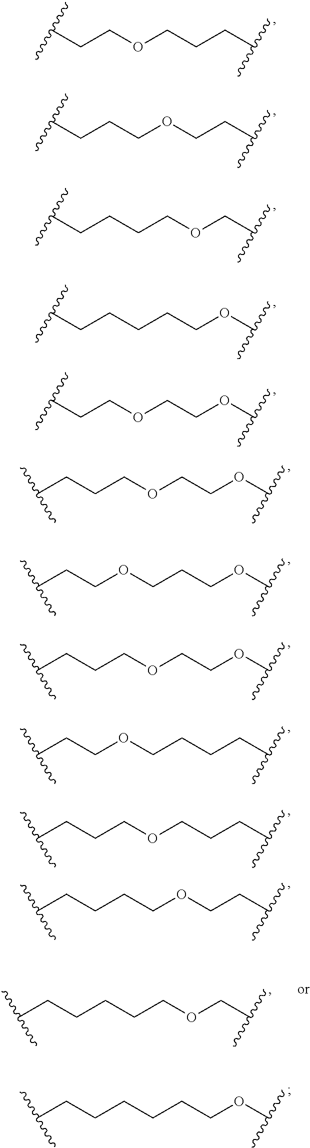

wherein a labels the site directly linked to the carbamate moiety of formulae (B), (D), (E), and (I); and b labels the site covalently linked (directly or via additional chemical moieties) to the oligonucleotide.

In some embodiments, L1 is:

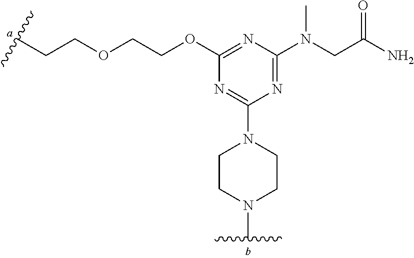 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating a subject having a dystrophinopathy, e.g., Duchenne muscular dystrophy. In some embodiments, complexes comprise a molecular payload that is an oligonucleotide, e.g., an antisense oligonucleotide that facilitates exon skipping of a pre-mRNA expressed from a mutated DMD allele.

In some embodiments, a subject may be a human subject, a non-human primate subject, a rodent subject, or any suitable mammalian subject. In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g. muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy.

In some embodiments, a subject has a mutation in a DMD gene that is amenable to exon 53 skipping. In some embodiments, a complex comprising a muscle-targeting agent covalently linked to a molecular payload as described herein is effective in treating a subject having a mutation in a DMD gene that is amenable to exon 53 skipping. In some embodiments, a complex comprises a molecular payload that is an oligonucleotide, e.g., an antisense oligonucleotide that facilitates skipping of exon 53 of a pre-mRNA, such as in a pre-mRNA encoded from a mutated DMD gene (e.g., a mutated DMD gene that is amenable to exon 53 skipping).

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with a dystrophinopathy, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan.

In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### Additional Embodiments

1. A complex comprising an anti-transferrin receptor 1 (TfR1) antibody covalently linked to a molecular payload configured for inducing skipping of exon 53 in a DMD pre-mRNA, wherein the anti-TfR1 antibody is an antibody identified in any one of Tables 2-7. 2. The complex of embodiment 1, wherein the anti-TfR1 antibody comprises: (i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.

3. The complex of embodiment 1 or embodiment 2, wherein the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.

4. The complex of any one of embodiments 1 to 3, wherein the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.

5. The complex of any one of embodiments 1 to 4, wherein the anti-TfR1 antibody is a Fab fragment, a Fab′ fragment, a F(ab′)₂ fragment, an scFv, an Fv, or a full-length IgG.

6. The complex of embodiment 5, wherein the anti-TfR1 antibody is a Fab fragment.

7. The complex of embodiment 6, wherein the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.

8. The complex of embodiment 6 or embodiment 7, wherein the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.

9. The complex of any one of embodiments 1 to 8, wherein the anti-TfR1 antibody does not specifically bind to the transferrin binding site of the transferrin receptor 1 and/or wherein the anti-TfR1 antibody does not inhibit binding of transferrin to the transferrin receptor 1.

10. The complex of any one of embodiments 1 to 9, wherein the molecular payload comprises an oligonucleotide.

11. The complex of embodiment 10, wherein the oligonucleotide promotes antisense-mediated exon skipping in the DMD pre-RNA.

12. The complex of embodiment 10 or 11, wherein the oligonucleotide comprises a region of complementarity to a splicing feature of the DMD pre-mRNA.

13. The complex of embodiment 12, wherein the splicing feature is an exonic splicing enhancer (ESE) of the DMD pre-mRNA.

14. The complex of embodiment 13, wherein the splicing feature is in exon 53 of the DMD pre-mRNA, optionally wherein the ESE comprises a sequence of any one of SEQ ID NOs: 689-715.

15. The complex of embodiment 12, wherein the splicing feature is a branch point, a splice donor site, or a splice acceptor site.

16. The complex of embodiment 15, wherein the splicing feature is across the junction of exon 52 and intron 52, in intron 52, across the junction of intron 52 and exon 53, across the junction of exon 53 and intron 53, in intron 53, or across the junction of intron 53 and exon 54 of the DMD pre-mRNA, optionally wherein the splicing feature comprises a sequence of any one of SEQ ID NOs: 685-688 and 716-718.

17. The complex of any one of embodiments 12 to 16, wherein the region of complementarity comprises at least 4 consecutive nucleosides complementary to the splicing feature.

18. The complex of any one of embodiments 1 to 9, wherein the molecular payload comprises an oligonucleotide comprising a sequence complementary to any one of SEQ ID NOs: 160-334 or comprising a sequence of any one of SEQ ID NOs: 335-684, wherein each thymine base (T) may independently and optionally be replaced with a uracil base (U), and each U may independently and optionally be replaced with a T.

19. The complex of any one of embodiments 1 to 9, wherein the molecular payload comprises an oligonucleotide comprising a sequence of any one of SEQ ID NOs: 627, 562, 521, 559, 557, 558, 556, 555, and 574, wherein each thymine base (T) may independently and optionally be replaced with a uracil base (U), and each U may independently and optionally be replaced with a T.

20. The complex of any one of embodiments 10 to 19, wherein the oligonucleotide comprises at least one modified internucleoside linkage.

21. The complex of embodiment 20, wherein the at least one modified internucleoside linkage is a phosphorothioate linkage.

22. The complex of any one of embodiments 10 to 21, wherein the oligonucleotide comprises one or more modified nucleosides.

23. The complex of embodiment 22, wherein the one or more modified nucleosides are 2′-modified nucleosides.

24. The complex of any one of embodiments 10 to 19, wherein the oligonucleotide comprises one or more phosphorodiamidate morpholinos, optionally wherein the oligonucleotide is a phosphorodiamidate morpholino oligomer (PMO).

25. The complex of any one of embodiments 1 to 24, wherein the anti-TfR1 antibody is covalently linked to the molecular payload via a cleavable linker.

26. The complex of embodiment 25, wherein the cleavable linker comprises a valine-citrulline sequence.

27. The complex of any one of embodiments 1 to 26, wherein the anti-TfR1 antibody is covalently linked to the molecular payload via conjugation to a lysine residue or a cysteine residue of the antibody.

28. A complex comprising an anti-TfR1 antibody covalently linked to an oligonucleotide configured for inducing skipping of exon 53 in a DMD pre-mRNA, wherein the oligonucleotide comprises a region of complementarity to any one of SEQ ID NOs: 160-334.

29. The complex of embodiment 28, wherein the anti-TfR1 antibody is an antibody identified in any one of Tables 2-7.

30. A complex comprising an anti-TfR1 antibody covalently linked to an oligonucleotide configured for inducing skipping of exon 53 in a DMD pre-mRNA, wherein the oligonucleotide comprises a region of complementarity to a splicing feature of the DMD pre-mRNA.

31. An oligonucleotide that targets DMD, wherein the oligonucleotide comprises a region of complementarity to any one of SEQ ID NOs: 160-334.

32. The oligonucleotide of embodiment 31, wherein the region of complementarity comprises at least 15 consecutive nucleosides complementary to any one of SEQ ID NOs: 160-334.

33. The oligonucleotide of embodiment 31 or 32, wherein the oligonucleotide comprises at least 15 consecutive nucleosides of any one of SEQ ID NOs: 335-684, optionally wherein the oligonucleotide comprises a sequence of any one of SEQ ID NOs: 335-684, wherein each thymine base (T) may independently and optionally be replaced with a uracil base (U), and each U may independently and optionally be replaced with a T.

34. The oligonucleotide of embodiment 33, wherein the oligonucleotide comprises a sequence of any one of SEQ ID NOs: 627, 562, 521, 559, 557, 558, 556, 555, and 574, wherein each thymine base (T) may independently and optionally be replaced with a uracil base (U), and each U may independently and optionally be replaced with a T.

35. A method of delivering a molecular payload to a cell, the method comprising contacting the cell with the complex of any one of embodiments 1 to 27.

36. A method of delivering an oligonucleotide to a cell, the method comprising contacting the cell with the complex of any one of embodiments 28 to 30.

37. A method of promoting the expression or activity of a dystrophin protein in a cell, the method comprising contacting the cell with the complex of any one of embodiments 1 to 27 in an amount effective for promoting internalization of the molecular payload to the cell, optionally wherein the cell is a muscle cell.

38. A method of promoting the expression or activity of a dystrophin protein in a cell, the method comprising contacting the cell with the complex of any one of embodiments 28 to 30 in an amount effective for promoting internalization of the oligonucleotide to the cell, optionally wherein the cell is a muscle cell.

39. The method of embodiment 37 or 38, wherein the cell is in vitro.

40. The method of embodiment 37 or 38, wherein the cell is in a subject.

41. The method of embodiment 40, wherein the subject is a human.

42. The method of embodiment 41, wherein the subject has a DMD gene that is amenable to skipping of exon 53.

43. The method of any one of embodiments 37 to 42, wherein the dystrophin protein is a truncated dystrophin protein.

44. A method of treating a subject having a mutated DMD allele that is associated with a dystrophinopathy, the method comprising administering to the subject an effective amount of the complex of any one of embodiments 1 to 30.

45. A method of promoting skipping of exon 53 of a DMD pre-mRNA transcript in a cell, the method comprising contacting the cell with an effective amount of the complex of any one of embodiments 1 to 30.

46. A method of treating a subject having a mutated DMD allele that is associated with a dystrophinopathy, the method comprising administering to the subject an effective amount of the complex of any one of embodiments 1 to 30.

### EXAMPLES

### Example 1. Exon-Skipping Activity of Anti-TfR1 Antibody Conjugates in Duchenne Muscular Dystrophy Patient Myotubes

In this study, the exon-skipping activities of anti-TfR1 antibody conjugates comprising an anti-TfR1 Fab (3M12 VH4/Vκ3) covalently linked to a DMD exon 51-skipping antisense oligonucleotide (ASO) were evaluated. The DMD exon 51-skipping ASO is a phosphorodiamidate morpholino oligomer (PMO) of 30 nucleotides in length and targets an ESE in DMD exon 51 having the sequence TGGAGGT (SEQ ID NO: 778). Immortalized human myoblasts bearing an exon 52 deletion in the DMD gene were thawed and seeded at a density of 1e6 cell/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50,000 cells/well. Cells were allowed to recover for 24 hours. Cells were induced to differentiate into myotubes by aspirating the growth media and replacing with differentiation media with no serum. Cells were then treated with the DMD exon 51-skipping oligonucleotide (not covalently linked to an antibody—“naked”) at 10 μM ASO or the anti-TfR1 Fab (3M12 VH4/Vκ3) covalently linked to the DMD exon 51-skipping oligonucleotide at 10 μM ASO equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in the cells. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

% Exon Skipping=Skipped Amplicon/(Skipped Amplicon +Unskipped Amplicon)*100.

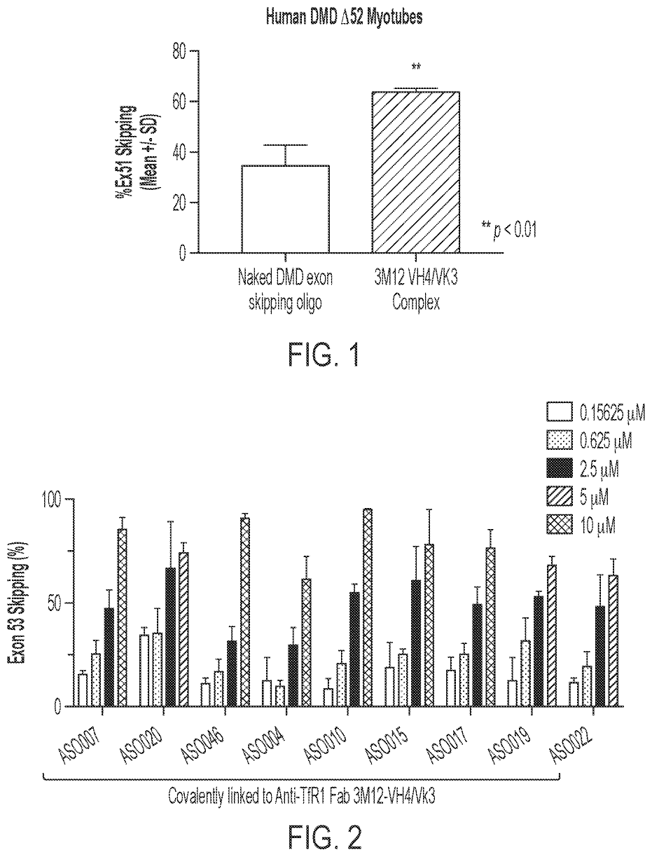

The results demonstrate that the conjugate resulted in enhanced exon skipping compared to the naked DMD exon 51-skipping oligonucleotide in patient myotubes (FIG. 1). This indicates that anti-TfR1 Fab 3M12 VH4/Vκ3 enabled cellular internalization of the conjugate into muscle cells resulting in activity of the exon 51-skipping oligonucleotide in the muscle cells. Similarly, an anti-TfR1 antibody (e.g., anti-TfR1 Fab 3M12 VH4/Vκ3) can enable internalization of a conjugate comprising the anti-TfR1 antibody covalently linked to other exon skipping oligonucleotides (e.g., an exon skipping oligonucleotide provided herein, such as an exon 53 skipping oligonucleotide) into muscle cells and facilitate activity of the exon skipping oligonucleotide in the muscle cells.

### Example 2. Exon Skipping Activity of Anti-TfR1 Fab-ASO Conjugate In Vivo in Cynomolgus Monkeys

Anti-TfR1 Fab 3M12 VH4/Vκ3 was covalently linked to the DMD exon 51-skipping antisense oligonucleotide (ASO) that was used in Example 1. The exon skipping activity of the conjugate was tested in vivo in healthy non-human primates. Naïve male cynomolgus monkeys (n=4-5 per group) were administered two doses of vehicle, 30 mg/kg naked ASO (i.e., not covalently linked to an antibody), or 122 mg/kg anti-TfR1 Fab (3M12 VH4/Vκ3) covalently linked to the DMD exon 51-skipping oligonucleotide (30 mg/kg ASO equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} * 100.$$

Calculated exon 51 skipping results are shown in Table 10.

TABLE 10
% Exon 51 skipping of DMD mRNA in cynomolgus monkey

| | Time | | | | | | | | | |
|---|---|---|---|---|---|---|---|---|---|---|
| | 2 weeks | | | | | | 4 weeks | | | |
| | Group | | | | | | | | | |
| | | | Naked | | | | Naked | | | |
| | Vehicle | | ASOª | | Conjugate | | ASOª | | Conjugate | |
| Conjugate dose^{b} | 0 | | n/a | | 122 | | n/a | | 122 | |
| ASO Dose^{c} | 0 | | 30 | | 30 | | 30 | | 30 | |
| Quadriceps ^{d} | 0.00 | (0.00) | 1.216 | (1.083) | 4.906 | (3.131) | 0.840 | (1.169) | 1.708 | (1.395) |
| Diaphragm ^{d} | 0.00 | (0.00) | 1.891 | (2.911) | 7.315 | (1.532) | 0.717 | (1.315) | 9.225 | (4.696) |
| Heart ^{d} | 0.00 | (0.00) | 0.043 | (0.096) | 3.42 | (1.192) | 0.00 | (0.00) | 4.525 | (1.400) |
| Biceps ^{d} | 0.00 | (0.00) | 0.607 | (0.615) | 3.129 | (0.912) | 1.214 | (1.441) | 4.863 | (3.881) |
| Tibialis anterior ^{d} | 0.00 | (0.00) | 0.699 | (0.997) | 1.042 | (0.685) | 0.384 | (0.615) | 0.816 | (0.915) |
| Gastrocnemius ^{d} | 0.00 | (0.00) | 0.388 | (0.573) | 2.424 | (2.329) | 0.00 | (0.00) | 5.393 | (2.695) |

ªASO = antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO dose.
^{d} Exon skipping values are mean % exon 51 skipping with standard deviations (n = 5) in parentheses.

Tissue ASO accumulation was also quantified using a hybridization ELISA with a probe complementary to the ASO sequence. A standard curve was generated and ASO levels (in ng/g) were derived from a linear regression of the standard curve. The ASO was distributed to all tissues evaluated at a higher level following the administration of the anti-TfR1 Fab VH4/Vκ3-ASO conjugate as compared to the administration of naked ASO. Intravenous administration of naked ASO resulted in levels of ASO that were close to background levels in all tissues evaluated at 2 and 4 weeks after the first does was administered. Administration of anti-TfR1 Fab VH4/Vκ3-ASO conjugate resulted in distribution of ASO through the tissues evaluated with a rank order of heart>diaphragm>bicep>quadriceps>gastrocnemius>tibialis anterior 2 weeks after first dosing. The duration of tissue concentration was also assessed. Concentrations of the ASO in quadriceps, bicep and diaphragm decreased by less than 50% over the time period evaluated (2 to 4 weeks), while levels of ASO in the heart, tibialis anterior, and gastrocnemius remained virtually unchanged (Table 11). This indicates that anti-TfR1 Fab 3M12 VH4/Vκ3 enabled cellular internalization of the conjugate into muscle cells in vivo, resulting in activity of the exon skipping oligonucleotide in the muscle cells. Similarly, an anti-TfR1 antibody (e.g., anti-TfR1 Fab 3M12 VH4/Vκ3) in vivo can enable internalization of a conjugate comprising the anti-TfR1 antibody covalently linked to other exon skipping oligonucleotides (e.g., an exon skipping oligonucleotide provided herein, such as an exon 53 skipping oligonucleotide) into muscle cells and facilitate activity of the exon skipping oligonucleotide in the muscle cells.

TABLE 11
Tissue distribution of DMD exon 51 skipping ASO in cynomolgus monkeys

| | Time | | | | | | | | | |
|---|---|---|---|---|---|---|---|---|---|---|
| | 2 weeks | | | | | | 4 weeks | | | |
| | Group | | | | | | | | | |
| | | | Naked | | | | Naked | | | |
| | Vehicle | | ASOª | | Conjugate | | ASOª | | Conjugate | |
| Conjugate dose^{b} | 0 | | n/a | | 122 | | n/a | | 122 | |
| ASO Dose^{c} | 0 | | 30 | | 30 | | 30 | | 30 | |
| Quadriceps ^{d} | 0 | (59.05) | 696.8 | (868.15) | 2436 | (954.0) | 197 | (134) | 682 | (281) |
| Diaphragm ^{d} | 0 | (144.3) | 580.02 | (360.11) | 6750 | (2256) | 60 | (120) | 3131 | (1618) |
| Heart ^{d} | 0 | (396.03) | 1449 | (1337) | 27138 | (6315) | 943 | (1803) | 30410 | (9247) |
| Biceps ^{d} | 0 | (69.58) | 615.63 | (335.17) | 2840 | (980.31) | 130 | (80) | 1326 | (623) |
| Tibialis anterior ^{d} | 0 | (76.31) | 564.71 | (327.88) | 1591 | (253.50) | 169 | (110) | 1087 | (514) |
| Gastrocnemius ^{d} | 0 | (41.15) | 705.47 | (863.75) | 2096 | (474.04) | 170 | (69) | 1265 | (272) |

ªASO = Antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO conjugate dose.
^{d} ASO values are mean concentrations of ASO in tissue as ng/g with standard deviations (n = 5) in parentheses.

### Example 3. Exon-Skipping Activity of Antisense Oligonucleotides in Duchenne Muscular Dystrophy Patient Myotubes

In this study, the exon-skipping activity of a panel of DMD exon 53-skipping antisense oligonucleotides (ASO) was evaluated. Each DMD exon 53-skipping ASO tested is a phosphorodiamidate morpholino oligomer (PMO) of 20-25 nucleotides in length and may target various splicing features in DMD exon 53 and the immediately preceding and following introns.

Immortalized human myoblasts bearing an exon 52 deletion were thawed and seeded at a density of 1×10⁶ cells/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cells were counted and seeded into Matrigel-coated wells of 96-well plates at a density of 50,000 cells/well. Cells were allowed to recover for 24 hours. Cells were induced to differentiate into myotubes by replacing the growth media with differentiation media containing no serum. Cells were then treated with each DMD exon 53-skipping oligonucleotide at a final concentration of 10 μM ASO, with each ASO tested in three replicates across three wells. Cells were incubated with ASO for ten days, then total RNA was harvested from the 96 well plates. cDNA synthesis was performed using 75 ng of total RNA, and mutation specific PCRs were performed to evaluate the degree of exon 53 skipping in the cells. Mutation-specific PCR products were run on a 4% agarose gel and visualized using SYBR gold. Densitometry was used to calculate the relative amounts of the skipped and unskipped amplicon and exon skipping was determined as a ratio of the Exon 53 skipped amplicon divided by the total amount of amplicon present:

$$\% Exon Skipping = \frac{Skipped Amplicon}{( Skipped Amplicon + Unskipped Amplicon )} * 100.$$

The results shown in Table 12 demonstrate that treatment with certain of the ASOs tested resulted in enhanced exon skipping.

TABLE 12
Exon 53 skipping antisense oligonucleotides

| | | | % Exon 53 | |
|---|---|---|---|---|
| | | SEQ | Skipping | |
| | | ID | | St. |
| ASO ID | ASO Sequence | NO: | Mean | Dev. |
| ASO001 | TTTGTGTGTCCCATGCTTGTTA | 569 | 2.26 | 0.16 |
| ASO002 | TTTGTGTGTCCCATGCTTGTT | 568 | 2.34 | 0.50 |
| ASO003 | TTGTGTGTCCCATGCTTGTT | 628 | 1.50 | 0.32 |
| ASO004 | TTGTACTTCATCCCACTGATT | 627 | 47.64 | 4.44 |
| ASO005 | TTGTGTTATGGCTAGGATGATGA | 629 | 0.72 | 0.45 |
| ASO006 | TTGTACTTCATCCCACTGAT | 626 | 9.10 | 2.10 |
| ASO007 | TTCTTGTACTTCATCCCACTGATT | 562 | 73.55 | 6.92 |
| ASO008 | TTGTGTGTCCCATGCTTGTTA | 565 | 2.04 | 0.44 |
| ASO009 | TTGTGTTATGGCTAGGATGATGAAC | 566 | 1.13 | 0.09 |
| ASO010 | TTGTACTTCATCCCACTGATTC | 564 | 53.11 | 3.97 |
| ASO011 | TTGTGTTATGGCTAGGATGATGAA | 684 | 0.85 | 0.25 |
| ASO012 | TTTGTGTGTCCCATGCTTGT | 567 | 1.48 | 0.47 |
| ASO013 | TTCTTGTACTTCATCCCACTGAT | 561 | 61.72 | 4.24 |
| ASO014 | TTCTTGTACTTCATCCCACTGA | 560 | 18.09 | 10.96 |
| ASO015 | TTCTTGTACTTCATCCCACTG | 559 | 52.26 | 9.47 |
| ASO016 | TTCTTGTACTTCATCCCACT | 625 | 35.53 | 2.96 |
| ASO017 | TGTTCTTGTACTTCATCCCACTGA | 557 | 45.01 | 6.11 |
| ASO018 | TTATGGCTAGGATGATGAAC | 683 | 1.91 | 0.36 |
| ASO019 | TGTTCTTGTACTTCATCCCACTGAT | 558 | 59.56 | 16.00 |
| ASO020 | TGTTCTTGTACTTCATCCCACTG | 556 | 81.55 | 2.95 |
| ASO021 | TGTTCTTGTACTTCATCCCAC | 624 | 2.51 | 0.15 |
| ASO022 | TGTTCTTGTACTTCATCCCACT | 555 | 67.30 | 3.26 |
| ASO023 | TGTGTTATGGCTAGGATGATGAAC | 554 | 1.23 | 1.27 |
| ASO024 | ATCTTTGATACTAACCTTGGTT | 580 | 4.30 | 2.19 |
| ASO025 | ATTATTCATTGTGTTATGGCTAGG | 640 | 0.81 | 0.31 |
| ASO026 | ATTATTCATTGTGTTATGGCTAG | 639 | 0.93 | 0.32 |
| ASO027 | ATCTTTGATACTAACCTTGGTTT | 638 | 3.46 | 1.65 |
| ASO028 | ATTATTCATTGTGTTATGGCTAGGA | 641 | 0.58 | 0.58 |
| ASO029 | AACCCACCTTTCAGGACAAACTTT | 573 | 2.26 | 0.69 |
| ASO030 | AAATGCTAGTCTGGAGGAGACATTT | 572 | 2.21 | 0.95 |
| ASO031 | AACCCACCTTTCAGGACAAACTT | 630 | 4.67 | 1.67 |
| ASO032 | AAATGCTAGTCTGGAGGAGACATT | 571 | 3.73 | 2.90 |
| ASO033 | ATAGGGACCCTCCTTCCATGACTC | 510 | 15.76 | 8.08 |
| ASO034 | AGGTATCTTTGATACTAACCTTGGT | 635 | 6.25 | 1.21 |
| ASO035 | ATCTACTGTATAGGGACCCTCC | 577 | 2.42 | 2.03 |
| ASO036 | ATAGGGACCCTCCTTCCATGACT | 576 | 48.79 | 38.26 |
| ASO037 | AGCCATTGTGTTGAATCCTT | 633 | 16.87 | 4.47 |
| ASO038 | ATCTTTGATACTAACCTTGGT | 579 | 2.43 | 0.34 |
| ASO039 | ATCCTCAGGTCAGAATACATATAT | 637 | 1.31 | 0.50 |
| ASO040 | AGGGACCCTCCTTCCATGACTC | 575 | 15.89 | 2.64 |
| ASO041 | AATTATTCATTGTGTTATGGCTAGG | 632 | 0.38 | 0.17 |
| ASO042 | AACCCACCTTTCAGGACAAACTTTT | 631 | 1.32 | 0.41 |
| ASO043 | ATCTACTGTATAGGGACCCTCCT | 578 | 2.11 | 0.37 |
| ASO044 | ATAGGGACCCTCCTTCCATGAC | 636 | 16.70 | 2.50 |
| ASO045 | AGCCATTGTGTTGAATCCTTTA | 634 | 16.56 | 1.07 |
| ASO046 | AACTGTTGCCTCCGGTTCTGAAGG | 574 | 91.78 | 2.46 |
| ASO047 | AAATGCTAGTCTGGAGGAGACAT | 570 | 8.38 | 5.76 |
| ASO048 | GCCATTGTGTTGAATCCTTTA | 661 | 22.22 | 1.27 |
| ASO049 | GTACTTCATCCCACTGATTC | 537 | 52.90 | 18.27 |
| ASO050 | GTCTACTGTTCATTTCAGCT | 673 | 0.87 | 0.61 |
| ASO051 | GACCCTCCTTCCATGACTCAA | 523 | 11.25 | 2.38 |
| ASO052 | TCTTGTACTTCATCCCACTGAT | 541 | 51.15 | 5.02 |
| ASO053 | TCTTGTACTTCATCCCACTGATT | 542 | 62.64 | 9.05 |
| ASO054 | TCCAGCCATTGTGTTGAATCCTT | 678 | 31.57 | 3.71 |
| ASO055 | TCCAGCCATTGTGTTGAATCCTTT | 679 | 26.62 | 6.68 |
| ASO056 | CATCTACTGTATAGGGACCCTCC | 511 | 4.69 | 3.83 |
| ASO057 | CATCTACTGTATAGGGACCCTCCT | 512 | 3.75 | 1.31 |
| ASO058 | CCTCCGGTTCTGAAGGTGTTCTTG | 585 | 89.21 | 1.52 |
| ASO059 | CTTCCAGCCATTGTGTTGAATCC | 656 | 44.29 | 4.59 |
| ASO060 | CTTCCAGCCATTGTGTTGAATCCTT | 658 | 63.55 | 27.70 |
| ASO061 | GCATCTACTGTATAGGGACCC | 660 | 4.13 | 1.03 |
| ASO062 | CTCCGGTTCTGAAGGTGTTCTT | 655 | 79.38 | 9.80 |
| ASO063 | CTCCGGTTCTGAAGGTGTTCTTG | 588 | 88.63 | 1.84 |
| ASO064 | CTCCGGTTCTGAAGGTGTTCTTGTA | 589 | 84.93 | 6.15 |
| ASO065 | CTCCTTCCATGACTCAAGCT | 519 | 3.05 | 1.00 |
| ASO066 | GCTTCCAGCCATTGTGTTGAATCCT | 665 | 32.20 | 12.34 |
| ASO067 | TGCCTCCGGTTCTGAAGGTGTTCT | 544 | 93.40 | 1.75 |
| ASO068 | TGCCTCCGGTTCTGAAGGTGTTCTT | 545 | 79.58 | 18.76 |
| ASO069 | TGTGTTATGGCTAGGATGATG | 621 | 1.27 | 0.90 |
| ASO070 | TGTGTTATGGCTAGGATGATGA | 622 | 0.92 | 0.44 |
| ASO071 | TGTGTTATGGCTAGGATGATGAA | 623 | 0.87 | 0.73 |
| ASO072 | TGTACTTCATCCCACTGATT | 620 | 50.15 | 4.67 |
| ASO073 | CCTCCGGTTCTGAAGGTGTTCT | 583 | 87.82 | 3.64 |
| ASO074 | CCCTCCTTCCATGACTCAAGCT | 514 | 11.09 | 6.70 |
| ASO075 | CCCTCCTTCCATGACTCAAG | 653 | 3.15 | 1.87 |
| ASO076 | CATCTACTGTATAGGGACCCTC | 645 | 3.44 | 0.62 |
| ASO077 | TCCAGCCATTGTGTTGAATCCTTTA | 680 | 59.05 | 20.32 |
| ASO078 | TCCAGCCATTGTGTTGAATCCT | 677 | 55.73 | 12.25 |
| ASO079 | CTTGTACTTCATCCCACTGATTC | 521 | 92.61 | 2.02 |
| ASO080 | CTTGTACTTCATCCCACTGATT | 593 | 77.61 | 10.11 |
| ASO081 | CTTGTACTTCATCCCACTGAT | 520 | 62.03 | 0.97 |
| ASO082 | CTTGTACTTCATCCCACTGA | 592 | 67.71 | 10.26 |
| ASO083 | CTCCGGTTCTGAAGGTGTTCT | 654 | 86.47 | 5.84 |
| ASO084 | CCTTAGCTTCCAGCCATTGTGTTGA | 518 | 41.65 | 11.35 |
| ASO085 | CCTTAGCTTCCAGCCATTGTGTTG | 517 | 58.30 | 8.49 |
| ASO086 | CCTTAGCTTCCAGCCATTGTGT | 586 | 18.78 | 4.58 |
| ASO087 | CCAGCCATTGTGTTGAATCCTTTA | 650 | 39.06 | 8.29 |
| ASO088 | CCAGCCATTGTGTTGAATCCTTT | 649 | 38.87 | 3.99 |
| ASO089 | CCAGCCATTGTGTTGAATCCTT | 648 | 52.62 | 7.79 |
| ASO090 | TCTTGTACTTCATCCCACTGATTC | 543 | 79.70 | 2.54 |
| ASO091 | TCTTGTACTTCATCCCACTGA | 540 | 49.66 | 6.71 |
| ASO092 | TCTTGTACTTCATCCCACTG | 539 | 66.75 | 6.16 |
| ASO093 | GGGACCCTCCTTCCATGACTCAAG | 669 | 3.18 | 2.25 |
| ASO094 | GGGACCCTCCTTCCATGACT | 604 | 8.01 | 2.02 |
| ASO095 | GCTTTGTGTGTCCCATGCTTGTTA | 532 | 2.44 | 1.95 |
| ASO096 | GCTTTGTGTGTCCCATGCTT | 528 | 16.63 | 18.02 |
| ASO097 | GCTAGTCTGGAGGAGACATTTTA | 600 | 18.15 | 6.20 |
| ASO098 | GACCCTCCTTCCATGACTCAAGCT | 659 | 1.83 | 1.83 |
| ASO099 | GACCCTCCTTCCATGACTCAAG | 524 | 3.13 | 0.00 |
| ASO100 | GACCCTCCTTCCATGACTCA | 522 | 15.94 | 15.94 |
| ASO101 | TGCTTTGTGTGTCCCATGCTTG | 548 | 1.82 | 1.54 |
| ASO102 | TGCTTTGTGTGTCCCATGCTT | 547 | 2.74 | 0.72 |
| ASO103 | TGCTTTGTGTGTCCCATGCT | 546 | 5.60 | 4.56 |
| ASO104 | GGACCCTCCTTCCATGACTCAAGCT | 668 | 21.61 | 8.35 |
| ASO105 | GGACCCTCCTTCCATGACTCAAGC | 667 | 1.98 | 2.80 |
| ASO106 | GGACCCTCCTTCCATGACTCAA | 602 | 6.27 | 6.32 |
| ASO107 | GGACCCTCCTTCCATGACTC | 533 | 13.82 | 7.50 |
| ASO108 | TTCTTGTACTTCATCCCACTGATTC | 563 | 83.98 | 5.29 |
| ASO109 | GGACCCTCCTTCCATGACTCA | 601 | 19.19 | 4.91 |
| ASO110 | TGCTTTGTGTGTCCCATGCTTGTTA | 551 | 5.83 | 0.76 |
| ASO111 | GACCCTCCTTCCATGACTCAAGC | 525 | 28.29 | 13.82 |
| ASO112 | GCTAGTCTGGAGGAGACATTTTAA | 662 | 3.49 | 0.84 |
| ASO113 | GGGACCCTCCTTCCATGACTCAA | 605 | 10.71 | 11.45 |
| ASO114 | GGGACCCTCCTTCCATGACTCAAGC | 670 | 67.65 | 32.35 |
| ASO115 | CCAGCCATTGTGTTGAATCCT | 647 | 58.35 | 10.19 |
| ASO116 | CCCTCCTTCCATGACTCAAGC | 513 | 11.02 | 0.00 |
| ASO117 | CCTCCGGTTCTGAAGGTGTTCTT | 584 | 94.25 | 1.82 |
| ASO118 | CTTCCAGCCATTGTGTTGAATCCT | 657 | 56.75 | 14.87 |
| ASO119 | GCATCTACTGTATAGGGACCCTC | 596 | 0.00 | 0.00 |
| ASO120 | GCATCTACTGTATAGGGACCCTCC | 527 | 3.50 | 1.57 |
| ASO121 | GCTTCCAGCCATTGTGTTGAATC | 663 | 26.42 | 0.00 |
| ASO122 | GCTTCCAGCCATTGTGTTGAATCC | 664 | 43.43 | 17.09 |
| ASO123 | TGCCTCCGGTTCTGAAGGTGTTC | 682 | 96.86 | 2.25 |
| ASO124 | ATTGTGTTATGGCTAGGATGATGA | 581 | 0.00 | 0.00 |
| ASO125 | ATTGTGTTATGGCTAGGATGATGAA | 642 | 7.29 | 10.31 |
| ASO126 | CCCACCTTTCAGGACAAACTTTTCA | 652 | 7.14 | 3.82 |
| ASO127 | CCTCCTTCCATGACTCAAGC | 515 | 7.17 | 5.08 |
| ASO128 | CCTCCTTCCATGACTCAAGCT | 516 | 8.37 | 1.63 |
| ASO129 | CTTAGCTTCCAGCCATTGTGTTG | 590 | 51.44 | 23.31 |
| ASO130 | CTTAGCTTCCAGCCATTGTGTTGA | 591 | 40.09 | 2.77 |
| ASO131 | GATTGCATCTACTGTATAGGGACC | 595 | 1.85 | 1.85 |
| ASO132 | GGATTGCATCTACTGTATAGGGACC | 534 | 3.57 | 3.57 |
| ASO133 | GTAACCCACCTTTCAGGACAAACT | 608 | 1.52 | 2.14 |
| ASO134 | GTAACCCACCTTTCAGGACAAACTT | 536 | 2.38 | 1.09 |
| ASO135 | TAAATGCTAGTCTGGAGGAGACAT | 612 | 4.40 | 1.74 |
| ASO136 | TAAATGCTAGTCTGGAGGAGACATT | 613 | 5.78 | 1.05 |
| ASO137 | TAACCCACCTTTCAGGACAAACTT | 614 | 0.90 | 0.69 |
| ASO138 | TAACCCACCTTTCAGGACAAACTTT | 615 | 1.63 | 2.30 |
| ASO139 | TATCTTTGATACTAACCTTGGT | 617 | 6.31 | 1.18 |
| ASO140 | TATCTTTGATACTAACCTTGGTT | 676 | 4.40 | 2.19 |
| ASO141 | CAAAGTCTACTGTTCATTTCAGCT | 643 | 35.59 | 45.59 |
| ASO142 | CAGCCATTGTGTTGAATCCTTTA | 644 | 68.26 | 13.46 |
| ASO143 | CCACCTTTCAGGACAAACTTTTCA | 646 | 2.94 | 2.70 |
| ASO144 | CTTTTGGATTGCATCTACTGTAT | 594 | 9.99 | 9.93 |
| ASO145 | GTAAATGCTAGTCTGGAGGAGAC | 671 | 0.00 | 0.00 |
| ASO146 | GTCCCATGCTTGTTAAAAAACTTAC | 672 | 1.60 | 1.15 |
| ASO147 | TAGCTTCCAGCCATTGTGTTGAATC | 675 | 58.00 | 17.17 |
| ASO148 | TAGGGACCCTCCTTCCATGACTC | 616 | 14.29 | 14.29 |
| ASO149 | TCCTCAGGTCAGAATACATATAT | 681 | 1.19 | 0.84 |
| ASO150 | TCTTTTGGATTGCATCTACTGTA | 618 | 2.17 | 1.03 |
| ASO151 | GGACCCTCCTTCCATGACTCAAG | 666 | 2.20 | 1.71 |
| ASO152 | TGCTTTGTGTGTCCCATGCTTGTT | 550 | 2.20 | 0.75 |
| ASO153 | GCTAGTCTGGAGGAGACATT | 597 | 14.24 | 7.06 |
| ASO154 | GCTAGTCTGGAGGAGACATTT | 598 | 28.85 | 14.86 |
| ASO155 | GCTAGTCTGGAGGAGACATTTT | 599 | 4.88 | 1.11 |
| ASO156 | GCTTTGTGTGTCCCATGCTTG | 529 | 0.84 | 0.35 |
| ASO157 | GGGACCCTCCTTCCATGACTCA | 535 | 24.78 | 10.92 |
| ASO158 | CCTTAGCTTCCAGCCATTGTGTT | 587 | 77.55 | 16.31 |
| ASO159 | GTGTTATGGCTAGGATGATGA | 609 | 3.03 | 1.90 |
| ASO160 | GTGTTATGGCTAGGATGATGAA | 610 | 12.82 | 2.73 |
| ASO161 | GTGTTATGGCTAGGATGATGAAC | 538 | 40.33 | 28.42 |
| ASO162 | GATTGCATCTACTGTATAGGGACCC | 526 | 17.32 | 9.95 |
| ASO163 | GGATTGCATCTACTGTATAGGGAC | 603 | 18.00 | 1.60 |
| ASO164 | CAACTGTTGCCTCCGGTTCTGAAGG | 582 | 91.41 | 7.19 |

### Example 4. Exon-Skipping Activity of Anti-TfR1 Antibody Conjugates in Duchenne Muscular Dystrophy Patient Myotubes

In this study, the exon-skipping activities of anti-TfR1 antibody conjugates comprising an anti-TfR1 Fab (3M12 VH4/Vκ3) covalently linked to a DMD exon 53-skipping antisense oligonucleotide (ASO) were evaluated. The DMD exon 53-skipping ASOs tested in this Example are a subset of those tested in Example 3. They are phosphorodiamidate morpholino oligomers (PMOs) of 21-25 nucleotides in length and may target various splicing features in DMD exon 53 and the immediately preceding and following introns. ASO007, ASO020, ASO046, ASO004, ASO010, ASO015, ASO017, ASO019, and ASO022 listed in Table 12 were covalently linked via a cleavable linker to anti-TfR1 Fab (3M12 VH4/Vκ3). Attempted linkage of ASO013, ASO016, and ASO036 to the anti-TfR1 Fab was unsuccessful.

Immortalized human myoblasts bearing an exon 52 deletion were thawed and seeded at a density of 1×10⁶ cells/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cells were counted and seeded into Matrigel-coated wells of 96-well plates at a density of 50,000 cells/well. Cells were allowed to recover for 24 hours. Cells were induced to differentiate into myotubes by replacing the growth media with differentiation media containing no serum. Cells were then treated with conjugates comprising DMD exon 53-skipping oligonucleotide covalently linked to anti-TfR1 Fab (3M12 VH4/Vk3) at a final concentration of 0.15625 μM, 0.625 μM, 2.5 μM, and a higher dose of either 5 μM or 10 μM ASO equivalent. Cells were incubated with conjugates for ten days, then total RNA was harvested from the 96 well plates. cDNA synthesis was performed using 75 ng of total RNA, and mutation specific PCRs were performed to evaluate the degree of exon 53 skipping in the cells. Mutation-specific PCR products were run on a 4% agarose gel and visualized using SYBR gold. Densitometry was used to calculate the relative amounts of the skipped and unskipped amplicon and exon skipping was determined as a ratio of the Exon 53 skipped amplicon divided by the total amount of amplicon present:

$$\% Exon Skipping = \frac{Skipped Amplicon}{( Skipped Amplicon + Unskipped Amplicon )} * 100.$$

The results shown in FIG. 2 demonstrate that treatment with certain of the anti-TfR1-ASO conjugates tested resulted in enhanced exon skipping. Five of the conjugates tested (comprising ASO007, ASO046, ASO010, ASO015, and ASO017 listed in Table 12, respectively) achieved exon 53 skipping in excess of 75% at the highest dose.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides or nucleosides (e.g., an RNA counterpart of a DNA nucleoside or a DNA counterpart of an RNA nucleoside) and/or (e.g., and) one or more modified nucleotides/nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor 1 (TfR1) antibody covalently linked to an oligonucleotide configured for inducing skipping of exon 53 in a DMD pre-mRNA, wherein the oligonucleotide comprises a region of complementarity that is complementary with at least 8 consecutive nucleotides of any one of SEQ ID NOs: 224, 206, 209, 212, 277, 214, 207, 208, 205, 160-204, 210, 211, 213, 215-223, 225-276, and 278-334.

2.-4. (canceled)

5. The complex of claim 1, wherein the anti-TfR1 antibody is a Fab fragment, a Fab′ fragment, a F(ab′)2 fragment, an scFv, an Fv, or a full-length IgG.

6. The complex of claim 5, wherein the anti-TfR1 antibody is a Fab fragment.

7.-8. (canceled)

9. The complex of claim 1, wherein the anti-TfR1 antibody does not specifically bind to the transferrin binding site of the transferrin receptor 1 and/or wherein the anti-TfR1 antibody does not inhibit binding of transferrin to the transferrin receptor 1.

10. The complex of claim 1, wherein the oligonucleotide is complementary to at least 4 consecutive nucleotides of a splicing feature of the DMD pre-mRNA.

11. The complex of claim 10, wherein the splicing feature is an exonic splicing enhancer (ESE) in exon 53 of the DMD pre-mRNA, optionally wherein the ESE comprises a sequence of any one of SEQ ID NOs: 689-715.

12. The complex of claim 10, wherein the splicing feature is a branch point, a splice donor site, or a splice acceptor site, optionally wherein the splicing feature is across the junction of exon 52 and intron 52, in intron 52, across the junction of intron 52 and exon 53, across the junction of exon 53 and intron 53, in intron 53, or across the junction of intron 53 and exon 54 of the DMD pre-mRNA, and further optionally wherein the splicing feature comprises a sequence of any one of SEQ ID NOs: 685-688 and 716-718.

13. The complex of claim 1, wherein the oligonucleotide comprises a sequence complementary to any one of SEQ ID NOs: 160-334 or comprises a sequence of any one of SEQ ID NOs: 335-684, wherein each thymine base (T) may independently and optionally be replaced with a uracil base (U), and each U may independently and optionally be replaced with a T.

14. The complex of claim 1, wherein the oligonucleotide comprises a sequence of any one of SEQ ID NOs: 574, 556, 559, 562, 627, 564, 557, 558, and 555, wherein each thymine base (T) may independently and optionally be replaced with a uracil base (U), and each U may independently and optionally be replaced with a T.

15. The complex of claim 1, wherein the oligonucleotide comprises one or more phosphorodiamidate morpholinos, optionally wherein the oligonucleotide is a phosphorodiamidate morpholino oligomer (PMO).

16. The complex of claim 1, wherein the anti-TfR1 antibody is covalently linked to the oligonucleotide via a cleavable linker, optionally wherein the cleavable linker comprises a valine-citrulline sequence.

17. The complex of claim 1, wherein the anti-TfR1 antibody is covalently linked to the oligonucleotide via conjugation to a lysine residue or a cysteine residue of the antibody.

18. An oligonucleotide that targets DMD, wherein the oligonucleotide comprises a region of complementarity to any one of SEQ ID NOs: 160-334, optionally wherein the region of complementarity comprises at least 15 consecutive nucleosides complementary to any one of SEQ ID NOs: 160-334.

19. The oligonucleotide of claim 18, wherein the oligonucleotide comprises at least 15 consecutive nucleosides of any one of SEQ ID NOs: 335-684, optionally wherein the oligonucleotide comprises a sequence of any one of SEQ ID NOs: 335-684, wherein each thymine base (T) may independently and optionally be replaced with a uracil base (U), and each U may independently and optionally be replaced with a T.

20. The oligonucleotide of claim 19, wherein the oligonucleotide comprises a sequence of any one of SEQ ID NOs: 574, 556, 559, 562, 627, 564, 557, 558, and 555, wherein each thymine base (T) may independently and optionally be replaced with a uracil base (U), and each U may independently and optionally be replaced with a T.

21. A method of delivering an oligonucleotide to a cell, the method comprising contacting the cell with the complex of claim 1.

22. A method of promoting the expression or activity of a dystrophin protein in a cell, the method comprising contacting the cell with the complex of claim 1 in an amount effective for promoting internalization of the oligonucleotide to the cell, optionally wherein the cell is a muscle cell.
